(19) United States
(12) Patent Application Publication
(10) Pub. No.: US 2023/0165872 A1
(43) Pub. Date: Jun. 1, 2023

(54) COMBINATION OF BCMA-DIRECTED T CELL THERAPY AND AN IMMUNOMODULATORY COMPOUND

(71) Applicant: Juno Therapeutics, Inc., Seattle, WA (US)

(72) Inventors: Michael PORTS, Seattle, WA (US); Oleksandr BATUREVYCH, Seattle, WA (US); Neha SONI, Seattle, WA (US); John-Michael WILLIFORD, Seattle, WA (US); Melissa WORKS, Seattle, WA (US)

(73) Assignee: Juno Therapeutics, Inc., Seattle, WA (US)

(21) Appl. No.: 17/921,614

(22) PCT Filed: Apr. 27, 2021
(86) PCT No.: PCT/US2021/029503

§ 371 Date: Oct. 26, 2022

Related U.S. Application Data
(60) Provisional application No. 63/016,983, filed on Apr. 28, 2020.

(51) Int. Cl. A61K 31/5377; A61K 35/17; A61P 35/00

(52) U.S. Cl. CPC A61K 31/5377; A61K 35/17; A61P 35/00

(57) ABSTRACT

Provided herein are methods, compositions and uses for treating subjects with diseases and conditions, such as those involving or associated with B cell maturation antigen (BCMA), involving administration of a T cell therapy, such as a BCMA-targeted T cell therapy, e.g. anti-BCMA CART cells, in combination with (S)-3-[4-(4-morpholin-4-ylmethyl-benzyloxy)-1-oxo-1,3-dihydro-isoindol-2-yl]-piperidine-2, 6-dione, or a pharmaceutically acceptable salt, solvate, hydrate, stereoisomer, tautomer or racemic mixtures thereof, and compositions thereof, or in combination with (S)-4-(4-(4-(((2-(2,6-dioxopiperidin-3-yl)-1-oxoisoindolin-4-yl)oxy)methyl)benzyl)piperazin-1-yl)-3-fluorobenzonitrile, or a pharmaceutically acceptable salt, solvate, hydrate, stereoisomer, tautomer or racemic mixtures thereof, and compositions thereof. The T cell therapy includes cells that express recombinant receptors such as chimeric antigen receptors (CARs) directed against BCMA. In some embodiments, the disease or condition is a multiple myeloma, such as relapsed or refractory multiple myeloma.

### CROSS-REFERENCE TO RELATED APPLICATIONS

This application claims priority from U.S. provisional application No. 63/016,983 filed Apr. 28, 2020, entitled “COMBINATION OF BCMA-DIRECTED T CELL THERAPY AND AN IMMUNOMODULATORY COMPOUND,” the contents of which are incorporated by reference in their entirety.

### INCORPORATION BY REFERENCE OF SEQUENCE LISTING

The present application is being filed with a Sequence Listing in electronic format. The Sequence Listing is provided as a file entitled 735042022940SeqList.txt, created on Apr. 26, 2021, which is 331,776 bytes in size. The information in electronic format of the Sequence Listing is incorporated by reference in its entirety.

### FIELD

The present disclosure relates in some aspects to methods, compositions and uses for treating subjects with diseases and conditions, such as those involving or associated with B cell maturation antigen (BCMA), involving administration of a T cell therapy, such as a BCMA-targeted T cell therapy, e.g. anti-BCMA CAR T cells, in combination with (S)-3-[4-(4-morpholin-4-ylmethyl-benzyloxy)-1-oxo-1,3-dihydro-isoindol-2-yl]-piperidine-2,6-dione, or a pharmaceutically acceptable salt, solvate, hydrate, stereoisomer, tautomer or racemic mixtures thereof, and compositions thereof, or in combination with (S)-4-(4-(4-(((2-(2,6-dioxopiperidin-3-yl)-1-oxoisoindolin-4-yl)oxy)methyl)benzyl)piperazin-1-yl)-3-fluorobenzonitrile, or a pharmaceutically acceptable salt, solvate, hydrate, stereoisomer, tautomer or racemic mixtures thereof, and compositions thereof. The T cell therapy includes cells that express recombinant receptors such as chimeric antigen receptors (CARs) directed against BCMA. In some embodiments, the disease or condition is a multiple myeloma, such as relapsed or refractory multiple myeloma.

### BACKGROUND

Various strategies are available for immunotherapy, for example administering engineered T cells for adoptive therapy. For example, strategies are available for engineering T cells expressing genetically engineered antigen receptors, such as CARs, and administering compositions containing such cells to subjects. Improved strategies are needed to improve efficacy of the cells, for example, improving the persistence, activity and/or proliferation of the cells upon administration to subjects. Provided are methods, compositions, kits, and systems that meet such needs.

### SUMMARY

Provided herein is a method of treating multiple myeloma, the method comprising: (a) administering a T cell therapy to a subject having a relapsed or refractory multiple myeloma (R/R MM), said T cell therapy comprising a dose of genetically engineered T cells expressing a chimeric antigen receptor (CAR) that specifically binds to BCMA; and (b) administering to the subject an immunomodulatory compound that is (S)-3-[4-(4-morpholin-4-ylmethyl-benzyloxy)-1-oxo-1,3-dihydro-isoindol-2-yl]-piperidine-2,6-dione having the following structure:

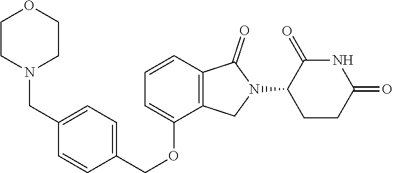 [SMILES: O=C1CC[C@H](N2Cc3c(OCc4ccc(CN5CCOCC5)cc4)cccc3C2=O)C(=O)N1]

or a pharmaceutically acceptable salt, solvate, hydrate, co-crystal, clathrate, or polymorph thereof, wherein administration of the immunomodulatory compound is initiated after administration of the T cell therapy.

Also provided is a method of treating multiple myeloma, the method comprising administering, to a subject having a relapsed or refractory multiple myeloma (R/R MM) that has been administered a cell therapy comprising a dose of genetically engineered T cells expressing a chimeric antigen receptor (CAR) that specifically binds to BCMA, an immunomodulatory compound that is (S)-3-[4-(4-morpholin-4-ylmethyl-benzyloxy)-1-oxo-1,3-dihydro-isoindol-2-yl]-piperidine-2,6-dione having the following structure:

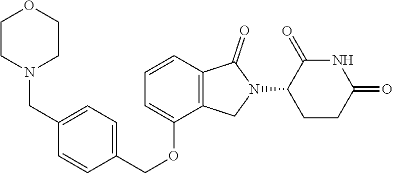 [SMILES: O=C1CC[C@H](N2Cc3c(OCc4ccc(CN5CCOCC5)cc4)cccc3C2=O)C(=O)N1]

or a pharmaceutically acceptable salt, solvate, hydrate, co-crystal, clathrate, or polymorph thereof, wherein administration of the immunomodulatory compound is initiated after administration of the T cell therapy.

In some of any embodiments, prior to initiation of the administration of the T cell therapy and the immunomodulatory compound, the subject has received one or more prior therapies for treating the R/R MM, said one or more prior therapies comprising an immunomodulatory agent.

Provided herein is a method of treating multiple myeloma, the method comprising: (a) administering a T cell therapy to a subject having a relapsed or refractory multiple myeloma (R/R MM), said T cell therapy comprising a dose of genetically engineered T cells expressing a chimeric antigen receptor (CAR) that specifically binds to BCMA; and (b) administering to the subject an immunomodulatory compound that is (S)-3-[4-(4-morpholin-4-ylmethyl-benzyloxy)-1-oxo-1,3-dihydro-isoindol-2-yl]-piperidine-2,6-dione having the following structure:

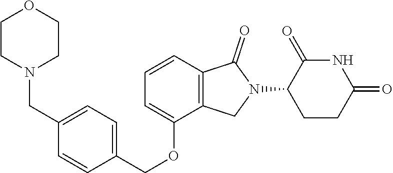 [SMILES: O=C1CC[C@H](N2Cc3c(OCc4ccc(CN5CCOCC5)cc4)cccc3C2=O)C(=O)N1]

or a pharmaceutically acceptable salt, solvate, hydrate, co-crystal, clathrate, or polymorph thereof; wherein prior to initiation of the administration of the T cell therapy and the immunomodulatory compound, the subject has received one or more prior therapies for treating the R/R MM, said one or more prior therapies comprising an immunomodulatory agent.

In some of any embodiments, the immunomodulatory compound is or comprises a pharmaceutically acceptable salt of (S)-3-[4-(4-morpholin-4-ylmethyl-benzyloxy)-1-oxo-1,3-dihydro-isoindol-2-yl]-piperidine-2,6-dione. In some of any embodiments, the immunomodulatory compound is or comprises a hydrate of (S)-3-[4-(4-morpholin-4-ylmethyl-benzyloxy)-1-oxo-1,3-dihydro-isoindol-2-yl]-piperidine-2,6-dione. In some of any embodiments, the immunomodulatory compound is or comprises a solvate of (S)-3-[4-(4-morpholin-4-ylmethyl-benzyloxy)-1-oxo-1,3-dihydro-isoindol-2-yl]-piperidine-2,6-dione. In some of any embodiments, the immunomodulatory compound is or comprises (S)-3-[4-(4-morpholin-4-ylmethyl-benzyloxy)-1-oxo-1,3-dihydro-isoindol-2-yl]-piperidine-2,6-dione.

Provided herein is a method of treating multiple myeloma, the method comprising: (a) administering a T cell therapy to a subject having a relapsed or refractory multiple myeloma (R/R MM), said T cell therapy comprising a dose of genetically engineered T cells expressing a chimeric antigen receptor (CAR) that specifically binds to BCMA; and (b) administering to the subject an immunomodulatory compound that is (S)-4-(4-(4-(((2-(2,6-dioxopiperidin-3-yl)-1-oxoisoindolin-4-yl)oxy)methyl)benzyl)piperazin-1-yl)-3-fluorobenzonitrile having the following structure:

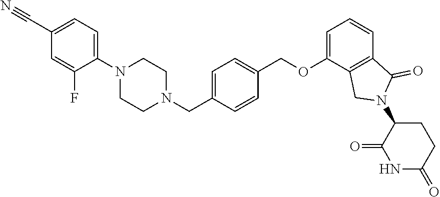 [SMILES: N#Cc1ccc(N2CCN(Cc3ccc(COc4cccc5c4CN([C@H]4CCC(=O)NC4=O)C5=O)cc3)CC2)c(F)c1]

or a pharmaceutically acceptable salt, solvate, hydrate, co-crystal, clathrate, or polymorph thereof, wherein administration of the immunomodulatory compound is initiated after administration of the T cell therapy.

Also provided is a method of treating multiple myeloma, the method comprising administering, to a subject having a relapsed or refractory multiple myeloma (R/R MM) that has been administered a cell therapy comprising a dose of genetically engineered T cells expressing a chimeric antigen receptor (CAR) that specifically binds to BCMA, an immunomodulatory compound that is (S)-4-(4-(4-(((2-(2,6-dioxopiperidin-3-yl)-1-oxoisoindolin-4-yl)oxy)methyl)benzyl)piperazin-1-yl)-3-fluorobenzonitrile having the following structure:

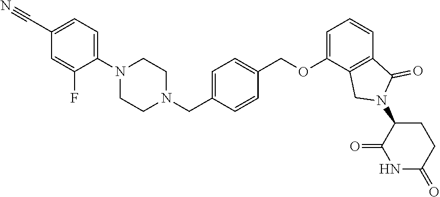 [SMILES: N#Cc1ccc(N2CCN(Cc3ccc(COc4cccc5c4CN([C@H]4CCC(=O)NC4=O)C5=O)cc3)CC2)c(F)c1]

or a pharmaceutically acceptable salt, solvate, hydrate, co-crystal, clathrate, or polymorph thereof, wherein administration of the immunomodulatory compound is initiated after administration of the T cell therapy.

In some of any embodiments, prior to initiation of the administration of the T cell therapy and the immunomodulatory compound, the subject has received one or more prior therapies for treating the R/R MM, said one or more prior therapies comprising an immunomodulatory agent.

In some of any embodiments, the immunomodulatory compound is or comprises a pharmaceutically acceptable salt of (S)-4-(4-(4-(((2-(2,6-dioxopiperidin-3-yl)-1-oxoisoindolin-4-yl)oxy)methyl)benzyl)piperazin-1-yl)-3-fluorobenzonitrile. In some of any embodiments, the immunomodulatory compound is or comprises a hydrate of (S)-4-(4-(4-(((2-(2,6-dioxopiperidin-3-yl)-1-oxoisoindolin-4-yl)oxy)methyl)benzyl)piperazin-1-yl)-3-fluorobenzonitrile. In some of any embodiments, the immunomodulatory compound is or comprises a solvate of (S)-4-(4-(4-(((2-(2,6-dioxopiperidin-3-yl)-1-oxoisoindolin-4-yl)oxy)methyl)benzyl)piperazin-1-yl)-3-fluorobenzonitrile. In some of any embodiments, the immunomodulatory compound is or comprises (S)-4-(4-(4-(((2-(2,6-dioxopiperidin-3-yl)-1-oxoisoindolin-4-yl)oxy)methyl)benzyl)piperazin-1-yl)-3-fluorobenzonitrile.

In some of any embodiments, the subject has relapsed or been refractory following at least 3 or at least 4 prior therapies for multiple myeloma. In some of any embodiments, the subject has received, and has relapsed or been refractory to, three or more therapies selected from among: autologous stem cell transplant (ASCT); an immunomodulatory agent; a proteasome inhibitor; and an anti-CD38 antibody; unless the subject was not a candidate for or was contraindicated for one or more of the therapies. In some of any embodiments, the immunomodulatory agent is selected from among thalidomide, lenalidomide and pomalidomide. In some of any embodiments, the proteasome inhibitor is selected from among bortezomib, carfilzomib and ixazomib. In some of any embodiments, the anti-CD38 antibody is or comprises daratumumab. In some of any embodiments, at the time of administration, the subject has been refractory to or not responded to bortezomib, carfilzomib, lenalidomide, pomalidomide and/or an anti-CD38 monoclonal antibody. In some of any embodiments, at the time of administration, the subject has IMWG high risk cytogenetics.

In some of any embodiments, administration of the immunomodulatory compound is initiated at or prior to peak expansion of the T cell therapy in the subject. In some of any embodiments, peak expansion of the T cell therapy is between at or about 11 days and at or about 15 days after administering the T cell therapy. In some of any embodiments, administration of the immunomodulatory compound is initiated between at or about 1 day and at or about 15 days, inclusive, after administering the T cell therapy. In some of any embodiments, administration of the immunomodulatory compound is initiated between at or about 1 day and at or about 11 days, inclusive, after administering the T cell therapy. In some of any embodiments, the administration of the immunomodulatory compound is initiated between at or about 8 days and at or about 15 days, inclusive, after administering the T cell therapy.

In some of any embodiments, administration of the immunomodulatory compound is initiated at or about 1 day after administering the T cell therapy. In some of any embodiments, administration of the immunomodulatory compound is initiated at or about 8 days after administering the T cell therapy. In some of any embodiments, the administration of the immunomodulatory compound is initiated at or about 15 days after administering the T cell therapy.

In some of any embodiments, the administration of the immunomodulatory compound is initiated about 14 to about 35 days after initiation of administration of the T cell therapy. In some of any embodiments, the administration of the immunomodulatory compound is initiated about 21 to about 35 days after initiation of administration of the T cell therapy. In some of any embodiments, the administration of the immunomodulatory compound is initiated about 21 to about 28 days after initiation of administration of the T cell therapy.

In some of any embodiments, the administration of the immunomodulatory compound is initiated at or about 21 days, at or about 22 days, at or about 23 days, at or about 24 days, at or about 25 days, at or about 26 days, at or about 27 days, or at or about 28 days after initiation of administration of the T cell therapy. In some of any embodiments, the administration of the immunomodulatory compound is initiated at or about 28 days after the initiation of the administration of the T cell therapy.

In some of any embodiments, the immunomodulatory compound is administered from or from about 0 to 30 days, 0 to 15 days, 0 to 6 days, 0 to 96 hours, 2 hours to 15 days, 2 hours to 6 days, 2 hours to 96 hours, 6 hours to 30 days, 6 hours to 15 days, 6 hours to 6 days, 6 hours to 96 hours, 12 hours to 30 days, 12 hours to 15 days, 12 hours to 6 days, or 12 hours to 96 hours prior to initiation of the T cell therapy. In some of any embodiments, the immunomodulatory compound is administered no more than about 96 hours, 72 hours, 48 hours, or 24 hours prior to initiation of the T cell therapy.

In some of any embodiments, the immunomodulatory compound is administered at least once daily in a cycle regimen. In some embodiments, the immunomodulatory compound is administered in a cycle regimen comprising the administration of the immunomodulatory compound for a plurality of consecutive days followed by a rest period during which the immunomodulatory compound is not administered. In some embodiments, the plurality of consecutive days is up to 21 days.

In some of any embodiments, the cycle regimen is a four-week (28-day) cycle wherein the immunomodulatory compound is administered daily in the four-week cycle. In some of any embodiments, the cycle regimen is a four-week (28-day) cycle wherein the immunomodulatory compound is administered daily for three consecutive weeks in the four-week cycle and is not administered for the last week. In some of any embodiments, the cycle regimen is a four-week (28-day) cycle wherein the immunomodulatory compound is administered daily for days 1 through 21 of each four-week cycle.

In some of any embodiments, the cycling regimen is repeated a plurality of times. In some of any embodiments, the plurality of times is between two and 12 cycling regimens. In some of any embodiments, the cycling regiment is repeated 3 times. In some of any embodiments, the cycling regimen is repeated 4 times. In some of any embodiments, the cycling regimen is repeated 5 times. In some of any embodiments, the cycling regimen is repeated 6 times.

In some of any embodiments, the immunomodulatory compound is administered up to at or about three months after initiation of administration of the T cell therapy. In some of any embodiments, the immunomodulatory compound is administered up to at or about six months after initiation of administration of the T cell therapy.

In some of any embodiments, the immunomodulatory compound is administered in an amount that is at or about 0.1 mg to about 1.0 mg per day. In some of any embodiments, the immunomodulatory compound is administered in an amount that is at or about 0.3 mg to about 0.6 mg. In some of any embodiments, the immunomodulatory compound is administered in an amount that is at or about 0.3 mg. In some of any embodiments, the immunomodulatory compound is administered in an amount that is at or about 0.45 mg. In some of any embodiments, the immunomodulatory compound is administered in an amount that is at or about 0.6 mg.

In some of any embodiments, the immunomodulatory compound is administered orally.

In some of any embodiments, at the time of the initiation of the administration of the immunomodulatory compound, the subject does not exhibit a severe toxicity following the administration of the T cell therapy. In some of any embodiments, the severe toxicity is severe cytokine release syndrome (CRS), optionally grade 3 or higher, prolonged grade 3 or higher or grade 4 or 5 CRS; and/or the severe toxicity is severe neurotoxicity, optionally grade 3 or higher, prolonged grade 3 or higher or grade 4 or 5 neurotoxicity.

In some of any embodiments, the administration of the immunomodulatory compound is suspended and/or the cycling regimen is modified if the subject exhibits a toxicity following the administration of the immunomodulatory compound, optionally a hematologic toxicity. In some of any embodiments, the toxicity is selected from severe neutropenia, optionally febrile neutropenia, prolonged grade 3 or higher neutropenia.

In some of any embodiments, the administration of the immunomodulatory compound: reverses an exhaustion phenotype in CAR-expressing T cells in the subject; prevents, inhibits or delays the onset of an exhaustion phenotype in CAR-expressing T cells in the subject; or reduces the level or degree of an exhaustion phenotype in CAR-expressing T cells in the subject; or reduces the percentage, of the total number of CAR-expressing T cells in the subject, that have an exhaustion phenotype.

In some of any embodiments, following administration of the immunomodulatory compound or initiation thereof, the subject exhibits a restoration or rescue of an antigen- or tumor-specific activity or function of CAR-expressing T cells in said subject, optionally wherein said restoration, rescue, and/or initiation of administration of said compound, is at a point in time after CAR-expressing T cells in the subject or the in the blood of the subject have exhibited an exhausted phenotype.

In some of any embodiments, the administration of the immunomodulatory compound comprises administration at an amount, frequency and/or duration effective to: (a) effect an increase in antigen-specific or antigen receptor-driven activity of naïve or non-exhausted T cells in the subject, which optionally comprise T cells expressing said CAR, following exposure of the T cells to BCMA or to an agonist of the CAR, optionally wherein the agonist is an anti-idiotypic antibody, as compared to the absence of said administration of said compound; or (b) prevent, inhibit or delay the onset of an exhaustion phenotype, in naïve or non-exhausted T cells T cells in the subject, which optionally comprise T cells expressing said CAR, following exposure of the T cells to BCMA or to an agonist of the CAR, optionally wherein the agonist is an anti-idiotypic antibody, as compared to the absence of said administration of said compound; or (c) reverse an exhaustion phenotype in exhausted T cells, optionally comprising T cells expressing said CAR, in the subject, as compared to the absence of said administration of said subject.

In some of any embodiments, at the time of the administration of the immunomodulatory compound an exhausted phenotype of one or more of the CAR-expressing T cells, or a marker or parameter indicative thereof, has been detected or measured in the subject or in a biological sample from the subject. In some of any embodiments, at least at or about 10%, at least at or about 20%, at least at or about 30%, at least at or about 40%, or at least at or about 50% of the total CAR-expressing T cells in a biological sample from the subject has an exhausted phenotype. In some of any embodiments, greater than at or about 10%, greater than at or about 20%, greater than at or about 30%, greater than at or about 40%, or greater than at or about 50% of the CAR-expressing T cells in a biological sample from the subject has an exhausted phenotype compared to the percentage of the CAR-expressing T cells having the exhausted phenotype in a comparable biological sample at a prior time point.

In some of any embodiments, the exhaustion phenotype, with reference to a T cell or population of T cells, comprises: an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or a decrease in the level or degree of an activity exhibited by said T cells or population of T cells upon exposure to BCMA or an agonist of the CAR, optionally wherein the agonist is an anti-idiotypic antibody, compared to a reference T cell population, under the same conditions. In some of any embodiments, the increase in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more. In some of any embodiments, the decrease in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

In some of any embodiments, the reference T cell population is a population of T cells known to have a non-exhausted phenotype, is a population of naïve T cells, is a population of central memory T cells, or is a population of stem central memory T cells, optionally from the same subject, or of the same species as the subject, from which the T cell or T cells having the exhausted phenotype are derived. In some of any embodiments, the reference T cell population (a) is a subject-matched population comprising bulk T cells isolated from the blood of the subject from which the T cell or T cells having the exhausted phenotype is derived, optionally wherein the bulk T cells do not express the CAR and/or (b) is obtained from the subject from which the T cell or T cells having the exhausted phenotype is derived, prior to receiving administration of a dose of T cells expressing the CAR. In some of any embodiments, the reference T cell population is a composition comprising a sample of the T cell therapy, or pharmaceutical composition comprising T cells expressing the CAR, prior to its administration to the subject, optionally wherein the composition is a cryopreserved sample.

In some of any embodiments, one or more of the one or more exhaustion marker is an inhibitory receptor. In some of any embodiments, one or more of the one or more exhaustion marker is selected from among PD-1, CTLA-4, TIM-3, LAG-3, BTLA, 2B4, CD160, CD39, VISTA, and TIGIT. In some of any embodiments, the activity or is one or more of proliferation, cytotoxicity or production of one or a combination of inflammatory cytokines, optionally wherein the one or a combination of cytokines is selected from the group consisting of IL-2, IFN-gamma and TNF-alpha.

In some of any embodiments, the exposure to BCMA or an agonist of the CAR, optionally wherein the agonist is an anti-idiotypic antibody, comprises incubation with BCMA or the agonist of the CAR. In some of any embodiments, the antigen is comprised on the surface of antigen-expressing target cells, optionally multiple myeloma cells or cell line.

In some of any embodiments, the dose of T cells is between at or about 5×10⁷ CAR+ T cells and at or about 1×10⁹ CAR+ T cells. In some of any embodiments, the dose of T cells is between at or about 1×10⁸ CAR+ T cells and at or about 1×10⁹ CAR+ T cells. In some of any embodiments, the dose of T cells is at or about 1.5×10⁸ cells or CAR+ T cells. In some of any embodiments, the dose of T cells is at or about 3×10⁸ cells or CAR+ T cells. In some of any embodiments, the dose of T cells is at or about 4.5×10⁸ cells or CAR+ T cells. In some of any embodiments, the dose of T cells is at or about 6×10⁸ cells or CAR+ T cells.

In some of any embodiments, the dose comprises CD3⁺ CAR-expressing T cells. In some of any embodiments, the dose comprises a combination of CD4⁺ T cells and CD8⁺ T cells and/or a combination of CD4⁺ CAR-expressing T cells and CD8⁺ CAR-expressing T cells. In some of any embodiments, the ratio of CD4⁺ CAR-expressing T cells to CD8⁺ CAR-expressing T cells and/or of CD4⁺ T cells to CD8⁺ T cells, is or is approximately 1:1 or is between at or approximately 1:3 and at or approximately 3:1.

In some of any embodiments, prior to the administration of the dose of T cells, the subject has received a lymphodepleting therapy comprising the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days. In some of any embodiments, the subject has received a lymphodepleting therapy comprising the administration of fludarabine at or about 30 mg/m² body surface area of the subject, daily, and cyclophosphamide at or about 300 mg/m² body surface area of the subject, daily, for 3 days.

In some of any embodiments, the CAR comprises an antigen binding domain that binds to BCMA, a transmembrane domain, and an intracellular signaling region comprising a CD3-zeta (CD3ζ) chain.

In some of any embodiments, the antigen binding domain is a single chain variable fragment (scFv).

In some of any embodiments, the antigen binding domain comprises a V_{H} and a V_{L} region, wherein the V_{H} region comprises a CDR-H1 set forth in SEQ ID NO: 56, a CDR-H2 set forth in SEQ ID NO:57 and a CDR-H3 set forth in SEQ ID NO:58, and the V_{L} region comprises a CDR-L1 set forth in SEQ ID NO: 59, a CDR-L2 set forth in SEQ ID NO:60 and a CDR-H3 set forth in SEQ ID NO:61. In some of any embodiments, the antigen binding domain comprises a V_{H} region that has the sequence of amino acids set forth in SEQ ID NO:36 or a sequence of amino acids that exhibits at least 90%, at least 91%, at least 92%, at least 93%, at least 94%, at least 95%, at least 96%, at least 97%, at least 98%, at least 99% to SEQ ID NO:36, and a V_{L} region has the sequence of amino acids set forth in SEQ ID NO:37 or a sequence of amino acids that exhibits at least 90%, at least 91%, at least 92%, at least 93%, at least 94%, at least 95%, at least 96%, at least 97%, at least 98%, at least 99% to SEQ ID NO:37. In some of any embodiments, the antigen binding domain comprises the V_{H} region sequence of amino acids set forth in SEQ ID NO:36 and the V_{L} region sequence of amino acids set forth in SEQ ID NO:37. In some of any embodiments, the antigen-binding domain is an scFv that has the sequence of amino acids set forth in SEQ ID NO:180 or a sequence of amino acids exhibits at least 90%, at least 91%, at least 92%, at least 93%, at least 94%, at least 95%, at least 96%, at least 97%, at least 98%, at least 99% to SEQ ID NO:180. In some of any embodiments, the antigen-binding domain is an scFv that has the sequence of amino acids set forth in SEQ ID NO:180.

In some of any embodiments, the anti-BCMA CAR comprises a V_{H} and a V_{L} region, wherein the V_{H} region comprises a CDR-H1 set forth in SEQ ID NO: 62, a CDR-H2 set forth in SEQ ID NO:63 and a CDR-H3 set forth in SEQ ID NO:64, and the V_{L} region comprises a CDR-L1 set forth in SEQ ID NO: 65, a CDR-L2 set forth in SEQ ID NO:66 and a CDR-H3 set forth in SEQ ID NO:67. In some of any embodiments, the antigen binding domain comprises a V_{H} region that has the sequence of amino acids set forth in SEQ ID NO:30 or a sequence of amino acids that exhibits at least 90%, at least 91%, at least 92%, at least 93%, at least 94%, at least 95%, at least 96%, at least 97%, at least 98%, at least 99% to SEQ ID NO:30, and the V_{L} region has the sequence of amino acids set forth in SEQ ID NO:31 or a sequence of amino acids that exhibits at least 90%, at least 91%, at least 92%, at least 93%, at least 94%, at least 95%, at least 96%, at least 97%, at least 98%, at least 99% to SEQ ID NO:31. In some of any embodiments, the antigen binding domain comprises the V_{H} region that has the sequence of amino acids set forth in SEQ ID NO:30 and the V_{L} region has the sequence of amino acids set forth in SEQ ID NO:31. In some of any embodiments, the antigen binding domain is an scFv that has the sequence of amino acids set forth in SEQ ID NO:68 or a sequence of amino acids exhibits at least 90%, at least 91%, at least 92%, at least 93%, at least 94%, at least 95%, at least 96%, at least 97%, at least 98%, at least 99% to SEQ ID NO:68. In some of any embodiments, the antigen binding domain is an scFv set forth in SEQ ID NO:68.

In some of any embodiments, the intracellular signaling region further comprises a costimulatory signaling domain. In some of any embodiments, the costimulatory signaling region comprises an intracellular signaling domain of CD28, 4-1BB, or ICOS, or a signaling portion thereof. In some of any embodiments, the costimulatory signaling region comprises an intracellular signaling domain of 4-1BB, optionally human 4-1BB. In some of any embodiments, the costimulatory signaling region is between the transmembrane domain and the cytoplasmic signaling domain of a CD3-zeta (CD3ζ) chain.

In some of any embodiments, the transmembrane domain is or comprises a transmembrane domain from human CD28. In some of any embodiments, the transmembrane domain is or comprises a transmembrane domain from human CD8.

In some of any embodiments, the CAR further comprises an extracellular spacer between the antigen binding domain and the transmembrane domain. In some of any embodiments, the spacer is between at or about 50 amino acids and at or about 250 amino acids. In some of any embodiments, the spacer is between at or about 125 amino acids and at or about 250 amino acids, optionally wherein the spacer is at or about 228 amino acids. In some of any embodiments, the spacer is an immunoglobulin spacer comprising all or a portion of an immunoglobulin constant domain or a modified form thereof. In some of any embodiments, the spacer comprises an IgG4/2 chimeric hinge or a modified IgG4 hinge; an IgG2/4 chimeric C_{H}2 region; and an IgG4 C_{H}3 region. In some of any embodiments, the spacer is set forth in SEQ ID NO: 29 or is encoded by a sequence of nucleotides set forth in SEQ ID NO:179. In some of any embodiments, the spacer is a CD8 hinge.

In some of any embodiments, the anti-BCMA CAR has a sequence set forth in any one of SEQ ID NOS: 126-177 or a sequence of amino acids that exhibits at least 90%, at least 91%, at least 92%, at least 93%, at least 94%, at least 95%, at least 96%, at least 97%, at least 98%, or at least 99% sequence identity to any one of SEQ ID NOS: 126-177.

In some of any embodiments, the anti-BCMA CAR has the sequence of amino acids set forth in SEQ ID NO:160 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:160.

In some of any embodiments, the CAR is encoded by the sequence of nucleotides set forth in SEQ ID NO:69.

In some of any embodiments, the anti-BCMA CAR has the sequence of amino acids set forth in SEQ ID NO:161 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:161.

In some of any embodiments, the anti-BCMA CAR has the sequence of amino acids set forth in SEQ ID NO:152 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:152.

In some of any embodiments, the anti-BCMA CAR has the sequence of amino acids set forth in SEQ ID NO:168 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:168.

In some of any embodiments, the anti-BCMA CAR has the sequence of amino acids set forth in SEQ ID NO:171 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:171.

In some of any embodiments, the anti-BCMA CAR binds BCMA, optionally wherein the BCMA is human BCMA. In some of any embodiments, the BCMA is membrane-bound BCMA expressed on the surface of a cell. In some of any embodiments, the anti-BCMA CAR has a greater binding affinity for membrane-bound BCMA than soluble BCMA, optionally wherein the ratio of dissociation constant (K_{D}) for soluble BCMA and the K_{D} for membrane-bound BCMA is more than 10, 15, 20, 25, 30, 40, 50, 60, 70, 80, 90, 100, 200, 500, 1000, 2000 or more.

Also provided are uses of an immunomodulatory compound and/or T cell therapy for treating a relapsed or refractory multiple myeloma (R/R MM) in accord with any of the provided methods. Also provided an immunomodulatory compound and/or T cell therapy for formulation of a medicament of use in treating a relapsed or refractory multiple myeloma (R/R MM) in accord with any of the provided methods.

Exemplary features of any of the provided methods are described herein, including in the exemplary embodiments.

### BRIEF DESCRIPTION OF THE DRAWINGS

FIG. 1 shows intracellular Ikaros and Aiolos expression in CD4+ anti-BCMA CAR-expressing T cells and CD8+ anti-BCMA CAR-expressing T cells after incubation with varying concentrations of Compound A.

FIG. 2 shows the amount of IFNγ, IL-2, and TNF-α observed in supernatants after incubation of RPMI-8226 target cells (FIG. 2A) and OPM-2 (FIG. 2B) anti-BCMA CAR T cells in the presence of increasing concentrations of Compound A.

FIG. 3A shows the cytolytic activity of anti-BCMA CAR+ T cells following re-challenge with RPMI target cells with concurrent treatment of Compound A or Compound B during chronic activation. FIG. 3B shows the cytokine production of anti-BCMA CAR+ T cells following re-challenge with RPMI target cells with concurrent treatment of Compound A or Compound B during chronic activation.

FIG. 4A shows the cytolytic activity of anti-BCMA CAR+ T cells following re-challenge with RPMI target cells with treatment of Compound A or Compound B during rechallenge. FIG. 4B shows the cytokine production of anti-BCMA CAR+ T cells following re-challenge with RPMI target cells with concurrent treatment of Compound A or Compound B during rechallenge.

FIG. 5A shows the analysis of population doublings of anti-BCMA CAR+ T cells in the presence of absence of Compound A for two donors. FIG. 5B shows the cytokine production in the cultures 24 hours after a first reset (day 5) or second reset (day 9) following replating with fresh target cells in the serial stimulation assay.

FIG. 6A shows the tumor volume and FIG. 6B shows the survival of iberdomide-sensitive mice (NOD.Cg-Prkdc^{scid}IL-2rg^{tm1Wj1}/SzJ mice (NSG; Jackson Labs)) that were administered Compound A in combination with anti-BCMA CAR T cells via concurrent or delayed dosing. FIG. 6C shows the numbers of CD3⁺ CAR+ T cells in the blood in mice having received the combination of anti-BCMA CAR+ T cells and Compound A in the concurrent regimen on days 6 and 14.

FIG. 7A shows the tumor volume and FIG. 7B shows the survival of iberdomide-resistant mice (NOD.Cg-Prkdc^{scid}IL-2rg^{tm1Wj1}/SzJ mice (NSG; Jackson Labs)) that were administered Compound A in combination with anti-BCMA CAR T cells via concurrent or delayed dosing. FIG. 7C, shows the numbers of CD3⁺ CAR+ T cells in the blood in mice having received anti-BCMA CAR+ T cells at both the low and high dose in combination with Compound A in the concurrent regimen on days 12 and 19.

FIG. 8 shows the viability and count of anti-BCMA CAR T cells from three donors in the presence of lenalidomide (1000 nM) or Compound A (0.1 nM, 1 nM, or 10 nM) after 7 days.

FIG. 9A shows cytolytic activity of anti-BCMA CAR T cells from three donors during long term stimulation in the presence of lenalidomide (1000 nM) or Compound A (1 nM or 10 nM). FIGS. 9B-D shows the production of IFN-gamma (FIG. 9B), IL-2 (FIG. 9C), and TNF-alpha (FIG. 9D) in anti-BCMA CAR T cells from three donors during long term stimulation in the presence of lenalidomide (1000 nM) or Compound A (1 nM or 10 nM).

FIG. 10A shows cytolytic activity of anti-BCMA CAR T cells from three donors during chronic stimulation for 7 days with BCMA-conjugated beads and re-challenged with BCMA-expressing RPMI-8226 MM cells in the presence of Compound A (1 nM or 10 nM). FIGS. 10B-D shows the production of IFN-gamma (FIG. 10B), IL-2 (FIG. 10C), and TNF-alpha (FIG. 10D) in anti-BCMA CAR T cells from three donors during chronic stimulation in the presence of Compound A (1 nM or 10 nM).

FIG. 11A shows cytolytic activity of anti-BCMA CAR T cells from three donors during chronic stimulation for 7 days with BCMA-conjugated beads in the presence of IMiD/CELMoD resistant cell line DF-15R and Compound A (1 nM or 10 nM). FIGS. 11B-D shows the production of IFN-gamma (FIG. 11B), IL-2 (FIG. 11C), and TNF-alpha (FIG. 11D) in anti-BCMA CAR T cells from three donors during chronic stimulation in the presence of IMiD/CELMoD resistant cell line DF-15R and Compound A (1 nM or 10 nM).

### DETAILED DESCRIPTION

Provided herein are combination therapies involving administration of an immunotherapy involving T cell function or activity, such as a T cell therapy, and (S)-3-[4-(4-morpholin-4-ylmethyl-benzyloxy)-1-oxo-1,3-dihydro-isoindol-2-yl]-piperidine-2,6-dione

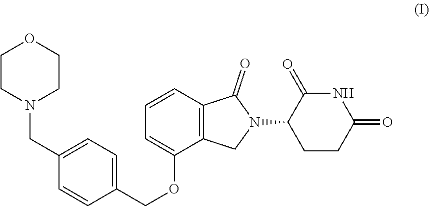 [SMILES: O=C1CC[C@H](N2Cc3c(OCc4ccc(CN5CCOCC5)cc4)cccc3C2=O)C(=O)N1]

or a pharmaceutically acceptable salt, solvate, hydrate, stereoisomer, tautomer or racemic mixtures thereof (Compound A), and compositions thereof, for the treatment of subjects with a cancer or proliferative disease.

Also provided are combination therapies involving administration of an immunotherapy involving T cell function or activity, such as a T cell therapy, and (S)-4-(4-(4-(((2-(2,6-dioxopiperidin-3-yl)-1-oxoisoindolin-4-yl)oxy)methyl)benzyl)piperazin-1-yl)-3-fluorobenzonitrile

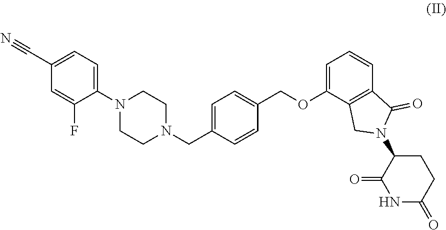 [SMILES: N#Cc1ccc(N2CCN(Cc3ccc(COc4cccc5c4CN([C@H]4CCC(=O)NC4=O)C5=O)cc3)CC2)c(F)c1]

or a pharmaceutically acceptable salt, solvate, hydrate, stereoisomer, tautomer or racemic mixtures thereof (Compound B), and compositions thereof, for the treatment of subjects with a cancer or proliferative disease.

Among the provided embodiments are combinations treatments involving a T cell therapy targeting or directed to BCMA and BCMA-expressing cells and diseases. It is observed that BCMA is expressed, e.g., heterogeneously expressed, on certain diseases and conditions such as malignancies or tissues or cells thereof, e.g., on malignant plasma cells such as from all relapsed or newly diagnosed myeloma patients, for example, with little expression on normal tissues. Among the provided embodiments are approaches useful in the treatment of such diseases and conditions and/or for targeting such cell types, including nucleic acid molecules that encode BCMA-binding receptors, including chimeric antigen receptors (CARs), and the encoded receptors such as the encoded CARs, and compositions and articles of manufacture comprising the same. The receptors generally can contain antigen-binding domains that include antibodies (including antigen-binding antibody fragments, such as heavy chain variable (V_{H}) regions, single domain antibody fragments and single chain fragments, including scFvs) specific for BCMA. Also provided are cells, such as engineered or recombinant cells expressing such BCMA-binding receptors, e.g., anti-BCMA CARs and/or containing nucleic acids encoding such receptors, and compositions and articles of manufacture and therapeutic doses containing such cells, for use in the provided methods.

Among the diseases to be treated is any disease or disorder associated with BCMA or any disease or disorder in which BCMA is specifically expressed and/or in which BCMA has been targeted for treatment (also referred to herein interchangeably as a “BCMA-associated disease or disorder”). Cancers associated with BCMA expression include hematologic malignancies such as multiple myeloma, Waldenstrom macroglobulinemia, as well as both Hodgkin's and non-Hodgkin's lymphomas. See Coquery et al., Crit Rev Immunol., 2012, 32(4):287-305 for a review of BCMA. Since BCMA has been implicated in mediating tumor cell survival, it is a potential target for cancer therapy. Chimeric antigen receptors containing mouse anti-human BCMA antibodies and cells expressing such chimeric receptors have been previously described. See Carpenter et al., Clin Cancer Res., 2013, 19(8):2048-2060.

In some aspects, the provided methods enhance or modulate proliferation and/or activity of T cell activity associated with administration of an immunotherapy or immunotherapeutic agent, such as a composition including cells for adoptive cell therapy, e.g., such as a T cell therapy (e.g. CAR-expressing T cells). In some embodiments, the combination therapy involves administration of an immunomodulatory compound, such as a structural or functional analog of thalidomide and/or an inhibitor of E3-ubiquitin ligase, and administration of the T cell therapy, such as a composition including cells for adoptive cell therapy, e.g., such as a T cell therapy (e.g. CAR-expressing T cells).

(S)-3-[4-(4-morpholin-4-ylmethyl-benzyloxy)-1-oxo-1,3-dihydro-isoindol-2-yl]-piperidine-2,6-dione (Compound A) is a cereblon E3 ligase modulatory compound (CELMoD). Compound A modulates CRBN, which induces ubiquitination of the transcription factors Aiolos and Ikaros, increasing their proteasome dependent degradation and augmenting T cell function. Compound A binds more potently to CRBN, is more efficient at degrading Aiolos and Ikaros than lenalidomide and pomalidomide, and has potent direct anti-proliferative effects on lymphoma cells. Compound A has direct anti-proliferative effects on lymphoma cells. As shown herein, Compound A also augments T cell function.

(S)-4-(4-(4-(((2-(2,6-dioxopiperidin-3-yl)-1-oxoisoindolin yl)oxy)methyl)benzyl)piperazin-1-yl)-3-fluorobenzonitrile (Compound B) is also a cereblon E3 ligase modulatory compound (CELMoD) that modulates CRBN and induces ubiquitination of the transcription factors Aiolos and Ikaros. Compound B induces loss of Aiolos and Ikaros in cultures of PBMCs and result in the activation of T cells and increased production of IL-2 and IFN-γ.

T cell-based therapies, such as adoptive T cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of cancer and other diseases and disorders. The engineered expression of recombinant receptors, such as chimeric antigen receptors (CARs), on the surface of T cells enables the redirection of T-cell specificity.

In certain contexts, available approaches to adoptive cell therapy may not always be entirely satisfactory. For example, in certain cases, although CAR T cell persistence can be detected in many subjects responses in some subjects are transient and subjects have been shown to relapse in the presence of persistent CART cells.

In some aspects, an explanation for this is the immunological exhaustion of circulating CAR-expressing T cells and/or changes in T lymphocyte populations. In some contexts, optimal efficacy can depend on the ability of the administered cells to recognize and bind to a target, e.g., target antigen, to traffic, localize to and successfully enter appropriate sites within the subject, tumors, and environments thereof. In some contexts, optimal efficacy can depend on the ability of the administered cells to become activated, expand, to exert various effector functions, including cytotoxic killing and secretion of various factors such as cytokines, to persist, including long-term, to differentiate, transition or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and avoid or reduce exhaustion, anergy, peripheral tolerance, terminal differentiation, and/or differentiation into a suppressive state.

In some embodiments, the exposure and persistence of engineered cells is reduced or declines after administration to the subject. Yet, observations indicate that, in some cases, increased exposure of the subject to administered cells expressing the recombinant receptors (e.g., increased number of cells or duration over time) may improve efficacy and therapeutic outcomes in adoptive cell therapy.

In some embodiments, following long-term stimulation or exposure to antigen and/or exposure under conditions in the tumor microenviroment, T cells can over time become hypofunctional and/or exhibit features associated with exhausted state. In some aspects, this reduces the persistence and efficacy of the T cells against antigen and limits their ability to be effective. There is a need for methods to improve the efficacy and function of CAR T cells, particularly to minimize, reduce, prevent or reverse hypofunctional or exhaustive states.

Compounds A and B have been shown to directly affect malignant lymphocyte survival through the degradation of Ikaros family transcription factors. The molecular target for such compounds has been identified as the protein Cereblon (CRBN), a substrate receptor of the Cullin 4 RING E3 ubiquitin ligase complex. Binding to a hydrophobic tri-tryptophan pocket within CRBN promotes the recruitment, ubiquitination, and subsequent proteasomal degradation of several protein substrates, including Aiolos (IKZF3) and Ikaros (IKZF1). Ikaros is expressed in immature stages of myeloid differentiation and regulates early neutrophil differentiation (Dumortier et al. (2003) Blood 101:2219). Thus, in some cases, depletion of Ikaros, such as by administration of an immunomodulatory compound, e.g. Compound A or Compound B, to subjects can, in some instances, result in neutropenia.

In addition to its cell autonomous activity against malignant B cells, E3 ligase modulatory compounds, such as Compound A or Compound B, also exerts co-stimulatory effects on immune cells such T and NK-cells. This activity also has been shown to be through CRBN mediated degradation of Aiolos and Ikaros, which are negative regulators of activation molecules and cytokines such as interleukin-2 (IL-2) expression. (Gandhi, Br J Haematol. 2014 March; 164(6):811-21, Krönke, Oncoimmunology, 2014; 3(7): e941742.).

The provided methods are based on observations that the immunomodulatory compound, such as Compound A and Compound B, improves T cell function, including functions related to the ability to produce one or more cytokines, cytotoxicity, expansion, proliferation, and persistence of T cells. In some aspects, the provided methods enhance or modulate proliferation and/or activity of T cell activity associated with administration of the T cell therapy (e.g. CAR-expressing T cells). It is found that such methods and uses provide for or achieve improved or greater T cell functionality, and thereby improved anti-tumor efficacy.

It also is found herein that, in addition to potentiating T cell function, such immunomodulatory compounds, e.g. Compound A or Compound B, exhibit effects to reverse, delay, or prevent T cell exhaustion, including by increasing T cell signaling and/or altering one or more genes that are differentially regulated following chronic (long-term) stimulation. Thus, while in some cases agents that increase or potentiate T cell activity may drive the cells to an exhausted state, it is found herein that activity of such immunomodulatory compounds, e.g. Compound A or Compound B, to exert a potentiating effect on T cell activity is decoupled from T cell exhaustion. In some embodiments, the provided methods involving compound administration of such immunomodulatory compounds, e.g. Compound A or Compound B, is capable of potentiating activity of naïve T cells and delaying, limiting, reducing, inhibiting or preventing exhaustion. Remarkably, results herein show that exposure of T cells, that have been chronically stimulated and exhibit features of exhausted T cells, to an immunomodulatory compound described herein, such as Compound A or Compound B, are able to recover activity or have their activity restored or partially restored. The observations herein support that the provided methods may also achieve improved or more durable responses as compared to certain alternative methods, such as in particular groups of subjects treated.

Moreover, observations herein show that the immunomodulatory compounds, e.g. Compound A or Compound B, exhibit activity to rescue T cells from T cell exhaustion, such as by restoring or partially restoring one or more T cell activities after a cell has shown features of exhaustion. Remarkably, results herein show that exposure of T cells, that have been chronically stimulated and exhibit features of exhausted T cells, to an immunomodulatory compound described herein, such as Compound A or Compound B, are able to recover activity or have their activity restored or partially restored. These results are not observed with interleukin 2 (IL-2), which, in some cases, is a downstream modulator induced by such immunomodulatory compounds. The observations herein support that the provided methods may also achieve improved or more durable responses as compared to certain alternative methods, such as in particular groups of subjects treated.

These observations were made using a chronic stimulation assay to render CAR T cells hypofunctional (e.g. reduced cytolysis and IL-2 secretion). Using this model, CAR T cells were examined to assess impact of immunoodulatory compounds that inhibit E3 ligase, e.g. Compound A or Compound B, on CAR T cell function when present during (concurrent) or following (rescue) exposure to conditions leading to a hypofunctional, exhaustive state. Upon rechallenge with antigen, the findings provided herein demonstrate that concurrent treatment of CAR T cells during such conditions reversed activity and phenotypes, including gene signatures, associated with CAR T cell hypofunctionality and preserved more effector function. Likewise, the results show that immunoodulatory compounds that inhibit E3 ligase, e.g. Compound A or Compound B, could rescue or restore T cell function, including cytokine production and cytolytic activity, of exhausted T cells. Further, the results were seen with different target antigens and different CARs.

In some embodiments, the effect on T cell exhaustion as observed by the immunomodulatory compounds, such as Compound A or Compound B, is not observed by or induced by IL-2. In some embodiments, such effect, such as the ability to reduce, prevent or delay T cell exhaustion or to rescue or restore T cell activity in exhausted T cells, is not induced by a physiologically relevant amount or a therapeutically effective amount of IL-2. In some embodiments, the effect induced by the immunomodulatory compound, e.g. Compound A or Compound B, such as the ability to reduce, prevent or delay T cell exhaustion or to rescue or restore T cell activity in exhausted T cells, is induced by greater than or greater than or about 1.2-fold, 2.0-fold, 3-fold, 4.0-fold, 5.0-fold, 6.0-fold, 7.0-fold, 10.0-fold, or more seen or induced by IL-2, such as a physiologically relevant amount or a therapeutically effective amount of IL-2.

Observations provided herein also demonstrate that immunomodulatory compounds that inhibit E3 ligase exhibit activity to increase effector cytokine production by CAR T cells, while at the same time slowing their proliferative rate. This results is not due to an effect of the compounds on viability of T cells. This effect on proliferation was observed at varied concentration, and was found to be due to accumulation of the T cells in G1 phase. This decoupling of effector cytokine production from proliferation rate could be clinically beneficial, such as by limiting differentiation of T cells in vivo which could limit efficacy.

The provided findings indicate that combination therapy of the immunomodulatory compound, such as a structural or functional analog or derivative of thalidomide and/or an inhibitor of E3 ubiquitin ligase, e.g. Compound A or Compound B, or in methods involving T cells, such as involving administration of adoptive T cell therapy, achieves improved function of the T cell therapy, such as by potentiating T cell activity and reducing, preventing or delaying T cell exhaustion or rescuing cells from T cell exhaustion. In some embodiments, combination of the cell therapy (e.g., administration of engineered T cells) with the immunomodulatory compound, e.g., Compound A or Compound B, improves or enhances one or more functions and/or effects of the T cell therapy, such as persistence, expansion, cytotoxicity, and/or therapeutic outcomes, e.g., ability to kill or reduce the burden of tumor or other disease or target cell.

In particular aspects, it is found herein that an immunomodulatory compound, such as a structural or functional analog or derivative of thalidomide and/or an inhibitor of E3 ubiquitin ligase, e.g. Compound A or Compound B, promotes continued function and/or survival of cells of a T cell therapy (e.g. CAR-T cells) after activation, including after encounter with antigen. In some aspects, Compound A or Compound B, increases the ability of such T cells to persist or function long-term, such as by preventing exhaustion or cell death. In particular embodiments, combination therapy with an immunomodulatory compound that is an inhibitor of an E3 ligase, e.g. Compound A or Compound B, may provide a useful therapeutic approach for enhancing and prolonging the activity of CAR T cells across B cell malignancies by modulating the tumor microenvironment, by improving persistent anti-tumor function of CAR T cells. In some cases, the compound may also have direct anti-tumor effects on lymphoma cells. In some embodiments, such improvements can result in a combination therapy exhibiting improved overall responses, e.g. reduction in tumor burden, and/or increased survival compared to in subjects treated with a monotherapy involving administration of the T cell therapy (e.g. CAR-T cell) or immunomodulatory compound (e.g. Compound A or Compound B) alone. In some aspects, the provided methods increase overall response and/or survival by or more than 1.5-fold, 2.0-fold, 3.0-fold, 4.0-fold, 5.0-fold, 10-fold or more compared to an alternative treatment, such as compared to a monotherapy involving administration of the T cell therapy (e.g. CAR-T cell) or immunomodulatory compound (e.g. Compound A or Compound B) alone.

The provided methods include administering an immunomodulatory compound as described, e.g. Compound A or Compound B, in an effective amount to exhibit a T cell modulatory effect. It is found herein that particular dosages of exemplary immunomodulatory compounds as described, e.g. Compound A or Compound B, increase or enhance T cell function of a T cell therapy, e.g. CAR-T cell therapy. In some cases, doses that are too high may negatively impact T cell function. As shown herein, prolonged treatment of Compound A at physiologically-relevant concentrations (10 or 100 nM) can increase long-term proliferative potential of CAR-expressing T cells while higher concentrations, e.g. such as at or about 500 mM, may be detrimental to long term product performance. In some embodiments, the dose of Compound A that is administered from or from about 1 mg to about 10 mg, such as from or from about 1 mg to about 5 mg. The dose can be administered daily in a course of treatment or cycling regimen.

In particular embodiments, Compound A or Compound B can be used in any of the provided methods. In some embodiments, the provided methods include administering Compound A or Compound B in an effective amount to exhibit a T cell modulatory effect. In some embodiments, the dose of Compound A that is administered is from or from about 0.1 mg to about 1 mg, such as from or from about 0.3 mg to about 0.6 mg. In some embodiments, the dose of Compound B that is administered is from or from about 0.1 mg to about 1 mg, such as from or from about 0.3 mg to about 0.6 mg. The dose can be administered daily in a course of treatment or cycling regimen.

In some embodiments, the combination with the immunomodulatory compound, while improving one or more outcomes or functional attributes, does not affect one or more side effects or unwanted changes in the T cells, such as does not reduce the ability of the cells to become activated, secrete one or more desired cytokines, expand and/or persist, e.g., as measured in an in vitro assay as compared to such cells cultured under conditions otherwise the same but in the absence of the immunomodulatory compound. Thus in some embodiments, provided are methods and combinations that result in improvements in T cell function or phenotype, e.g., in intrinsic T cell functionality and/or intrinsic T cell phenotype, generally without compromising one or more other desired properties of functionality, e.g., of CAR-T cell functionality.

In some embodiments, the provided methods can potentiate T cell therapy, e.g. CAR-T cell therapy, which, in some aspects, can improve outcomes for treatment. In some embodiments, the methods are particularly advantageous in subjects in which the cells of the T cell therapy exhibit weak expansion, have become exhausted, exhibit a reduced or decreased persistence in the subject and/or in subjects that have a cancer that is resistant or refractory to other therapies, is an aggressive or high-risk cancer, and/or that is or is likely to exhibit a relatively lower response rate to a CAR-T cell therapy administered without the immunomodulatory compound compared to another type of cancer or compared to administration with a different CAR-T cell therapy.

In some embodiments, the provided methods are used at a time at which a T cell therapy (e.g. CAR T cells) may exhibit or are likely to exhibit features of exhaustion. In some embodiments, an exhaustive phenotype is evident after T cells, having reached peak expansion, begin to decline in number in the blood of the subject. In some embodiments, the methods of exposing or contacting T cells of a T cell therapy (CAR T cells) with an immunomodulatory compound that inhibits E3 ligase, e.g. Compound A or Compound B, are carried out at a time at which the T cells exhibit an increase in a hypofunctional or exhaustive state compared to at the time just prior to exposure of the T cells to an antigen (baseline) or to a time point at which the cells have been exposed to the antigen but are continuing to proliferate and have not yet reached peak expansion. In some embodiments, an increase in hypofunctional or exhaustive state can be determined by increased expression of an exhaustion marker compared to the previous earlier timepoint. In some embodiments, the increase in the hypofunctional or exhaustive state, such as increase in expression of an exhaustion marker, is at a time following administration of the T cell therapy (e.g., CAR T cells) to a subject having a disease or condition associated with the antigen targeted by the T cell therapy. The T cells, such as T cells in peripheral blood after administration to a subject, can be monitored for markers of T cell activation or exhaustion such as PD-1. TIM-3 and LAG-3.

In some aspects, the provided methods can enhance, increase or potentiate T cell therapy, such as to overcome lack of persistence and/or exhaustion of T cells, e.g. in subjects in which, at or about day 12-15 days after initiation of administration of the T cell therapy, less than 10 μL, such as less than 5 μL or less than 1 μL of such cells, or a CD8+ or CD3+ subset thereof, are detectable in the blood. In some embodiments, a subject having received administration of a T cell therapy, e.g. CAR-T cell, is monitored for the presence, absence or level of T cells of the therapy in the subject, such as in a biological sample of the subject, e.g. in the blood of the subject. In some embodiments, an immunomodulatory compound, such as a structural or functional analog or derivative of thalidomide and/or an inhibitor of E3 ubiquitin ligase, e.g. Compound A or Compound B, is administered to a subject having received the T cell therapy (e.g. CAR-T cells) but in which such cells have weakly expanded and/or are at or below a threshold level in a sample of the subject, e.g. blood sample, at a time when strong or robust expansion of the CAR-T cells in the subject is typically observed in a plurality of subjects administered a T cell therapy (e.g. CAR-T), in some cases, this same T cell therapy (e.g. same CAR-T cells). In some aspects, an immunomodulatory compound, such as a structural or functional analog or derivative of thalidomide and/or an inhibitor of E3 ubiquitin ligase, e.g., Compound A or Compound B, is administered if, at or about day 12-15 after initiation of administration of the T cell therapy, less than 10 μL, such as less than 5 μL or less than 1 μL of such cells, or a CD8+ or CD3+ subset thereof, are detectable in the blood.

In certain aspects, the provided methods can enhance, increase or potentiate T cell therapy in subjects in which a peak response to the T cell therapy has been observed but in which the response, e.g. presence of T cells and/or reduction in tumor burden, has become reduced or is no longer detectable. In some aspects, an immunomodulatory compound, such as a structural or functional analog or derivative of thalidomide and/or an inhibitor of E3 ubiquitin ligase, e.g. Compound A or Compound B, is administered to a subject within a week, such as within 1, 2 or 3 days after: (i) peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the T cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (iv) the subject exhibits increased tumor burden as compared to tumor burden at a time prior to or after administration of the T cells and prior to initiation of administration of the immunomodulatory compound.

In some embodiments, the methods can be used for treating a disease or condition, e.g. a multiple myeloma, such as a relapsed/refractory multiple myeloma. In some embodiments, the methods can be used to treat such diseases, conditions or malignancies in which responses, e.g. complete response, to treatment with the T cell therapy alone, such as a composition including cells for adoptive cell therapy, e.g., such as a T cell therapy (e.g. CAR-expressing T cells), is relatively low compared to treatment with other T cell therapies or treatment of other diseases or malignancies (e.g. a CR in a less than or less than about 60%, less than about 50% or less than about 45% of the subjects so treated) and/or in which the subject is not responsive to treatment with the immunomodulatory compound, such as a structural or functional analog or derivative of thalidomide and/or an inhibitor of E3 ubiquitin ligase, e.g. Compound A or Compound B, alone.

In some embodiments, the combination therapy provided herein is for use in a subject having a cancer in which after initiation of administration of the T cell therapy, such as a composition including cells for adoptive cell therapy, e.g., CAR-expressing T cells, the subject has relapsed following remission after treatment with the T cell therapy. In some embodiments, subjects that have relapsed following such remission are administered an immunomodulatory compound, such as a structural or functional analog or derivative of thalidomide and/or an inhibitor of E3 ubiquitin ligase, e.g. Compound A or Compound B. In some embodiments, the combination therapy provided herein is for use in a subject having a disease or condition, e.g. cancer, in which the amount of the immunomodulatory compound administered is insufficient, as a single agent and/or in the absence of administration of the T cell therapy, to ameliorate, reduce or prevent the disease or condition or a symptom or outcome thereof, such as is insufficient to ameliorate, reduce or prevent the disease or condition in the subject or a symptom or outcome thereof. In some embodiments, the method thereby reduces or ameliorates a symptom or outcome or burden of the disease or condition to a degree that is greater than the combination of (i) the degree of reduction or amelioration effected by the administration of the immunomodulatory agent alone, optionally on average in a population of subjects having the disease or condition, and (ii) the degree of reduction or amelioration by the administration of the T cell therapy alone, optionally on average in a population of subjects having the disease or condition. In some embodiment, the method reduces or ameliorates such symptoms, outcomes or burdens of the disease, e.g. compared to on average in a population of subjects having the disease or condition, by greater than or greater than about 1.5-fold, 2.0-fold, 3.0-fold, 4.0-fold, 5.0-fold, 6.0-fold, 7.0-fold, 8.0-fold, 9.0-fold, 10.0 fold, 20.0-fold, 30.0-fold, 40.0-fold, 50.0-fold or more.

In some embodiments of the provided methods, one or more properties of administered genetically engineered cells can be improved or increased or greater compared to administered cells of a reference composition, such as increased or longer expansion and/or persistence of such administered cells in the subject or an increased or greater recall response upon restimulation with antigen. In some embodiments, the increase can be at least a 1.2-fold, at least 1.5-fold, at least 2-fold, at last 3-fold, at least 4-fold, at least 5-fold, at least 6-fold, at least 7-fold, at least 8-fold, at least 9-fold, or at least 10-fold increase in such property or feature compared to the same property or feature upon administration of a reference cell composition. In some embodiments, the increase in one or more of such properties or features can be observed or is present within 7 days, 14 days, 21 days, within one months, two months, three months, four months, five months, six months, or 12 months after administration of the genetically engineered cells and the initiation of administration of the immunomodulatory compound, such as a structural or functional analog or derivative of thalidomide and/or an inhibitor of E3 ubiquitin ligase, e.g., Compound A or Compound B.

In some embodiments, Compound A is administered in an amount between at or about 0.1 mg and at or about 1 mg. The dose can be administered daily over a cycling regimen. In some aspects, the provided methods are carried out by administering an amount of the compound that is or is less than 1 mg per day, such as is or is about 0.9 mg, 0.8 mg, 0.7 mg, 0.6 mg, 0.5 mg, 0.4 mg, 0.3 mg, 0.2 mg, or 0.1 mg, or any value between any of the foregoing In some embodiments, Compound A is administered at or at about 0.3 mg per day. In some embodiments, Compound A is administered at or at about 0.45 mg per day. In some embodiments, Compound A is administered at or about 0.6 mg per day.

In some embodiments, Compound A is administered to the subject a sufficient time after receiving a lymphodepleting therapy, such that myelosuppressive effects of Compound A and the lymphodepleting therapy are minimized.

In some embodiments, the provided methods are used at a time at which a T cell therapy (e.g. CAR T cells) may exhibit or are likely to exhibit features of exhaustion. In some embodiments, an exhaustive phenotype is evident after T cells, having reached peak expansion, begin to decline in number in the blood of the subject. In some embodiments, the methods of exposing or contacting T cells of a T cell therapy (CAR T cells) with Compound A are carried out at a time at which the T cells exhibit an increase in a hypofunctional or exhaustive state compared to at the time just prior to exposure of the T cells to an antigen (baseline) or to a time point at which the cells have been exposed to the antigen but are continuing to proliferate and have not yet reached peak expansion. In some embodiments, an increase in hypofunctional or exhaustive state can be determined by increased expression of an exhaustion marker compared to the previous earlier timepoint. In some embodiments, the increase in the hypofunctional or exhaustive state, such as increase in expression of an exhaustion marker, is at a time following administration of the T cell therapy (e.g. CAR T cells) to a subject having a disease or condition associated with the antigen targeted by the T cell therapy. The T cells, such as T cells in peripheral blood after administration to a subject, can be monitored for markers of T cell activation or exhaustion such as PD-1. TIM-3 and LAG-3.

In some embodiments, the administration of Compound A is initiated at a time that is or that is suspected or likely to be before or about at a time peak CAR-T cells are present in the blood of the subject, e.g. within 21 days after initiation of administration of the T cell. In some cases, peak CAR-T cells present within 11-15 days following administration of CAR T cells. In some embodiments, the administration of Compound A is initiated at a time that is 1 to 15 days, e.g. at or about 1 day or 8 days or 15 days after initiation of administration of the cell therapy. In some embodiments, Compound A is administered at a time when the subject does not exhibit a severe toxicity following the administration of the cell therapy.

In some aspects, in any of the provided methods, the administration of the compound begins (or is initiated) within 21 days after administering the T cell therapy and is carried out in a cycling regimen comprising: a first administration period during which the compound is administered daily at about 0.1 mg to about 1.0 mg per day for up to three consecutive weeks, a pause period beginning at the end of the first administration period for at least one week during which the compound is not administered, and a second administration period comprising four-week cycles during which the compound is administered daily at about 0.1 mg to about 1.0 mg per day for three consecutive weeks in the four week period. In some embodiments, the compound is administered at about 0.30 mg, 0.45 mg, or 0.60 mg per day during the first administration period and the second administration period.

In some embodiments, the provided methods do not result in a high rate or likelihood of toxicity or toxic outcomes, or reduces the rate or likelihood of toxicity or toxic outcomes, such as neurotoxicity (NT), cytokine release syndrome (CRS), or hematological toxicities, such as neutropenia, such as compared to certain other cell therapies or immunomodulatory drug regimens.

In some embodiments, the methods do not result in, or do not increase the risk of, certain hematological toxicities, such as neutropenia or thrombocytopenia. In some embodiments, no more than 50% of subjects exhibit a neutropenia higher than grade 3, such as a prolonged grade 3 neutropenia or a grade 4 neutropenia, and/or a thrombocytopenia higher than grade 3, such as a grade 3 or grade 4 thrombocytopenia. In some embodiments, at least 50% of subjects treated according to the method (e.g. at least 60%, at least 70%, at least 80%, at least 90% or more of the subjects treated) do not exhibit a severe neutropenia or a severe thrombocytopenia of grade 3 or higher than grade 3.

In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxicity. In some embodiments, no more than 50% of subjects treated (e.g. at least 60%, at least 70%, at least 80%, at least 90% or more of the subjects treated) exhibit a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells.

In some cases, Compound A is administered at a time that it can efficiently/effectively boost or prime the cells. In some embodiments, the administration of Compound A is initiated at or before peak or maximum level of the cells of the cell therapy is detectable in the blood of the subject. In some embodiments, the provided methods can potentiate T cell therapy, e.g. CAR-T cell therapy, which, in some aspects, can improve outcomes for treatment. In some embodiments, the methods are particularly advantageous in subjects in which the cells of the T cell therapy exhibit weak expansion, have become exhausted, exhibit a reduced or decreased persistence in the subject and/or in subjects that have a cancer that is resistant or refractory to other therapies, and/or is an aggressive or high-risk cancer.

In some embodiments, a subject having received administration of a T cell therapy, e.g. CAR-T cell, is monitored for the presence, absence or level of T cells of the therapy in the subject, such as in a biological sample of the subject, e.g. in the blood of the subject. In some embodiments, the provided methods result in genetically engineered cell with increased persistence and/or better potency in a subject to which it is administered. In some embodiments, the persistence of genetically engineered cells, such as CAR-expressing T cells, in the subject is greater as compared to that which would be achieved by alternative methods, such as those involving administration of a T cell therapy but in the absence of administration of Compound A. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the recombinant receptor (e.g., CAR-expressing cells) in the blood or serum or organ or tissue (e.g., disease site) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the recombinant receptor (e.g. CAR-expressing cells) can be used to distinguish the administered cells from endogenous cells in a subject.

In some embodiments, Compound A is administered for a period of time to enhance, increase or optimize durability of response. In some aspects, the provided methods are based on observations that subjects who achieve or are in complete remission (CR) at 3 months, such as generally at 6 months, are more likely to sustain the response longer term, such as survive or survive without progression for greater than or greater than about three months, four months, five months, six months, seven months, eight months, nine months, ten months, eleven months or twelve months after ending the treatment or after first achieving a complete response (CR) following administration of the combination therapy. In some aspects, the methods are carried out to administer Compound A, such as in a particular cycling regimen as described, for a period of time that is at least 3 months, such as at least four months, at least five months or at least six months after initiation of administration of the T cell therapy. In some embodiments, Compound A is administered, such as in a particular cycling regimen as described, for at least six months or at least 180 days after initiation of administration of the T cell therapy. In some embodiments, at the end of the period, administration of Compound A is ended or stopped if the subject exhibits a CR or if the disease or condition has progressed or relapsed in the subject following remission after receiving the treatment (combination therapy). In some aspects, continued administration of Compound A can be carried out in subjects who, at the end of the period of time (e.g. at or about 6 months) exhibit a partial response (PR) or stable disease (SD). In other aspects, the period of time is a fixed duration and no further administration of Compound A is carried out.

In some aspects, the provided methods and uses provide for or achieve improved or more durable responses or efficacy as compared to certain alternative methods, e.g. methods that include administration of the T cell therapy or Compound A as a monotherapy or without administration as a combination therapy together as described herein, such as in particular groups of subjects treated. In some embodiments, the methods are advantageous by virtue of administering T cell therapy, such as a composition including cells for adoptive cell therapy, e.g., such as a T cell therapy (e.g. CAR-expressing T cells), and Compound A. In some embodiments, such responses are observed in high risk patients with poor prognosis, such as multiple myeloma that has relapsed or is refractory (R/R) to standard therapy or has a poor prognosis.

In some embodiments, at least 35%, at least 40%, at least 50%, at least 55%, at least 60%, at least 65%, at least 70%, or at least 75% or more of the subjects treated according to the provided methods, and/or with the provided articles of manufacture, kits or compositions, achieve a complete response (CR). In some embodiments, the subject is in CR and exhibits minimum residual disease (MRD). In some embodiments, the subject is in CR and is MRD−. In some embodiments, at least 50%, at least 60%, at least 70%, at least 80%, or at least 90% of the subjects treated according to the provided methods, and/or with the provided articles of manufacture, kits or compositions, achieve an objective response of a partial response (PR). In some embodiments, at least 60%, at least 70%, at least 80%, at least 90%, at least 95% or more of the subjects treated according to the provided methods, and/or with the provided articles of manufacture, kits or compositions, achieve a CR or PR at six months, at seven months, at eight months, at nine months, at ten months, at eleven months or a year after initiation of administration of the cell therapy.

In some embodiments, by three months, four months, five months, six months, seven months, eight months, nine months, ten months, eleven months or twelve months or more after initiation of administration of the cell therapy, at least 60%, at least 70%, at least 80%, at least 90%, at least 95% or more of the subjects treated according to the provided methods, and/or with the provided articles of manufacture, kits or compositions, remain in response, such as remain in CR or an objective response (OR). In some embodiments, such response, such as CR or OR, is durable for at least three months, four months, five months, six months, seven months, eight months, nine months, ten months, eleven months, twelve months or more such as in at least or about at least 60%, at least 70%, at least 80%, at least 90%, at least 95% or more of the subjects treated according to the provided methods or in such subjects who achieve a CR by three months, four months, five months or six months. In some embodiments, at least 60%, at least 70%, at least 80%, at least 90%, at least 95% or more of the subjects treated according to the provided methods, and/or with the provided articles of manufacture, kits or compositions, or such subjects who achieve a CR by three months, four months, five months or six months survive or survive without progression for greater than or greater than about six months, seven months, eight months, nine months, ten months, eleven months, twelve months or longer.

In some embodiments, a reference cell composition can be a composition of T cells from the blood of a subject not having or not suspected of having the cancer or is a population of T cells obtained, isolated, generated, produced, incubated and/or administered under the same or substantially the conditions, except not having been incubated or administered in the presence of the immunomodulatory compound. In some embodiments, the reference cell composition contains genetically engineered cells that are substantially the same, including expression of the same recombinant receptor, e.g., CAR. In some aspects, such T cells are treated identically or substantially identically, such as manufactured similarly, formulated similarly, administered in the same or about the same dosage amount and other similar factors.

In some embodiments, the provided methods result in genetically engineered cell with increased persistence and/or better potency in a subject to which it is administered. In some embodiments, the persistence of genetically engineered cells, such as CAR-expressing T cells, in the subject is greater as compared to that which would be achieved by alternative methods, such as those involving administration of a reference cell composition, e.g. administration of the T cell therapy but in the absence of administration of the immunomodulatory compound. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the recombinant receptor (e.g., CAR-expressing cells) in the blood or serum or organ or tissue (e.g., disease site) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the recombinant receptor (e.g. CAR-expressing cells) can be used to distinguish the administered cells from endogenous cells in a subject.

Also provided are methods for engineering, preparing, and producing the cells, compositions containing the cells and/or immunomodulatory compound, and kits and devices containing and for using, producing and administering the cells and/or immunomodulatory compound, such as in accord with the provided combination therapy methods.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### I. COMBINATION THERAPY

Provided are methods and uses of engineered cells, such as T cells (e.g., CAR-T cells) in combination with (S)-3-[4-(4-morpholin-4-ylmethyl-benzyloxy)-1-oxo-1,3-dihydro-isoindol-2-yl]-piperidine-2,6-dione or a compound of formula I

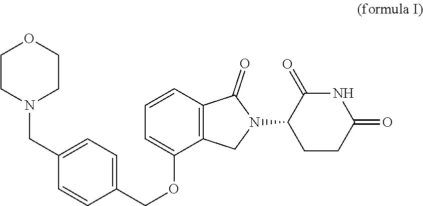 [SMILES: O=C1CC[C@H](N2Cc3c(OCc4ccc(CN5CCOCC5)cc4)cccc3C2=O)C(=O)N1]

or a pharmaceutically acceptable salt, solvate, hydrate, stereoisomer, tautomer or racemic mixtures thereof (Compound A), including compositions thereof, for the treatment of subjects with cancer. In particular embodiments, the methods are for treating multiple myeloma. In some embodiments, the multiple myeloma is a relapsed or refractory multiple myeloma.

Also provided are methods and uses of engineered cells, such as T cells (e.g., CAR-T cells) in combination with (S)-4-(4-(4-(((2-(2,6-dioxopiperidin-3-yl)-1-oxoisoindolin yl)oxy)methyl)benzyl)piperazin-1-yl)-3-fluorobenzonitrile or a compound of formula II

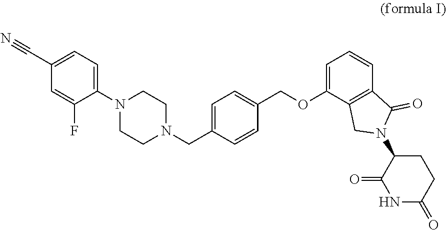 [SMILES: N#Cc1ccc(N2CCN(Cc3ccc(COc4cccc5c4CN([C@H]4CCC(=O)NC4=O)C5=O)cc3)CC2)c(F)c1]

or a pharmaceutically acceptable salt, solvate, hydrate, stereoisomer, tautomer or racemic mixtures thereof (Compound B), including compositions thereof, for the treatment of subjects with cancer. In particular embodiments, the methods are for treating multiple myeloma. In some embodiments, the multiple myeloma is a relapsed or refractory multiple myeloma.

In some embodiments, the cell therapy is adoptive cell therapy. In some embodiments, the cell therapy is or comprises a tumor infiltrating lymphocytic (TIL) therapy, a transgenic TCR therapy or a recombinant-receptor expressing cell therapy (optionally T cell therapy), which optionally is a chimeric antigen receptor (CAR)-expressing cell therapy. In some embodiments, the therapy is a B cell targeted therapy. In some embodiments, the therapy targets B cell maturation antigen (BCMA). In some embodiments, the cells and dosage regimens for administering the cells can include any as described in the following subsection A under “Administration of T Cell therapy.”

In some embodiments, the dosage regimens for administering the immunomodulatory compound can include any as described in the following subsection B under “Administration of the Immunomodulatory Compound.”

In some embodiments, the T cell therapy (e.g. CAR-expressing T cells) and immunomodulatory compound are provided as pharmaceutical compositions for administration to the subject. In some embodiments, the pharmaceutical compositions contain therapeutically effective amounts of one or both of the agents for combination therapy, e.g., T cells for adoptive cell therapy and an immunomodulatory compound as described. In some embodiments, the agents are formulated for administration in separate pharmaceutical compositions. In some embodiments, any of the pharmaceutical compositions provided herein can be formulated in dosage forms appropriate for each route of administration.

In some embodiments, the combination therapy, which includes administering the T cell therapy, including engineered cells, such as CAR-T cell therapy, and the immunomodulatory compound is administered to a subject or patient having a disease or condition to be treated (e.g. cancer) or at risk for having the disease or condition (e.g. cancer). In some aspects, the methods treat, e.g., ameliorate one or more symptom of, the disease or condition, such as by lessening tumor burden in a cancer expressing an antigen recognized by the immunotherapy or immunotherapeutic agent, e.g. recognized by an engineered T cell.

In some embodiments, the disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by bacterial, viral or other pathogens. Exemplary antigens, which include antigens associated with various diseases and conditions that can be treated, include any of antigens described herein. In particular embodiments, the recombinant receptor expressed on engineered cells of a combination therapy, including a chimeric antigen receptor or transgenic TCR, specifically binds to an antigen associated with the disease or condition.

In some embodiments the cancer or proliferative disease expresses BCMA. In some embodiments, the provided methods employ a recombinant receptor-expressing T cell (e.g. CAR-T cell) that targets BCMA.

In some embodiments, the methods and uses include 1) administering to the subject a T cell therapy involving T cells expressing genetically engineered cell surface receptors (e.g., recombinant antigen receptor), which generally are chimeric receptors such as chimeric antigen receptors (CARs), directed against or targeting BCMA, and 2) administering to the subject a Compound A. In some embodiments, administration of Compound A is initiated after (subsequently) to administering the T cell therapy or after (subsequently) to initiating administration of the T cell therapy. In some cases, Compound A is administered to a subject that has received administration of a T cell therapy. The methods generally involve administering one or more doses of the cells and more than one dose of a Compound A to the subject.

In some embodiments, the methods and uses include 1) administering to the subject a T cell therapy involving T cells expressing genetically engineered cell surface receptors (e.g., recombinant antigen receptor), which generally are chimeric receptors such as chimeric antigen receptors (CARs), directed against or targeting BCMA, and 2) administering to the subject a Compound B. In some embodiments, administration of Compound B is initiated after (subsequently) to administering the T cell therapy or after (subsequently) to initiating administration of the T cell therapy. In some cases, Compound B is administered to a subject that has received administration of a T cell therapy. The methods generally involve administering one or more doses of the cells and more than one dose of Compound B to the subject.

The combination therapy, e.g., including engineered cells expressing a recombinant receptor, such as a chimeric antigen receptor (CAR) and immunomodulatory compound (e.g. Compound A or Compound B) or compositions comprising the engineered cells and/or the immunomodulatory compound (e.g. Compound A or Compound B) described herein are useful in a variety of therapeutic, diagnostic and prophylactic indications. For example, the combinations are useful in treating a variety of diseases and disorders in a subject. Such methods and uses include therapeutic methods and uses, for example, involving administration of the engineered cells and immunomodulatory compound (e.g. Compound A or Compound B) and/or compositions containing one or both, to a subject having a disease, condition, or disorder, such as a tumor or cancer. In some embodiments, the engineered cells and the immunomodulatory compound (e.g. Compound A or Compound B) and/or compositions containing one or both are administered in an effective amount to effect treatment of the disease or disorder. Uses include uses of the engineered cells and the immunomodulatory compound (e.g. Compound A or Compound B) and/or compositions containing one or both in such methods and treatments, and in the preparation of a medicament in order to carry out such therapeutic methods. In some embodiments, the methods are carried out by administering the engineered cells and the immunomodulatory compound (e.g. Compound A or Compound B), and/or compositions containing one or both, to the subject having or suspected of having the disease or condition. In some embodiments, the methods thereby treat the disease or condition or disorder in the subject.

Among the diseases to be treated is any disease or disorder associated with BCMA or any disease or disorder in which BCMA is specifically expressed and/or in which BCMA has been targeted for treatment (also referred to herein interchangeably as a “BCMA-associated disease or disorder”). Cancers associated with BCMA expression include hematologic malignancies such as multiple myeloma, Waldenstrom macroglobulinemia, as well as both Hodgkin's and non-Hodgkin's lymphomas. See Coquery et al., Crit Rev Immunol., 2012, 32(4):287-305 for a review of BCMA. Since BCMA has been implicated in mediating tumor cell survival, it is a potential target for cancer therapy. Chimeric antigen receptors containing mouse anti-human BCMA antibodies and cells expressing such chimeric receptors have been previously described. See Carpenter et al., Clin Cancer Res., 2013, 19(8):2048-2060.

In some embodiments, the disease or disorder associated with BCMA is a B cell-related disorder. In some embodiments, the disease or disorder associated with BCMA is one or more diseases or conditions from among glioblastoma, lymphomatoid granulomatosis, post-transplant lymphoproliferative disorder, an immunoregulatory disorder, heavy-chain disease, primary or immunocyte-associated amyloidosis, or monoclonal gammopathy of undetermined significance.

In some embodiments, the disease or disorder associated with BCMA is an autoimmune disease or disorder. Such autoimmune diseases or disorder include, but are not limited to, systemic lupus erythematosus (SLE), lupus nephritis, inflammatory bowel disease, rheumatoid arthritis (e.g., juvenile rheumatoid arthritis), ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, polyarteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

Among the diseases, disorders or conditions associated with BCMA are cancers (e.g., a BCMA-expressing cancer), Cancers, e.g. BCMA-expressing cancers, that can be treated include, but are not limited to, neuroblastoma, renal cell carcinoma, colon cancer, colorectal cancer, breast cancer, epithelial squamous cell cancer, melanoma, myeloma (e.g., multiple myeloma), stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer.

In certain diseases and conditions, BCMA is expressed on malignant cells and cancers. In some embodiments, the cancer (e.g., a BCMA-expressing cancer) is a B cell malignancy. In some embodiments, the cancer (e.g., a BCMA-expressing cancer) is a lymphoma, a leukemia, or a plasma cell malignancy. Lymphomas contemplated herein include, but are not limited to, Burkitt lymphoma (e.g., endemic Burkitt's lymphoma or sporadic Burkitt's lymphoma), non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL). Leukemias contemplated here, include, but are not limited to, chronic lymphocytic leukemia (CLL), plasma cell leukemia or acute lymphocytic leukemia (ALL). Also contemplated herein are plasma cell malignancies including, but not limited to, multiple myeloma (e.g., non-secretory multiple myeloma, smoldering multiple myeloma) or plasmacytoma.

In some embodiments, the disease or condition is a plasmacytoma, such as extramedullary plasmacytoma. In some embodiments, the subject does not have a plasmacytoma, such as extramedullary plasmacytoma.

In some embodiments the disease or condition is multiple myeloma (MM), such as relapsed and/or refractory multiple myeloma (R/R MM).

In some embodiments, the methods may identify a subject who has, is suspected to have, or is at risk for developing a BCMA-associated disease or disorder. Hence, provided are methods for identifying subjects with diseases or disorders associated with elevated BCMA expression and selecting them for treatment with a BCMA-directed T cell therapy (e.g. anti-BCMA CAR T cells.

In some aspects, for example, a subject may be screened for the presence of a disease or disorder associated with elevated BCMA expression, such as a BCMA-expressing cancer. In some embodiments, the methods include screening for or detecting the presence of a BCMA-associated disease, e.g. a tumor or a cancer, such as multiple myeloma. Thus, in some aspects, a sample may be obtained from a patient suspected of having a disease or disorder associated with elevated BCMA expression and assayed for the expression level of BCMA. In some aspects, a subject who tests positive for a BCMA-associated disease or disorder may be selected for treatment by the present methods, and may be administered a therapeutically effective amount of a BCMA-directed T cell therapy (e.g. anti-BCMA CAR T cells) or a pharmaceutical composition thereof as described herein.

In some aspects, a subject may be screened for the level of soluble BCMA (sBCMA), e.g., from a biological sample from the subject, such as the blood or serum. In some aspects, a subject may be screened for the level of sBCMA prior to treatment with the cell therapy. In some aspects, the methods include screening for or detecting the level or amount of sBCMA in a subject that has a disease or disorder associated with BCMA expression, e.g., a tumor or a cancer, such as multiple myeloma. In some aspects, a sample may be obtained from a patient suspected of having a disease or disorder associated with BCMA and assayed for the level or amount of sBCMA, for example, using an assay to detect soluble protein levels, such as an enzyme-linked immunosorbent assay (ELISA). In some aspects, in subjects having a multiple myeloma (MM), sBCMA levels can correlate with the proportion of plasma cells in bone marrow biopsies. In some aspects, in subjects having a multiple myeloma (MM), sBCMA levels can correlate with reduced response to treatment or shorter overall survival or progression free survival (see, e.g., Ghermezi et al., Haematologica 2017, 102(4): 785-795). In some aspects, a subject who exhibits low sBCMA levels may be selected for treatment by the present methods, and may be administered a therapeutically effective amount of a BCMA-directed T cell therapy (e.g. anti-BCMA CAR T cells) or a pharmaceutical composition thereof as described herein.

In some embodiments, the disease or condition associated with BCMA is one that has relapsed in the subject to one or more prior therapies for treating the disease and/or is one in which a subject has not responded to one or more other prior therapies for treating the disease and thus is refractory to treatment with the one or more prior therapies. In particular embodiments, the disease or condition is multiple myeloma that is a relapsed or refractory disease (hereinafter also called relapsed or refractory multiple myeloma or R/R multiple myeloma). In some embodiments, the subject has persistent or relapsed disease, e.g., following treatment with another BCMA-specific antibody and/or cells expressing a BCMA-targeting chimeric receptor and/or other therapy, including chemotherapy, radiation, and/or hematopoietic stem cell transplantation (HSCT), e.g., allogeneic HSCT or autologous HSCT. In some embodiments, the subject is resistant to or refractory to treatment, i.e. does not respond following treatment, with another BCMA-specific antibody and/or cells expressing a BCMA-targeting chimeric receptor and/or other therapy, In some embodiments, the administration of the T cell therapy (e.g. anti-BCMA CAR T cells) in the provided methods effectively treats the subject despite the subject having become resistant or refractory to another BCMA-targeted therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the subject is one that is eligible for a transplant, such as is eligible for a hematopoietic stem cell transplantation (HSCT), e.g., allogeneic HSCT or autologous HSCT. In some such embodiments, the subject has not previously received a transplant, despite being eligible, prior to administration of the a BCMA-directed T cell therapy (e.g. anti-BCMA CAR T cells) and/or compositions comprising the same, as provided herein.

In some embodiments, the subject is one that is not eligible for a transplant, such as is not eligible for a hematopoietic stem cell transplantation (HSCT), e.g., allogenic HSCT or autologous HSCT. In some such embodiments, such a subject is administered a BCMA-directed T cell therapy (e.g. anti-BCMA CAR T cells) and/or compositions comprising the same, according to the provided embodiments herein.

In some embodiments, prior to the initiation of administration of the engineered cells, the subject has received one or more prior therapies for treating the disease or disorder, e.g. multiple myeloma. In some embodiments, the subject has received at least 1, 2, 3, 4, 5, 6, 7, 8, 9, 10, 11, 12, 13, 14, 15, 16, 17, 18, 19 or 20 or more prior therapies. In some embodiments, the subject has received at least 3, 4, 5, 6, 7, 8, 9, 10 or more prior therapies. In some embodiments, the subject has relapsed or is refractory to treatment with two or more prior therapies. In some embodiments, the subject has relapsed or is refractory to treatment with three or more prior therapies. In some embodiments, the subject has relapsed or is refractory to treatment with four or more prior therapies. In some embodiment, the one more prior therapy may include an autologous stem cell transplant (ASCT), an anti-CD38 antibody, such as daratumumab; an immunomodulatory agent or compounds that as thalidomide, lenalidomide or pomalidomide; a proteasome inhibitor such as bortezomib, carfilzomib or ixazomib; or two or more of any of the above. In some aspects, the subject has relapsed or has been refractory to the one or more prior therapies. For example, the subject has R/R multiple myeloma.

In some aspects, the prior therapies include treatment with autologous stem cell transplant (ASCT); an immunomodulatory agent; a proteasome inhibitor; and an anti-CD38 antibody; unless the subject was not a candidate for or was contraindicated for one or more of the therapies. In some aspects, the subject has relapsed or has been refractory to three or more prior therapies, including treatment with three or more therapies selected from (1) an autologous stem cell transplantation, (2) a proteasome inhibitor and an immunomodulatory agent, either alone or in combination, and (3) an anti-CD38 monoclonal antibody, as a part of a combination therapy or a monotherapy; unless the subject was not a candidate for or was contraindicated for one or more of the therapies. In some embodiments, the immunomodulatory agent is selected from among thalidomide, lenalidomide or pomalidomide. In some embodiments, the proteasome inhibitor is selected from among bortezomib, carfilzomib or ixazomib. In some embodiments, the anti-CD38 antibody is or comprises daratumumab. In some embodiments, the subject must have undergone at least 2 consecutive cycles of treatment for each regimen unless progressive disease was the best response to the regimen.

In some embodiments, the method can involve including or excluding particular subjects for therapy with the a BCMA-directed T cell therapy (e.g. anti-BCMA CAR T cells) or a composition comprising the same, based on particular criteria, diagnosis or indication. In some embodiments, at the time of administration of the dose of cells or pre-treatment lymphodepleting chemotherapy, the subject has not had active or history of plasma cell leukemia (PCL). In some embodiments, if the subject had active or a history of PCL at the time of administration, the subject can be excluded from being treated according to the provided methods. In some embodiments, if the subject develops a PCL, such as secondary PCL, at the time of administration, the subject can be excluded from being treated according to the provided methods. In some embodiments, the assessment for the criteria, diagnosis or indication can be performed at the time of screening the subjects for eligibility or suitability of treatment according to the provided methods, at various steps of the treatment regimen, at the time of receiving lymphodepleting therapy, and/or at or immediately prior to the initiation of administration of the engineered cells or composition thereof.

For the prevention or treatment of disease, the appropriate dosage of immunomodulatory compound (e.g., Compound A or Compound B) and/or immunotherapy, such as a T cell therapy (e.g. CAR-expressing T cells), may depend on the type of disease to be treated, the particular immunomodulatory compound, cells and/or recombinant receptors expressed on the cells, the severity and course of the disease, route of administration, whether the immunomodulatory compound and/or the T cell therapy are administered for preventive or therapeutic purposes, previous therapy, frequency of administration, the subject's clinical history and response to the cells, and the discretion of the attending physician. The compositions and cells are in some embodiments suitably administered to the subject at one time or over a series of treatments. Exemplary dosage regimens and schedules for the provided combination therapy are described.

In some embodiments, the T cell therapy and the immunomodulatory compound are administered as part of a further combination treatment, which can be administered simultaneously with or sequentially to, in any order, another therapeutic intervention. In some contexts, the T cell therapy, e.g. engineered T cells, such as CAR-expressing T cells, are co-administered with another therapy sufficiently close in time such that the T cell therapy enhances the effect of one or more additional therapeutic agents, or vice versa. In some embodiments, the cells are administered prior to the one or more additional therapeutic agents. In some embodiments, the T cell therapy, e.g. engineered T cells, such as CAR-expressing T cells, are administered after the one or more additional therapeutic agents. In some embodiments, the combination therapy methods further include a lymphodepleting therapy, such as administration of a chemotherapeutic agent. In some embodiments, the combination therapy further comprises administering another therapeutic agent, such as an anti-cancer agent, a checkpoint inhibitor, or another immune modulating agent. Uses include uses of the combination therapies in such methods and treatments, and uses of such compositions in the preparation of a medicament in order to carry out such combination therapy methods. In some embodiments, the methods and uses thereby treat the disease or condition or disorder, such as a cancer or proliferative disease, in the subject.

Prior to, during or following administration of the immunotherapy (e.g. T cell therapy, such as CAR-T cell therapy) and/or an immunomodulatory compound, the biological activity of the T cell therapy, e.g. the biological activity of the engineered cell populations, in some embodiments is measured, e.g., by any of a number of known methods. Parameters to assess include the ability of the engineered cells to destroy target cells, persistence and other measures of T cell activity, such as measured using any suitable method known in the art, such as assays described further below in Section III. In some embodiments, the biological activity of the cells, e.g., T cells administered for the T cell based therapy, is measured by assaying cytotoxic cell killing, expression and/or secretion of one or more cytokines, proliferation or expansion, such as upon restimulation with antigen. In some aspects the biological activity is measured by assessing the disease burden and/or clinical outcome, such as reduction in tumor burden or load. In some embodiments, administration of one or both agents of the combination therapy and/or any repeated administration of the therapy, can be determined based on the results of the assays before, during, during the course of or after administration of one or both agents of the combination therapy.

In some embodiments, the combined effect of the immunomodulatory compound in combination with the cell therapy can be synergistic compared to treatments involving only the immunomodulatory compound or monotherapy with the cell therapy. For example, in some embodiments, the methods provided herein result in an increase or an improvement in a desired therapeutic effect, such as an increased or an improvement in the reduction or inhibition of one or more symptoms associated with cancer.

In some embodiments, the immunomodulatory compound increases the expansion or proliferation of the engineered T cells, such as CAR T-Cells. In some embodiments, the increase in expansion or proliferation is observed in vivo upon administration to a subject. In some embodiments, the increase in the number of engineered T cells, e.g. CAR-T cells, is increased by greater than or greater than about 1.2-fold, 1.5-fold, 2.0-fold, 3.0-fold, 4.0-fold, 5.0-fold, 6.0-fold, 7.0-fold, 8.0-fold, 9.0-fold, 10.0 fold or more.

A. Administration of T Cell Therapy

In some embodiments of the methods, compositions, combinations, kits and uses provided herein, the combination therapy includes administering to a subject an immune cell therapy, such as a T cell therapy (e.g. CAR-expressing T cells). In particular embodiments, the cell therapy is a T cell therapy directed against BCMA. For example, the T cell therapy is an anti-BCMA CAR T cell therapy. Administration of such therapies can be initiated prior to, subsequent to, simultaneously with administration of one or more immunomodulatory compound as described.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

In some embodiments, the cell-based therapy is or comprises administration of cells, such as immune cells, for example T cell or NK cells, that target a molecule expressed on the surface of a lesion, such as a tumor or a cancer. In some embodiments, the cells express a recombinant receptor, e.g. CAR, that contains an extracellular ligand-binding domain that specifically binds to an antigen. In some embodiments, the recombinant receptor is a CAR that contains an extracellular antigen-recognition domain that specifically binds to an antigen. In some embodiments, the recombinant receptor specifically binds to an antigen, such as one associated with a disease or condition, e.g. associated with or expressed on a cell of a tumor or cancer. In particular embodiments, the antigen is BCMA. In some embodiments, the immune cells express a recombinant receptor, such as a transgenic TCR or a chimeric antigen receptor (CAR). In some embodiments, the T cell therapy includes administering T cells engineered to express a chimeric antigen receptor (CAR). In particular embodiments, the cell therapy, e.g. anti-BCMA CAR T cell therapy, is for treating a multiple myeloma, such as a relapsed/refractory (R/R multiple myeloma). In some embodiments, the cells are autologous to the subject. In some embodiments, the cells are allogeneic to the subject. Exemplary engineered cells for administering as a cell therapy in the provided methods are described in Section II.

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4 vs CD8 T cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. Exemplary dosages for use or administration in accord with the provided methods are provided in Section I.A.2 below.

The cells can be administered by any suitable means. The cells are administered in a dosing regimen to achieve a therapeutic effect, such as a reduction in tumor burden. Dosing and administration may depend in part on the schedule of administration of the immunomodulatory compound, which can be administered prior to, subsequent to and/or simultaneously with initiation of administration of the T cell therapy. Various dosing schedules of the T cell therapy include but are not limited to single or multiple administrations over various time-points, bolus administration, and pulse infusion.

Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

1. Compositions and Formulations

In some embodiments, the dose of cells of the T cell therapy, such a T cell therapy comprising cells engineered with a recombinant antigen receptor, e.g. CAR or TCR, is provided as a composition or formulation, such as a pharmaceutical composition or formulation. Such compositions can be used in accord with the provided methods, such as in the prevention or treatment of diseases, conditions, and disorders, such as in the treatment of multiple myeloma, for example a relapsed or refractory multiple myeloma.

In some embodiments, the T cell therapy, such as engineered T cells (e.g. CAR T cells), are formulated with a pharmaceutically acceptable carrier. In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

In some cases, the cell therapy is administered as a single pharmaceutical composition comprising the cells. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

2. Dosage Schedule and Administration

In some embodiments, a dose of cells is administered to subjects in accord with the provided combination therapy methods. In some embodiments, the size or timing of the doses is determined as a function of the particular disease or condition in the subject. One may empirically determine the size or timing of the doses for a particular disease in view of the provided description.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the dose of cells is a flat dose of cells or fixed dose of cells such that the dose of cells is not tied to or based on the body surface area or weight of a subject.

In some embodiments, the cell therapy comprises administration of a dose comprising a number of cell from or from about 1×10⁵ to 2×10⁹ total recombinant receptor-expressing cells, total T cells, or total peripheral blood mononuclear cells (PBMCs), from or from about 5×10⁵ to 1×10⁹ total recombinant receptor-expressing cells, total T cells, or total peripheral blood mononuclear cells (PBMCs) or from or from about 1×10⁶ to 1×10⁹ total recombinant receptor-expressing cells, total T cells, or total peripheral blood mononuclear cells (PBMCs), each inclusive. In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸, at least or about at least 1×10⁹ of such cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 1.0×10⁷ to at or about 1.2×10⁹ such cells, such as at or about 1.0×10⁷, 1.5×10⁷, 2.0×10⁷, 2.5×10⁷, 5×10⁷, 1.5×10⁸, 3×10⁸, 4.5×10⁸, 6×10⁸, 8×10⁸ or 1.2×10⁹ total such cells, or the range between any two of the foregoing values. In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1.5×10⁸, 3×10⁸, 4.5×10⁸, 6×10⁸, 8×10⁸ or 1.2×10⁹ total such cells, or the range between any two of the foregoing values. In some embodiments, for example, where the subject is a human, the dose includes at or about 1.0×10⁷ total recombinant receptor (e.g., CAR)-expressing cells, T cells, or peripheral blood mononuclear cells (PBMCs). In some embodiments, for example, where the subject is a human, the dose includes at or about 1.5×10⁷ total recombinant receptor (e.g., CAR)-expressing cells, T cells, or peripheral blood mononuclear cells (PBMCs). In some embodiments, for example, where the subject is a human, the dose includes at or about 2.0×10⁷ total recombinant receptor (e.g., CAR)-expressing cells, T cells, or peripheral blood mononuclear cells (PBMCs). In some embodiments, for example, where the subject is a human, the dose includes at or about 2.5×10⁷ total recombinant receptor (e.g., CAR)-expressing cells, T cells, or peripheral blood mononuclear cells (PBMCs). In some embodiments, for example, where the subject is a human, the dose includes at or about 5×10⁷ total recombinant receptor (e.g., CAR)-expressing cells, T cells, or peripheral blood mononuclear cells (PBMCs). In some embodiments, for example, where the subject is a human, the dose includes at or about 1.5×10⁸ total recombinant receptor (e.g., CAR)-expressing cells, T cells, or peripheral blood mononuclear cells (PBMCs). In some embodiments, for example, where the subject is a human, the dose includes at or about 3×10⁸ total recombinant receptor (e.g., CAR)-expressing cells, T cells, or peripheral blood mononuclear cells (PBMCs). In some embodiments, for example, where the subject is a human, the dose includes at or about 4.5×10⁸ total recombinant receptor (e.g., CAR)-expressing cells, T cells, or peripheral blood mononuclear cells (PBMCs). In some embodiments, for example, where the subject is a human, the dose includes at or about 6×10⁸ total recombinant receptor (e.g., CAR)-expressing cells, T cells, or peripheral blood mononuclear cells (PBMCs). In some embodiments, for example, where the subject is a human, the dose includes at or about 8×10⁸ total recombinant receptor (e.g., CAR)-expressing cells, T cells, or peripheral blood mononuclear cells (PBMCs). In some embodiments, for example, where the subject is a human, the dose includes at or about 1.2×10⁹ total recombinant receptor (e.g., CAR)-expressing cells, T cells, or peripheral blood mononuclear cells (PBMCs).

In some embodiments, the dose of genetically engineered cells comprises from at or about 1×10⁵ to at or about 2×10⁹ total CAR-expressing (CAR+) T cells, from at or about 1×10⁵ to at or about 5×10⁸ total CAR-expressing T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells. In some embodiments, the dose of genetically engineered cells comprises from at or about 1.0×10⁷ to at or about 8×10⁸ total CAR-expressing (CAR+) T cells, from at or about 1.0×10⁷ to at or about 6.5×10⁸ total CAR+ T cells, from at or about 1.5×10⁷ to at or about 6.5×10⁸ total CAR+ T cells, from at or about 1.5×10⁷ to at or about 6.0×10⁸ total CAR+ T cells, from at or about 2.5×10⁷ to at or about 6.0×10⁸ total CAR+ T cells, or from at or about 5.0×10⁷ to at or about 6.0×10⁸ total CAR+ T cells.

In some embodiments, the dose of genetically engineered cells comprises between at or about 2.5×10⁷ CAR-expressing (CAR+) T cells, total T cells, or total peripheral blood mononuclear cells (PBMCs) and at or about 1.2×10⁹ CAR-expressing T cells, total T cells, or total PBMCs, between at or about 5.0×10⁷ CAR-expressing T cells, total T cells, or total peripheral blood mononuclear cells (PBMCs) and at or about 6.0×10⁸ CAR-expressing T cells, total T cells, or total PBMCs, between at or about 5.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 2.5×10⁷ to or to about 1.2×10⁹ CD3+ or CD8+ total T cells or CD3+ or CD8+ CAR-expressing cells, from or from about 5.0×10⁷ to or to about 6.0×10⁸ CD3+ or CD8+ total T cells or CD3+ or CD8⁺ CAR-expressing cells, from or from about 5.0×10⁷ to or to about 4.5×10⁸ CD3+ or CD8+ total T cells or CD3+ or CD8⁺ CAR-expressing cells, or from or from about 1.5×10⁸ to or to about 3.0×10⁸ CD3+ or CD8+ total T cells or CD3+ or CD8⁺ CAR-expressing cells, each inclusive.

In some embodiments, the dose of genetically engineered cells is with reference to the total number of CD3⁺ CAR-expressing (CAR+) or CD4+/CD8⁺ CAR-expressing (CAR+) cells. In some embodiments, the dose comprises a number of genetically engineered cells from or from about 1.0×10⁷ to or to about 1.2×10⁹ CD3+ or CD4+/CD8+ total T cells or CD3+ CAR-expressing or CD4+/CD8⁺ CAR-expressing cells, from or from about 1.5×10⁷ to or to about 1.2×10⁹ CD3+ or CD4+/CD8+ total T cells or CD3⁺ CAR-expressing or CD4+/CD8+ CAR-expressing cells, from or from about 2.0×10⁷ to or to about 1.2×10⁹ CD3+ or CD4+/CD8+ total T cells or CD3⁺ CAR-expressing or CD4+/CD8⁺ CAR-expressing cells, from or from about 2.5×10⁷ to or to about 1.2×10⁹ CD3+ or CD4+/CD8+ total T cells or CD3⁺ CAR-expressing or CD4+/CD8⁺ CAR-expressing cells, from or from about 5.0×10⁷ to or to about 6.0×10⁸ CD3+ or CD4+/CD8+ total T cells or CD3⁺ CAR-expressing or CD4+/CD8⁺ CAR-expressing cells, from or from about 5.0×10⁷ to or to about 4.5×10⁸ CD3+ or CD4+/CD8+ total T cells or CD3⁺ CAR-expressing or CD4+/CD8⁺ CAR-expressing cells, or from or from about 1.5×10° to or to about 3.0×10° CD3+ or CD4+/CD8+ total T cells or CD3⁺ CAR-expressing or CD4+/CD8⁺ CAR-expressing cells, each inclusive. In some embodiments, the dose comprises at or about 1.0×10⁷, 1.5×10⁷, 2.0×10⁷, 2.5×10⁷, 5×10⁷, 1.5×10⁸, 3×10⁸, 4.5×10⁸, 6×10⁸, 8×10⁸ or 1.2×10⁹ CD3+ or CD4+/CD8+ total T cells or CD3⁺ CAR-expressing or CD4+/CD8⁺ CAR-expressing cells. In some embodiments, the dose comprises at or about 2.5×10⁷, 5×10⁷, 1.5×10⁸, 3×10⁸, 4.5×10⁸, 6×10⁸, 8×10⁸ or 1.2×10⁹ CD3⁺ CAR-expressing cells. In some embodiments, the dose comprises at or about 1.0×10⁷, 1.5×10⁷, 2.0×10⁷, 2.5×10⁷, 5×10⁷, 1.5×10⁸, 3×10⁸, 4.5×10⁸, 6×10⁸, 8×10⁸ or 1.2×10⁹ CD4+/CD8⁺ CAR-expressing cells.

In some embodiments, the dose is at or about 1.0×10⁷ CD4+/CD8⁺ CAR-expressing cells. In some embodiments, the dose is at or about 1.5×10⁷ CD4+/CD8⁺ CAR-expressing cells. In some embodiments, the dose is at or about 2.0×10⁷ CD4+/CD8⁺ CAR-expressing cells. In some embodiments, the dose is at or about 2.5×10⁷ CD4+/CD8⁺ CAR-expressing cells. In some embodiments, the dose is at or about 5×10⁷ CD4+/CD8⁺ CAR-expressing cells. In some embodiments, the dose is at or about 1.5×10⁸ CD4+/CD8⁺ CAR-expressing cells. In some embodiments, the dose is at or about 3×10⁸ CD4+/CD8⁺ CAR-expressing cells. In some embodiments, the dose is at or about 4.5×10⁸ CD4+/CD8⁺ CAR-expressing cells. In some embodiments, the dose is at or about 6×10⁸ CD4+/CD8⁺ CAR-expressing cells. In some embodiments, the dose is at or about 8×10⁸ CD4+/CD8⁺ CAR-expressing cells. In some embodiments, the dose is at or about 1.2×10⁹ CD4+/CD8⁺ CAR-expressing cells. In some embodiments, the dose is at or about 2.5×10⁷ CD4+ or CD8⁺ CAR-expressing cells. In some embodiments, the dose is at or about 5×10⁷ CD4+ or CD8⁺ CAR-expressing cells. In some embodiments, the dose is at or about 1.5×10⁸ CD4+ or CD8⁺ CAR-expressing cells. In some embodiments, the dose is at or about 3×10⁸ CD4+ or CD8⁺ CAR-expressing cells. In some embodiments, the dose is at or about 4.5×10⁸ CD4+ or CD8⁺ CAR-expressing cells. In some embodiments, the dose is at or about 6×10⁸ CD4+ or CD8⁺ CAR-expressing cells. In some embodiments, the dose is at or about 6.5×10⁸ CD4+ or CD8⁺ CAR-expressing cells. In some embodiments, the dose is at or about 8×10⁸ CD4+ or CD8⁺ CAR-expressing cells. In some embodiments, the dose is at or about 1.2×10⁹ CD4+ or CD8⁺ CAR-expressing cells.

In some embodiments, the T cells of the dose include CD4+ T cells, CD8⁺ T cells or CD4+ and CD8⁺ T cells.

In some embodiments, for example, where the subject is human, the total of CD4⁺ T cells and CD8⁺ T cells of the dose includes between at or about 1×10⁶ and at or about 2×10⁹ total CAR-expressing CD4+ cells and CAR-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 1.0×10⁷, at or about 2.5×10⁷, at or about 2.0×10⁷, at or about 2.5×10⁷, at or about 5×10⁷, at or about 1.5×10⁸, at or about 3×10⁸, at or about 4.5×10⁸, at or about 6×10⁸, at or about 6.5×10⁸, at or about 8×10⁸, or at or about 1.2×10⁹ total such cells, or the range between any two of the foregoing values. In some embodiments, for example, where the subject is human, the CD8⁺ T cells of the dose, including in a dose including CD4+ T cells and CD8⁺ T cells, includes between at or about 1×10⁶ and at or about 2×10⁹ total recombinant receptor (e.g., CAR)-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 2.5×10⁷, at or about 5×10⁷, at or about 1.5×10⁸, at or about 3×10⁸, at or about 4.5×10⁸, at or about 6×10⁸, at or about 8×10⁸, or at or about 1.2×10⁹ total such cells, or the range between any two of the foregoing values.

In some embodiments, the dose of cells, e.g., recombinant receptor-expressing T cells, is administered to the subject as a single dose or is administered only one time within a period of two weeks, one month, three months, six months, 1 year or more. In some embodiments, the patient is administered multiple doses, and each of the doses or the total dose can be within any of the foregoing values. In some embodiments, the engineered cells for administration or composition of engineered cells for administration, exhibits properties indicative of or consistent with cell health. In some embodiments, at or about or at least at or about 70, 75, 80, 85, or 90% CAR+ cells of such dose exhibit one or more properties or phenotypes indicative of cell health or biologically active CAR cell, such as absence expression of an apoptotic marker.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death, including blebbing, cell shrinkage, nuclear fragmentation, chromatin condensation, chromosomal DNA fragmentation, and global mRNA decay. In some aspects, early stages of apoptosis can be indicated by activation of certain caspases, e.g., 2, 8, 9, and 10. In some aspects, middle to late stages of apoptosis are characterized by further loss of membrane integrity, chromatin condensation and DNA fragmentation, include biochemical events such as activation of caspases 3, 6, and 7.

In particular embodiments, the phenotype is negative expression of one or more factors associated with programmed cell death, for example pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In certain embodiments, the phenotype is the absence of an indicator, e.g., an Annexin V molecule or by TUNEL staining, that will preferentially bind to cells undergoing apoptosis when incubated with or contacted to a cell composition. In some embodiments, the phenotype is or includes the expression of one or more markers that are indicative of an apoptotic state in the cell. In some embodiments, the phenotype is lack of expression and/or activation of a caspase, such as caspase 3. In some aspects, activation of caspase-3 is indicative of an increase or revival of apoptosis. In certain embodiments, caspase activation can be detected by known methods. In some embodiments, an antibody that binds specifically to an activated caspase (i.e., binds specifically to the cleaved polypeptide) can be used to detect caspase activation. In particular embodiments, the phenotype is or includes active caspase 3−. In some embodiments, the marker of apoptosis is a reagent that detects a feature in a cell that is associated with apoptosis. In certain embodiments, the reagent is an annexin V molecule.

In some embodiments, the compositions containing the engineered cells for administration contain a certain number or amount of cells that exhibit phenotypes indicative of or consistent with cell health. In some of any embodiments, less than about 25%, 20%, 15%, 10%, 9%, 8%, 7%, 6%, 5%, 4%, 3%, 2% or 1% of the CAR-expressing T cells in the dose of engineered T cells express a marker of apoptosis, optionally Annexin V or active Caspase 3. In some of any embodiments, less than 5%, 4%, 3%, 2% or 1% of the CAR-expressing T cells in the dose of engineered T cells express Annexin V or active Caspase 3.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days.

Thus, the dose of cells may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, the dose or composition of cells includes a defined or target ratio of CD4+ cells expressing a recombinant receptor to CD8+ cells expressing a recombinant receptor and/or of CD4+ cells to CD8+ cells that is approximately 1:1 or is between approximately 1:3 and approximately 3:1, such as approximately 1:1.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, administration of the immunomodulatory compound in combination with the cells is able to significantly increase the expansion or proliferation of the cells, and thus a lower dose of cells can be administered to the subject. In some cases, the provided methods allow a lower dose of such cells to be administered, to achieve the same or better efficacy of treatment as the dose in a method in which the cell therapy is administered without administering the immunomodulatory compound, such as at least 1.5-fold, 2-fold, 3-fold, 4-fold, 5-fold or 10-fold less than the dose in a method in which the cell therapy is administered without administering the immunomodulatory compound, e.g., Compound A or Compound B.

In some embodiments, for example, the dose contains between or between about 5.0×10⁶ and 2.25×10⁷, 5.0×10⁶ and 2.0×10⁷, 5.0×10⁶ and 1.5×10⁷, 5.0×10⁶ and 1.0×10⁷, 5.0×10⁶ and 7.5×10⁶, 7.5×10⁶ and 2.25×10⁷, 7.5×10⁶ and 2.0×10⁷, 7.5×10⁶ and 1.5×10⁷, 7.5×10⁶ and 1.0×10⁷, 1.0×10⁷ and 2.25×10⁷, 1.0×10⁷ and 2.0×10⁷, 1.0×10⁷ and 1.5×10⁷, 1.5×10⁷ and 2.25×10⁷, 1.5×10⁷ and 2.0×10⁷, 2.0×10⁷ and 2.25×10⁷. In some embodiments, the dose of cells contains a number of cells, that is between at least or at least about 5×10⁶, 6×10⁶, 7×10⁶, 8×10⁶, 9×10⁶, 10×10⁶ and about 15×10⁶ recombinant-receptor expressing cells, such as recombinant-receptor expressing cells that are CD8+. In some embodiments, such dose, such as such target number of cells refers to the total recombinant-receptor expressing cells in the administered composition.

In some embodiments, for example, the lower dose contains less than about 5×10⁶ cells, recombinant receptor (e.g. CAR)-expressing cells, T cells, and/or PBMCs per kilogram body weight of the subject, such as less than about 4.5×10⁶, 4×10⁶, 3.5×10⁶, 3×10⁶, 2.5×10⁶, 2×10⁶, 1.5×10⁶, 1×10⁶, 5×10⁵, 2.5×10⁵, or 1×10⁵ such cells per kilogram body weight of the subject. In some embodiments, the lower dose contains less than about 1×10⁵, 2×10⁵, 5×10⁵, or 1×10⁶ of such cells per kilogram body weight of the subject, or a value within the range between any two of the foregoing values. In some embodiments, such values refer to numbers of recombinant receptor-expressing cells; in other embodiments, they refer to number of T cells or PBMCs or total cells administered.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses. In some embodiments, one or more subsequent dose of cells can be administered to the subject. In some embodiments, the subsequent dose of cells is administered greater than or greater than about 7 days, 14 days, 21 days, 28 days or 35 days after initiation of administration of the first dose of cells. The subsequent dose of cells can be more than, approximately the same as, or less than the first dose. In some embodiments, administration of the T cell therapy, such as administration of the first and/or second dose of cells, can be repeated.

In some embodiments, initiation of administration of the cell therapy, e.g. the dose of cells or a first dose of a split dose of cells, is administered before (prior to), concurrently with or after (subsequently or subsequent to) the administration of the immunomodulatory compound, e.g., Compound A or Compound B.

In some embodiments, the dose of cells, or the subsequent dose of cells, is administered concurrently with initiating administration of the immunomodulatory compound in accord with the combination therapy methods. In some embodiments, the dose of cells, or the subsequent dose of cells, is administered on the same day as initiating administration of the immunomodulatory compound in accord with the combination therapy methods. In some embodiments, the dose of cells, or the subsequent dose of cells, is administered within 1 day, within 2 days, within 3 days, within 4 days, within 5 days, within 6 days, or within 7 days of initiating administration of the immunomodulatory compound in accord with the combination therapy methods.

In some embodiments, the dose of cells, or the subsequent dose of cells, is administered prior to starting or initiating administration of the immunomodulatory compound in accord with the provided combination therapy. In some embodiments, the dose of cells is administered at least or at least about 1 hour, at least or at least about 2 hours, at least or at least about 3 hours, at least or at least about 6 hours, at least or at least about 12 hours, at least or at least about 1 day, at least or at least about 2 days, at least or at least about 3 days, at least or about at least 4 days, at least or at least about 5 days, at least or about at least 6 days, at least or at least about 7 days, at least or about at least 12 days, at least or at least about 14 days, at least or about at least 15 days, at least or at least about 21 days, at least or at least about 28 days, at least or about at least 30 days, at least or at least about 35 days, at least or at least about 42 days, at least or about at least 60 days or at least or about at least 90 days prior to administering the immunomodulatory compound in accord with the provided combination therapy.

In some embodiments, the administration of the immunomodulatory compound (e.g., Compound A or Compound B) in accord with the provided combination therapy is at a time in which the prior administration of the immunotherapy (e.g., T cell therapy, such as CAR-T cell therapy) is associated with, or is likely to be associated with, a decreased functionality of the T cells compared to the functionality of the T cells at a time just prior to initiation of the immunotherapy (e.g., T cell therapy, such as CAR-T cell therapy) or at a preceding time point after initiation of the T cell therapy. In some embodiments, the method involves, subsequent to administering the dose of cells of the T cell therapy, e.g., adoptive T cell therapy, but prior to administering the immunomodulatory compound, assessing a sample from the subject for one or more function of T cells, such as expansion or persistence of the cells, e.g. as determined by level or amount in the blood, or other phenotypes or desired outcomes as described herein, e.g., such as those described in Section III. In some embodiments, the method involves, subsequent to administering the dose of cells of the T cell therapy, e.g., adoptive T cell therapy, but prior to administering the immunomodulatory compound, assessing a sample from the subject for expression of one or more exhaustion markers. Various parameters for determining or assessing the regimen of the combination therapy are described in Section III.

B. Administration of the Immunomodulatory Compound

The provided combination therapy methods, compositions, combinations, kits and uses involve administration of an immunomodulatory compound, such as a structural or functional analog or derivative of thalidomide and/or an inhibitor of E3 ubiquitin ligase, e.g. Compound A (iberdomide, (S)-3-[4-(4-morpholin-4-ylmethyl-benzyloxy)-1-oxo-1,3-dihydro-isoindol-2-yl]-piperidine-2,6-dione or Compound B ((S)-4-(4-(4-(((2-(2,6-dioxopiperidin-3-yl)-1-oxoisoindolin-4-yl)oxy)methyl)benzyl)piperazin-1-yl)-3-fluorobenzonitrile), which can be administered prior to, subsequently to, during, simultaneously or near simultaneously, sequentially and/or intermittently with administration of the T cell therapy, e.g., administration of T cells expressing a chimeric antigen receptor (CAR).

1. Compositions and Formulations

In some embodiments of the combination therapy methods, compositions, combinations, kits and uses provided herein, the combination therapy can be administered in one or more compositions, e.g., a pharmaceutical composition containing an immunomodulatory compound, e.g., Compound A or Compound B.

In some embodiments, the composition, e.g., a pharmaceutical composition containing the immunomodulatory compound, e.g., Compound A or Compound B, can include carriers such as a diluent, adjuvant, excipient, or vehicle with which the immunomodulatory compound, e.g., Compound A or Compound B, and/or the cells are administered. Examples of suitable pharmaceutical carriers are described in “Remington's Pharmaceutical Sciences” by E. W. Martin. Such compositions will contain a therapeutically effective amount of the immunomodulatory compound, e.g. Compound A or Compound B, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular inhibitor and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. The compositions containing the immunomodulatory compound, e.g., Compound A or Compound B can also be lyophilized.

In some embodiments, the pharmaceutical compositions can be formulated for administration by any known route including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjunctival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration. In some embodiments, it is administered by multiple bolus administrations, for example, over a period of no more than 3 days, or by continuous infusion administration.

In some embodiments, the administration can be local, topical or systemic depending upon the locus of treatment. In some embodiments local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. In some embodiments, compositions also can be administered with other biologically active agents, either sequentially, intermittently or in the same composition. In some embodiments, administration also can include controlled release systems including controlled release formulations and device controlled release, such as by means of a pump. In some embodiments, the administration is oral.

In some embodiments, pharmaceutically and therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition containing the immunomodulatory compound, e.g., Compound A or Compound B, is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition containing the immunomodulatory compound, e.g., Compound A or Compound B, in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The compositions containing the immunomodulatory compound, e.g., Compound A or Compound B, can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, the composition containing the immunomodulatory compound, e.g., Compound A or Compound B, are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

2. Immunomodulatory Compound Dosage Schedule

In some embodiments, the provided combination therapy method involves administering to the subject a therapeutically effective amount of an immunomodulatory drug (immunomodulatory compound), e.g., Compound A or Compound B, and the cell therapy, such as a T cell therapy (e.g. CAR-expressing T cells).

In some embodiments, the administration of the immunomodulatory compound, e.g., Compound A or Compound B, is initiated prior to, subsequently to, during, during the course of, simultaneously, near simultaneously, sequentially and/or intermittently with the administration of the cell therapy, such as a T cell therapy (e.g. CAR-expressing T cells). In some embodiments, the method involves initiating the administration of the immunomodulatory compound, e.g., Compound A or Compound B prior to administration of the T cell therapy. In other embodiments, the method involves initiating the administration of the immunomodulatory compound, e.g., Compound A or Compound B, after administration of the T cell therapy. In some embodiments, the dosage schedule comprises initiating the administration of the immunomodulatory compound, e.g., Compound A or Compound B, concurrently or simultaneously with the administration of the T cell therapy.

In some embodiments, the immunomodulatory compound, e.g., Compound A or Compound B, is administered in a cycle. In some embodiments, the cycle comprises an administration period in which the immunomodulatory compound, e.g., Compound A or Compound B, is administered followed by a rest period during which the immunomodulatory compound, e.g., Compound A or Compound B, is not administered. In some embodiments, the total number of days of the cycle, e.g. from the beginning of initiating administration of the immunomodulatory compound, is greater than or greater than about or is about 21 days, 28 days, 30 days, 40 days, 50 days, 60 days or more.

In some embodiments, the initiation of the administration of the immunomodulatory compound, e.g., Compound A or Compound B, is carried out in at least one cycle and initiation of administration of the T cell therapy are carried out on the same day, optionally concurrently. In some embodiments, the initiation of the administration of the immunomodulatory compound, e.g., Compound A or Compound B, in at least one cycle is prior to initiation of administration of the T cell therapy. In some embodiments, the initiation of the administration of the immunomodulatory compound, e.g., Compound A or Compound B, in at least one cycle is concurrent with or on the same day as initiation of administration of the T cell therapy. In some embodiments, the immunomodulatory compound, e.g., Compound A or Compound B, is administered from or from about 0 to 30 days, such as 0 to 15 days, 0 to 6 days, 0 to 96 hours, 0 to 24 hours, 0 to 12 hours, 0 to 6 hours, or 0 to 2 hours, 2 hours to 15 days, 2 hours to 6 days, 2 hours to 96 hours, 2 hours to 24 hours, 2 hours to 12 hours, 2 hours to 6 hours, 6 hours to 30 days, 6 hours to 15 days, 6 hours to 6 days, 6 hours to 96 hours, 6 hours to 24 hours, 6 hours to 12 hours, 12 hours to 30 days, 12 hours to 15 days, 12 hours to 6 days, 12 hours to 96 hours, 12 hours to 24 hours, 24 hours to 30 days, 24 hours to 15 days, 24 hours to 6 days, 24 hours to 96 hours, 96 hours to 30 days, 96 hours to 15 days, 96 hours to 6 days, 6 days to 30 days, 6 days to 15 days, or 15 days to 30 days prior to initiation of the T cell therapy. In some aspects, the immunomodulatory compound, e.g., Compound A or Compound B, is administered no more than about 96 hours, 72 hours, 48 hours, 24 hours, 12 hours, 6 hours, 2 hours or 1 hour prior to initiation of the T cell therapy.

In some of any such embodiments in which the immunomodulatory compound, e.g., Compound A or Compound B, is given prior to the cell therapy (e.g. T cell therapy, such as CAR-T cell therapy), the administration of the immunomodulatory compound, e.g., Compound A or Compound B, continues at regular intervals until the initiation of the cell therapy and/or for a time after the initiation of the cell therapy.

In some embodiments, the immunomodulatory compound, e.g., Compound A or Compound B, is administered, or is further administered, after administration of the cell therapy (e.g. T cell therapy, such as CAR-T cell therapy). In some embodiments, the immunomodulatory compound, e.g., Compound A or Compound B, is administered within or within about 1 hours, 2 hours, 6 hours, 12 hours, 24 hours, 48 hours, 96 hours, 4 days, 5 days, 6 days or 7 days, 14 days, 15 days, 21 days, 24 days, 28 days, 30 days, 36 days, 42 days, 60 days, 72 days or 90 days after initiation of administration of the cell therapy (e.g. T cell therapy). In some embodiments, the provided methods involve continued administration, such as at regular intervals, of the immunomodulatory compound after initiation of administration of the cell therapy.

In some embodiments, the immunomodulatory compound, e.g., Compound A or Compound B, is administered up to or up to about 1 day, up to or up to about 2 days, up to or up to about 3 days, up to or up to about 4 days, up to or up to about 5 days, up to or up to about 6 days, up to or up to about 7 days, up to or up to about 12 days, up to or up to about 14 days, up to or up to about 21 days, up to or up to about 24 days, up to or up to about 28 days, up to or up to about 30 days, up to or up to about 35 days, up to or up to about 42 days, up to or up to about 60 days or up to or up to about 90 days, up to or up to about 120 days, up to or up to about 180 days, up to or up to about 240 days, up to or up about 360 days, or up to or up to about 720 days or more after the initiation of administration of the cell therapy (e.g. T cell therapy, such as CAR-T cell therapy).

In some of any such above embodiments, the immunomodulatory compound, e.g., Compound A or Compound B, is administered prior to and after initiation of administration of the cell therapy (e.g. T cell therapy, such as CAR-T cell therapy).

In some embodiments, the initiation of the administration of the immunomodulatory compound, e.g., Compound A or Compound B, is carried out at or after, optionally immediately after or within 1 to 3 days after: (i) peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the T cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (iv) the subject exhibits increased tumor burden as compared to tumor burden at a time prior to or after administration of the T cells and prior to initiation of administration of the immunomodulatory compound.

In some embodiments, the initiation of the administration of the immunomodulatory compound, e.g., Compound A or Compound B, in at least one cycle is after initiation of administration of the T cell therapy. In some embodiments, the initiation of the administration of the immunomodulatory compound, e.g., Compound A or Compound B, is at least or about at least 1 day, at least or about at least 2 days, at least or about at least 3 days, at least or about at least 4 days, at least or about at least 5 days, at least or about at least 6 days, at least or about at least 7 days, at least or about at least 8 days, at least or about at least 9 days, at least or about at least 10 days, at least or at least about 12 days, at least or about at least 14 days, at least or at least about 15 days, at least or about at least 21 days, at least or at least about 24 days, at least or about at least 28 days, at least or about at least 30 days, at least or about at least 35 days or at least or about at least 42 days, at least or about at least 60 days, or at least or about at least 90 days after initiation of the administration of the T cell therapy. In some embodiments, the initiation of the administration of the immunomodulatory compound, e.g., Compound A or Compound B, is carried out at least 2 days after, at least 1 week after, at least 2 weeks after, at least 3 weeks after, or at least 4 weeks after, the initiation of the administration of the T cell therapy. In some embodiments, the initiation of the administration of the immunomodulatory compound, e.g., Compound A or Compound B, is carried out 2 to 28 days or 7 to 21 days after initiation of administration of the T cell therapy. In some embodiments, the initiation of the administration of the immunomodulatory compound, e.g., Compound A or Compound B, is carried out at a time that is greater than or greater than about 14 days, 15 days, 16 days, 17 days, 18 days, 19, days, 20 days, 21 days, 24 days, or 28 days after initiation of the administration of the T cell therapy. In some embodiments, the immunomodulatory compound, e.g., Compound A or Compound B, is administered several times a day, twice a day, daily, every other day, three times a week, twice a week, or once a week after initiation of the cell therapy. In some embodiments, the immunomodulatory compound, e.g., Compound A or Compound B, is administered daily. In some embodiments the immunomodulatory compound, e.g., Compound A or Compound B, is administered twice a day. In some embodiments, the immunomodulatory compound, e.g., Compound A or Compound B, is administered three times a day. In other embodiments, the immunomodulatory compound, e.g., Compound A or Compound B, is administered every other day. In some embodiments, the immunomodulatory compound, e.g., Compound A or Compound B, is administered daily. In some embodiments, the immunomodulatory compound, e.g., Compound A or Compound B, is administered during the administration period for a plurality of consecutive days, such as for up to about 7, 8, 9, 10, 11, 12, 13, 14, 15, 16, 17, 18, 19, 20, 21, 22, 23, 24, 25, 26, 27, 28, 29, 30, or more than 30 consecutive days. In some embodiments, the immunomodulatory compound, e.g., Compound A or Compound B, is administered for greater than or greater than about 7 consecutive days, greater than or greater than about 14 consecutive days, greater than or greater than about 21 consecutive days, greater than or greater than about 21 consecutive days, or greater than or greater than about 28 consecutive days. In some embodiments, the immunomodulatory compound, e.g., Compound A or Compound B, is administered during the administration period for up to 21 consecutive days. In some embodiments, the immunomodulatory compound, e.g., Compound A or Compound B, is administered during the administration period for up to 21 consecutive days, wherein the cycle comprises greater than 30 days beginning upon initiation of the administration of the immunomodulatory compound.

In some embodiments, the immunomodulatory compound, e.g., Compound A or Compound B, is administered during the administration period for no more than about 7, 8, 9, 10, 11, 12, 13, 14, 15, 16, 17, 18, 19, 20, 21, 22, 23, 24, 25, 26, 27, 28, 29, 30, or no more than 30 consecutive days. In certain embodiments, the immunomodulatory compound, e.g., Compound A or Compound B, is administered once daily for 14 days over a 21 day treatment cycle. In certain embodiments, the immunomodulatory compound, e.g., Compound A or Compound B, is administered once daily for 21 days over a 28 day treatment cycle. In some embodiments, the immunomodulatory compound, e.g., Compound A or Compound B, is administered during the administration period for no more than 14 consecutive days.

In some embodiments, the immunomodulatory compound, e.g., Compound A or Compound B, is administered in a cycle, wherein the cycle comprises the administration of the immunomodulatory compound, e.g., Compound A or Compound B, for a plurality of consecutive days followed by a rest period during which the immunomodulatory compound is not administered. In some embodiments, the rest period is greater than about 1 day, greater than about 3 consecutive days, greater than about 5 consecutive days, greater than about 7 consecutive days, greater than about 8 consecutive days, greater than about 9 consecutive days, greater than about 10 consecutive days, greater than about 11 consecutive days, greater than about 12 consecutive days, greater than about 13 consecutive days, greater than about 14 consecutive days, greater than about 15 consecutive days, greater than about 16 consecutive days, greater than about 17 consecutive days, greater than about 18 consecutive days, greater than about 19 consecutive days, greater than about 20 consecutive days, or greater than about 21 or more consecutive days. In some embodiments, the rest period is greater than 7 consecutive days, greater than 14 consecutive days, greater than 21 days, or greater than 28 days. In some embodiments, the rest period is greater than about 14 consecutive days. In some embodiments, the cycle of administration of the immunomodulatory compound does not contain a rest period.

In some embodiments, the immunomodulatory compound, e.g., Compound A or Compound B, is administered in a cycle, wherein the cycle is repeated at least one time. In some embodiments, the immunomodulatory compound, e.g., Compound A or Compound B, is administered for at least 2 cycles, at least 3 cycles, at least 4 cycles, at least 5 cycles, at least 6 cycles, at least 7 cycles, at least 8 cycles, at least 9 cycles, at least 10 cycles, at least 11 cycles, or at least 12 cycles. In some embodiments, the immunomodulatory compound, e.g., Compound A or Compound B, is administered for 1, 2, 3, 4, 5, 6, 7, 8, 9, 10, 11, 12, 13, 14, 15, 16, 17, 18, 19, 20, 21, 22, 23, or 24 cycles.

In some embodiments, the immunomodulatory compound, e.g., Compound A or Compound B, is administered six times daily, five times daily, four times daily, three times daily, twice daily, once daily, every other day, every three days, twice weekly, once weekly or only one time prior to or subsequently to initiation of administration of the T cell therapy. In some embodiments, the immunomodulatory compound, e.g., Compound A or Compound B, is administered in multiple doses in regular intervals prior to, during, during the course of, and/or after the period of administration of the T cell therapy. In some embodiments, the immunomodulatory compound, e.g., Compound A or Compound B, is administered in one or more doses in regular intervals prior to the administration of the T cell therapy. In some embodiments, the immunomodulatory compound, e.g., Compound A or Compound B, is administered in one or more doses in regular intervals after the administration of the T cell therapy. In some embodiments, one or more of the doses of the immunomodulatory compound, e.g., Compound A or Compound B, can occur simultaneously with the administration of a dose of the T cell therapy.

In some embodiments, the dose, frequency, duration, timing and/or order of administration of the immunomodulatory compound, e.g., Compound A or Compound B, is determined, based on particular thresholds or criteria of results of the screening step and/or assessment of treatment outcomes described herein, e.g., those described in Section III herein.

In some embodiments, the method involves administering the cell therapy to a subject that has been previously administered a therapeutically effective amount of the immunomodulatory compound. In some embodiments, the immunomodulatory compound is administered to a subject before administering a dose of cells expressing a recombinant receptor to the subject. In some embodiments, the treatment with the immunomodulatory compound occurs at the same time as the administration of the dose of cells. In some embodiments, the immunomodulatory compound is administered after the administration of the dose of cells.

In some embodiments, the immunomodulatory compound, e.g., Compound A or Compound B, is administered daily for 7, 8, 9, 10, 11, 12, 13, 14, 15, 16, 17, 18, 19, 20, 21, or more than 21 days. In some embodiments, the immunomodulatory compound, e.g., Compound A or Compound B, is administered twice a day for 7, 8, 9, 10, 11, 12, 13, 14, 15, 16, 17, 18, 19, 20, 21, or more than 21 days. In some embodiments, the immunomodulatory compound, e.g., Compound A or Compound B, is administered three times a day for 7, 8, 9, 10, 11, 12, 13, 14, 15, 16, 17, 18, 19, 20, 21, or more than 21 days. In some embodiments, the immunomodulatory compound, e.g., Compound A or Compound B, is administered every other day for 7, 8, 9, 10, 11, 12, 13, 14, 15, 16, 17, 18, 19, 20, 21, or more than 21 days.

In some embodiments of the methods provided herein, the immunomodulatory compound, e.g., Compound A or Compound B, and the T cell therapy are administered simultaneously or near simultaneously.

In some embodiments, immunomodulatory compound, e.g. Compound A or Compound B, is administered at a dose of from or from about 0.1 mg to about 100 mg, from or from about 0.1 mg to 50 mg, from or from about 0.1 mg to 25 mg, from or from about 0.1 mg to 10 mg, from or from about 0.1 mg to 5 mg, from or from about 0.1 mg to 1 mg, from or from about 1 mg to 100 mg, from or from about 1 mg to 50 mg, from or from about 1 mg to 25 mg, from or from about 1 mg to 10 mg, from or from about 1 mg to 5 mg, from or from about 5 mg to 100 mg, from or from about 5 mg to 50 mg, from or from about 5 mg to 25 mg, from or from about 5 mg to 10 mg, from or from about 10 mg to 100 mg, from or from about 10 mg to 50 mg, from or from 10 mg to 25 mg, from or from about 25 mg to 100 mg, from or from about 25 mg to 50 mg or from or from about 50 mg to 100 mg, each inclusive. In some embodiments, the amount is a once daily amount of the immunomodulatory compound, e.g. Compound A or Compound B.

In some embodiments, the immunomodulatory compound, e.g. Compound A or Compound B, is administered at a dosage of from about 1 mg to about 20 mg, e.g., from about 1 mg to about 10 mg, from about 2.5 mg to about 7.5 mg, from about 5 mg to about 15 mg, such as about 5 mg, 10 mg, 15 mg or 20 mg. In some embodiments, the immunomodulatory compound, e.g. Compound A or Compound B is administered at a dose of from about 10 μg/kg to 5 mg/kg, e.g., about 100 μg/kg to about 2 mg/kg, about 200 μg/kg to about 1 mg/kg, about 400 μg/kg to about 600 μg/kg, such as about 500 μg/kg. In some embodiments, the amount is a once daily amount of the immunomodulatory compound, e.g. Compound A or Compound B. In some embodiments, the immunomodulatory compound is Compound A. In some embodiments, the immunomodulatory compound is Compound B. In some embodiments, the immunomodulatory compound, e.g., Compound A or Compound B, is administered at a total daily dosage amount of at least or at least about 0.1 mg per day, 0.5 mg per day, 1.0 mg per day, 2.5 mg per day, 5 mg per day, 10 mg per day, 25 mg per day, 50 mg per day or 100 mg per day. In some embodiments, the dose of the immunomodulatory compound, e.g. Compound A or Compound B is or is about 25 mg per day. In particular embodiments, the dose of the immunomodulatory compound, e.g. Compound A or Compound B is or is about 10 mg per day. In some embodiments, the immunomodulatory compound is Compound A. In some embodiments, the immunomodulatory compound is Compound B.

In some embodiments, the immunomodulatory compound, e.g. Compound A or Compound B, is administered in an amount greater than or greater than about 1 mg, 2.5 mg, 5 mg, 7.5 mg, 10 mg, 15 mg and less than 25 mg. In some embodiments, the immunomodulatory compound, e.g., Compound A or Compound B, is administered in an amount greater than or greater than about 1 mg per day, 2.5 mg per day, 5 mg per day, 7.5 mg per day, 10 mg per day, 15 mg per day and less than 25 mg per day. In some embodiments, the immunomodulatory compound is Compound A. In some embodiments, the immunomodulatory compound is Compound B.

In some embodiments, the provided methods include administering an effective amount of Compound A per day to a subject to modulate activity and/or function of the T cell therapy. In some embodiments, Compound A is administered at a dosage of from or from about 0.1 mg to at or about 1 mg. In some embodiments, the amount is at or about 0.1 mg, at or about 0.2 mg, at or about 0.3 mg, at or about 0.4 mg, at or about 0.5 mg, at or about 0.6 mg, at or about 0.7 mg, at or about 0.8 mg, at or about 0.9 mg or at or about 1.0 mg, or any value between any of the foregoing. In some embodiments, the amount of Compound A is administered in a cycling regimen involving daily administration for three weeks in a four week period or cycle. The administration of Compound A is carried out for a period of time, such as generally for more than one week, such as for at or greater than one month, at or greater than two months, at or greater than three months, at or greater than four months, at or greater than five months or at or greater than six months. Exemplary dosing regimens are described herein.

In some aspects, the provided methods minimize or avoid toxicity following administration of the T cell therapy and/or immunomodulatory compound, e.g. Compound A or Compound B, to a subject. In some aspects, the methods provided herein involve administering doses that are substantially lower than doses identified to be the MTD for the compound.

In some of any of the embodiments, the methods and uses include administration of Compound A or Compound B. In some embodiments, the administration of Compound A or Compound B is initiated after (subsequent to) the initiation of the cell therapy, such as a T cell therapy (e.g., CAR-expressing T cells). In some embodiments, administration of Compound A or Compound B is initiated at or after peak or maximum level of the cells of the T cell therapy is detectable in the blood of the subject. In some cases, initiation of administration Compound A or Compound B is carried out at or within a week, such as within 1, 2 or 3 days after (i) a time in which peak or maximum level of the cells of the T cell therapy are detectable in the blood of the subject; (ii) the number of cells of the T cell therapy detectable in the blood, after having been detectable in the blood, is not detectable or is reduced, optionally reduced compared to a preceding time point after administration of the T cell therapy; (iii) the number of cells of the T cell therapy detectable in the blood is decreased by or more than 1.5-fold, 2.0-fold, 3.0-fold, 4.0-fold, 5.0-fold, 10-fold or more the peak or maximum number cells of the T cell therapy detectable in the blood of the subject after initiation of administration of the T cell therapy; (iv) at a time after a peak or maximum level of the cells of the T cell therapy are detectable in the blood of the subject, the number of cells of or derived from the cells detectable in the blood from the subject is less than less than 10%, less than 5%, less than 1% or less than 0.1% of total peripheral blood mononuclear cells (PBMCs) in the blood of the subject; (v) the subject exhibits disease progression and/or has relapsed following remission after treatment with the T cell therapy; and/or (iv) the subject exhibits increased tumor burden as compared to tumor burden at a time prior to or after administration of the cells and prior to initiation of administration of Compound A or Compound B. In certain aspects, the provided methods are carried out to enhance, increase or potentiate T cell therapy in subjects in which a peak response to the T cell therapy has been observed but in which the response, e.g. presence of T cells and/or reduction in tumor burden, has become reduced or is no longer detectable.

In some embodiments, the administration of Compound A or Compound B is initiated at or about 14 to about 35 days after initiation of administration of the T cell therapy. In some embodiments, the administration of Compound A or Compound B is initiated about 21 to about 35 days after initiation of administration of the T cell therapy. In some embodiments, the administration of Compound A or Compound B is initiated about 21 to about 28 days after initiation of administration of the T cell therapy. In some embodiments, the administration of Compound A or Compound B is initiated at or about 14 days, at or about 15 days, at or about 16 days, at or about 17 days, at or about 18 days, at or about 19 days, at or about 20 days, at or about 21 days, at or about 22 days, at or about 23 days, at or about 24 days, at or about 25 days, at or about 26 days, at or about 27 days, at or about 28 days, at or about 29 days, at or about 30 days, at or about 31 days, at or about 32 days, at or about 33 days, at or about 34 days, or at or about 35 days after initiation of administration of the T cell therapy.

In some embodiments, at the time at which the subject is first administered Compound A or Compound B and/or at any subsequent time after initiation of the administration, the subject does not exhibit a sign or symptom of a severe toxicity, such as severe cytokine release syndrome (CRS) or severe toxicity. In some embodiments, the administration of Compound A or Compound B is at a time at which the subject does not exhibit a sign or symptom of severe CRS and/or does not exhibit grade 3 or higher CRS, such as prolonged grade 3 CRS or grade 4 or 5 CRS. In some embodiments, the administration of Compound A or Compound B is at a time at which the subject does not exhibit a sign or symptom of severe neurotoxicity and/or does not exhibit grade 3 or higher neurotoxicity, such as prolonged grade 3 neurotoxicity or grade 4 or grade 5 neurotoxicity. In some aspects, between the time of the initiation of the administration of the T cell therapy and the time of the administration of Compound A or Compound B, the subject has not exhibited severe CRS and/or has not exhibited grade 3 or higher CRS, such as prolonged grade 3 CRS or grade 4 or 5 CRS. In some instances, between the time of the initiation of the administration of the T cell therapy and the time of the administration of Compound A or Compound B, the subject has not exhibited severe neurotoxicity and/or does not exhibit grade 3 or higher neurotoxicity, such as prolonged grade 3 neurotoxicity or grade 4 or 5 neurotoxicity.

In some embodiments, administration of Compound A or Compound B per day it is administered is at an amount of from or from about 0.1 mg to 5 mg. In some embodiments, administration of Compound A or Compound B per day it is administered is at an amount of about 0.1 mg to about 5 mg, about 0.5 mg to about 5 mg, about 1 mg to about 5 mg, about 1.5 mg to about 5 mg, about 2 mg to about 5 mg, about 2.5 mg to about 5 mg, about 3 mg to about 5 mg, about 0.1 mg to about 4 mg, about 0.1 mg to about 4 mg, about 1 mg to about 4 mg, about 1.5 mg to about 4 mg, about 2 mg to about 4 mg, about 2.5 mg to about 4 mg, about 3 mg to about 4 mg, about 0.1 mg to about 3.5 mg, about 0.5 mg to about 3.5 mg, about 1 mg to about 3.5 mg, about 1.5 mg to about 3.5 mg, about 2 mg to about 3.5 mg, about 2.5 mg to about 3.5 mg, about 3 mg to about 3.5 mg, about 0.1 mg to about 3 mg, about 0.5 mg to about 3 mg, about 1 mg to about 3 mg, about 1.5 mg to about 3 mg, about 2 mg to about 3 mg, about 2.5 mg to about 3 mg, about 0.1 mg to about 2.5 mg, about 0.5 mg to about 2.5 mg, about 1 mg to about 2.5 mg, about 1.5 mg to about 2.5 mg, about 2 mg to about 2.5 mg, about 0.1 mg to about 2 mg, about 0.5 mg to about 2 mg, about 1 mg to about 2 mg, about 1.5 mg to about 2 mg, about 0.1 mg to about 1.5 mg, about 0.5 mg to about 1.5 mg, about 1 mg to about 1.5 mg, about 0.1 mg to about 1 mg, or about 0.5 mg to about 1 mg.

In some embodiments, administration of Compound A or Compound B per day it is administered is at an amount of about or at least about, or at or at least at 0.5 mg. In some embodiments, administration of Compound A or Compound B per day it is administered is at an amount of about or at least about, or at or at least at 1 mg. In some embodiments, administration of Compound A or Compound B per day it is administered is at an amount of about or at least about, or at or at least at 1.5 mg. In some embodiments, administration of Compound A or Compound B per day it is administered is at an amount of about or at least about, or at or at least at 2 mg. In some embodiments, administration of Compound A or Compound B per day it is administered is at an amount of about or at least about, or at or at least at 2.5 mg. In some embodiments, administration of Compound A or Compound B per day it is administered is at an amount of about or at least about, or at or at least at 3 mg. In some of any such embodiments, administration of Compound A or Compound B per day it is administered is at an amount of no more than about 5 mg. In some embodiments, administration of Compound A or Compound B per day it is administered is at an amount of no more than about 4.5 mg. In some embodiments, administration of Compound A or Compound B per day it is administered is at an amount of no more than about 4 mg. In some embodiments, administration of Compound A or Compound B per day it is administered is at an amount of no more than about 3.5 mg. In some embodiments, administration of Compound A or Compound B per day it is administered is at an amount of no more than about 3 mg. In some embodiments, administration of Compound A or Compound B per day it is administered is at an amount of no more than about 2.5 mg. In some embodiments, administration of Compound A or Compound B per day it is administered is at an amount of no more than about 2 mg. In some embodiments, administration of Compound A or Compound B per day it is administered is at an amount of no more than about 1.5 mg. In some embodiments, administration of Compound A or Compound B per day it is administered is at an amount of no more than about 1 mg.

In some embodiments, administration of Compound A or Compound B per day it is administered is at an amount of at or about 3 mg. In some embodiments, administration of Compound A or Compound B per day it is administered is at an amount of at or about 2.5 mg. In some embodiments, administration of Compound A or Compound B per day it is administered is at an amount of at or about 2 mg. In some embodiments, administration of Compound A or Compound B per day it is administered is at an amount of at or about 1.5 mg. In some embodiments, administration of Compound A or Compound B per day it is administered is at an amount of at or about 1 mg per day.

In some embodiments, Compound A or Compound B is administered in an amount that achieves a maximum concentration (Cₘₐₓ) of Compound A or Compound B in the blood, such as for each week of a cycling regimen or for at least one week of a cycling regimen, in a range of about 10 nM to about 500 nM, about 40 nM to about 500 nM, about 60 nM to about 500 nM, about 80 nM to about 500 nM, about 100 nM to about 500 nM, about 150 nM to about 500 nM, about 200 nM to about 500 nM, about 250 nM to about 500 nM, about 300 nM to about 500 nM, about 350 nM to about 500 nM, about 400 nM to about 500 nM, 10 nM to about 400 nM, about 40 nM to about 400 nM, about 60 nM to about 400 nM, about 80 nM to about 400 nM, about 100 nM to about 400 nM, about 150 nM to about 400 nM, about 200 nM to about 400 nM, about 250 nM to about 400 nM, about 300 nM to about 400 nM, about 350 nM to about 400 nM, 10 nM to about 350 nM, about 40 nM to about 350 nM, about 60 nM to about 350 nM, about 80 nM to about 350 nM, about 100 nM to about 350 nM, about 150 nM to about 350 nM, about 200 nM to about 350 nM, about 250 nM to about 350 nM, about 300 nM to about 350 nM, about 10 nM to about 300 nM, about 40 nM to about 300 nM, about 60 nM to about 300 nM, about 80 nM to about 300 nM, about 100 nM to about 300 nM, about 150 nM to about 300 nM, about 200 nM to about 300 nM, about 250 nM to about 250 nM, about 10 nM to about 250 nM, about 40 nM to about 250 nM, about 60 nM to about 250 nM, about 80 nM to about 250 nM, about 100 nM to about 250 nM, about 150 nM to about 250 nM, about 200 nM to about 250 nM, about 10 nM to about 200 nM, about 40 nM to about 200 nM, about 60 nM to about 200 nM, about 80 nM to about 200 nM, about 100 nM to about 200 nM, about 150 nM to about 200 nM, about 10 nM to about 150 nM, about 40 nM to about 150 nM, about 60 nM to about 150 nM, about 80 nM to about 150 nM, about 100 nM to about 150 nM, about 10 nM to about 100 nM, about 40 nM to about 100 nM, about 60 nM to about 100 nM, or about 80 nM to about 100 nM. In some embodiments, Compound A or Compound B is administered at an amount that maintains the Cₘₐₓ in the range for at least about 30 minutes, 1 hour, 2 hours, 4 hours, 8 hours, 16 hours or 24 hours

In some embodiments, Compound A or Compound B is administered at an amount that achieves a Cₘₐₓ of Compound A or Compound B in the blood at about or at least about 40 nM. In some embodiments, Compound A or Compound B is administered at an amount that achieves a Cₘₐₓ of Compound A or Compound B in the blood at about or at least about 60 nM. In some embodiments, Compound A or Compound B is administered at an amount that achieves a Cₘₐₓ of Compound A or Compound B in the blood, such as for each week of a cycling regimen or for at least one week of a cycling regimen, of at about or at least about 80 nM. In some embodiments, Compound A or Compound B is administered at an amount that achieves a Cₘₐₓ of Compound A or Compound B in the blood, such as for each week of a cycling regimen or for at least one week of a cycling regimen, of at about or at least about 90 nM. In some embodiments, Compound A or Compound B is administered at an amount that achieves a Cₘₐₓ of Compound A or Compound B in the blood, such as for each week of a cycling regimen or for at least one week of a cycling regimen, of at about or at least about 100 nM. In some embodiments, Compound A or Compound B is administered at an amount that maintains the Cₘₐₓ for at least about 30 minutes, 1 hour, 2 hours, 4 hours, 8 hours, 16 hours or 24 hours.

In some embodiments, Compound A or Compound B is administered at an amount that achieves a Cₘₐₓ of Compound A or Compound B in the blood, such as for each week of a cycling regimen or for at least one week of a cycling regimen, of at no more than about 500 nM. In some embodiments, Compound A or Compound B is administered at an amount that achieves a Cₘₐₓ of Compound A or Compound B in the blood, such as for each week of a cycling regimen or for at least one week of a cycling regimen, of at no more than about 400 nM. In some embodiments, Compound A or Compound B is administered at an amount that achieves a Cₘₐₓ of Compound A or Compound B in the blood, such as for each week of a cycling regimen or for at least one week of a cycling regimen, of at no more than about 350 nM. In some embodiments, Compound A or Compound B is administered at an amount that achieves a Cₘₐₓ of Compound A or Compound B in the blood, such as for each week of a cycling regimen or for at least one week of a cycling regimen, of at no more than about 300 nM. In some embodiments, Compound A or Compound B is administered at an amount that achieves a Cₘₐₓ of Compound A or Compound B in the blood, such as for each week of a cycling regimen or for at least one week of a cycling regimen, of at no more than about 250 nM. In some embodiments, Compound A or Compound B is administered at an amount that achieves a Cₘₐₓ of Compound A or Compound B in the blood, such as for each week of a cycling regimen or for at least one week of a cycling regimen, of at no more than about 200 nM. In some embodiments, Compound A or Compound B is administered at an amount that achieves a Cₘₐₓ of Compound A or Compound B in the blood, such as for each week of a cycling regimen or for at least one week of a cycling regimen, of at no more than about 150 nM.

In some embodiments, Compound A or Compound B is administered in a cycling regimen that involves repeated dosing of the compound for a specified period or duration. In some embodiments, Compound A or Compound B is administered in a cycling regimen in which, for each week of the cycling regimen or for at least one week of the cycling regimen, the compound is administered in an effective amount, such as an amount described above, on each of no more than 5 days per week for a period of more than one week. In some embodiments, the amount of Compound A or Compound B for each administration or per day it is administered is no more than 3 mg (e.g., no more than 3 mg, 2.5 mg, 2 mg, 1.5 mg, 1 mg, 0.5 mg). In some embodiments, the amount of Compound A or Compound B for each administration or per day it is administered is at or about 3 mg, at or about 2.5 mg, at or about 2 mg, at or about 1.5 mg, at or about 1 mg, at or about 0.5 mg. In some embodiments, the amount of Compound A or Compound B for each administration or per day it is administered is about 1 mg to about 2 mg (e.g., at or about 1 mg, at or about 2 mg).

In some embodiments, each week of a cycling regimen comprises administering Compound A or Compound B each of no more than 5 days per week. In some embodiments, each week of a cycling regimen comprises administering Compound A or Compound B for each of no more than 4 days per week. In some embodiments, each week of a cycling regimen comprises administering Compound A or Compound B for each of no more than 3 days per week.

In some embodiments, each week of a cycling regimen comprises administering Compound A or Compound B for 3 to 5 days per week. In some embodiments, each week of a cycling regimen comprises administering Compound A or Compound B for 4 to 5 days per week. In some embodiments, each week of a cycling regimen comprises administering Compound A or Compound B for 3 to 4 days per week.

In some embodiments, the each week of a cycling regimen, or at least one week of a cycling regimen, comprises administering Compound A or Compound B on each of no more than 5 consecutive days per week followed by a rest period for the remainder of the week during which the compound is not administered. In some embodiments, each week of a cycling regimen, or at least one week of a cycling regimen, comprises administering Compound A or Compound B for 3 to 5 consecutive days per week followed by a rest period for the remainder of the week during which the compound is not administered. In some embodiments, each week of the cycling regimen, or at least one week of the cycling regimen, comprises administering Compound A or Compound B on each of 3 consecutive days per week followed by a rest period of 4 days during which the compound is not administered. In some embodiments, each week of a cycling regimen, or at least one week of a cycling regimen, comprises administering Compound A or Compound B on each of 4 consecutive days per week followed by a rest period of 3 days during which the compound is not administered. In some embodiments, each week of a cycling regimen, or at least one week of a cycling regimen, comprises administering Compound A or Compound B one each of 5 consecutive days per week followed by a rest period of 2 days during which the compound is not administered.

In some embodiments, the cycling regimen for administering Compound A or Compound B is carried out for a period of time subsequent to initiation of administration of the T cell therapy. In some embodiments, administration of Compound A or Compound B extends for a period of more than one week after initiation of administration of the T cell therapy. In some embodiments, administration of Compound A or Compound B extends for a period of about or at least about one month after initiation of administration of the T cell therapy. In some embodiments, administration of Compound A or Compound B extends for a period of about or at least about two months after initiation of administration of the T cell therapy. In some embodiments, administration of Compound A or Compound B extends for a period of about or at least about three months after initiation of administration of the T cell therapy. In some embodiments, administration of Compound A or Compound B extends for a period of about or at least about four months after initiation of administration of the T cell therapy. In some embodiments, administration of Compound A or Compound B extends for a period of about or at least about five months after initiation of administration of the T cell therapy.

In some embodiments, administration of Compound A or Compound B extends for a period of at least three months. In some embodiments, administration of Compound A or Compound B extends for a period of at or about 90 days, at or about 100 days, at or about 105 days, at or about 110 days, at or about 115 days, at or about 120 days, at or about 125 days, at or about 130 days, at or about 135 days, at or about 140 days, at or about 145 days, at or about 150 days, at or about 155 days, at or about 160 days, at or about 165 days, at or about 170 days, at or about 175 days, at or about 180 days, at or about 185 days, at or about 190 days, at or about 195 days, at or about 200 days or more after initiation of administration of the T cell therapy.

In some embodiments, administration of Compound A or Compound B extends for a period of at or about 90 days or at or about three months after initiation of administration of the T cell therapy (e.g., CAR T cell therapy). In some embodiments, administration of Compound A or Compound B extends for a period of at or about 120 days or four months after initiation of administration of the T cell therapy (e.g., CAR T cell therapy). In some embodiments, administration of Compound A or Compound B extends for a period of at or about 150 days or five months after initiation of administration of the T cell therapy (e.g., CAR T cell therapy). In some embodiments, administration of Compound A or Compound B extends for a period of at or about 180 days or six months after initiation of administration of the T cell therapy (e.g., CAR T cell therapy).

In some embodiments, administration of Compound A or Compound B is ended or stopped at the end of the period (e.g. at or about 3, 4, 5, or 6 months) after initiation of administration of the T cell therapy (e.g., CAR T cell therapy) if the subject has, prior to or at or about 6 months, achieved a complete response (CR) following the treatment or the cancer (e.g. B cell malignancy) has progressed or relapsed following remission after the treatment. In some embodiments, the period is of a fixed duration such that the administration of Compound A or Compound B is continued for the period even if the subject has achieved a complete response (CR) at a time point prior to the end of the period. In some embodiments the subject is has a CR with minimal residual disease (MRD). In some embodiments, the subject has a CR that is MRD−.

In some embodiments, administration of Compound A or Compound B is continued after the end of the period, e.g. continued for longer than at or about 3, 4, 5 or 6 months after initiation of administration of the T cell therapy (e.g. CAR T cells), if the subject exhibits a partial response (PR) or stable disease (SD) after the treatment. In some embodiments, administration of Compound A or Compound B is continued for greater than 6 months after initiation of administration of the T cell therapy (e.g., CAR T cell therapy). In some embodiments, for subjects that exhibited a PR or SD at the end of the initial period, administration of Compound A or Compound B is continued until the subject has achieved a complete response (CR) following the treatment or until the cancer (e.g. multiple myeloma, such as relapsed/refractory multiple myeloma) has progressed or relapsed following remission after the treatment.

In some embodiments, administration of Compound A or Compound B is carried out in a cycling regimen comprising administering Compound A or Compound B in an amount of no more than about 3 mg (e.g., 1 to 3 mg, 1 mg, 2 mg, or 3 mg) per day for no more than 5 days (e.g., 3 days, 4 day or 5 days) per week for a period of more than one week. In some embodiments, each week of the cycling regimen involves administration of the compound for each of 3 consecutive days, 4 consecutive days or 5 consecutive days followed by a rest period for the remainder of the week during which the compound is not administered. In some embodiments, the each week of the cycling regimen comprises administration of the compound for 5 days followed by a rest period of two days during which the compound is not administered. In some embodiments, the administration of Compound A or Compound B is initiated greater than about 14 to about 35 days (e.g., about 21 to about 35 days) after initiation of the administration of the cell therapy. In some embodiments, at the time of administering Compound A or Compound B, the subject does not exhibit a severe toxicity following administration of the T cell therapy (e.g. CAR T cells).

In some embodiments, administration of Compound A or Compound B is carried out in a cycling regimen comprising administering an effective amount of Compound A or Compound B for no more than 5 days (e.g., 3 days, 4 day or 5 days) per week for a period that extends at or about or greater than 3 months, at or about or greater than 4 months, at or about or greater than 5 months or at or about or greater than 6 months after initiation of administration of the cell therapy (e.g., T cell therapy). In some embodiments, the period extends for at or about 3 months, at or about 4 months, at or about 5 months or at or about 6 months. In some embodiments, each week of the cycling regimen involves administration of the compound for each of 3 consecutive days, 4 consecutive days or 5 consecutive days followed by a rest period for the remainder of the week during which the compound is not administered. In some embodiments, each week of cycling regimen comprises administration of the compound on each of 5 consecutive days followed by a rest period of two days during which the compound is not administered. In some embodiments, the administration of Compound A or Compound B is initiated greater than about 14 to about 35 days (e.g., about 21 to about 35 days, such as at or about 28 days) after initiation of the administration of the cell therapy. In some embodiments, at the time of administering Compound A or Compound B, the subject does not exhibit a severe toxicity following administration of the cell therapy. In some embodiments, the administration of Compound A or Compound B is ended or stopped, if the subject has, prior to at or about the end of the period, achieved a complete response (CR) following the treatment or the cancer, e.g. B cell malignancy, has progressed or relapsed following remission after the treatment. In some embodiments, administration of Compound A or Compound B is continued for the period even if the subject has achieved a complete response (CR) at a time point prior to the end of the period. In some embodiments, the administration of Compound A or Compound B is continued after the end of the initial period if, after initiation of administration of the T cell therapy, the subject exhibits a partial response (PR) or stable disease (SD) after the treatment. In some embodiments, the administration of Compound A or Compound B is repeated until the subject has achieved a complete response (CR) following the treatment or until the cancer, e.g. multiple myeloma, such as relapsed/refractory multiple myeloma, has progressed or relapsed following remission after the treatment.

In some embodiments, administration of Compound A or Compound B is carried out in a cycling regimen comprising administering Compound A or Compound B in an amount of no more than about 3 mg (e.g., 1 to 3 mg, 1 mg, 2 mg, or 3 mg) per day on each of no more than 5 days (e.g., 3 days, 4 day or 5 days) per week for a period of about or greater than three months (e.g., for a period of at or about three months, four months, five months, or six months) after initiation of administration of the T cell therapy (e.g., CAR T cell therapy). In some embodiments, each week of the cycling regimen involves administration of the compound on each of 3 consecutive days, 4 consecutive days or 5 consecutive days followed by a rest period for the remainder of the week during which the compound is not administered. In some embodiments, each week of the cycling regimen comprises administration of the compound on each of 5 days followed by a rest period of two days during which the compound is not administered. In some embodiments, the administration of Compound A or Compound B is initiated greater than about 14 to about 35 days (e.g., about 21 to about 35 days, e.g. at or about 28 days) after initiation of the administration of the cell therapy. In some embodiments, at the time of administering Compound A or Compound B, the subject does not exhibit a severe toxicity following administration of the cell therapy. In some embodiments, the cancer is multiple myeloma, such as relapsing/refractory multiple myeloma. In some embodiments, administration of Compound A or Compound B is ended or stopped at or about 6 months after initiation of administration of the T cell therapy if the subject has, prior to at or about 6 months, achieved a complete response (CR) following the treatment or the cancer, e.g. B cell malignancy, has progressed or relapsed following remission after the treatment. In some embodiments, the cycling regimen is continued for the entire period even if the subject has achieved a complete response (CR) at a time point prior to the end of the period. In some embodiments, the administration of Compound A or Compound B is further continued after the end of the period, such as is continued for greater than 6 months after initiation of administration of the cell therapy, if, at or about six months, the subject exhibits a partial response (PR) or stable disease (SD) after the treatment. In some embodiments, the administration of Compound A or Compound B is continued until the subject has achieved a complete response (CR) following the treatment or until the cancer, e.g. multiple myeloma, such as relapsed/refractory multiple myeloma, has progressed or relapsed following remission after the treatment.

In some embodiments, administration of Compound A or Compound B is carried out in a cycling regimen comprising administering Compound A or Compound B at an amount of about 1 mg to about 3 mg (e.g., 1 mg, 2 mg or 3 mg) per day on each of 5 consecutive days per week followed by a rest period of 2 days during which the compound is not administered for a period of at or about or greater than six months after initiation of the cell therapy (e.g., T cell therapy). In some embodiments, the administration of Compound A or Compound B is initiated greater than about 14 to about 35 days (e.g., about 21 to about 35 days, e.g. at or about 28 days) after initiation of the administration of the cell therapy. In some embodiments, at the time of administering Compound A or Compound B, the subject does not exhibit a severe toxicity following administration of the cell therapy. In some embodiments, administration of Compound A or Compound B is stopped at or about 6 months after initiation of administration of the cell therapy if the subject has, at or about 6 months, achieved a complete response (CR) following the treatment or the cancer, e.g. B cell malignancy, has progressed or relapsed following remission after the treatment. In some embodiments, administration of Compound A or Compound B is continued for the period even if the subject has achieved a complete response (CR) at a time point prior to at or about 6 months. In some embodiments, administration of Compound A or Compound B is further administered for greater than 6 months after initiation of administration of the T cell therapy if, at or about six months, the subject exhibits a partial response (PR) or stable disease (SD) after the treatment. In some embodiments, the administration is continued until the subject has achieved a complete response (CR) following the treatment or until the disease or condition, e.g. multiple myeloma, such as relapsed/refractory multiple myeloma, has progressed or relapsed following remission after the treatment.

In some cases, the cycling regimen can be interrupted at any time, and/or for one or more times. In some cases, the cycling regimen is interrupted or modified if the subject develops one or more adverse event, dose-limiting toxicity (DLT), neutropenia or febrile neutropenia, thrombocytopenia, cytokine release syndrome (CRS) and/or neurotoxicity (NT), such as those as described in Section IV. In some embodiments, the amount of Compound A or Compound B for each administration or per day in certain days of a week is altered after the subject develops one or more adverse event, dose-limiting toxicity (DLT), neutropenia or febrile neutropenia, thrombocytopenia, cytokine release syndrome (CRS) and/or neurotoxicity (NT).

In any of the aforementioned embodiments, the immunomodulatory compound, e.g. Compound A or Compound B, may be administered orally. In some embodiments, the immunomodulatory compound, e.g. Compound A or Compound B, is administered as a tablet or capsule.

In some embodiments, dosages, such as daily dosages, are administered in one or more divided doses, such as 2, 3, or 4 doses, or in a single formulation. The immunomodulatory compound, e.g., Compound A or Compound B, can be administered alone, in the presence of a pharmaceutically acceptable carrier, or in the presence of other therapeutic agents.

It is understood that higher or lower dosages of the immunomodulatory compound could be used, for example depending on the particular agent and the route of administration. In some embodiments, the immunomodulatory compound may be administered alone or in the form of a pharmaceutical composition wherein the compound is in admixture or mixture with one or more pharmaceutically acceptable carriers, excipients, or diluents. In some embodiments, the immunomodulatory compound may be administered either systemically or locally to the organ or tissue to be treated. Exemplary routes of administration include, but are not limited to, topical, injection (such as subcutaneous, intramuscular, intradermal, intraperitoneal, intratumoral, and intravenous), oral, sublingual, rectal, transdermal, intranasal, vaginal and inhalation routes. In some embodiments, the route of administration is oral, parenteral, rectal, nasal, topical, or ocular routes, or by inhalation. In some embodiments, the immunomodulatory compound is administered orally. In some embodiments, the immunomodulatory compound is administered orally in solid dosage forms, such as capsules, tablets and powders, or in liquid dosage forms, such as elixirs, syrups and suspensions.

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. If symptoms have been alleviated to an appropriate level, treatment may cease. Patients may, however, require intermittent treatment on a long-term basis upon any recurrence of symptoms. Patients may also require chronic treatment on a long-term basis.

C. Lymphodepleting Treatment

In some aspects, the provided methods can further include administering one or more lymphodepleting therapies, such as prior to or simultaneous with initiation of administration of the T cell therapy. In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., Science, 298, 850-54 (2002); Rosenberg et al., Clin Cancer Res, 17(13):4550-4557 (2011). Likewise, in the context of CAR+ T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47 (2013); Kochenderfer et al., Blood, 119: 2709-2720 (2012); Kalos et al., Sci Transl Med, 3(95):95ra73 (2011); Clinical Trial Study Record Nos.: NCT02315612; NCT01822652.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the dose of cells. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In one exemplary dosage regime, prior to receiving the first dose, subjects receive an immunomodulatory compound 1 day before the administration of cells and an lymphodepleting preconditioning chemotherapy of cyclophosphamide and fludarabine (CY/FLU), which is administered at least two days before the first dose of CAR-expressing cells and generally no more than 7 days before administration of cells. In another exemplary dosage regime, subjects receive the immunomodulatory compound concurrently with the administration of cells, such as on the same day. In yet another exemplary dosage regime, subjects receive the immunomodulatory compound several days after the administration of cells, such as 7, 8, 9, 10, 11, 12, 13, 14, or more than 14 days after. In some cases, for example, cyclophosphamide is given from 24 to 27 days after the administration of the immunomodulatory compound, e.g., Compound A or Compound B. After preconditioning treatment, subjects are administered the dose of CAR-expressing T cells as described above.

In some embodiments, the administration of the preconditioning agent prior to infusion of the dose of cells improves an outcome of the treatment. For example, in some aspects, preconditioning improves the efficacy of treatment with the dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to infusion of the dose of cells improves an outcome of the treatment such as by improving the efficacy of treatment with the dose or increases the persistence of the recombinant receptor-expressing cells (e.g., CAR-expressing cells, such as CAR-expressing T cells) in the subject. Therefore, in some embodiments, the dose of preconditioning agent given in the method which is a combination therapy with the immunomodulatory compound and cell therapy is higher than the dose given in the method without the immunomodulatory compound.

### II. T CELL THERAPY AND ENGINEERING CELLS

In some embodiments, the T cell therapy for use in accord with the provided combination therapy methods includes administering engineered cells expressing recombinant receptors designed to recognize and/or specifically bind to molecules associated with the disease or condition, such as a cancer, e.g. multiple myeloma, for example relapsed or refractory multiple myeloma. In some embodiments, binding to the antigen results in a response, such as an immune response against such molecules upon binding to such molecules. In some embodiments, the cells contain or are engineered to contain an engineered receptor, e.g., an engineered antigen receptor, such as a chimeric antigen receptor (CAR), or a T cell receptor (TCR). The recombinant receptor, such as a CAR, generally includes an extracellular antigen (or ligand) binding domain that is directed against BCMA, linked to one or more intracellular signaling components, in some aspects via linkers and/or transmembrane domain(s). In some aspects, the engineered cells are provided as pharmaceutical compositions and formulations suitable for administration to a subjects, such as for adoptive cell therapy. Also provided are therapeutic methods for administering the cells and compositions to subjects, e.g., patients.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

A. Recombinant Receptors, e.g. Chimeric Antigen Receptors (CARs)

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

In some embodiments of the provided methods and uses, the engineered cells, such as T cells, express a chimeric receptors, such as a chimeric antigen receptors (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32.34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IMGT or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the antigen binding domains included in the CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; heavy chain variable (V_{H}) regions, single-chain antibody molecules such as scFvs and single-domain antibodies comprising only the V_{H} region; and multispecific antibodies formed from antibody fragments. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb), or a single domain antibody (sdAb), such as sdFv, nanobody, V_{H}H and V_{NAR}. In some embodiments, an antigen-binding fragment comprises antibody variable regions joined by a flexible linker.

In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is an scFv comprising a heavy chain variable (V_{H}) region and a light chain variable (V_{L}) region.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the anti-BCMA antibodies included in the provided CARs are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the antibodies included in the provided CARs are those that are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

In some embodiments, the CAR includes a BCMA-binding portion or portions of the antibody molecule, such as a heavy chain variable (V_{H}) region and/or light chain variable (V_{L}) region of the antibody, e.g., an scFv antibody fragment. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the provided BCMA-binding CARs contain an antibody, such as an anti-BCMA antibody, or an antigen-binding fragment thereof that confers the BCMA-binding properties of the provided CAR. In some embodiments, the antibody or antigen-binding domain can be any anti-BCMA antibody described or derived from any anti-BCMA antibody described. See, e.g., Carpenter et al., Clin Cancer Res., 2013, 19(8):2048-2060; U.S. Pat. Nos. 9,034,324 9,765,342; U.S. Patent publication No. US2016/0046724, US20170183418; and International published PCT App. No. WO 2016090320, WO2016090327, WO2016094304, WO2016014565, WO106014789, WO2010104949, WO2017/025038, or WO2017173256. Any of such anti-BCMA antibodies or antigen-binding fragments can be used in the provided CARs. In some embodiments, the anti-BCMA CAR contains an antigen-binding domain that is an scFv containing a variable heavy (V_{H}) and/or a variable light (V_{L}) region. In some embodiments, the scFv containing a variable heavy (V_{H}) and/or a variable light (V_{L}) region is derived from an antibody described in WO 2016090320 or WO2016090327.

In some embodiments, the antibody, e.g., the anti-BCMA antibody or antigen-binding fragment, contains a heavy and/or light chain variable (V_{H} or V_{L}) region sequence as described, or a sufficient antigen-binding portion thereof. In some embodiments, the anti-BCMA antibody, e.g., antigen-binding fragment, contains a V_{H} region sequence or sufficient antigen-binding portion thereof that contains a CDR-H1, CDR-H2 and/or CDR-H3 as described. In some embodiments, the anti-BCMA antibody, e.g., antigen-binding fragment, contains a V_{L} region sequence or sufficient antigen-binding portion that contains a CDR-L1, CDR-L2 and/or CDR-L3 as described. In some embodiments, the anti-BCMA antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, about 91%, about 92%, about 93%, about 94%, about 95%, about 96%, about 97%, about 98%, or about 99% identical to such a sequence.

In some embodiments, the antibody is a single domain antibody (sdAb) comprising only a V_{H} region sequence or a sufficient antigen-binding portion thereof, such as any of the above described V_{H} sequences (e.g., a CDR-H1, a CDR-H2, a CDR-H3 and/or a CDR-H4).

In some embodiments, an antibody provided herein (e.g., an anti-BCMA antibody) or antigen-binding fragment thereof comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human BCMA antibodies, and cells expressing such chimeric receptors have been previously described. See Carpenter et al., Clin Cancer Res., 2013, 19(8):2048-2060, U.S. Pat. No. 9,765,342, WO 2016/090320, WO2016090327, WO2010104949A2, WO2016/0046724, WO2016/014789, WO2016/094304, WO2017/025038, and WO2017173256. In some embodiments, the anti-BCMA CAR contains an antigen-binding domain, such as an scFv, containing a variable heavy (V_{H}) and/or a variable light (V_{L}) region derived from an antibody described in WO 2016/090320 or WO2016090327. In some embodiments, the antigen-binding domain is an antibody fragment containing a variable heavy chain (V_{H}) and a variable light chain (V_{L}) region. In some aspects, the V_{H} region is or includes an amino acid sequence having at least 90%, 91%, 92%, 93%, 94%, 95%, 96%, 97%, 98% or 99% sequence identity to the V_{H} region amino acid sequence set forth in any of SEQ ID NOs: 30, 32, 34, 36, 38, 40, 42, 77, 79, 81, 83, 85, 87, 89, 91, 93, 95, 97, 99, 101, 103, 105, 107, 109, 111, 47, 49, 51 and 53; and/or the V_{L} region is or includes an amino acid sequence having at least 90%, 91%, 92%, 93%, 94%, 95%, 96%, 97%, 98%, or 99% sequence identity to the V_{L} region amino acid sequence set forth in any of SEQ ID NOs: 31, 33, 35, 37, 39, 41, 43, 78, 80, 82, 84, 86, 88, 90, 92, 94, 96, 98, 100, 102, 104, 106, 108, 110, 112, 48, 50, 52 and 54.

In some embodiments, the antigen-binding domain in the anti-BCMA CAR, such as an scFv, comprises a V_{H} and a V_{L} region. In some embodiments, the V_{H} region comprises a CDR-H1, a CDR-H2 and a CDR-H3 contained within the V_{H} set forth in SEQ ID NO: 30 and the V_{L} region comprises a CDR-L1, a CDR-L2 and a CDR-L3 contained within the V_{L} set forth in SEQ ID NO:31. In some embodiments, the V_{H} region comprises a CDR-H1, a CDR-H2 and a CDR-H3 contained within the V_{H} set forth in SEQ ID NO: 32 and the V_{L} region comprises a CDR-L1, a CDR-L2 and a CDR-L3 contained within the V_{L} set forth in SEQ ID NO:33. In some embodiments, the V_{H} region comprises a CDR-H1, a CDR-H2 and a CDR-H3 contained within the V_{H} set forth in SEQ ID NO: 34 and the V_{L} region comprises a CDR-L1, a CDR-L2 and a CDR-L3 contained within the V_{L} set forth in SEQ ID NO: 35. In some embodiments, the V_{H} region comprises a CDR-H1, a CDR-H2 and a CDR-H3 contained within the V_{H} set forth in SEQ ID NO: 36 and the V_{L} region comprises a CDR-L1, a CDR-L2 and a CDR-L3 contained within the V_{L} set forth in SEQ ID NO:37. In some embodiment the V_{H} region comprises a CDR-H1, a CDR-H2 and a CDR-H3 contained within the V_{H} set forth in SEQ ID NO: 38 and the V_{L} region comprises a CDR-L1, a CDR-L2 and a CDR-L3 contained within the V_{L} set forth in SEQ ID NO: 39. In some embodiments, the V_{H} region comprises a CDR-H1, a CDR-H2 and a CDR-H3 contained within the V_{H} set forth in SEQ ID NO: 40 and the V_{L} region comprises a CDR-L1, a CDR-L2 and a CDR-L3 contained within the V_{L} set forth in SEQ ID NO: 41. In some embodiments, the V_{H} region comprises a CDR-H1, a CDR-H2 and a CDR-H3 contained within the V_{H} set forth in SEQ ID NO: 42 and the V_{L} region comprises a CDR-L1, a CDR-L2 and a CDR-L3 contained within the V_{L} set forth in SEQ ID NO: 43. In some embodiments, the V_{H} region comprises a CDR-H1, a CDR-H2 and a CDR-H3 contained within the V_{H} set forth in SEQ ID NO: 77 and the V_{L} region comprises a CDR-L1, a CDR-L2 and a CDR-L3 contained within the V_{L} set forth in SEQ ID NO: 78. In some embodiments, the V_{H} region comprises a CDR-H1, a CDR-H2 and a CDR-H3 contained within the V_{H} set forth in SEQ ID NO: 79 and the V_{L} region comprises a CDR-L1, a CDR-L2 and a CDR-L3 contained within the V_{L} set forth in SEQ ID NO: 80. In some embodiments, the V_{H} region comprises a CDR-H1, a CDR-H2 and a CDR-H3 contained within the V_{H} set forth in SEQ ID NO: 81 and the V_{L} region comprises a CDR-L1, a CDR-L2 and a CDR-L3 contained within the V_{L} set forth in SEQ ID NO: 82. In some embodiments, the V_{H} region comprises a CDR-H1, a CDR-H2 and a CDR-H3 contained within the V_{H} set forth in SEQ ID NO: 83 and the V_{L} region comprises a CDR-L1, a CDR-L2 and a CDR-L3 contained within the V_{L} set forth in SEQ ID NO: 84. In some embodiments, the V_{H} region comprises a CDR-H1, a CDR-H2 and a CDR-H3 contained within the V_{H} set forth in SEQ ID NO: 85 and the V_{L} region comprises a CDR-L1, a CDR-L2 and a CDR-L3 contained within the V_{L} set forth in SEQ ID NO: 86. In some embodiments, the V_{H} region comprises a CDR-H1, a CDR-H2 and a CDR-H3 contained within the V_{H} set forth in SEQ ID NO: 87 and the V_{L} region comprises a CDR-L1, a CDR-L2 and a CDR-L3 contained within the V_{L} set forth in SEQ ID NO: 88. In some embodiments, the V_{H} region comprises a CDR-H1, a CDR-H2 and a CDR-H3 contained within the V_{H} set forth in SEQ ID NO: 89 and the V_{L} region comprises a CDR-L1, a CDR-L2 and a CDR-L3 contained within the V_{L} set forth in SEQ ID NO: 90. In some embodiments, the V_{H} region comprises a CDR-H1, a CDR-H2 and a CDR-H3 contained within the V_{H} set forth in SEQ ID NO: 91 and the V_{L} region comprises a CDR-L1, a CDR-L2 and a CDR-L3 contained within the V_{L} set forth in SEQ ID NO: 92. In some embodiments, the V_{H} region comprises a CDR-H1, a CDR-H2 and a CDR-H3 contained within the V_{H} set forth in SEQ ID NO: 93 and the V_{L} region comprises a CDR-L1, a CDR-L2 and a CDR-L3 contained within the V_{L} set forth in SEQ ID NO: 94. In some embodiments, the V_{H} region comprises a CDR-H1, a CDR-H2 and a CDR-H3 contained within the V_{H} set forth in SEQ ID NO: 95 and the V_{L} region comprises a CDR-L1, a CDR-L2 and a CDR-L3 contained within the V_{L} set forth in SEQ ID NO: 96. In some embodiments, the V_{H} region comprises a CDR-H1, a CDR-H2 and a CDR-H3 contained within the V_{H} set forth in SEQ ID NO: 97 and the V_{L} region comprises a CDR-L1, a CDR-L2 and a CDR-L3 contained within the V_{L} set forth in SEQ ID NO: 98. In some embodiments, the V_{H} region comprises a CDR-H1, a CDR-H2 and a CDR-H3 contained within the V_{H} set forth in SEQ ID NO: 99 and the V_{L} region comprises a CDR-L1, a CDR-L2 and a CDR-L3 contained within the V_{L} set forth in SEQ ID NO: 100. In some embodiments, the V_{H} region comprises a CDR-H1, a CDR-H2 and a CDR-H3 contained within the V_{H} set forth in SEQ ID NO: 101 and the V_{L} region comprises a CDR-L1, a CDR-L2 and a CDR-L3 contained within the V_{L} set forth in SEQ ID NO: 102. In some embodiments, the V_{H} region comprises a CDR-H1, a CDR-H2 and a CDR-H3 contained within the V_{H} set forth in SEQ ID NO: 103 and the V_{L} region comprises a CDR-L1, a CDR-L2 and a CDR-L3 contained within the V_{L} set forth in SEQ ID NO: 104. In some embodiments, the V_{H} region comprises a CDR-H1, a CDR-H2 and a CDR-H3 contained within the V_{H} set forth in SEQ ID NO: 105 and the V_{L} region comprises a CDR-L1, a CDR-L2 and a CDR-L3 contained within the V_{L} set forth in SEQ ID NO: 106. In some embodiments, the V_{H} region comprises a CDR-H1, a CDR-H2 and a CDR-H3 contained within the V_{H} set forth in SEQ ID NO: 107 and the V_{L} region comprises a CDR-L1, a CDR-L2 and a CDR-L3 contained within the V_{L} set forth in SEQ ID NO: 106. In some embodiments, the V_{H} region comprises a CDR-H1, a CDR-H2 and a CDR-H3 contained within the V_{H} set forth in SEQ ID NO: 30 and the V_{L} region comprises a CDR-L1, a CDR-L2 and a CDR-L3 contained within the V_{L} set forth in SEQ ID NO: 108. In some embodiments, the V_{H} region comprises a CDR-H1, a CDR-H2 and a CDR-H3 contained within the V_{H} set forth in SEQ ID NO: 109 and the V_{L} region comprises a CDR-L1, a CDR-L2 and a CDR-L3 contained within the V_{L} set forth in SEQ ID NO: 110. In some embodiments, the V_{H} region comprises a CDR-H1, a CDR-H2 and a CDR-H3 contained within the V_{H} set forth in SEQ ID NO: 111 and the V_{L} region comprises a CDR-L1, a CDR-L2 and a CDR-L3 contained within the V_{L} set forth in SEQ ID NO: 112.

In some embodiments, the antigen-binding domain, such as an scFv, contains a V_{H} set forth in SEQ ID NO: 30 and a V_{L} set forth in SEQ ID NO:31. In some embodiments, the antigen-binding domain, such as an scFv, contains a V_{H} set forth in SEQ ID NO: 32 and a V_{L} set forth in SEQ ID NO:33. In some embodiments, the antigen-binding domain, such as an scFv, contains a V_{H} set forth in SEQ ID NO: 34 and a V_{L} set forth in SEQ ID NO: 35. In some embodiments, the antigen-binding domain, such as an scFv, contains a V_{H} set forth in SEQ ID NO: 36 and a V_{L} set forth in SEQ ID NO:37. In some embodiment the antigen-binding domain, such as an scFv, contains a V_{H} set forth in SEQ ID NO: 38 and a V_{L} set forth in SEQ ID NO: 39. In some embodiments, the antigen-binding domain, such as an scFv, contains a V_{H} set forth in SEQ ID NO: 40 and a V_{L} set forth in SEQ ID NO: 41. In some embodiments, the antigen-binding domain, such as an scFv, contains a V_{H} set forth in SEQ ID NO: 42 and a V_{L} set forth in SEQ ID NO: 43. In some embodiments, the antigen-binding domain, such as an scFv, contains a V_{H} set forth in SEQ ID NO: 77 and a V_{L} set forth in SEQ ID NO: 78. In some embodiments, the antigen-binding domain, such as an scFv, contains a V_{H} set forth in SEQ ID NO: 79 and a V_{L} set forth in SEQ ID NO: 80. In some embodiments, the antigen-binding domain, such as an scFv, contains a V_{H} set forth in SEQ ID NO: 81 and a V_{L} set forth in SEQ ID NO: 82. In some embodiments, the antigen-binding domain, such as an scFv, contains a V_{H} set forth in SEQ ID NO: 83 and a V_{L} set forth in SEQ ID NO: 84. In some embodiments, the antigen-binding domain, such as an scFv, contains a V_{H} set forth in SEQ ID NO: 85 and a V_{L} set forth in SEQ ID NO: 86. In some embodiments, the antigen-binding domain, such as an scFv, contains a V_{H} set forth in SEQ ID NO: 87 and a V_{L} set forth in SEQ ID NO: 88. In some embodiments, the antigen-binding domain, such as an scFv, contains a V_{H} set forth in SEQ ID NO: 89 and a V_{L} set forth in SEQ ID NO: 90. In some embodiments, the antigen-binding domain, such as an scFv, contains a V_{H} set forth in SEQ ID NO: 91 and a V_{L} set forth in SEQ ID NO: 92. In some embodiments, the antigen-binding domain, such as an scFv, contains a V_{H} set forth in SEQ ID NO: 93 and a V_{L} set forth in SEQ ID NO: 94. In some embodiments, the antigen-binding domain, such as an scFv, contains a V_{H} set forth in SEQ ID NO: 95 and a V_{L} set forth in SEQ ID NO: 96. In some embodiments, the antigen-binding domain, such as an scFv, contains a V_{H} set forth in SEQ ID NO: 97 and a V_{L} set forth in SEQ ID NO: 98. In some embodiments, the antigen-binding domain, such as an scFv, contains a V_{H} set forth in SEQ ID NO: 99 and a V_{L} set forth in SEQ ID NO: 100. In some embodiments, the antigen-binding domain, such as an scFv, contains a V_{H} set forth in SEQ ID NO: 101 and a V_{L} set forth in SEQ ID NO: 102. In some embodiments, the antigen-binding domain, such as an scFv, contains a V_{H} set forth in SEQ ID NO: 103 and a V_{L} set forth in SEQ ID NO: 104. In some embodiments, the antigen-binding domain, such as an scFv, contains a V_{H} set forth in SEQ ID NO: 105 and a V_{L} set forth in SEQ ID NO: 106. In some embodiments, the antigen-binding domain, such as an scFv, contains a V_{H} set forth in SEQ ID NO: 107 and a V_{L} set forth in SEQ ID NO: 106. In some embodiments, the antigen-binding domain, such as an scFv, contains a V_{H} set forth in SEQ ID NO: 30 and a V_{L} set forth in SEQ ID NO: 108. In some embodiments, the antigen-binding domain, such as an scFv, contains a V_{H} set forth in SEQ ID NO: 109 and a V_{L} set forth in SEQ ID NO: 110. In some embodiments, the antigen-binding domain, such as an scFv, contains a V_{H} set forth in SEQ ID NO: 111 and a V_{L} set forth in SEQ ID NO: 112. In some embodiments, the antigen-binding domain, such as an scFv, contains a V_{H} set forth in SEQ ID NO: 47 and a V_{L} set forth in SEQ ID NO: 48. In some embodiments, the antigen-binding domain, such as an scFv, contains a V_{H} set forth in SEQ ID NO: 49 and a V_{L} set forth in SEQ ID NO: 50. In some embodiments, the antigen-binding domain, such as an scFv, contains a V_{H} set forth in SEQ ID NO: 51 and a V_{L} set forth in SEQ ID NO: 52. In some embodiments, the antigen-binding domain, such as an scFv, contains a V_{H} set forth in SEQ ID NO: 53 and a V_{L} set forth in SEQ ID NO: 54. In some embodiments, the V_{H} or V_{L} has a sequence of amino acids that exhibits at least 85%, 86%, 87%, 88%, 89%, 90%, 91%, 92%, 93%, 94%, 95%, 96%, 97%, 98%, 99% or more sequence identity to any of the foregoing V_{H} or V_{L} sequences, and retains binding to BCMA. In some embodiments, the V_{H} region is amino-terminal to the V_{L} region. In some embodiments, the V_{H} region is carboxy-terminal to the V_{L} region. In some embodiments, the variable heavy and variable light chains are connected by a linker. In some embodiments, the linker is set forth in SEQ ID NO: 70, 72, 73, 74 or 55.

In some embodiments, the antibody is an antigen-binding fragment, such as an scFv, that includes one or more linkers joining two antibody domains or regions, such as a heavy chain variable (V_{H}) region and a light chain variable (V_{L}) region. Accordingly, the antibodies include single-chain antibody fragments, such as scFvs and diabodies, particularly human single-chain antibody fragments, typically comprising linker(s) joining two antibody domains or regions, such V_{H} and V_{L} regions. The linker typically is a peptide linker, e.g., a flexible and/or soluble peptide linker, such as one rich in glycine and serine. Among the linkers are those rich in glycine and serine and/or in some cases threonine. In some embodiments, the linkers further include charged residues such as lysine and/or glutamate, which can improve solubility. In some embodiments, the linkers further include one or more proline.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO:19) or GGGS (3GS; SEQ ID NO:71), such as between 2, 3, 4, and 5 repeats of such a sequence. Exemplary linkers include those having or consisting of an sequence set forth in SEQ ID NO:72 (GGGGSGGGGSGGGGS), SEQ ID NO:55 (ASGGGGSGGRASGGGGS), SEQ ID NO:73 (GSTSGSGKPGSGEGSTKG) or SEQ ID NO: 74 (SRGGGGSGGGGSGGGGSLEMA).

In some embodiments, the antigen-binding domain in the anti-BCMA CAR, such as an scFv, comprises a V_{H} and a V_{L} region. In some embodiments, the V_{H} region comprises a CDR-H1 set forth in SEQ ID NO: 56, a CDR-H2 set forth in SEQ ID NO:57 and a CDR-H3 set forth in SEQ ID NO:58, and the V_{L} region comprises a CDR-L1 set forth in SEQ ID NO: 59, a CDR-L2 set forth in SEQ ID NO:60 and a CDR-H3 set forth in SEQ ID NO:61. In some embodiments, the V_{H} region has the sequence of amino acids set forth in SEQ ID NO:36 or a sequence of amino acids that exhibits at least 90%, at least 91%, at least 92%, at least 93%, at least 94%, at least 95%, at least 96%, at least 97%, at least 98%, at least 99% to SEQ ID NO:36, and the V_{L} region has the sequence of amino acids set forth in SEQ ID NO:37 or a sequence of amino acids that exhibits at least 90%, at least 91%, at least 92%, at least 93%, at least 94%, at least 95%, at least 96%, at least 97%, at least 98%, at least 99% to SEQ ID NO:37. In some embodiments, the V_{H} region has the sequence of amino acids set forth in SEQ ID NO:36 and the V_{L} region has the sequence of amino acids set forth in SEQ ID NO:37. In some embodiments, the V_{H} region is amino-terminal to the V_{L} region. In some embodiments, the V_{H} region is carboxy-terminal to the V_{L} region. In some embodiments, the antigen-binding domain is an scFv that has the sequence of amino acids set forth in SEQ ID NO:180 or a sequence of amino acids exhibits at least 90%, at least 91%, at least 92%, at least 93%, at least 94%, at least 95%, at least 96%, at least 97%, at least 98%, at least 99% to SEQ ID NO:180. In particular embodiments, any of the above antigen-binding domains bind BCMA.

In some embodiments, the antigen-binding domain in the anti-BCMA CAR, such as an scFv, comprises a V_{H} and a V_{L} region. In some embodiments, the V_{H} region comprises a CDR-H1 set forth in SEQ ID NO: 62, a CDR-H2 set forth in SEQ ID NO:63 and a CDR-H3 set forth in SEQ ID NO:64, and the V_{L} region comprises a CDR-L1 set forth in SEQ ID NO: 65, a CDR-L2 set forth in SEQ ID NO:66 and a CDR-H3 set forth in SEQ ID NO:67. In some embodiments, the V_{H} region has the sequence of amino acids set forth in SEQ ID NO:30 or a sequence of amino acids that exhibits at least 90%, at least 91%, at least 92%, at least 93%, at least 94%, at least 95%, at least 96%, at least 97%, at least 98%, at least 99% to SEQ ID NO:30, and the V_{L} region has the sequence of amino acids set forth in SEQ ID NO:31 or a sequence of amino acids that exhibits at least 90%, at least 91%, at least 92%, at least 93%, at least 94%, at least 95%, at least 96%, at least 97%, at least 98%, at least 99% to SEQ ID NO:31. In some embodiments, the V_{H} region has the sequence of amino acids set forth in SEQ ID NO:30 and the V_{L} region has the sequence of amino acids set forth in SEQ ID NO:31. In some embodiments, the V_{H} region is amino-terminal to the V_{L} region. In some embodiments, the V_{H} region is carboxy-terminal to the V_{L} region. In some embodiments, the antigen-binding domain is an scFv that has the sequence of amino acids set forth in SEQ ID NO:68 or a sequence of amino acids exhibits at least 90%, at least 91%, at least 92%, at least 93%, at least 94%, at least 95%, at least 96%, at least 97%, at least 98%, at least 99% to SEQ ID NO:68. In particular embodiments, any of the above antigen-binding domains bind BCMA.

In some embodiments, the recombinant receptor such as the CAR, such as the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a C_{H}1/C_{L}, and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer.

Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is a spacer having at least a particular length, such as having a length that is at least 100 amino acids, such as at least 110, 125, 130, 135, 140, 145, 150, 160, 170, 180, 190, 200, 210, 220, 230, 240, or 250 amino acids in length. Exemplary spacers include IgG4 hinge alone, IgG4 hinge linked to C_{H}2 and C_{H}3 domains, or IgG4 hinge linked to the C_{H}3 domain. Exemplary spacers include IgG4 hinge alone, IgG4 hinge linked to C_{H}2 and C_{H}3 domains, or IgG4 hinge linked to the C_{H}3 domain. Exemplary spacers include, but are not limited to, those described in Hudecek et al., Clin. Cancer Res., 19:3153 (2013), Hudecek et al. (2015) Cancer Immunol Res. 3(2): 125-135, international patent application publication number WO2014031687, U.S. Pat. No. 8,822,647 or published app. No. US2014/0271635. In some embodiments, the spacer includes a sequence of an immunoglobulin hinge region, a C_{H}2 and C_{H}3 region. In some embodiments, one of more of the hinge, C_{H}2 and C_{H}3 is derived all or in part from IgG4 or IgG2. In some cases, the hinge, C_{H}2 and C_{H}3 is derived from IgG4. In some aspects, one or more of the hinge, C_{H}2 and C_{H}3 is chimeric and contains sequence derived from IgG4 and IgG2. In some examples, the spacer contains an IgG4/2 chimeric hinge, an IgG2/4 C_{H}2, and an IgG4 C_{H}3 region.

In some embodiments, the spacer, which can be a constant region or portion thereof of an immunoglobulin, is of a human IgG, such as IgG4 or IgG1. In some embodiments, the spacer has the sequence ESKYGPPCPPCP (set forth in SEQ ID NO: 1). In some embodiments, the spacer has the sequence set forth in SEQ ID NO: 3. In some embodiments, the spacer has the sequence set forth in SEQ ID NO: 4. In some embodiments, the encoded spacer is or contains the sequence set forth in SEQ ID NO: 29. In some embodiments, the constant region or portion is of IgD. In some embodiments, the spacer has the sequence set forth in SEQ ID NO: 5. In some embodiments, the spacer has the sequence set forth in SEQ ID NO: 125.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence or an N176Q. at position 176, in the C_{H}2 region, of the full-length IgG4 Fc sequence.

In some embodiments, the spacer can be of a length that provides for increased responsiveness of the cell following antigen binding, as compared to in the absence of the spacer and/or in the presence of a different spacer, such as one different only in length. In some embodiments, the spacer is at least 100 amino acids in length, such as at least 110, 125, 130, 135, 140, 145, 150, 160, 170, 180, 190, 200, 210, 220, 230, 240, or 250 amino acids in length. In some examples, the spacer is at or about 12 amino acids in length or is no more than 12 amino acids in length. Exemplary spacers include those having at least about 10 to 300 amino acids, about 10 to 200 amino acids, about 50 to 175 amino acids, about 50 to 150 amino acids, about 10 to 125 amino acids, about 50 to 100 amino acids, about 100 to 300 amino acids, about 100 to 250 amino acids, about 125 to 250 amino acids, or about 200 to 250 amino acids, and including any integer between the endpoints of any of the listed ranges. In some embodiments, a spacer or a spacer region is at least about 12 amino acids, at least about 119 amino acids or less, at least about 125 amino acids, at least about 200 amino acids, or at least about 220 amino acids, or at least about 225 amino acids in length.

In some embodiments, the spacer has a length of 125 to 300 amino acids in length, 125 to 250 amino acids in length, 125 to 230 amino acids in length, 125 to 200 amino acids in length, 125 to 180 amino acids in length, 125 to 150 amino acids in length, 150 to 300 amino acids in length, 150 to 250 amino acids in length, 150 to 230 amino acids in length, 150 to 200 amino acids in length, 150 to 180 amino acids in length, 180 to 300 amino acids in length, 180 to 250 amino acids in length, 180 to 230 amino acids in length, 180 to 200 amino acids in length, 200 to 300 amino acids in length, 200 to 250 amino acids in length, 200 to 230 amino acids in length, 230 to 300 amino acids in length, 230 to 250 amino acids in length or 250 to 300 amino acids in length. In some embodiments, the spacer is at least or at least about or is or is about 130, 140, 150, 160, 170, 180, 190, 200, 210, 220, 221, 222, 223, 224, 225, 226, 227, 228 or 229 amino acids in length, or a length between any of the foregoing.

Exemplary spacers include those containing portion(s) of an immunoglobulin constant region such as those containing an Ig hinge, such as an IgG hinge domain. In some aspects, the spacer includes an IgG hinge alone, an IgG hinge linked to one or more of a C_{H}2 and C_{H}3 domain, or IgG hinge linked to the C_{H}3 domain. In some embodiments, the IgG hinge, C_{H}2 and/or C_{H}3 can be derived all or in part from IgG4 or IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C_{H}2 region is chimeric and comprises a C_{H}2 region from human IgG4 and human IgG2; and/or the C_{H}3 region is chimeric and comprises a C_{H}3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C_{H}2 region; and a human IgG4 C_{H}3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence set forth in SEQ ID NO: 182 or an N176Q. at position 176, in the C_{H}2 region, of the full-length IgG2 Fc sequence set forth in SEQ ID NO: 181. In some embodiments, the spacer is or comprises an IgG4/2 chimeric hinge or a modified IgG4 hinge; an IgG2/4 chimeric C_{H}2 region; and an IgG4 C_{H}3 region and optionally is about 228 amino acids in length; or a spacer set forth in SEQ ID NO: 29. In some embodiments, the spacer comprises the amino acid sequence

(SEQ ID NO: 29)
ESKYGPPCPPCPAPPVAGPSVFLFPPKPKDTLMISRTPEVTCVVVDVSQ
EDPEVQFNWYVDGVEVHNAKTKPREEQFQSTYRVVSVLTVLHQDWLNGK
EYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKS
RWQEGNVFSCSVMHEALHNHYTQKSLSLSLGK

In some embodiments, the spacer is encoded by a polynucleotide that has been optimized for codon expression and/or to eliminate splice sites such as cryptic splice sites. In some embodiments, the coding sequence for the spacer comprises the nucleic acid sequence set forth in SEQ ID NO: 183. In some embodiments, the coding sequence for the spacer comprises the nucleic acid sequence set forth in SEQ ID NO: 179.

Other exemplary spacer regions include hinge regions derived from CD8a, CD28, CTLA4, PD-1, or FcγRIIIa. In some embodiments, the spacer contains a truncated extracellular domain or hinge region of a CD8a, CD28, CTLA4, PD-1, or FcγRIIIa. In some embodiments, the spacer is a truncated CD28 hinge region. In some embodiments, a short oligo- or polypeptide linker, for example, a linker of between 2 and 10 amino acids in length, such as one containing alanines or alanine and arginine, e.g., alanine triplet (AAA) or RAAA (SEQ ID NO: 46), is present and forms a linkage between the scFv and the spacer region of the CAR.

In some embodiments, the spacer is derived from CD28. In some embodiments, the spacer is a CD28 hinge. In some embodiments, the spacer has the sequence set forth in SEQ ID NO: 114. In some embodiments, the spacer has the sequence set forth in SEQ ID NO: 116.

In some embodiments the spacer is derived from CD8. In some embodiments, the spacer is a CD8 hinge sequence. In some embodiments, the spacer has the sequence set forth in any of SEQ ID NOs: 117. In some embodiments, the spacer has the sequence set forth in SEQ ID NO:118. In some embodiments, the spacer has the sequence set forth in SEQ ID NO:119.

In some embodiments, the spacer is derived from CTLA-4. In some embodiments, the spacer is a CD28 hinge. In some embodiments, the spacer has the sequence set forth in SEQ ID NO: 120.

In some embodiments, the spacer is derived from PD-1. In some embodiments, the spacer is a PD-1 hinge. In some embodiments, the spacer has the sequence set forth in SEQ ID NO: 122.

In some embodiments, the spacer is derived from Fc(gamma)RIIIa. In some embodiments, the spacer is a Fc(gamma)RIIIa hinge. In some embodiments, the spacer has the sequence set forth in SEQ ID NO: 124.

In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1, 3, 4, 5, 29, 114, 116, 117, 118, 119, 120, 122, 124, or 125.

This antigen recognition domain generally is linked to one or more intracellular signaling components, such as signaling components that mimic stimulation and/or activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD8a, CD9, CD16, CD22, CD33, CD37, CD64, CD80, CD86, CD 134, CD137 (4-1BB), CD154, CTLA-4, or PD-1. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). Exemplary sequences of transmembrane domains are or comprise the sequences set forth in SEQ ID NOs: 8, 115, 121, 123, 44, 45, 115, or 178.

In some embodiments, the transmembrane domain is a transmembrane domain from CD8. In some embodiments, the transmembrane domain as the sequence set forth in SEQ ID NO:44. In some embodiments, the transmembrane domain as the sequence set forth in SEQ ID NO:45. In some embodiments, the transmembrane domain as the sequence set forth in SEQ ID NO:115. In some embodiment, the transmembrane domain has the sequence set forth in SEQ ID NO:178.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell stimulation and/or activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25 or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell stimulation and/or activation is in some aspects described as being mediated by two classes of cytoplasmic signaling sequences: those that initiate antigen-dependent primary stimulation and/or activation through the TCR (primary cytoplasmic signaling regions, domains or sequences), and those that act in an antigen-independent manner to provide a secondary or co-stimulatory signal (secondary cytoplasmic signaling regions, domains or sequences). In some aspects, the CAR includes one or both of such signaling components.

In some aspects, the CAR includes a primary cytoplasmic signaling regions, domains or sequence that regulates primary activation of the TCR complex. Primary cytoplasmic signaling regions, domains or sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CD8, CD22, CD79a, CD79b and CD66d. In some embodiments, cytoplasmic signaling molecule(s) in the CAR contain(s) a cytoplasmic signaling domain, portion thereof, or sequence derived from CD3 zeta. In some embodiments, the CAR includes a signaling region and/or transmembrane portion of a costimulatory receptor, such as CD28, 4-1BB, OX40 (CD134), CD27, DAP10, DAP12, ICOS and/or other costimulatory receptors. In some aspects, the same CAR includes both the primary cytoplasmic signaling region and costimulatory signaling components.

In some embodiments, one or more different recombinant receptors can contain one or more different intracellular signaling region(s) or domain(s). In some embodiments, the primary cytoplasmic signaling region is included within one CAR, whereas the costimulatory component is provided by another receptor, e.g., another CAR recognizing another antigen. In some embodiments, the CARs include activating or stimulatory CARs, and costimulatory CARs, both expressed on the same cell (see WO2014/055668).

In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and primary cytoplasmic signaling region, in the cytoplasmic portion. Exemplary CARs include intracellular components, such as intracellular signaling region(s) or domain(s), of CD3-zeta, CD28, CD137 (4-1BB), OX40 (CD134), CD27, DAP10, DAP12, NKG2D and/or ICOS. In some embodiments, the chimeric antigen receptor contains an intracellular signaling region or domain of a T cell costimulatory molecule, e.g., from CD28, CD137 (4-1BB), OX40 (CD134), CD27, DAP10, DAP12, NKG2D and/or ICOS, in some cases, between the transmembrane domain and intracellular signaling region or domain. In some aspects, the T cell costimulatory molecule is one or more of CD28, CD137 (4-1BB), OX40 (CD134), CD27, DAP10, DAP12, NKG2D and/or ICOS.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 4-1BB.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P10747.1) or CD8a (Accession No. P01732.1) or variant thereof, such as a transmembrane domain that comprises the sequence of amino acids set forth in SEQ ID NO: 8, 115, 44, or 45 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8, 115, 44, or 45; in some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the costimulatory signaling domain is set forth in SEQ ID NO:10. In some embodiments, the costimulatory signaling domain is set forth in SEQ ID NO:11.

In some embodiments, the intracellular signaling component(s) of the recombinant receptor, e.g. the CAR, contains an intracellular costimulatory signaling domain of human 4-1BB or a functional variant or portion thereof. In some embodiments, the intracellular domain comprises an intracellular costimulatory signaling domain of 4-1BB (e.g. Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12. In some embodiments, the costimulatory signaling domain is set forth in SEQ ID NO:12.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3 (Accession No. P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15. In some embodiments, the CD3 zeta signaling domain is set forth in SEQ ID NO:13. In some embodiments, the CD3 zeta signaling domain is set forth in SEQ ID NO:14. In some embodiments, the CD3 zeta signaling domain is set forth in SEQ ID NO:15.

In some aspects, the spacer contains only a hinge region of an IgG, such as only a hinge of IgG4 or IgG1, such as the hinge only spacer set forth in SEQ ID NO: 1 or SEQ ID NO: 125. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 4. In some embodiments, the spacer is an Ig hinge, e.g., an IgG4 hinge, linked to a CH3 domain only, such as set forth in SEQ ID NO: 3. In some embodiments, the spacer is or comprises a glycine-serine rich sequence or other flexible linker such as known flexible linkers. In some embodiments, the spacer is a CD8a hinge, such as set forth in any of SEQ ID NOs: 117-119, an FcγRIIIa hinge, such as set forth in SEQ ID NO: 124, a CTLA4 hinge, such as set forth in SEQ ID NO: 120, or a PD-1 hinge, such as set forth in SEQ ID NO: 122.

For example, in some embodiments, the CAR includes an antibody such as an antibody fragment, such as an scFv, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. Such sequences can include any as described herein. In some embodiments, the CAR has the sequence of amino acids set forth in SEQ ID NO:161 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:161. In some embodiments, the CAR has the sequence set forth in SEQ ID NO:161. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain. Such sequences can include any as described herein. In some embodiments, the CAR has the sequence of amino acids set forth in SEQ ID NO:160 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:160. In some embodiments, the CAR has the sequence set forth in SEQ ID NO:160. In some embodiments, the CAR is encoded by a sequence of nucleotides set forth in SEQ ID NO:69.

in some embodiments, the CAR includes an antibody such as an antibody fragment, such as an scFv, a spacer, such as a spacer containing a CD8 hinge, a transmembrane domain containing all or a portion of a CD8-derived transmembrane domain, a 4-1BB-derived intracellular signaling domain, and a CD3 zeta signaling domain. Such sequences can include any as described herein. In some embodiments, the CAR has the sequence of amino acids set forth in SEQ ID NO:152 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:152. In some embodiments, the CAR has the sequence set forth in SEQ ID NO:152. In some embodiments, the CAR has the sequence of amino acids set forth in SEQ ID NO:168 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:168. In some embodiments, the CAR has the sequence set forth in SEQ ID NO:168. In some embodiments, the CAR has the sequence of amino acids set forth in SEQ ID NO:171 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:171. In some embodiments, the CAR has the sequence set forth in SEQ ID NO:171.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition. Non-limiting exemplary CAR sequences are set forth in any one of SEQ ID NOs: 126-177.

In some embodiments, the encoded CAR can sequence can further include a signal sequence or signal peptide that directs or delivers the CAR to the surface of the cell in which the CAR is expressed. In some embodiments, the signal peptide is derived from a transmembrane protein. In some examples the signal peptide is derived from CD8a, CD33, or an IgG. Exemplary signal peptides include the sequences set forth in SEQ ID NOs: 21, 75 and 76 or variant thereof.

In some embodiments, such CAR constructs further includes a T2A ribosomal skip element and/or a tEGFR sequence, e.g., downstream of the CAR.

B. Cells and Preparation of Cells for Genetic Engineering

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4⁺ and/or of CD8⁺ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al., Blood. 1:72-82 (2012); Wang et al., J Immunother. 35(9):689-701 (2012). In some embodiments, combining TCM-enriched CD8⁺ T cells and CD4⁺ T cells further enhances efficacy.

In embodiments, memory T cells are present in both CD62L⁺ and CD62L− subsets of CD8⁺ peripheral blood lymphocytes. PBMC can be enriched for or depleted of CD62L-CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO−, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L− and CD45RO−.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in PCT Pub. Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al., J Immunother. 35(9): 651-660 (2012), Terakura et al., Blood. 1:72-82 (2012), and Wang et al., J Immunother. 35(9):689-701 (2012).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al., Lab Chip 10, 1567-1573 (2010); and Godin et al., J Biophoton. 1(5):355-376 (2008). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al., J Immunother. 35(9): 651-660 (2012), Terakura et al., Blood. 1:72-82 (2012), and/or Wang et al., J Immunother. 35(9):689-701 (2012).

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4⁺ and/or CD8⁺ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

C. Nucleic Acids, Vectors and Methods for Genetic Engineering

In some embodiments, the cells, e.g., T cells, are genetically engineered to express a recombinant receptor. In some embodiments, the engineering is carried out by introducing nucleic acid molecules that encode the recombinant receptor. Also provided are nucleic acid molecules encoding a recombinant receptor, and vectors or constructs containing such nucleic acids and/or nucleic acid molecules.

In some cases, the nucleic acid sequence encoding the recombinant receptor, e.g., chimeric antigen receptor (CAR), contains a signal sequence that encodes a signal peptide. In some aspects, the signal sequence may encode a signal peptide derived from a native polypeptide. In other aspects, the signal sequence may encode a heterologous or non-native signal peptide. In some embodiments, the signal peptide is derived from a transmembrane protein. In some examples the signal peptide is derived from CD8a, CD33, or an IgG. Non-limiting exemplary examples of signal peptides include, for example, the CD33 signal peptide set forth in SEQ ID NO:21, CD8a signal peptide set forth in SEQ ID NO:75, or the signal peptide set forth in SEQ ID NO:76 or modified variant thereof.

In some embodiments, the nucleic acid molecule encoding the recombinant receptor contains at least one promoter that is operatively linked to control expression of the recombinant receptor. In some examples, the nucleic acid molecule contains two, three, or more promoters operatively linked to control expression of the recombinant receptor. In some embodiments, nucleic acid molecule can contain regulatory sequences, such as transcription and translation initiation and termination codons, which are specific to the type of host (e.g., bacterium, fungus, plant, or animal) into which the nucleic acid molecule is to be introduced, as appropriate and taking into consideration whether the nucleic acid molecule is DNA- or RNA-based. In some embodiments, the nucleic acid molecule can contain regulatory/control elements, such as a promoter, an enhancer, an intron, a polyadenylation signal, a Kozak consensus sequence, and splice acceptor or donor. In some embodiments, the nucleic acid molecule can contain a nonnative promoter operably linked to the nucleotide sequence encoding the recombinant receptor and/or one or more additional polypeptide(s). In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1a promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the nucleic acid molecule does not include a regulatory element, e.g. promoter.

In some embodiments, the nucleic acid molecule encoding the recombinant receptor, e.g., CAR or other antigen receptor, further includes nucleic acid sequences encoding a marker and/or cells expressing the CAR or other antigen receptor further includes a marker, e.g., a surrogate marker, such as a cell surface marker, which may be used to confirm transduction or engineering of the cell to express the receptor, such as a truncated version of a cell surface receptor, such as truncated EGFR (tEGFR). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the nucleic acid molecule, e.g., a nucleic acid molecule encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same nucleic acid molecule that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:11 or 76) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some aspects, the marker, e.g. surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 28, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 28. An exemplary T2A linker sequence comprises the sequence of amino acids set forth in SEQ ID NO: 6 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 7 or 28, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 28.

In some embodiments, a single promoter may direct expression of an RNA that contains, in a single open reading frame (ORF), two or three genes (e.g. encoding the molecule involved in modulating a metabolic pathway and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein, without limitation, 2A sequences from the foot-and-mouth disease virus (F2A, e.g., SEQ ID NO: 27), equine rhinitis A virus (E2A, e.g., SEQ ID NO: 26), Thosea asigna virus (T2A, e.g., SEQ ID NO: 6 or 23), and porcine teschovirus-1 (P2A, e.g., SEQ ID NO: 24 or 25) as described in U.S. Patent Publication No. 20070116690.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Introduction of the nucleic acid molecules encoding the recombinant receptor in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some embodiments, prior to or during gene transfer, the cells are incubated or cultured in the presence of an immunomodulatory compound, e.g., Compound A or Compound B, including any as described herein. In some embodiments, the immunomodulatory compound, e.g., Compound A or Compound B, is added during the cell manufacturing process, for example, during the process of engineering CAR-T cells. In some aspects, the presence of the immunomodulatory compound can improve the quality of the population of cells produced. In some aspects, the immunomodulatory compound, e.g., Compound A or Compound B, may increase the proliferation or expansion of cells or may alter one or more signaling pathways thereby resulting in cells with a less-differentiated or less activated surface phenotype, despite exhibiting substantial expansion and/or effector function.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29 (11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### III. EXEMPLARY TREATMENT OUTCOMES AND METHODS FOR ASSESSING SAME

In some embodiments of the methods, compositions, combinations, kits and uses provided herein, the provided combination therapy results in one or more treatment outcomes, such as a feature associated with any one or more of the parameters associated with the therapy or treatment, as described below. In some embodiments, the method includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells, e.g. T cell therapy, before or after administering the combination therapy provided herein.

In some embodiments, the combination therapy can further include one or more screening steps to identify subjects for treatment with the combination therapy and/or continuing the combination therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the combination therapy provided herein.

In some embodiments, any of the screening steps and/or assessment of treatment of outcomes described herein can be used prior to, during, during the course of, or subsequent to administration of one or more steps of the provided combination therapy, e.g., administration of the T cell therapy (e.g. CAR-expressing T cells), and/or an immunomodulatory compound, e.g., Compound A or Compound B. In some embodiments, assessment is made prior to, during, during the course of, or after performing any of the methods provided herein. In some embodiments, the assessment is made prior to performing the methods provided herein. In some embodiments, assessment is made after performing one or more steps of the methods provided herein. In some embodiments, the assessment is performed prior to administration of administration of one or more steps of the provided combination therapy, for example, to screen and identify patients suitable and/or susceptible to receive the combination therapy. In some embodiments, the assessment is performed during, during the course of, or subsequent to administration of one or more steps of the provided combination therapy, for example, to assess the intermediate or final treatment outcome, e.g., to determine the efficacy of the treatment and/or to determine whether to continue or repeat the treatments and/or to determine whether to administer the remaining steps of the combination therapy.

In some embodiments, treatment of outcomes includes improved immune function, e.g., immune function of the T cells administered for cell based therapy and/or of the endogenous T cells in the body. In some embodiments, exemplary treatment outcomes include, but are not limited to, enhanced T cell proliferation, enhanced T cell functional activity, changes in immune cell phenotypic marker expression, such as such features being associated with the engineered T cells, e.g. CAR-T cells, administered to the subject. In some embodiments, exemplary treatment outcomes include decreased disease burden, e.g., tumor burden, improved clinical outcomes and/or enhanced efficacy of therapy.

In some embodiments, the screening step and/or assessment of treatment of outcomes includes assessing the survival and/or function of the T cells administered for cell based therapy. In some embodiments, the screening step and/or assessment of treatment of outcomes includes assessing the levels of cytokines or growth factors. In some embodiments, the screening step and/or assessment of treatment of outcomes includes assessing disease burden and/or improvements, e.g., assessing tumor burden and/or clinical outcomes. In some embodiments, either of the screening step and/or assessment of treatment of outcomes can include any of the assessment methods and/or assays described herein and/or known in the art, and can be performed one or more times, e.g., prior to, during, during the course of, or subsequently to administration of one or more steps of the combination therapy. Exemplary sets of parameters associated with a treatment outcome, which can be assessed in some embodiments of the methods provided herein, include peripheral blood immune cell population profile and/or tumor burden.

In some embodiments, the methods affect efficacy of the cell therapy in the subject. In some embodiments, the persistence, expansion, and/or presence of recombinant receptor-expressing, e.g., CAR-expressing, cells in the subject following administration of the dose of cells in the method with the immunomodulatory compound is greater as compared to that achieved via a method without the administration of the immunomodulatory compound. In some embodiments of the immunotherapy methods provided herein, such as a T cell therapy (e.g. CAR-expressing T cells), assessment of the parameter includes assessing the expansion and/or persistence in the subject of the administered T cells for the immunotherapy, e.g., T cell therapy, as compared to a method in which the immunotherapy is administered to the subject in the absence of the immunomodulatory compound. In some embodiments, the methods result in the administered T cells exhibiting increased or prolonged expansion and/or persistence in the subject as compared to a method in which the T cell therapy is administered to the subject in the absence of the immunomodulatory compound.

In some embodiments, the administration of the immunomodulatory compound, e.g., Compound A or Compound B decreases disease burden, e.g., tumor burden, in the subject as compared to a method in which the dose of cells expressing the recombinant receptor is administered to the subject in the absence of the immunomodulatory compound. In some embodiments, the administration of the immunomodulatory compound, e.g., Compound A or Compound B decreases blast marrow in the subject as compared to a method in which the dose of cells expressing the recombinant receptor is administered to the subject in the absence of the immunomodulatory compound. In some embodiments, the administration of the immunomodulatory compound, e.g., Compound A or Compound B, results in improved clinical outcomes, e.g., objective response rate (ORR), progression-free survival (PFS) and overall survival (OS), compared to a method in which the dose of cells expressing the recombinant receptor is administered to the subject in the absence of the immunomodulatory compound.

In some embodiments, the subject can be screened prior to the administration of one or more steps of the combination therapy. For example, the subject can be screened for characteristics of the disease and/or disease burden, e.g., tumor burden, prior to administration of the combination therapy, to determine suitability, responsiveness and/or susceptibility to administering the combination therapy. In some embodiments, the screening step and/or assessment of treatment outcomes can be used to determine the dose, frequency, duration, timing and/or order of the combination therapy provided herein.

In some embodiments, the subject can be screened after administration of one of the steps of the combination therapy, to determine and identify subjects to receive the remaining steps of the combination therapy and/or to monitor efficacy of the therapy. In some embodiments, the number, level or amount of administered T cells and/or proliferation and/or activity of the administered T cells is assessed prior to administration and/or after administration of the immunomodulatory compound, e.g., Compound A or Compound B.

In some embodiments, the immunomodulatory compound, e.g., Compound A or Compound B is administered until the concentration or number of engineered cells in the blood of the subject is (i) at least at or about 10 engineered cells per microliter, (ii) at least 20%, 30%, 40% or 50% of the total number of peripheral blood mononuclear cells (PBMCs), (iii) at least or at least about 1×10⁵ engineered cells; or (iv) at least 5,000 copies of recombinant receptor-encoding DNA per micrograms DNA; and/or at day 90 following the initiation of the administration in (a), CAR-expressing cells are detectable in the blood or serum of the subject; and/or at day 90 following the initiation of the administration in (a), the blood of the subject contains at least 20% CAR-expressing cells, at least 10 CAR-expressing cells per microliter or at least 1×10⁴ CAR-expressing cells.

In some embodiments, the immunomodulatory compound, e.g., Compound A or Compound B is administered until there is a clinical benefit to the treatment, such as at least or greater than a 50% decrease in the total tumor volume or a complete response (CR) in which detectable tumor has disappeared, progression free survival or disease free survival for greater than 6 months or greater than 1 year or more.

In some embodiments, a change and/or an alteration, e.g., an increase, an elevation, a decrease or a reduction, in levels, values or measurements of a parameter or outcome compared to the levels, values or measurements of the same parameter or outcome in a different time point of assessment, a different condition, a reference point and/or a different subject is determined or assessed. For example, in some embodiments, a fold change, e.g., an increase or decrease, in particular parameters, e.g., number of engineered T cells in a sample, compared to the same parameter in a different condition, e.g., before or after administration of the immunomodulatory compound, e.g., Compound A or Compound B can be determined. In some embodiments, the levels, values or measurements of two or more parameters are determined, and relative levels are compared. In some embodiments, the determined levels, values or measurements of parameters are compared to the levels, values or measurements from a control sample or an untreated sample. In some embodiments, the determined levels, values or measurements of parameters are compared to the levels from a sample from the same subject but at a different time point. The values obtained in the quantification of individual parameter can be combined for the purpose of disease assessment, e.g., by forming an arithmetical or logical operation on the levels, values or measurements of parameters by using multi-parametric analysis. In some embodiments, a ratio of two or more specific parameters can be calculated.

A. T Cell Exposure, Persistence and Proliferation

In some embodiments, the parameter associated with therapy or a treatment outcome, which include parameters that can be assessed for the screening steps and/or assessment of treatment of outcomes and/or monitoring treatment outcomes, is or includes assessment of the exposure, persistence and proliferation of the T cells, e.g., T cells administered for the T cell based therapy. In some embodiments, the increased exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells used for the immunotherapy, e.g. T cell therapy, before or after administering one or more steps of the combination therapy provided herein.

In some embodiments, the administration of the immunomodulatory compound, e.g., Compound A or Compound B, are designed to promote exposure of the subject to the cells, e.g., T cells administered for T cell based therapy, such as by promoting their expansion and/or persistence over time. In some embodiments, the T cell therapy exhibits increased or prolonged expansion and/or persistence in the subject as compared to a method in which the T cell therapy is administered to the subject in the absence of the immunomodulatory compound, e.g., Compound A or Compound B.

In some embodiments, the provided methods increase exposure of the subject to the administered cells (e.g., increased number of cells or duration over time) and/or improve efficacy and therapeutic outcomes of the immunotherapy, e.g. T cell therapy. In some aspects, the methods are advantageous in that a greater and/or longer degree of exposure to the cells expressing the recombinant receptors, e.g., CAR-expressing cells, improves treatment outcomes as compared with other methods. Such outcomes may include patient survival and remission, even in individuals with severe tumor burden.

In some embodiments, the administration of the immunomodulatory compound, e.g., Compound A or Compound B can increase the maximum, total, and/or duration of exposure to the cells, e.g. T cells administered for the T cell based therapy, in the subject as compared to administration of the T cells alone in the absence of the immunomodulatory compound. In some aspects, administration of the immunomodulatory compound, e.g., Compound A or Compound B, in the context of high disease burden (and thus higher amounts of antigen) and/or a more aggressive or resistant cancer enhances efficacy as compared with administration of the T cells alone in the absence of the immunomodulatory compound in the same context, which may result in immunosuppression, anergy and/or exhaustion which may prevent expansion and/or persistence of the cells.

In some embodiments, the presence and/or amount of cells expressing the recombinant receptor (e.g., CAR-expressing cells administered for T cell based therapy) in the subject following the administration of the T cells and before, during and/or after the administration of the immunomodulatory compound, e.g., Compound A or Compound B is detected. In some aspects, quantitative PCR (qPCR) is used to assess the quantity of cells expressing the recombinant receptor (e.g., CAR-expressing cells administered for T cell based therapy) in the blood or serum or organ or tissue sample (e.g., disease site, e.g., tumor sample) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample.

In some embodiments, the cells are detected in the subject at or at least at 4, 14, 15, 27, or 28 days following the administration of the T cells, e.g., CAR-expressing T cells. In some aspects, the cells are detected at or at least at 2, 4, or 6 weeks following, or 3, 6, or 12, 18, or 24, or 30 or 36 months, or 1, 2, 3, 4, 5, or more years, following the administration of the T cells, e.g., CAR-expressing T cells and/or the immunomodulatory compound, e.g., Compound A or Compound B.

In some embodiments, the persistence of receptor-expressing cells (e.g. CAR-expressing cells) in the subject by the methods, following the administration of the T cells, e.g., CAR-expressing T cells and/or the immunomodulatory compound, e.g., Compound A or Compound B, is greater as compared to that which would be achieved by alternative methods such as those involving the administration of the immunotherapy alone, e.g., administration the T cells, e.g., CAR-expressing T cells, in the absence of the immunomodulatory compound.

The exposure, e.g., number of cells, e.g. T cells administered for T cell therapy, indicative of expansion and/or persistence, may be stated in terms of maximum numbers of the cells to which the subject is exposed, duration of detectable cells or cells above a certain number or percentage, area under the curve for number of cells over time, and/or combinations thereof and indicators thereof. Such outcomes may be assessed using known methods, such as qPCR to detect copy number of nucleic acid encoding the recombinant receptor compared to total amount of nucleic acid or DNA in the particular sample, e.g., blood, serum, plasma or tissue, such as a tumor sample, and/or flow cytometric assays detecting cells expressing the receptor generally using antibodies specific for the receptors. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor.

In some aspects, increased exposure of the subject to the cells includes increased expansion of the cells. In some embodiments, the receptor expressing cells, e.g. CAR-expressing cells, expand in the subject following administration of the T cells, e.g., CAR-expressing T cells, and/or following administration of immunomodulatory compound, e.g., Compound A or Compound B. In some aspects, the methods result in greater expansion of the cells compared with other methods, such as those involving the administration of the T cells, e.g., CAR-expressing T cells, in the absence of administering the immunomodulatory compound, e.g., Compound A or Compound B.

In some aspects, the method results in high in vivo proliferation of the administered cells, for example, as measured by flow cytometry. In some aspects, high peak proportions of the cells are detected. For example, in some embodiments, at a peak or maximum level following the administration of the T cells, e.g., CAR-expressing T cells and/or the immunomodulatory compound, e.g., Compound A or Compound B, in the blood or disease-site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some embodiments, the method results in a maximum concentration, in the blood or serum or other bodily fluid or organ or tissue of the subject, of at least 100, 500, 1000, 1500, 2000, 5000, 10,000 or 15,000 copies of or nucleic acid encoding the receptor, e.g., the CAR, per microgram of DNA, or at least 0.1, 0.2, 0.3, 0.4, 0.5, 0.6, 0.7, 0.8, or 0.9 receptor-expressing, e.g., CAR,-expressing cells per total number of peripheral blood mononuclear cells (PBMCs), total number of mononuclear cells, total number of T cells, or total number of microliters. In some embodiments, the cells expressing the receptor are detected as at least 10, 20, 30, 40, 50, or 60% of total PBMCs in the blood of the subject, and/or at such a level for at least 1, 2, 3, 4, 5, 6, 7, 8, 9, 10, 11, 12, 24, 36, 48, or 52 weeks following the T cells, e.g., CAR-expressing T cells and/or the immunomodulatory compound, e.g., Compound A or Compound B, or for 1, 2, 3, 4, or 5, or more years following such administration.

In some aspects, the method results in at least a 2-fold, at least a 4-fold, at least a 10-fold, or at least a 20-fold increase in copies of nucleic acid encoding the recombinant receptor, e.g., CAR, per microgram of DNA, e.g., in the serum, plasma, blood or tissue, e.g., tumor sample, of the subject.

In some embodiments, cells expressing the receptor are detectable in the serum, plasma, blood or tissue, e.g., tumor sample, of the subject, e.g., by a specified method, such as qPCR or flow cytometry-based detection method, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more days following administration of the T cells, e.g., CAR-expressing T cells, or after administration of the immunomodulatory compound, e.g., Compound A or Compound B, for at least at or about 2, 3, 4, 5, 6, 7, 8, 9, 10, 11, 12, 13, 14, 15, 16, 17, 18, 19, 20, 21, 22, 23, or 24 or more weeks following the administration of the T cells, e.g., CAR-expressing T cells, and/or the immunomodulatory compound, e.g., Compound A or Compound

B.

In some aspects, at least about 1×10², at least about 1×10³, at least about 1×10⁴, at least about 1×10⁵, or at least about 1×10⁶ or at least about 5×10⁶ or at least about 1×10⁷ or at least about 5×10⁷ or at least about 1×10⁸ recombinant receptor-expressing, e.g., CAR-expressing cells, and/or at least 10, 25, 50, 100, 200, 300, 400, or 500, or 1000 receptor-expressing cells per microliter, e.g., at least 10 per microliter, are detectable or are present in the subject or fluid, plasma, serum, tissue, or compartment thereof, such as in the blood, e.g., peripheral blood, or disease site, e.g., tumor, thereof. In some embodiments, such a number or concentration of cells is detectable in the subject for at least about 20 days, at least about 40 days, or at least about 60 days, or at least about 3, 4, 5, 6, 7, 8, 9, 10, 11, or 12 months, or at least 2 or 3 years, following administration of the T cells, e.g., CAR-expressing T cells, and/or following the administration of the immunomodulatory compound, e.g., Compound A or Compound B. Such cell numbers may be as detected by flow cytometry-based or quantitative PCR-based methods and extrapolation to total cell numbers using known methods. See, e.g., Brentjens et al., Sci Transl Med. 2013 5(177), Park et al, Molecular Therapy 15(4):825-833 (2007), Savoldo et al., JCI 121(5):1822-1826 (2011), Davila et al., (2013) PLoS ONE 8(4):e61338, Davila et al., Oncoimmunology 1(9):1577-1583 (2012), Lamers, Blood 2011 117:72-82, Jensen et al., Biol Blood Marrow Transplant 2010 September; 16(9): 1245-1256, Brentjens et al., Blood 2011 118(18):4817-4828.

In some aspects, the copy number of nucleic acid encoding the recombinant receptor, e.g., vector copy number, per 100 cells, for example in the peripheral blood or bone marrow or other compartment, as measured by immunohistochemistry, PCR, and/or flow cytometry, is at least 0.01, at least 0.1, at least 1, or at least 10, at about 1 week, about 2 weeks, about 3 weeks, about 4 weeks, about 5 weeks, or at least about 6 weeks, or at least about 2, 3, 4, 5, 6, 7, 8. 9, 10, 11, or 12 months or at least 2 or 3 years following administration of the cells, e.g., CAR-expressing T cells, and/or the immunomodulatory compound, e.g., Compound A or Compound B. In some embodiments, the copy number of the vector expressing the receptor, e.g. CAR, per microgram of genomic DNA is at least 100, at least 1000, at least 5000, or at least 10,000, or at least 15,000 or at least 20,000 at a time about 1 week, about 2 weeks, about 3 weeks, or at least about 4 weeks following administration of the T cells, e.g., CAR-expressing T cells, or immunomodulatory compound, e.g., Compound A or Compound B, or at least 2, 3, 4, 5, 6, 7, 8, 9, 10, 11, or 12 months or at least 2 or 3 years following such administration.

In some aspects, the receptor, e.g. CAR, expressed by the cells, is detectable by quantitative PCR (qPCR) or by flow cytometry in the subject, plasma, serum, blood, tissue and/or disease site thereof, e.g., tumor site, at a time that is at least about 3 months, at least about 6 months, at least about 12 months, at least about 1 year, at least about 2 years, at least about 3 years, or more than 3 years, following the administration of the cells, e.g., following the initiation of the administration of the T cells, e.g., CAR-expressing T cells, and/or the immunomodulatory compound, e.g., Compound A or Compound B.

In some embodiments, the area under the curve (AUC) for concentration of receptor- (e.g., CAR−) expressing cells in a fluid, plasma, serum, blood, tissue, organ and/or disease site, e.g. tumor site, of the subject over time following the administration of the T cells, e.g., CAR− expressing T cells and/or immunomodulatory compound, e.g., Compound A or Compound B, is greater as compared to that achieved via an alternative dosing regimen where the subject is administered the T cells, e.g., CAR-expressing T cells, in the absence of administering the immunomodulatory compound.

In some aspects, the method results in high in vivo proliferation of the administered cells, for example, as measured by flow cytometry. In some aspects, high peak proportions of the cells are detected. For example, in some embodiments, at a peak or maximum level following the T cells, e.g., CAR-expressing T cells and/or immunomodulatory compound, e.g., Compound A or Compound B, in the blood, plasma, serum, tissue or disease site of the subject or white blood cell fraction thereof, e.g., PBMC fraction or T cell fraction, at least about 10%, at least about 20%, at least about 30%, at least about 40%, at least about 50%, at least about 60%, at least about 70%, at least about 80%, or at least about 90% of the cells express the recombinant receptor, e.g., the CAR.

In some aspects, the increased or prolonged expansion and/or persistence of the dose of cells in the subject administered with the immunomodulatory compound, e.g., Compound A or Compound B is associated with a benefit in tumor related outcomes in the subject. In some embodiments, the tumor related outcome includes a decrease in tumor burden or a decrease in blast marrow in the subject. In some embodiments, the tumor burden is decreased by or by at least at or about 10, 20, 30, 40, 50, 60, 70, 80, 90, or 100 percent after administration of the method. In some embodiments, disease burden, tumor size, tumor volume, tumor mass, and/or tumor load or bulk is reduced following the dose of cells by at least at or about 50%, 60%, 70%, 80%, 90% or more compared a subject that has been treated with a method that does not involve the administration of an immunomodulatory compound, e.g., Compound A or Compound B.

B. T Cell Functional Activity

In some embodiments, parameters associated with therapy or a treatment outcome, which include parameters that can be assessed for the screening steps and/or assessment of treatment of outcomes and/or monitoring treatment outcomes, includes one or more of activity, phenotype, proliferation or function of T cells. In some embodiments, any of the known assays in the art for assessing the activity, phenotypes, proliferation and/or function of the T cells, e.g., T cells administered for T cell therapy, can be used. Prior to and/or subsequent to administration of the cells and/or immunomodulatory compound, e.g., Compound A or Compound B, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al., J. Immunological Methods, 285(1): 25-40 (2004).

In some embodiments, T cells, such as recombinant-expressing (e.g. CAR) T cells, can be assessed prior to and/or subsequent to administration of the cells and/or immunomodulatory compound, e.g., Compound A or Compound B, to assess or determine if the T cells exhibit features of exhaustion. In some cases, exhaustion can be assessed by monitoring loss of T cell function, such as reduced or decreased antigen-specific or antigen receptor-driven activity, such as a reduced or decreased ability to produce cytokines or to drive cytolytic activity against target antigen. In some cases, exhaustion also can be assessed by monitoring expression of surface markers on T cells (e.g. CD4 and/or CD4 T cells) that are associated with an exhaustion phenotype. Among exhaustion markers are inhibitory receptors such as PD-1, CTLA-4, LAG-3 and TIM-3.

In some embodiments, such a reduced or decreased activity is observed over time following administration to the subject and/or following long-term exposure to antigen.

In particular embodiments, the provided methods (i) to effect said increase in antigen-specific or antigen receptor-driven activity and (ii) to prevent, inhibit or delay said onset of exhaustion phenotype and/or to reverse said exhaustion phenotype. In some embodiments, the amount, duration and/or frequency is effective (i) to effect said increase in antigen-specific or antigen receptor-driven activity and (ii) to prevent, inhibit or delay said onset of exhaustion phenotype. In other embodiments, the amount, duration and/or frequency is effective (i) to effect said increase in antigen-specific or antigen receptor-driven activity and (ii) to prevent, inhibit or delay said onset of exhaustion phenotype and to reverse said exhaustion phenotype.

wherein the exhaustion phenotype, with reference to a T cell or population of T cells, comprises: an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or a decrease in the level or degree of an activity exhibited by said T cells or population of T cells upon exposure to an antigen or antigen receptor-specific agent, compared to a reference T cell population, under the same conditions. an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or a decrease in the level or degree of an activity exhibited by said T cells or population of T cells upon exposure to an antigen or antigen receptor-specific agent, compared to a reference T cell population, under the same conditions.

In certain embodiments, the biological activity of the cells is measured by assaying expression and/or secretion of one or more cytokines, such as CD107a, IFNγ, IL-2, GM-CSF and TNFα, and/or by assessing cytolytic activity.

In some embodiments, assays for the activity, phenotypes, proliferation and/or function of the T cells, e.g., T cells administered for T cell therapy include, but are not limited to, ELISPOT, ELISA, cellular proliferation, cytotoxic lymphocyte (CTL) assay, binding to the T cell epitope, antigen or ligand, or intracellular cytokine staining, proliferation assays, lymphokine secretion assays, direct cytotoxicity assays, and limiting dilution assays. In some embodiments, proliferative responses of the T cells can be measured, e.g. by incorporation of ³H-thymidine, BrdU (5-Bromo-2′-Deoxyuridine) or 2′-deoxy-5-ethynyluridine (EdU) into their DNA or dye dilution assays, using dyes such as carboxyfluorescein diacetate succinimmunomodulatory compoundyl ester (CFSE), CellTrace Violet, or membrane dye PKH26.

In some embodiments, assessing the activity, phenotypes, proliferation and/or function of the T cells, e.g., T cells administered for T cell therapy, include measuring cytokine production from T cells, and/or measuring cytokine production in a biological sample from the subject, e.g., plasma, serum, blood, and/or tissue samples, e.g., tumor samples. In some cases, such measured cytokines can include, without limitation, interleukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). Assays to measure cytokines are well known in the art, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample.

In some embodiments, assessing the activity, phenotypes, proliferation and/or function of the T cells, e.g., T cells administered for T cell therapy, include assessing cell phenotypes, e.g., expression of particular cell surface markers. In some embodiments, the T cells, e.g., T cells administered for T cell therapy, are assessed for expression of T cell activation markers, T cell exhaustion markers, and/or T cell differentiation markers. In some embodiments, the cell phenotype is assessed before administration. In some embodiments, the cell phenotype is assessed after administration. T cell activation markers, T cell exhaustion markers, and/or T cell differentiation markers for assessment include any markers known in the art for particular subsets of T cells, e.g., CD25, CD38, human leukocyte antigen-DR (HLA-DR), CD69, CD44, CD137, KLRG1, CD62L^{low}, CCR7^{low}, CD71, CD2, CD54, CD58, CD244, CD160, programmed cell death protein 1 (PD-1), lymphocyte activation gene 3 protein (LAG-3), T-cell immunoglobulin domain and mucin domain protein 3 (TIM-3), cytotoxic T lymphocyte antigen-4 (CTLA-4), band T lymphocyte attenuator (BTLA) and/or T-cell immunoglobulin and immunoreceptor tyrosine-based inhibitory motif domain (TIGIT) (see, e.g., Liu et al., Cell Death and Disease (2015) 6, e1792). In some embodiments, the exhaustion marker is any one or more of PD-1, CTLA-4, TIM-3, LAG-3, BTLA, 2B4, CD160, CD39, VISTA, and TIGIT. In some embodiments, the assessed cell surface marker is CD25, PD-1 and/or TIM-3. In some embodiments, the assessed cell surface marker is CD25.

In some aspects, detecting the expression levels includes performing an in vitro assay. In some embodiments, the in vitro assay is an immunoassay, an aptamer-based assay, a histological or cytological assay, or an mRNA expression level assay. In some embodiments, the parameter or parameters for one or more of each of the one or more factors, effectors, enzymes and/or surface markers are detected by an enzyme linked immunosorbent assay (ELISA), immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay. In some embodiments, detection of cytokines and/or surface markers is determined using a binding reagent that specifically binds to at least one biomarker. In some cases, the binding reagent is an antibody or antigen-binding fragment thereof, an aptamer or a nucleic acid probe.

In some embodiments, the administration of the immunomodulatory compound, e.g., Compound A or Compound B increases the level of circulating CAR T cells.

C. Disease Burden

In some embodiments, parameters associated with therapy or a treatment outcome, which include parameters that can be assessed for the screening steps and/or assessment of treatment of outcomes and/or monitoring treatment outcomes, includes tumor or disease burden. The administration of the immunotherapy, such as a T cell therapy (e.g. CAR-expressing T cells) and/or the immunomodulatory compound, e.g., Compound A or Compound B, can reduce or prevent the expansion or burden of the disease or condition in the subject. For example, where the disease or condition is a tumor, the methods generally reduce tumor size, bulk, metastasis, percentage of blasts in the bone marrow or molecularly detectable cancer and/or improve prognosis or survival or other symptom associated with tumor burden.

In some embodiments, the provided methods result in a decreased tumor burden in treated subjects compared to alternative methods in which the immunotherapy, such as a T cell therapy (e.g. CAR-expressing T cells) is given without administration of the immunomodulatory compound, e.g., Compound A or Compound B. It is not necessary that the tumor burden actually be reduced in all subjects receiving the combination therapy, but that tumor burden is reduced on average in subjects treated, such as based on clinical data, in which a majority of subjects treated with such a combination therapy exhibit a reduced tumor burden, such as at least 50%, 60%, 70%, 80%, 90%, 95% or more of subjects treated with the combination therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In the case of MM, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/1); renal insufficiency attributable to myeloma; anemia (hemoglobin <10 g/dl); and/or bone lesions (lytic lesions or osteoporosis with compression fractures)).

Exemplary methods for assessing disease status or disease burden include: measurement of M protein in biological fluids, such as blood and/or urine, by electrophoresis and immunofixation; quantification of sFLC (κ and λ) in blood; skeletal survey; and imaging by positron emission tomography (PET)/computed tomography (CT) in subjects with extramedullary disease. In some embodiments, disease status can be evaluated by bone marrow examination. In some examples, efficacy of the T cell therapy following its administration to the subject is determined by the expansion and persistence of the cells (e.g. CAR-expressing cells) in the blood and/or bone marrow. In some embodiments, efficacy of the T cell therapy is determined based on the antitumor activity of the administered cell (e.g. CAR-expressing cells). In some embodiments antitumor activity is determined by the overall response rate (ORR) and/or International Myeloma Working Group (IMWG) Uniform Response Criteria (see Kumar et al. (2016) Lancet Oncol 17(8):e328-346). In some embodiments, response is evaluated using minimal residual disease (MRD) assessment. In some embodiments, MRD can be assessed by methods such as flow cytometry and high-throughput sequencing, e.g., deep sequencing. In some aspects, subjects that have a MRD-negative disease include those exhibiting Absence of aberrant clonal plasma cells on bone marrow aspirate, ruled out by an assay with a minimum sensitivity of 1 in 10⁵ nucleated cells or higher (i.e., 10⁻⁵ sensitivity), such as flow cytometry (next-generation flow cytometry; NGF) or high-throughput sequencing, e.g., deep sequencing or next-generation sequencing (NGS).

In some aspects, sustained MRD-negative includes subjects that exhibit MRD negativity in the marrow (NGF or NGS, or both) and by imaging as defined below, confirmed minimum of 1 year apart. Subsequent evaluations can be used to further specify the duration of negativity (e.g., MRD-negative at 5 years). In some aspects, flow MRD-negative includes subjects that exhibit an absence of phenotypically aberrant clonal plasma cells by NGF on bone marrow aspirates using the EuroFlow standard operation procedure for MRD detection in multiple myeloma (or validated equivalent method) with a minimum sensitivity of 1 in 10⁵ nucleated cells or higher. In some aspects, sequencing MRD-negative includes subjects that exhibit an absence of clonal plasma cells by NGS on bone marrow aspirate in which presence of a clone is defined as less than two identical sequencing reads obtained after DNA sequencing of bone marrow aspirates using the LymphoSIGHT platform (or validated equivalent method) with a minimum sensitivity of 1 in 10⁵ nucleated cells or higher. In some aspects, imaging plus MRD-negative includes subjects that exhibit MRD negativity as assessed by NGF or NGS plus disappearance of every area of increased tracer uptake found at baseline or a preceding PET/CT or decrease to less mediastinal blood pool SUV or decrease to less than that of surrounding normal tissue (see Kumar et al. (2016) Lancet Oncol 17(8):e328-346).

In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of tumor burden is specified. In some embodiments, exemplary parameters for determination include particular clinical outcomes indicative of amelioration or improvement in the tumor. Such parameters include: duration of disease control, including objective response (OR), complete response (CR), stringent complete response (sCR), very good partial response (VGPR), partial response (PR), minimal response (MR), Stable disease (SD), Progressive disease (PD) or relapse (see, e.g., International Myeloma Working Group (IMWG) Uniform Response Criteria; see Kumar et al. (2016) Lancet Oncol 17(8):e328-346), objective response rate (ORR), progression-free survival (PFS) and overall survival (OS). In some embodiments, response is evaluated using minimal residual disease (MRD) assessment. Specific thresholds for the parameters can be set to determine the efficacy of the methods provided herein. In some embodiments, the disease or disorder to be treated is multiple myeloma. In some embodiments, measurable disease criteria for multiple myeloma can include (1) serum M-protein 1 g/dL or greater; (2) Urine M-protein 200 mg or greater/24 hour; (3) involved serum free light chain (sFLC) level 10 mg/dL or greater, with abnormal κ to λ ratio. In some cases, light chain disease is acceptable only for subjects without measurable disease in the serum or urine.

In some embodiments, response is evaluated based on the duration of response following administration of the T cell therapy, e.g. CAR-expressing T cells. In some aspects, the response to the therapy, e.g., according to the provided embodiments, can be measured at a designated timepoint after the initiation of administration of the cell therapy. In some embodiments, the designated timepoint is at or about 1, 2, 3, 6, 9, 12, 18, 24, 30 or 36 months following initiation of the administration, or within a range defined by any of the foregoing. In some embodiments, the designated time point is 4, 8, 12, 16, 20, 24, 28, 32, 36, 48 or 52 weeks months following initiation of the administration, or within a range defined by any of the foregoing. In some embodiments, the designated timepoint is at or about 1 month following initiation of the administration. In some embodiments, the designated timepoint is at or about 3 months following initiation of the administration. In some embodiments, the designated timepoint is at or about 6 months following initiation of the administration. In some embodiments, the designated timepoint is at or about 9 months following initiation of the administration. In some embodiments, the designated timepoint is at or about 12 months following initiation of the administration.

In some embodiments, the response or outcome determined at or about 3, 6, 9 or 12 months after the designated timepoint is equal to or improved compared to the response or outcome determined at the initial designated timepoint. For example, in some aspects, if the response or outcome determined at the initial designated timepoint is stable disease (SD), Progressive disease (PD) or relapse, the subject treated according to the provided embodiments can show an equal or improved response or outcome (e.g., exhibiting a better response outcome according to the International Myeloma Working Group (IMWG) Uniform Response Criteria; see Kumar et al. (2016) Lancet Oncol 17(8):e328-346) at a subsequent time point, after at or about 3, 6, 9 or 12 months after the initial designated timepoint, that is equal to the response or outcome at the initial designated timepoint, or a response or outcome that is objective response (OR), complete response (CR), stringent complete response (sCR), very good partial response (VGPR) or partial response (PR). In some aspects, subjects treated according to the provided embodiments can show a response or outcome that is improved between two time point of determination. In some aspects, the subject can exhibit a PR or VGPR in the initial designated timepoint for assessment, e.g., at 4 weeks after the initiation of administration, then exhibit an improved response, such as a CR or an sCR, at a later time point, e.g., at 12 weeks after the initiation of administration. In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR; also known in some cases as overall response rate) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response or outcome is durable for greater than at or about 3, 6, 9 or 12 months.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of self-care; see e.g., Sorensen et al., (1993) Br J Cancer 67(4) 773-775. In some embodiments, the subject that are to be administered according to the methods or treatment regimen provided herein include those with an ECOG performance status of 0 or 1.

In some embodiments, the methods and/or administration of an immunotherapy, such as a T cell therapy (e.g. CAR-expressing T cells) and/or immunomodulatory compound, e.g., Compound A or Compound B decrease(s) disease burden as compared with disease burden at a time immediately prior to the administration of the immunotherapy, e.g., T cell therapy and/or immunomodulatory compound.

In some aspects, administration of the immunotherapy, e.g. T cell therapy and/or immunomodulatory compound, e.g., Compound A or Compound B, may prevent an increase in disease burden, and this may be evidenced by no change in disease burden.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives immunotherapy, e.g. T cell therapy alone, in the absence of administration of the immunomodulatory compound, e.g., Compound A or Compound B. In some embodiments, disease burden is reduced to a greater extent or for a greater duration following the combination therapy of administration of the immunotherapy, e.g., T cell therapy, and the immunomodulatory compound, e.g., Compound A or Compound B, compared to the reduction that would be effected by administering each of the agent alone, e.g., administering the immunomodulatory compound to a subject having not received the immunotherapy, e.g. T cell therapy; or administering the immunotherapy, e.g. T cell therapy, to a subject having not received the immunomodulatory compound.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the mass of a solid tumor and/or the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). Specific thresholds for the parameters can be set to determine the efficacy of the method of combination therapy provided herein.

In some embodiments, the subjects treated according to the method achieve a more durable response. In some cases, a measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months, greater than 6 months, or great than 12 months. In some particular embodiments, the subjects treated according to the method achieve a more durable response after the subject previously relapsed following remission in response to the administration of the genetically engineered cells.

In some aspects, disease burden is measured or detected prior to administration of the immunotherapy, e.g. T cell therapy, following the administration of the immunotherapy, e.g. T cell therapy but prior to administration of the immunomodulatory compound, e.g., Compound A or Compound B, following administration of the immunomodulatory compound but prior to the administration of the immunotherapy, e.g., T cell therapy, and/or following the administration of both the immunotherapy, e.g. T cell therapy and the immunomodulatory compound. In the context of multiple administration of one or more steps of the combination therapy, disease burden in some embodiments may be measured prior to or following administration of any of the steps, doses and/or cycles of administration, or at a time between administration of any of the steps, doses and/or cycles of administration.

In some embodiments, the burden is decreased by or by at least at or about 10, 20, 30, 40, 50, 60, 70, 80, 90, or 100 percent by the provided methods compared to immediately prior to the administration of the immunomodulatory compound, e.g., Compound A or Compound B and the immunotherapy, e.g. T cell therapy. In some embodiments, disease burden, tumor size, tumor volume, tumor mass, and/or tumor load or bulk is reduced following administration of the immunotherapy, e.g. T cell therapy and the immunomodulatory compound, by at least at or about 10, 20, 30, 40, 50, 60, 70, 80, 90% or more compared to that immediately prior to the administration of the immunotherapy, e.g. T cell therapy and/or the immunomodulatory compound.

In some embodiments, reduction of disease burden by the method comprises an induction in morphologic complete remission, for example, as assessed at 1 month, 2 months, 3 months, or more than 3 months, after administration of, e.g., initiation of, the combination therapy.

In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of combination therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the method of combination therapy, is less than about 80%, less than about 70%, less than about 60%, less than about 50%, less than about 40%, less than about 30%, less than about 20%, or less than about 10%.

In some embodiments, the administration can treat the subject despite the subject having become resistant to another therapy. In some embodiments, when administered to subjects according to the embodiments described herein, the dose or the composition is capable of achieving objective response (OR), in at least 50%, at least 60%, at least 70%, at least 80%, at least 90%, or at least 95% of subjects that were administered. In some embodiments, OR includes subjects who achieve stringent complete response (sCR), complete response (CR), very good partial response (VGPR), partial response (PR) and minimal response (MR). In some embodiments, when administered to subjects according to the embodiments described herein, the dose or the composition is capable of achieving stringent complete response (sCR), complete response (CR), very good partial response (VGPR) or partial response (PR), in at least 50%, 60%, 70%, 80%, or 85% of subjects that were administered. In some embodiments, when administered to subjects according to the embodiments described herein, the dose or the composition is capable of achieving stringent complete response (sCR) or complete response (CR) at least 20%, 30%, 40% 50%, 60% or 70% of subjects that were administered. In some embodiments, exemplary doses include about 1.0×10⁷, 1.5×10⁷, 2.0×10⁷, 2.5×10⁷, 5.0×10⁷, 1.5×10⁸, 3.0×10⁸, 4.5×10⁸, 6.0×10⁸ or 8.0×10⁸ CAR-expressing (CAR+) T cells. In some embodiments, exemplary doses include about 5.0×10⁷, 1.5×10⁸, 3.0×10⁸, 4.5×10⁸, 6.0×10⁸ or 8.0×10⁸ CAR-expressing (CAR+) T cells. In some embodiments, exemplary doses include about 5.0×10⁷, 1.5×10⁸, 3.0×10⁸ or 4.5×10⁸ CAR-expressing (CAR+) T cells. In some aspects, particular response to the treatment, e.g., according to the methods provided herein, can be assessed based on the International Myeloma Working Group (IMWG) Uniform Response Criteria (see Kumar et al. (2016) Lancet Oncol 17(8):e328-346).

### IV. TOXICITY AND ADVERSE OUTCOMES

In embodiments of the provided methods, the subject is monitored for toxicity or other adverse outcome, including treatment related outcomes, e.g., development of neutropenia, cytokine release syndrome (CRS) or neurotoxicity (NT), in subjects administered the provided combination therapy comprising a cell therapy (e.g., a T cell therapy) and an immunomodulatory compound, e.g. Compound A or Compound B. In some embodiments, the provided methods are carried out to reduce the risk of a toxic outcome or symptom, toxicity-promoting profile, factor, or property, such as a symptom or outcome associated with or indicative of severe neutropenia, severe cytokine release syndrome (CRS) or severe neurotoxicity.

In some embodiments, the methods do not result in, or do not increase the risk of, certain hematological toxicities, such as neutropenia or thrombocytopenia. In some embodiments, no more than 50% of subjects exhibit a neutropenia higher than grade 3, such as a prolonged grade 3 neutropenia or a grade 4 neutropenia, and/or a thrombocytopenia higher than grade 3, such as a grade 3 or grade 4 thrombocytopenia. In some embodiments, at least 50% of subjects treated according to the method (e.g. at least 60%, at least 70%, at least 80%, at least 90% or more of the subjects treated) do not exhibit a severe neutropenia or a severe thrombocytopenia of grade 3 or higher than grade 3

In some embodiments, the provided methods do not result in a high rate or likelihood of toxicity or toxic outcomes, or reduces the rate or likelihood of toxicity or toxic outcomes, such as severe neurotoxicity (NT) or severe cytokine release syndrome (CRS), such as compared to certain other cell therapies. In some embodiments, the methods do not result in, or do not increase the risk of, severe NT (sNT), severe CRS (sCRS), macrophage activation syndrome, tumor lysis syndrome, fever of at least at or about 38 degrees Celsius for three or more days and a plasma level of CRP of at least at or about 20 mg/dL. In some embodiments, greater than or greater than about 30%, 35%, 40%, 50%, 55%, 60% or more of the subjects treated according to the provided methods do not exhibit any grade of CRS or any grade of neurotoxicity. In some embodiments, no more than 50% of subjects treated (e.g. at least 60%, at least 70%, at least 80%, at least 90% or more of the subjects treated) a cytokine release syndrome (CRS) higher than grade 2 and/or a neurotoxicity higher than grade 2. In some embodiments, at least 50% of subjects treated according to the method (e.g. at least 60%, at least 70%, at least 80%, at least 90% or more of the subjects treated) do not exhibit a severe toxic outcome (e.g. severe CRS or severe neurotoxicity), such as do not exhibit grade 3 or higher neurotoxicity and/or does not exhibit severe CRS, or does not do so within a certain period of time following the treatment, such as within a week, two weeks, or one month of the administration of the cells.

A. Cytokine Release Syndrome (CRS) and Neurotoxicity

In some aspects, the subject is monitored for and/or the methods reduce the risk for a toxic outcome that is or is associated with or indicative of cytokine release syndrome (CRS) or severe CRS (sCRS). CRS, e.g., sCRS, can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure. CRS may be treated using anti-inflammatory therapy such as an anti-IL-6 therapy, e.g., anti-IL-6 antibody, e.g., tocilizumab, or antibiotics or other agents as described.

Outcomes, signs and symptoms of CRS are known and include those described herein. In some embodiments, where a particular dosage regimen or administration effects or does not effect a given CRS-associated outcome, sign, or symptom, particular outcomes, signs, and symptoms and/or quantities or degrees thereof may be specified.

In the context of administering CAR-expressing cells, CRS typically occurs 6-20 days after infusion of cells that express a CAR. See Xu et al., Cancer Letters 343 (2014) 172-78. In some cases, CRS occurs less than 6 days or more than 20 days after CAR T cell infusion. The incidence and timing of CRS may be related to baseline cytokine levels or tumor burden at the time of infusion. Commonly, CRS involves elevated serum levels of interferon (IFN)-γ, tumor necrosis factor (TNF)-α, and/or interleukin (IL)-2. Other cytokines that may be rapidly induced in CRS are IL-1β, IL-6, IL-8, and IL-10.

Exemplary outcomes associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly.

In some embodiments, outcomes associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures).

Exemplary CRS-related outcomes include increased or high serum levels of one or more factors, including cytokines and chemokines and other factors associated with CRS. Exemplary outcomes further include increases in synthesis or secretion of one or more of such factors. Such synthesis or secretion can be by the T cell or a cell that interacts with the T cell, such as an innate immune cell or B cell.

In some embodiments, the CRS-associated serum factors or CRS-related outcomes include inflammatory cytokines and/or chemokines, including interferon gamma (IFN-γ), TNF-a, IL-1β, IL-2, IL-6, IL-7, IL-8, IL-10, IL-12, sIL-2Ra, granulocyte macrophage colony stimulating factor (GM-CSF), macrophage inflammatory protein (MIP)-1, tumor necrosis factor alpha (TNFα), IL-6, and IL-10, IL-1β, IL-8, IL-2, MIP-1, Flt-3L, fracktalkine, and/or IL-5. In some embodiments, the factor or outcome includes C reactive protein (CRP). In addition to being an early and easily measurable risk factor for CRS, CRP also is a marker for cell expansion. In some embodiments, subjects that are measured to have high levels of CRP, such as ≥15 mg/dL, have CRS. In some embodiments, subjects that are measured to have high levels of CRP do not have CRS. In some embodiments, a measure of CRS includes a measure of CRP and another factor indicative of CRS.

In some embodiments, one or more inflammatory cytokines or chemokines are monitored before, during, or after CAR treatment and/or treatment with Compound A or Compound B. In some aspects, the one or more cytokines or chemokines include IFN-γ, TNF-α, IL-2, IL-1β, IL-6, IL-7, IL-8, IL-10, IL-12, sIL-2Ra, granulocyte macrophage colony stimulating factor (GM-CSF), or macrophage inflammatory protein (MIP). In some embodiments, IFN-γ, TNF-α, and IL-6 are monitored.

CRS criteria that appear to correlate with the onset of CRS to predict which patients are more likely to be at risk for developing sCRS have been developed (see Davilla et al. Science translational medicine. 2014; 6(224):224ra25). Factors include fevers, hypoxia, hypotension, neurologic changes, elevated serum levels of inflammatory cytokines, such as a set of seven cytokines (IFNγ, IL-5, IL-6, IL-10, Flt-3L, fractalkine, and GM-CSF) whose treatment-induced elevation can correlate well with both pretreatment tumor burden and sCRS symptoms. Other guidelines on the diagnosis and management of CRS are known (see e.g., Lee et al, Blood. 2014; 124(2):188-95). In some embodiments, the criteria reflective of CRS grade are those detailed in Table 2 below.

TABLE 2
Exemplary Grading Criteria for CRS

| Grade | Description of Symptoms |
|---|---|
| 1 | Not life-threatening, require only symptomatic |
| Mild | treatment such as antipyretics and anti-emetics |
| | (e.g., fever, nausea, fatigue, headache, |
| | myalgias, malaise) |
| 2 | Require and respond to moderate intervention: |
| Moderate | Oxygen requirement <40%, or |
| | Hypotension responsive to fluids or low dose |
| | of a single vasopressor, or |
| | Grade 2 organ toxicity (by CTCAE v4.0) |
| 3 | Require and respond to aggressive intervention: |
| Severe | Oxygen requirement ≥40%, or |
| | Hypotension requiring high dose of a single |
| | vasopressor (e.g., norepinephrine ≥20 μg/kg/min, |
| | dopamine ≥10 μg/kg/min, |
| | phenylephrine ≥200 μg/kg/min, or |
| | epinephrine ≥10 μg/kg/min), or |
| | Hypotension requiring multiple vasopressors (e.g., |
| | vasopressin + one of the above agents, or |
| | combination vasopressors equivalent to ≥20 μg/kg/min |
| | norepinephrine), or |
| | Grade 3 organ toxicity or Grade 4 transaminitis |
| | (by CTCAE v4.0) |
| 4 | Life-threatening: |
| Life- | Requirement for ventilator support, or |
| threatening | Grade 4 organ toxicity (excluding transaminitis) |
| 5 | Death |
| Fatal | |

In some embodiments, a subject is deemed to develop “severe CRS” (“sCRS”) in response to or secondary to administration of a cell therapy or dose of cells thereof, if, following administration, the subject displays: (1) fever of at least 38 degrees Celsius for at least three days; (2) cytokine elevation that includes either (a) a max fold change of at least 75 for at least two of the following group of seven cytokines compared to the level immediately following the administration: interferon gamma (IFNγ), GM-CSF, IL-6, IL-10, Flt-3L, fracktalkine, and IL-5 and/or (b) a max fold change of at least 250 for at least one of the following group of seven cytokines compared to the level immediately following the administration: interferon gamma (IFNγ), GM-CSF, IL-6, IL-10, Flt-3L, fracktalkine, and IL-5; and (c) at least one clinical sign of toxicity such as hypotension (requiring at least one intravenous vasoactive pressor) or hypoxia (PO₂<90%) or one or more neurologic disorder(s) (including mental status changes, obtundation, and/or seizures). In some embodiments, severe CRS includes CRS with a grade of 3 or greater, such as set forth in Table 2.

In some embodiments, outcomes associated with severe CRS or grade 3 CRS or greater, such as grade 4 or greater, include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines, such as a max fold change, e.g., of at least at or about 75, compared to pre-treatment levels of at least two cytokines (e.g., at least two of the group consisting of interferon gamma (IFNγ), GM-CSF, IL-6, IL-10, Flt-3L, fracktalkine, and IL-5, and/or tumor necrosis factor alpha (TNFα)), or a max fold change, e.g., of at least at or about 250 of at least one of such cytokines; and/or at least one clinical sign of toxicity, such as hypotension (e.g., as measured by at least one intravenous vasoactive pressor); hypoxia (e.g., plasma oxygen (P02) levels of less than at or about 90%); and/or one or more neurologic disorders (including mental status changes, obtundation, and seizures). In some embodiments, severe CRS includes CRS that requires management or care in the intensive care unit (ICU).

In some embodiments, the CRS, such as severe CRS, encompasses a combination of (1) persistent fever (fever of at least 38 degrees Celsius for at least three days) and (2) a serum level of CRP of at least at or about 20 mg/dL. In some embodiments, the CRS encompasses hypotension requiring the use of two or more vasopressors or respiratory failure requiring mechanical ventilation. In some embodiments, the dosage of vasopressors is increased in a second or subsequent administration.

In some embodiments, severe CRS or grade 3 CRS encompasses an increase in alanine aminotransferase, an increase in aspartate aminotransferase, chills, febrile neutropenia, headache, left ventricular dysfunction, encephalopathy, hydrocephalus, and/or tremor.

The method of measuring or detecting the various outcomes may be specified.

In some aspects, the toxic outcome of a therapy, such as a cell therapy, is or is associated with or indicative of neurotoxicity or severe neurotoxicity. In some embodiments, symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03).

In some instances, neurologic symptoms may be the earliest symptoms of sCRS. In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, a subject is deemed to develop “severe neurotoxicity” in response to or secondary to administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table 3. In some embodiments, a severe neurotoxicity is deemed to be a prolonged grade 3 if symptoms or grade 3 neurotoxicity last for 10 days or longer.

TABLE 3
Exemplary Grading Criteria for neurotoxicity

| Grade | Description of Symptoms |
|---|---|
| 1 | Mild or asymptomatic symptoms |
| Asymptomatic | |
| or Mild | |
| 2 | Presence of symptoms that limit instrumental |
| Moderate | activities of daily living (ADL), such as |
| | preparing meals, shopping for groceries or |
| | clothes, using the telephone, managing money |
| 3 | Presence of symptoms that limit self-care ADL, |
| Severe | such as bathing, dressing and undressing, feeding |
| | self, using the toilet, taking medications |
| 4 | Symptoms that are life-threatening, requiring |
| Life- | urgent intervention |
| threatening | |
| 5 | Death |
| Fatal | |

In some embodiments, the methods reduce symptoms associated with CRS or neurotoxicity compared to other methods. In some aspects, the provided methods reduce symptoms, outcomes or factors associated with CRS, including symptoms, outcomes or factors associated with severe CRS or grade 3 or higher CRS, compared to other methods. For example, subjects treated according to the present methods may lack detectable and/or have reduced symptoms, outcomes or factors of CRS, e.g. severe CRS or grade 3 or higher CRS, such as any described, e.g. set forth in Table 2. In some embodiments, subjects treated according to the present methods may have reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods. In some embodiments, subjects treated according to the present methods may have reduced symptoms associated with peripheral motor neuropathy, peripheral sensory neuropathy, dysethesia, neuralgia or paresthesia.

In some embodiments, the methods reduce outcomes associated with neurotoxicity including damages to the nervous system and/or brain, such as the death of neurons. In some aspects, the methods reduce the level of factors associated with neurotoxicity such as beta amyloid (Aβ), glutamate, and oxygen radicals.

In some embodiments, the toxicity outcome is a dose-limiting toxicity (DLT). In some embodiments, the toxic outcome is the absence of a dose-limiting toxicity. In some embodiments, a dose-limiting toxicity (DLT) is defined as any grade 3 or higher toxicity as described or assessed by any known or published guidelines for assessing the particular toxicity, such as any described above and including the National Cancer Institute (NCI) Common Terminology Criteria for Adverse Events (CTCAE) version 4.0. In some embodiments, a dose-limiting toxicity (DLT) is defined when any of the events discussed below occurs following administration of the cell therapy (e.g., T cell therapy) and/or Compound A or Compound B, the events including a) febrile neutropenia; b) Grade 4 neutropenia lasting about or more than about 7 days; c) Grade 3 or 4 thrombocytopenia with clinically significant bleeding; and d) Grade 4 thrombocytopenia lasting more than 24 hours.

In some embodiments, the provided embodiments result in a low rate or risk of developing a toxicity, e.g. CRS or neurotoxicity or severe CRS or neurotoxicity, e.g. grade 3 or higher CRS or neurotoxicity, such as observed with administering a dose of T cells in accord with the provided combination therapy, and/or with the provided articles of manufacture or compositions. In some cases, this permits administration of the cell therapy on an outpatient basis. In some embodiments, the administration of the cell therapy, e.g. dose of T cells (e.g. CAR+ T cells) in accord with the provided methods, and/or with the provided articles of manufacture or compositions, is performed on an outpatient basis or does not require admission to the subject to the hospital, such as admission to the hospital requiring an overnight stay.

In some aspects, subjects administered the cell therapy, e.g. dose of T cells (e.g. CAR+ T cells) in accord with the provided methods, and/or with the provided articles of manufacture or compositions, including subjects treated on an outpatient basis, are not administered an intervention for treating any toxicity prior to or with administration of the cell dose, unless or until the subject exhibits a sign or symptom of a toxicity, such as of a neurotoxicity or CRS.

In some embodiments, if a subject administered the cell therapy, e.g. dose of T cells (e.g. CAR+ T cells), including subjects treated on an outpatient basis, exhibits a fever the subject is given or is instructed to receive or administer a treatment to reduce the fever. In some embodiments, the fever in the subject is characterized as a body temperature of the subject that is (or is measured at) at or above a certain threshold temperature or level. In some aspects, the threshold temperature is that associated with at least a low-grade fever, with at least a moderate fever, and/or with at least a high-grade fever. In some embodiments, the threshold temperature is a particular temperature or range. For example, the threshold temperature may be at or about or at least at or about 38, 39, 40, 41, or 42 degrees Celsius, and/or may be a range of at or about 38 degrees Celsius to at or about 39 degrees Celsius, a range of at or about 39 degrees Celsius to at or about 40 degrees Celsius, a range of at or about 40 degrees Celsius to at or about 41 degrees, or a range of at or about 41 degrees Celsius to at or about 42 degrees Celsius.

In some embodiments, the treatment designed to reduce fever includes treatment with an antipyretic. An antipyretic may include any agent, composition, or ingredient, that reduces fever, such as one of any number of agents known to have antipyretic effects, such as NSAIDs (such as ibuprofen, naproxen, ketoprofen, and nimesulide), salicylates, such as aspirin, choline salicylate, magnesium salicylate, and sodium salicylate, paracetamol, acetaminophen, Metamizole, Nabumetone, Phenaxone, antipyrine, febrifuges. In some embodiments, the antipyretic is acetaminophen. In some embodiments, acetaminophen can be administered at a dose of 12.5 mg/kg orally or intravenously up to every four hours. In some embodiments, it is or comprises ibuprofen or aspirin.

In some embodiments, if the fever is a sustained fever, the subject is administered an alternative treatment for treating the toxicity. For subjects treated on an outpatient basis, the subject is instructed to return to the hospital if the subject has and/or is determined to or to have a sustained fever. In some embodiments, the subject has, and/or is determined to or considered to have, a sustained fever if he or she exhibits a fever at or above the relevant threshold temperature, and where the fever or body temperature of the subject is not reduced, or is not reduced by or by more than a specified amount (e.g., by more than 1° C., and generally does not fluctuate by about, or by more than about, 0.5° C., 0.4° C., 0.3° C., or 0.2° C.), following a specified treatment, such as a treatment designed to reduce fever such as treatment with an antipyreticm, e.g. NSAID or salicylates, e.g. ibuprofen, acetaminophen or aspirin. For example, a subject is considered to have a sustained fever if he or she exhibits or is determined to exhibit a fever of at least at or about 38 or 39 degrees Celsius, which is not reduced by or is not reduced by more than at or about 0.5° C., 0.4° C., 0.3° C., or 0.2° C., or by at or about 1%, 2%, 3%, 4%, or 5%, over a period of 6 hours, over a period of 8 hours, or over a period of 12 hours, or over a period of 24 hours, even following treatment with the antipyretic such as acetaminophen. In some embodiments, the dosage of the antipyretic is a dosage ordinarily effective in such as subject to reduce fever or fever of a particular type such as fever associated with a bacterial or viral infection, e.g., a localized or systemic infection.

In some embodiments, the subject has, and/or is determined to or considered to have, a sustained fever if he or she exhibits a fever at or above the relevant threshold temperature, and where the fever or body temperature of the subject does not fluctuate by about, or by more than about, 1° C., and generally does not fluctuate by about, or by more than about, 0.5° C., 0.4° C., 0.3° C., or 0.2° C. Such absence of fluctuation above or at a certain amount generally is measured over a given period of time (such as over a 24-hour, 12-hour, 8-hour, 6-hour, 3-hour, or 1-hour period of time, which may be measured from the first sign of fever or the first temperature above the indicated threshold). For example, in some embodiments, a subject is considered to or is determined to exhibit sustained fever if he or she exhibits a fever of at least at or about or at least at or about 38 or 39 degrees Celsius, which does not fluctuate in temperature by more than at or about 0.5° C., 0.4° C., 0.3° C., or 0.2° C., over a period of 6 hours, over a period of 8 hours, or over a period of 12 hours, or over a period of 24 hours.

In some embodiments, the fever is a sustained fever; in some aspects, the subject is treated at a time at which a subject has been determined to have a sustained fever, such as within one, two, three, four, five six, or fewer hours of such determination or of the first such determination following the initial therapy having the potential to induce the toxicity, such as the cell therapy, such as dose of T cells, e.g. CAR+ T cells.

In some embodiments, one or more interventions or agents for treating the toxicity, such as a toxicity-targeting therapies, is administered at a time at which or immediately after which the subject is determined to or confirmed to (such as is first determined or confirmed to) exhibit sustained fever, for example, as measured according to any of the aforementioned embodiments. In some embodiments, the one or more toxicity-targeting therapies is administered within a certain period of time of such confirmation or determination, such as within 30 minutes, 1 hour, 2 hours, 3 hours, 4 hours, 6 hours, or 8 hours thereof.

B. Hematologic Toxicity

In some aspects, the subject is monitored for and/or the methods reduce the risk for a toxic outcome that is or is associated with or indicative of a hematologic toxicity, such as thrombocytopenia and/or neutropenia. In some cases, hematological toxicities, including thrombocytopenia and neutropenia, are graded according to Common Terminology Criteria for Adverse Events (Version 4.03; US National Cancer Institute, Bethesda, Md., USA). In some cases, a hematological toxicity, such as thrombocytopenia and/or neutropenia, is monitored before, during, and after the administration(s) of the immunomodulatory compound, e.g. Compound A or Compound B. In some cases, a hematological toxicity, such as thrombocytopenia and/or neutropenia, is monitored prior to each administration of the immunomodulatory compound, e.g. Compound A or Compound B. In some cases, a hematological toxicity, such as thrombocytopenia and/or neutropenia, is monitored at least every 1, 2, 3, 4, 5, 6, or 7 days after administration of the immunomodulatory compound, e.g. Compound A or Compound B.

In some embodiments, a complete blood count is carried out to monitor levels of leukocytes (white blood cells) in the subject, including neutrophils and platelets. A variety of methods can be used carry out a complete blood cell (CBC) count and/or a leukocyte differential count. In some embodiments, a hematology analyzer is used.

Neutropenia is characterized by a reduction in the blood neutrophil count, often leading to increased susceptibility to bacterial and fungal infections. Common symptoms of neutropenia in patients include, for example, fever, mouth sores, and ear infections. Patients with profound neutropenia often suffer from pyogenic infections such as septicemia, cutaneous cellulitis, liver abscesses, furunculosis, pneumonia, stomatitis, gingivitis, perirectal inflammation, colitis, sinusitis, and otitis media.

In some embodiments, the Absolute Neutrophil Count (ANC) is used to define levels of neutropenia. The ANC can be calculated from components of the complete blood count. In some embodiments, severity of neutropenia is classified based on the absolute neutrophil count (ANC) measured in cells per microliter of blood: a) mild neutropenia (1000 to 1500 cells/mL); b) moderate neutropenia (grade 3; 500 to 1000 cells/mL); c) severe neutropenia (grade 4; <500 cells/mL). In some embodiments, neutropenia can be graded according to criteria set forth in Table 4. Subjects with severe neutropenia often have severe risk of infection.

TABLE 4
Neutropenia grading

| Grade | ANC |
|---|---|
| Grade 1 | <2.0 × 10⁹/L (<2000/mm³) and >1.5 × 10⁹/L (>1500/mm³) |
| Grade 2 | <1.5 × 10⁹/L (<1500/mm³) and >1.0 × 10⁹/L (>1000/mm³) |
| Grade 3 | <1.0 × 10⁹/L (<1000/mm³) and >0.5 × 10⁹/L (>500/mm³) |
| Grade 4 | <0.5 × 10⁹/L (<500/mm³) |

In some cases, neutropenia is a febrile neutropenia (also called neutropenic fever or neutropenic sepsis). Febrile neutropenia occurs when a patient has a temperature greater than 38° C. and low levels of neutrophils or neutropenia. In some embodiments, febrile neutropenia can be graded according to criteria set forth in Table 5.

TABLE 5
Exemplary Grading Criteria for Febrile Neutropenia

| Grade | Description of symptoms |
|---|---|
| Grade 3 | ANC <1000/mm³ and a single temperature of >38.3 degrees C. |
| | (101 degrees F.) or a sustained temperature of >=38 degrees C. |
| | (100.4 degrees F.) for more than one hour |
| Grade 4 | life-threatening consequences and indicated urgent intervention |
| Grade 5 | death |

In some embodiments, a subject is monitored for thrombocytopenia. Thrombocytopenia is characterized by a platelet count of less than 150,000 cells per microliter (μL). Presentation of thrombocytopenia, particularly among patients with more severe grades, may include bleeding, ecchymoses, petechiae, purpura, and hypersplenism. Thrombocytopenia may be characterized as grade 1 thrombocytopenia (i.e., platelet count of 75,000 to 150,000/μL), grade 2 (i.e., platelet count of 50,000 to <75,000/μL), grade 3 (platelet count of 25,000 to <50,000/μL), or grade 4 (i.e., platelet count of below 25,000/μL).

In some embodiments of the provided methods, if a subject is determined to exhibit a hematological toxicity, such as thrombocytopenia and/or neutropenia or a particular grade thereof, the cycling therapy with the immunomodulatory compound, e.g. Compound A or Compound B can be altered. In some aspects, the cycling therapy is altered if, after administration of the immunomodulatory compound, e.g. Compound A or Compound B, the subject has a grade 3 or higher thrombocytopenia; a grade 3 neutropenia; a grade 3 neutropenia that is sustained (such as at least more than 3, 5, or 7 days); a grade 4 neutropenia; a Grade 3 or higher febrile neutropenia. In some embodiments, administration of the immunomodulatory compound, e.g. Compound A or Compound B is halted permanently or suspended until signs or symptoms of the toxicity is resolved, lessened or reduced. Continued monitoring of the subject can be carried out to assess one or more signs or symptoms of the toxicity, such as by CBC or differential leukocyte analysis. In some cases, if the toxicity resolves or is reduced, administration of Compound A or Compound B can be restarted at the same dose or dosing regimen prior to suspending the cycling therapy, at a lower or reduced dose, and/or in a dosing regimen involving less frequent dosing. In some embodiments, in instances of restarting the cycling therapy, the dose is lowered or reduced at least or at least about or about 10%, 15%, 20%, 25%, 30%, 40%, 50%, or 60%. In some embodiments, if the dose prior to suspending the cell therapy is 2 mg (e.g. given 5/7 days), the dose is reduced to 1 mg (given 5/7 days). In some aspects, if a hematological toxicity is of such severity that suspension of the cycling therapy is for greater than 4 weeks, the cycling therapy can be permanently discontinued.

In some embodiments, one or more agents can be administered to the subject to treat, ameliorate or lessen one or more symptoms associated with the hematological toxicity. In some cases, a myeloid growth factors, such as G-CSF or GM-CSF, is administered to the subject until the hematological toxicity improves. Examples of such therapies include filgrastim or pegfilgrastim. In some aspects, such agents are administered to subjects experiencing severe neutropenia or febrile neutropenia, including any grade 3 or greater neutropenia of any duration.

C. Non-Hematologic Toxicity

In some aspects, the toxic outcome is or is associated with or indicative of one or more non-hematologic toxicity following administration of the immunomodulatory compound, e.g. Compound A or Compound B. Examples of non-hematologic toxicities include, but are not limited to, tumor flare reaction, infections, tumor lysis syndrome, cardiac laboratory abnormalities, thromboembolic event(s) (such as deep vein thrombosis and pulmonary embolism), and/or pneumonitis.

In some aspects, the non-hematologic toxicity is tumor flare reaction (TFR) (sometimes also referred to pseudoprogression). TFR is a sudden increase in the size of the disease-bearing sites, including the lymph nodes, spleen and/or the liver often accompanied by a low-grade fever, tenderness and swelling, diffuse rash and in some cases, an increase in the peripheral blood lymphocyte counts. In some embodiments, TFR is graded according to Common Terminology Criteria for Adverse Events (Version 3.0; US National Cancer Institute, Bethesda, Md., USA). In some embodiments, TFR is graded as follows: grade 1, mild pain not interfering with function; grade 2, moderate pain, pain or analgesics interfering with function but not interfering with activities of daily living (ADL); grade 3, severe pain, pain or analgestics interfering with function and interfering with ADL; grade 4, disabling; grade 5, death. In some embodiments, one or more agents can be administered to the subject to treat, ameliorate or lessen one or more symptoms associated with TFR, such as corticosteroids, NSAIDs and/or narcotic analgesic.

In some aspects, the non-hematologic toxicity is tumor lysis syndrome (TLS). In some embodiments, TLS can be graded according to criteria specified by the Cairo-Bishop grading system (Cairo and Bishop (2004) Br J Haematol, 127:3-11). In some embodiments, subjects can be given intravenous hydration to reduce hyperuricemia.

In some embodiments, subjects can be monitored for cardiac toxicity, such as by monitoring ECGS, LVEF and monitoring levels of troponin-T and BNP. In some embodiments, a cardiac toxicity that potentially may necessitate holding or suspending Compound A or Compound B may occur if elevated levels of troponin-T and/or BNP with one or more cardiac symptoms is observed.

In some embodiments of the provided methods, if a subject is determined to exhibit a non-hematological toxicity, such as TFR or other non-hematological toxicity or a particular grade thereof, the cycling therapy with the immunomodulatory compound, e.g. Compound A or Compound B can be altered. In some aspects, the cycling therapy is altered if, after administration of the immunomodulatory compound, e.g. Compound A or Compound B, the subject has a grade 3 or higher non-hematological toxicity, such as grade 3 or higher TFR. In some embodiments, administration of the immunomodulatory compound, e.g. Compound A or Compound B is halted permanently or suspended until signs or symptoms of the toxicity is resolved, lessened or reduced. Continued monitoring of the subject can be carried out to assess one or more signs or symptoms of the toxicity. In some cases, if the toxicity resolves or is reduced, administration of the immunomodulatory compound, e.g. Compound A or Compound B can be restarted at the same dose or dosing regimen prior to suspending the cycling therapy, at a lower or reduced dose, and/or in a dosing regimen involving less frequent dosing. In some embodiments, in instances of restarting the cycling therapy, the dose is lowered or reduced at least or at least about or about 10%, 15%, 20%, 25%, 30%, 40%, 50%, or 60%. In some embodiments, for Compound A or Compound B, if the dose prior to suspending the cell therapy is 2 mg (e.g. given 5/7 days), the dose is reduced to 1 mg (given 5/7 days). In some embodiments, if a grade 3 toxicity recurs even after a dose reduction, the dose can be further reduced. In some embodiments, if a grade 4 toxicity recurs even after a dose reduction, the cycling therapy can be permanently discontinued. In some aspects, if a hematological toxicity is of such severity that suspension of the cycling therapy is for greater than 4 weeks, the cycling therapy can be permanently discontinued.

### V. ARTICLES OF MANUFACTURE AND KITS

Also provided are articles of manufacture containing an immunomodulatory drug (immunomodulatory compound), such as Compound A or Compound B, and components for the immunotherapy, e.g., antibody or antigen binding fragment thereof or T cell therapy, e.g. engineered cells, and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition includes the antibody or engineered cells used for the immunotherapy, e.g. T cell therapy; and (b) a second container with a composition contained therein, wherein the composition includes the second agent, such as an immunomodulatory compound, e.g., Compound A or Compound B. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, a “subject” is a mammal, such as a human or other animal, and typically is human. In some embodiments, the subject, e.g., patient, to whom the immunomodulatory polypeptides, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or engineered cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering the immunomodulatory polypeptides, engineered cells, or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a “subject” or an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject is human.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways, for instance, using publicly available computer software such as BLAST, BLAST-2, ALIGN or Megalign (DNASTAR) software. Appropriate parameters for aligning sequences, including any algorithms needed to achieve maximal alignment over the full length of the sequences being compared, can be determined.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof.

### VII. EXEMPLARY EMBODIMENTS

Among the provided embodiments are:

1. A method of treating multiple myeloma, the method comprising:

(a) administering a T cell therapy to a subject having a relapsed or refractory multiple myeloma (R/R MM), said T cell therapy comprising a dose of genetically engineered T cells expressing a chimeric antigen receptor (CAR) that specifically binds to BCMA; and

(b) administering to the subject an immunomodulatory compound that is (S)-3-[4-(4-morpholin-4-ylmethyl-benzyloxy)-1-oxo-1,3-dihydro-isoindol-2-yl]-piperidine-2,6-dione having the following structure:

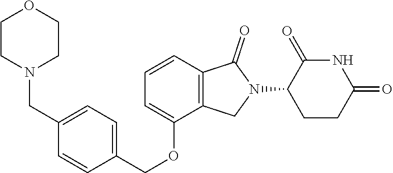 [SMILES: O=C1CC[C@H](N2Cc3c(OCc4ccc(CN5CCOCC5)cc4)cccc3C2=O)C(=O)N1]

or a pharmaceutically acceptable salt, solvate, hydrate, co-crystal, clathrate, or polymorph thereof,

wherein administration of the immunomodulatory compound is initiated after administration of the T cell therapy.

2. The method of embodiment 1, wherein the compound is or comprises a pharmaceutically acceptable salt of (S)-3-[4-(4-morpholin-4-ylmethyl-benzyloxy)-1-oxo-1,3-dihydro-isoindol-2-yl]-piperidine-2,6-dione.

3. The method of embodiment 1, wherein the compound is or comprises a hydrate of (S)-3-[4-(4-morpholin-4-ylmethyl-benzyloxy)-1-oxo-1,3-dihydro-isoindol-2-yl]-piperidine-2,6-dione.

4. The method of embodiment 1, wherein the compound is or comprises a solvate of (S)-3-[4-(4-morpholin-4-ylmethyl-benzyloxy)-1-oxo-1,3-dihydro-isoindol-2-yl]-piperidine-2,6-dione.

5. The method of embodiment 1, wherein the compound is or comprises (S)-3-[4-(4-morpholin-4-ylmethyl-benzyloxy)-1-oxo-1,3-dihydro-isoindol-2-yl]-piperidine-2,6-dione.

6. A method of treating multiple myeloma, the method comprising:

(a) administering a T cell therapy to a subject having a relapsed or refractory multiple myeloma (R/R MM), said T cell therapy comprising a dose of genetically engineered T cells expressing a chimeric antigen receptor (CAR) that specifically binds to BCMA; and

(b) administering to the subject an immunomodulatory compound that is (S)-4-(4-(4-(((2-(2,6-dioxopiperidin-3-yl)-1-oxoisoindolin-4-yl)oxy)methyl)benzyl)piperazin-1-yl)-3-fluorobenzonitrile having the following structure:

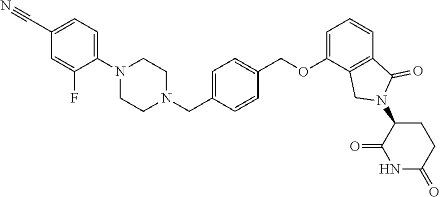 [SMILES: N#Cc1ccc(N2CCN(Cc3ccc(COc4cccc5c4CN([C@H]4CCC(=O)NC4=O)C5=O)cc3)CC2)c(F)c1]

or a pharmaceutically acceptable salt, solvate, hydrate, co-crystal, clathrate, or polymorph thereof,

wherein administration of the immunomodulatory compound is initiated after administration of the T cell therapy.

7. The method of embodiment 6, wherein the compound is or comprises a pharmaceutically acceptable salt of (S)-4-(4-(4-(((2-(2,6-dioxopiperidin-3-yl)-1-oxoisoindolin-4-yl)oxy)methyl)benzyl)piperazin-1-yl)-3-fluorobenzonitrile.

8. The method of embodiment 6, wherein the compound is or comprises a hydrate of (S)-4-(4-(4-(((2-(2,6-dioxopiperidin-3-yl)-1-oxoisoindolin-4-yl)oxy)methyl)benzyl)piperazin-1-yl)-3-fluorobenzonitrile.

9. The method of embodiment 6, wherein the compound is or comprises a solvate of (S)-4-(4-(4-(((2-(2,6-dioxopiperidin-3-yl)-1-oxoisoindolin-4-yl)oxy)methyl)benzyl)piperazin-1-yl)-3-fluorobenzonitrile.

10. The method of embodiment 7, wherein the compound is or comprises (S)-4-(4-(4-(((2-(2,6-dioxopiperidin-3-yl)-1-oxoisoindolin-4-yl)oxy)methyl)benzyl)piperazin-1-yl)-3-fluorobenzonitrile.

11. The method of any of embodiments 1-10, wherein the subject has relapsed or been refractory following at least 3 or at least 4 prior therapies for multiple myeloma.

12. The method of any of embodiments 1-11, wherein the subject has received, and has relapsed or been refractory to, three or more therapies selected from among:

autologous stem cell transplant (ASCT);

an immunomodulatory agent;

a proteasome inhibitor; and

an anti-CD38 antibody; unless the subject was not a candidate for or was contraindicated for one or more of the therapies.

13. The method of embodiment 12, wherein the immunomodulatory agent is selected from among thalidomide, lenalidomide and pomalidomide.

14. The method of embodiment 12, wherein the proteasome inhibitor is selected from among bortezomib, carfilzomib and ixazomib.

15. The method of embodiment 12, wherein the anti-CD38 antibody is or comprises daratumumab.

16. The method of any of embodiments 1-15, wherein, at the time of administration, the subject has been refractory to or not responded to bortezomib, carfilzomib, lenalidomide, pomalidomide and/or an anti-CD38 monoclonal antibody.

17. The method of any of embodiments 1-16, wherein, at the time of administration, the subject has IMWG high risk cytogenetics.

18. The method of any of embodiments 1-17, wherein administration of the compound is initiated at or prior to peak expansion of the T cell therapy in the subject.

19. The method of embodiment 18, wherein peak expansion of the T cell therapy is between at or about 11 days and at or about 15 days after administering the T cell therapy.

20. The method of any of embodiments 1-19, wherein administration of the compound is initiated between at or about 1 day and at or about 15 days, inclusive, after administering the T cell therapy.

21. The method of any of embodiments 1-20, wherein administration of the compound is initiated between at or about 1 day and at or about 11 days, inclusive, after administering the T cell therapy.

23. The method of any of embodiments 1-21, wherein the administration of the compound is initiated between at or about 8 days and at or about 15 days, inclusive, after administering the T cell therapy.

24. The method of any of embodiments 1-23, wherein administration of the compound is initiated at or about 1 day after administering the T cell therapy.

25. The method of any of embodiments 1-23, wherein administration of the compound is initiated at or about 8 days after administering the T cell therapy.

26. The method of any of embodiments 1-23, wherein the administration of the compound is initiated at or about 15 days after administering the T cell therapy.

27. The method of any of embodiments 1-17, wherein the administration of the compound is initiated about 14 to about 35 days after initiation of administration of the T cell therapy.

28. The method of any of embodiments 1-17 and 27, wherein the administration of the compound is initiated about 21 to about 35 days after initiation of administration of the T cell therapy.

29. The method of any of embodiments 1-17, 27 and 28, wherein the administration of the compound is initiated about 21 to about 28 days after initiation of administration of the T cell therapy.

30. The method of any of embodiments 1-17 and 27-29, wherein the administration of the compound is initiated at or about 21 days, at or about 22 days, at or about 23 days, at or about 24 days, at or about 25 days, at or about 26 days, at or about 27 days, or at or about 28 days after initiation of administration of the T cell therapy.

31. The method of any of embodiments 1-17 and 27-30, wherein the administration of the compound is initiated at or about 28 days after the initiation of the administration of the T cell therapy.

32. The method of any of embodiments 1-31, wherein the compound is administered at least once daily in a cycle regimen.

33. The method of embodiment 32, wherein the cycle regimen is a four-week (28-day) cycle wherein the compound is administered daily in the four-week cycle.

34. The method of embodiment 32, wherein the cycle regimen is a four-week (28-day) cycle wherein the compound is administered daily for three consecutive weeks in the four-week cycle.

35. The method of embodiment 32, wherein the cycle regimen is a four-week (28-day) cycle wherein the compound is administered daily for days 1 through 21 of each four-week cycle.

36. The method of any of embodiments 32-35, wherein the cycling regimen is repeated a plurality of times.

37. The method of embodiment 36, wherein the plurality of times is between two and 12 cycling regimens.

38. The method of embodiment 36 or embodiment 37, wherein the cycling regiment is repeated 3 times.

39. The method of embodiment 36 or embodiment 37, wherein the cycling regimen is repeated 4 times.

40. The method of embodiment 36 or embodiment 37, wherein the cycling regimen is repeated 5 times.

41. The method of embodiment 36 or embodiment 37, wherein the cycling regimen is repeated 6 times.

42. The method of any of embodiments 1-41, wherein the immunomodulatory compound is administered up to at or about three months after initiation of administration of the T cell therapy.

43. The method of any of embodiments 1-41, wherein the immunomodulatory compound is administered up to at or about six months after initiation of administration of the T cell therapy.

44. The method of any of embodiments 1-43, wherein the immunomodulatory compound is administered in an amount that is at or about 0.1 mg to about 1.0 mg per day.

45. The method of any of embodiments 1-44, wherein the immunomodulatory compound is administered in an amount that is at or about 0.3 mg to about 0.6 mg.

46. The method of any of embodiments 1-45, wherein the immunomodulatory compound is administered in an amount that is at or about 0.3 mg.

47. The method of any of embodiments 1-45, wherein the immunomodulatory compound is administered in an amount that is at or about 0.45 mg.

48. The method of any of embodiments 1-45, wherein the immunomodulatory compound is administered in an amount that is at or about 0.6 mg.

49. The method of any of embodiments 1-48, wherein the compound is administered orally.

50. The method of any of embodiments 1-49, wherein at the time of the initiation of the administration of the compound, the subject does not exhibit a severe toxicity following the administration of the T cell therapy.

51. The method of embodiment 50, wherein:

the severe toxicity is severe cytokine release syndrome (CRS), optionally grade 3 or higher, prolonged grade 3 or higher or grade 4 or 5 CRS; and/or

the severe toxicity is severe neurotoxicity, optionally grade 3 or higher, prolonged grade 3 or higher or grade 4 or 5 neurotoxicity.

52. The method of any one of embodiments 1-51, wherein the administration of the compound is suspended and/or the cycling regimen is modified if the subject exhibits a toxicity following the administration of the compound, optionally a hematologic toxicity.

53. The method of embodiment 52, wherein the toxicity is selected from severe neutropenia, optionally febrile neutropenia, prolonged grade 3 or higher neutropenia.

54. The method of any of embodiments 1-53, wherein the administration of the compound:

reverses an exhaustion phenotype in CAR-expressing T cells in the subject;

prevents, inhibits or delays the onset of an exhaustion phenotype in CAR-expressing T cells in the subject;

or reduces the level or degree of an exhaustion phenotype in CAR-expressing T cells in the subject; or

reduces the percentage, of the total number of CAR-expressing T cells in the subject, that have an exhaustion phenotype.

55. The method of any of embodiments 1-54, wherein following administration of the compound or initiation thereof, the subject exhibits a restoration or rescue of an antigen- or tumor-specific activity or function of CAR-expressing T cells in said subject, optionally wherein said restoration, rescue, and/or initiation of administration of said compound, is at a point in time after CAR-expressing T cells in the subject or the in the blood of the subject have exhibited an exhausted phenotype.

56. The method of any of embodiments 1-55, wherein the administration of the compound comprises administration at an amount, frequency and/or duration effective to:

(a) effect an increase in antigen-specific or antigen receptor-driven activity of naïve or non-exhausted T cells in the subject, which optionally comprise T cells expressing said CAR, following exposure of the T cells to BCMA or to an agonist of the CAR, optionally wherein the agonist is an anti-idiotypic antibody, as compared to the absence of said administration of said compound; or

(b) prevent, inhibit or delay the onset of an exhaustion phenotype, in naïve or non-exhausted T cells T cells in the subject, which optionally comprise T cells expressing said CAR, following exposure of the T cells to BCMA or to an agonist of the CAR, optionally wherein the agonist is an anti-idiotypic antibody, as compared to the absence of said administration of said compound; or

(c) reverse an exhaustion phenotype in exhausted T cells, optionally comprising T cells expressing said CAR, in the subject, as compared to the absence of said administration of said subject.

57. The method of any of embodiments 1-56, wherein at the time of the administration of the compound an exhausted phenotype of one or more of the CAR-expressing T cells, or a marker or parameter indicative thereof, has been detected or measured in the subject or in a biological sample from the subject.

58. The method of embodiment 57, wherein at least at or about 10%, at least at or about 20%, at least at or about 30%, at least at or about 40%, or at least at or about 50% of the total CAR-expressing T cells in a biological sample from the subject has an exhausted phenotype.

59. The method of embodiment 57 or embodiment 58, wherein greater than at or about 10%, greater than at or about 20%, greater than at or about 30%, greater than at or about 40%, or greater than at or about 50% of the CAR-expressing T cells in a biological sample from the subject has an exhausted phenotype compared to the percentage of the CAR-expressing T cells having the exhausted phenotype in a comparable biological sample at a prior time point.

60. The method of any of embodiments 57-59, wherein the exhaustion phenotype, with reference to a T cell or population of T cells, comprises:

an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or

a decrease in the level or degree of an activity exhibited by said T cells or population of T cells upon exposure to BCMA or an agonist of the CAR, optionally wherein the agonist is an anti-idiotypic antibody, compared to a reference T cell population, under the same conditions.

61. The method of embodiment 60, wherein the increase in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

62. The method of embodiment 60, wherein the decrease in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

63. The method of any of embodiments 60-62, wherein the reference T cell population is a population of T cells known to have a non-exhausted phenotype, is a population of naïve T cells, is a population of central memory T cells, or is a population of stem central memory T cells, optionally from the same subject, or of the same species as the subject, from which the T cell or T cells having the exhausted phenotype are derived.

64. The method of any of embodiments 60-63, wherein the reference T cell population (a) is a subject-matched population comprising bulk T cells isolated from the blood of the subject from which the T cell or T cells having the exhausted phenotype is derived, optionally wherein the bulk T cells do not express the CAR and/or (b) is obtained from the subject from which the T cell or T cells having the exhausted phenotype is derived, prior to receiving administration of a dose of T cells expressing the CAR.

65. The method of any of embodiments 60-64, wherein the reference T cell population is a composition comprising a sample of the T cell therapy, or pharmaceutical composition comprising T cells expressing the CAR, prior to its administration to the subject, optionally wherein the composition is a cryopreserved sample.

66. The method of any of embodiments 60-65, wherein one or more of the one or more exhaustion marker is an inhibitory receptor.

67. The method of any of embodiments 60-66, wherein one or more of the one or more exhaustion marker is selected from among PD-1, CTLA-4, TIM-3, LAG-3, BTLA, 2B4, CD160, CD39, VISTA, and TIGIT.

68. The method of any of embodiments 60-67, wherein the activity or is one or more of proliferation, cytotoxicity or production of one or a combination of inflammatory cytokines, optionally wherein the one or a combination of cytokines is selected from the group consisting of IL-2, IFN-gamma and TNF-alpha.

69. The method of any of embodiments 60-68, wherein the exposure to BCMA or an agonist of the CAR, optionally wherein the agonist is an anti-idiotypic antibody, comprises incubation with BCMA or the agonist of the CAR.

70. The method of embodiment 69, wherein the antigen is comprised on the surface of antigen-expressing target cells, optionally multiple myeloma cells or cell line.

71. The method of any of embodiments 1-70, wherein the dose of T cells is between at or about 5×10⁷ CAR+ T cells and at or about 1×10⁹ CAR+ T cells.

72. The method of any of embodiments 1-70, wherein the dose of T cells is between at or about 1×10⁸ CAR+ T cells and at or about 1×10⁹ CAR+ T cells.

73. The method of any of embodiments 1-70, wherein the dose of T cells is at or about 1.5×10⁸ cells or CAR+ T cells.

74. The method of any of embodiments 1-70, wherein the dose of T cells is at or about 3×10⁸ cells or CAR+ T cells.

75. The method of any of embodiments 1-70, wherein the dose of T cells is at or about 4.5×10⁸ cells or CAR+ T cells.

76. The method of any of embodiments 1-70, wherein the dose of T cells is at or about 6×10⁸ cells or CAR+ T cells.

77. The method of any of embodiments 1-76, wherein the dose comprises CD3+ CAR-expressing T cells.

78. The method of any of embodiments 1-77, wherein the dose comprises a combination of CD4⁺ T cells and CD8⁺ T cells and/or a combination of CD4⁺ CAR-expressing T cells and CD8⁺ CAR-expressing T cells.

79. The method of embodiment 78, wherein the ratio of CD4⁺ CAR-expressing T cells to CD8⁺ CAR-expressing T cells and/or of CD4⁺ T cells to CD8⁺ T cells, is or is approximately 1:1 or is between at or approximately 1:3 and at or approximately 3:1.

80. The method of any of embodiments 1-79, wherein prior to the administration of the dose of T cells, the subject has received a lymphodepleting therapy comprising the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.

81. The method of any of embodiments 1-80, wherein the subject has received a lymphodepleting therapy comprising the administration of fludarabine at or about 30 mg/m² body surface area of the subject, daily, and cyclophosphamide at or about 300 mg/m² body surface area of the subject, daily, for 3 days.

82. The method of any of embodiments 1-81, wherein the CAR comprises an antigen binding domain that binds to BCMA, a transmembrane domain, and an intracellular signaling region comprising a CD3-zeta (CD3ζ) chain.

83. The method of embodiment 82, wherein the antigen binding domain is a single chain variable fragment (scFv).

84. The method of embodiment 82 or embodiment 83, wherein the antigen binding domain comprises a V_{H} and a V_{L} region, wherein the V_{H} region comprises a CDR-H1 set forth in SEQ ID NO: 56, a CDR-H2 set forth in SEQ ID NO:57 and a CDR-H3 set forth in SEQ ID NO:58, and the V_{L} region comprises a CDR-L1 set forth in SEQ ID NO: 59, a CDR-L2 set forth in SEQ ID NO:60 and a CDR-H3 set forth in SEQ ID NO:61.

85. The method of any of embodiments 82-84, wherein the antigen binding domain comprises a V_{H} region that has the sequence of amino acids set forth in SEQ ID NO:36 or a sequence of amino acids that exhibits at least 90%, at least 91%, at least 92%, at least 93%, at least 94%, at least 95%, at least 96%, at least 97%, at least 98%, at least 99% to SEQ ID NO:36, and a V_{L} region has the sequence of amino acids set forth in SEQ ID NO:37 or a sequence of amino acids that exhibits at least 90%, at least 91%, at least 92%, at least 93%, at least 94%, at least 95%, at least 96%, at least 97%, at least 98%, at least 99% to SEQ ID NO:37.

86. The method of any of embodiments 82-84, wherein the antigen binding domain comprises the V_{H} region sequence of amino acids set forth in SEQ ID NO:36 and the V_{L} region sequence of amino acids set forth in SEQ ID NO:37.

87. The method of any of embodiments 82-86, wherein the antigen-binding domain is an scFv that has the sequence of amino acids set forth in SEQ ID NO:180 or a sequence of amino acids exhibits at least 90%, at least 91%, at least 92%, at least 93%, at least 94%, at least 95%, at least 96%, at least 97%, at least 98%, at least 99% to SEQ ID NO:180.

88. The method of any of embodiments 82-87, wherein the antigen-binding domain is an scFv that has the sequence of amino acids set forth in SEQ ID NO:180.

89. The method of embodiment 82 or embodiment 83, wherein the anti-BCMA CAR comprises a V_{H} and a V_{L} region, wherein the V_{H} region comprises a CDR-H1 set forth in SEQ ID NO: 62, a CDR-H2 set forth in SEQ ID NO:63 and a CDR-H3 set forth in SEQ ID NO:64, and the V_{L} region comprises a CDR-L1 set forth in SEQ ID NO: 65, a CDR-L2 set forth in SEQ ID NO:66 and a CDR-H3 set forth in SEQ ID NO:67.

90. The method of embodiment 82, 83 or 89, wherein the antigen binding domain comprises a V_{H} region that has the sequence of amino acids set forth in SEQ ID NO:30 or a sequence of amino acids that exhibits at least 90%, at least 91%, at least 92%, at least 93%, at least 94%, at least 95%, at least 96%, at least 97%, at least 98%, at least 99% to SEQ ID NO:30, and the V_{L} region has the sequence of amino acids set forth in SEQ ID NO:31 or a sequence of amino acids that exhibits at least 90%, at least 91%, at least 92%, at least 93%, at least 94%, at least 95%, at least 96%, at least 97%, at least 98%, at least 99% to SEQ ID NO:31.

91. The method of embodiment 82, 83, 89 or 90, wherein the antigen binding domain comprises the V_{H} region that has the sequence of amino acids set forth in SEQ ID NO:30 and the V_{L} region has the sequence of amino acids set forth in SEQ ID NO:31.

92. The method of any of embodiments 82, 83 and 89-91, wherein the antigen binding domain is an scFv that has the sequence of amino acids set forth in SEQ ID NO:68 or a sequence of amino acids exhibits at least 90%, at least 91%, at least 92%, at least 93%, at least 94%, at least 95%, at least 96%, at least 97%, at least 98%, at least 99% to SEQ ID NO:68.

93. The method of any of embodiments 82, 83 and 89-91, wherein the antigen binding domain is an scFv set forth in SEQ ID NO:68.

94. The method of any of embodiments 82-93, wherein the intracellular signaling region further comprises a costimulatory signaling domain.

95. The method of embodiment 94, wherein the costimulatory signaling region comprises an intracellular signaling domain of CD28, 4-1BB, or ICOS, or a signaling portion thereof.

96. The method of embodiment 94 or embodiment 95, wherein the costimulatory signaling region comprises an intracellular signaling domain of 4-1BB, optionally human 4-1BB.

97. The method of any of embodiments 94-96, wherein the costimulatory signaling region is between the transmembrane domain and the cytoplasmic signaling domain of a CD3-zeta (CD3ζ) chain.

98. The method of any of embodiments 82-97, wherein the transmembrane domain is or comprises a transmembrane domain from human CD28.

99. The method of any of embodiments 82-97, wherein the transmembrane domain is or comprises a transmembrane domain from human CD8.

100. The method of any of embodiments 82-99, wherein the CAR further comprises an extracellular spacer between the antigen binding domain and the transmembrane domain.

101. The method of any of embodiment 100, wherein the spacer is between at or about 50 amino acids and at or about 250 amino acids.

102. The method of embodiment 100 or embodiment 101, wherein the spacer is between at or about 125 amino acids and at or about 250 amino acids, optionally wherein the spacer is at or about 228 amino acids.

103. The method of any of embodiments 100-102, wherein the spacer is an immunoglobulin spacer comprising all or a portion of an immunoglobulin constant domain or a modified form thereof.

104. The method of any of embodiments 100-103, wherein the spacer comprises an IgG4/2 chimeric hinge or a modified IgG4 hinge; an IgG2/4 chimeric C_{H}2 region; and an IgG4 C_{H}3 region.

105. The method of any of embodiments 100-104, wherein the spacer is set forth in SEQ ID NO: 29 or is encoded by a sequence of nucleotides set forth in SEQ ID NO:179.

106. The method of embodiment 100 or embodiment 101, wherein the spacer is a CD8 hinge.

107. The method of any of embodiments 1-106, wherein the anti-BCMA CAR has a sequence set forth in any one of SEQ ID NOS: 126-177 or a sequence of amino acids that exhibits at least 90%, at least 91%, at least 92%, at least 93%, at least 94%, at least 95%, at least 96%, at least 97%, at least 98%, or at least 99% sequence identity to any one of SEQ ID NOS: 126-177.

108. The method of any of embodiments 1-107, wherein the anti-BCMA CAR has the sequence of amino acids set forth in SEQ ID NO:160 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:160.

109. The method of any of embodiments 1-108, wherein the CAR is set forth in SEQ ID NO:160.

110. The method of any of embodiments 1-109, wherein the CAR is encoded by the sequence of nucleotides set forth in SEQ ID NO:69.

111. The method of any of embodiments 1-107, wherein the anti-BCMA CAR has the sequence of amino acids set forth in SEQ ID NO:161 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:161.

112. The method of any of embodiments 1-107, wherein the anti-BCMA CAR has the sequence of amino acids set forth in SEQ ID NO:152 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:152.

113. The method of any of embodiments 1-107, wherein the anti-BCMA CAR has the sequence of amino acids set forth in SEQ ID NO:168 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:168.

114. The method of any of embodiments 1-107, wherein the anti-BCMA CAR has the sequence of amino acids set forth in SEQ ID NO:171 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:171.

115. The method of any of embodiments 1-114, wherein the anti-BCMA CAR binds BCMA, optionally wherein the BCMA is human BCMA.

116. The method of embodiment 115, wherein the BCMA is membrane-bound BCMA expressed on the surface of a cell.

117. The method of embodiment 115 or embodiment 116, wherein the anti-BCMA CAR has a greater binding affinity for membrane-bound BCMA than soluble BCMA, optionally wherein the ratio of dissociation constant (K_{D}) for soluble BCMA and the K_{D} for membrane-bound BCMA is more than 10, 15, 20, 25, 30, 40, 50, 60, 70, 80, 90, 100, 200, 500, 1000, 2000 or more.

### VIII. EXAMPLES

The following examples are included for illustrative purposes only and are not intended to limit the scope of the invention

### Example 1 Assessment of Pharmacodynamic Response of Aiolos and Ikaros Transcription Factor in Anti-BCMA CAR-Expressing T Cells in the Presence of Cell Immunomodulatory Compounds

T cell compositions containing anti-BCMA CAR-expressing cells were generated from T cells of donors. The exemplary anti-BCMA CAR was encoded by a polynucleotide construct that contained nucleic acid encoding a human IgG-kappa signaling sequence, a human anti-BCMA scFv, a modified IgG4-hinge C_{H}2-C_{H}3 (SEQ ID NO:29, encoded by SEQ ID NO:179 or 183) spacer (which spacer may in some instances be referred to as “LS”); a human CD28 transmembrane domain; a human 4-1BB-derived intracellular co-signaling sequence; and a human CD3-zeta derived intracellular signaling domain. The exemplary human anti-BCMA scFv contained an scFv with the following sequences:

TABLE El
Sequence identifier (SEQ ID NO) for Exemplary scFv

| Antigen-binding | CDR- | CDR- | CDR- | CDR- | CDR- | CDR- | | | |
|---|---|---|---|---|---|---|---|---|---|
| domain | H1 | H2 | H3 | L1 | L2 | L3 | V_{H} | V_{L} | scFv |
| BCMA-55 | 56 | 57 | 58 | 59 | 60 | 61 | 36 | 37 | 180 |

cDNA clones encoding such CARs, were linked to a downstream ribosomal skip element (such as T2A) followed by a truncated receptor-encoding sequence for use as a surrogate marker, and cloned into a lentiviral expression vector.

To generate anti-BCMA CAR T cells, leukapheresis samples from donors were collected and were cryofrozen. CD4+ and CD8+ T cells were separately selected by immunoaffinity-based selection from the samples, resulting in two compositions, enriched for CD8+ and CD4+ cells, respectively. The selected cell compositions were subsequently thawed and mixed at a ratio of 1:1 of viable CD4⁺ T cells to viable CD8⁺ T cells. Approximately 300×10⁶ T cells (150×10⁶ CD4 and 150×10⁶ CD8⁺ T cells) of the mixed composition were stimulated in the presence of paramagnetic polystyrene-coated beads with attached anti-CD3 and anti-CD28 antibodies at a 1:1 bead to cell ratio in serum free media containing recombinant IL-2, IL-7 and IL-15 for between 18 to 30 hours. Following the incubation, approximately 100×10⁶ viable cells from the stimulated cell composition were concentrated in the serum free media containing recombinant IL-2, IL-7 and IL-15 The cells were transduced, by spinoculation at approximately 1600 g for 60 minutes, with a lentiviral vector encoding the anti-BCMA CAR. After spinoculation, the cells were resuspended in the serum free media containing recombinant IL-2, IL-7 and IL-15, and incubated for about 18 to 30 hours at about 37° C. The cells were then cultivated for expansion by transfer to a bioreactor (e.g. a rocking motion bioreactor) in about 500 mL of a serum free media containing twice the concentration of IL-2, IL-7 and IL-15 as used during the incubation and transduction steps. When a set viable cell density was achieved, perfusion was initiated, where media was replaced by semi-continuous perfusion with continual mixing. The cells were cultivated the next day in the bioreactor until a threshold cell density of about 3×10⁶ cells/mL was achieved, which typically occurred in a process involving 6-7 days of expansion. The anti-CD3 and anti-CD28 antibody conjugated paramagnetic beads were removed from the cell composition by exposure to a magnetic field. The cells where then collected, formulated and cryoprotected.

Anti-BCMA CAR+ T cells were stimulated with 50 μg BCMA-coated beads at a ratio of T cells to beads of 1:1 in the presence of lenalidomide (1000 nM), (S)-3-[4-(4-morpholin-4-ylmethyl-benzyloxy)-1-oxo-1,3-dihydro-isoindol-2-yl]-piperidine-2,6-dione (Iberdomide, Compound A) (1 nM, 10 nM, or 100 nM), (S)-4-(4-(4-(((2-(2,6-dioxopiperidin-3-yl)-1-oxoisoindolin-4-yl)oxy)methyl)benzyl)piperazin-1-yl)-3-fluorobenzonitrile (Compound B) (0.1 nM, 1 nM or 10 nM), or a vehicle control for 24 hours or 72 hours at 37° C., 5% CO₂. A BCMA-Fc fusion protein containing the extracellular domain of human BCMA and a human IgG1Fc was conjugated to beads by covalently coupling to the surface of commercially available tosyl-activated magnetic beads having a diameter of approximately 4.5 μM (M-450 beads, ThermoFisher, Waltham Mass.) (see e.g. International published PCT App. No. WO2019/027850). Following incubation, anti-BCMA CAR-expressing T cells were stained with antibodies and analyzed by flow cytometry to assess intracellular levels of Ikaros and Aiolos in CD4+CAR+ or CD8+CAR+ cells, as measured by median fluorescence intensity (MFI). Median fluorescence intensity (MFI) values for Ikaros and Aiolos were normalized and calculated as a percentage relative to vehicle control.

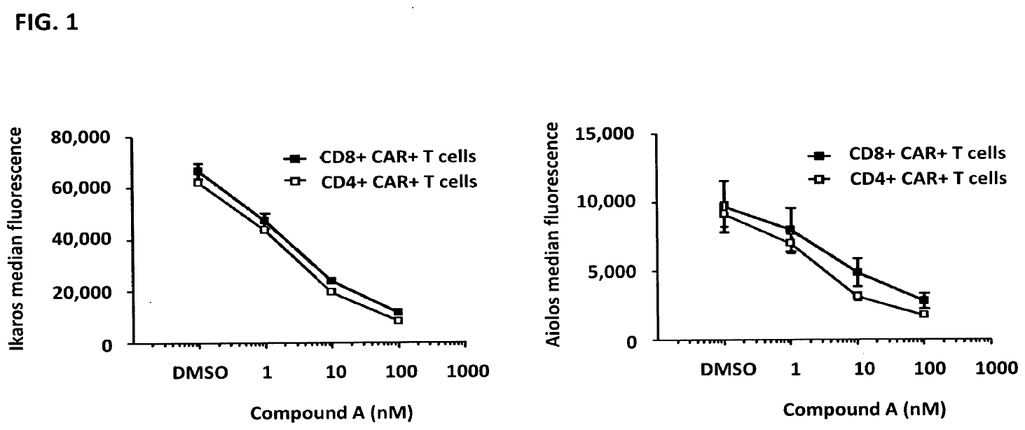

A concentration dependent decrease in intracellular Ikaros and Aiolos expression was observed in the anti-BCMA stimulated CAR-expressing T cells after incubation with lenalidomide, Compound A, or Compound B. The results demonstrated that Compound A and Compound B were more potent than lenalidomide. As shown in FIG. 1, Iberdomide resulted in similar levels of degradation of Ikaros and Aiolos in stimulated CD4+ T cells and CD8+ T cells from donors.

### Example 2 Effect of Cell Immunomodulatory Compounds on Acute CAR T-Cell Function

Anti-tumor effects of anti-BCMA CAR T cells, alone and in combination with cell immunomodulatory compounds Compound A (iberdomide, (S)-3-[4-(4-morpholin-4-ylmethyl-benzyloxy)-1-oxo-1,3-dihydro-isoindol-2-yl]-piperidine-2,6-dione) or Compound B ((S)-4-(4-(4-(((2-(2,6-dioxopiperidin-3-yl)-1-oxoisoindolin-4-yl)oxy)methyl)benzyl)piperazin-1-yl)-3-fluorobenzonitrile), were assessed in two different BCMA-expressing target multiple myeloma cell lines RPMI-8226 or OPM-2. Anti-BCMA CAR+ T cells were generated as described in Example 1.

The anti-BCMA CAR-expressing T cells were incubated with target cells, RPMI 8226 (BCMA^{med} human multiple myeloma cell line) or OPM2 cells (BCMA^{med} human multiple myeloma cell line) target cells, at an E:T ratio of 1:1. The co-culture incubation with target cells was carried out in the presence of Compound A (0.01 nM, 0.1 nM, 1 nM, 10 nM, 100 nM, 1000 nM, or 10,000 nM) or Compound B (0.01 nM, 0.1 nM, 1 nM, 10 nM, 100 nM, 1000 nM, or 10,000 nM).

To assess cytolytic activity, the target cells were labeled with NucLight Red (NLR) to permit tracking by fluorescent microscopy. Killing activity was assessed by measuring the loss of viable target cells over 200 hours, as determined by loss of fluorescent signal over time by kinetic fluorescence microscopy (using the INCUCYTE® Live Cell Analysis System, Essen Bioscience). The area under the curve (AUC) for target fluorescence over time was determined. The results showed that the presence of the cell immunomodulatory compounds did not lead to an improvement in the killing of anti-BCMA CAR+ T cells in the acute assay.

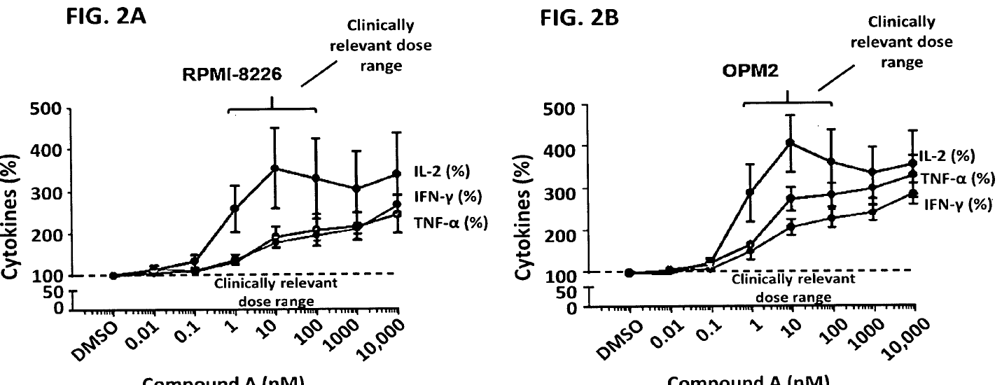

Cytokine secretion of IL-2, TNFα and IFN-gamma cytokines from cell culture supernatant of the co-cultures after 24 hours of incubation with targets cells was determined. Co-culture of anti-BCMA CAR+ T cells with Compound A enhanced the CAR T cytokine production both against RPMI-8226 target cells (FIG. 2A) and OPM2 target cells (FIG. 2B). Similar studies also showed that addition of Compound B also increased CAR T cell cytokine production. Notably Compound B exhibited enhanced effects at lower concentrations compared to Compound A.

### Example 3 Functionality of Anti-BCMA CAR T Cells Following Rechallenge after Concurrent Treatment with Cell Immunomodulatory Compound During Chronic Activation

Cryofrozen anti-BCMA CAR T cells, produced substantially as described in Example 1 and formulated at a 1:1 ratio of CD4+ and CD8⁺ T cells, were thawed. To subject cells to chronic stimulation conditions, the anti-BCMA CAR+ T cells were stimulated with 50 μg BCMA conjugated beads (diameter about 4.5 μm from a 50 μg/ml BCMA-conjugated bead composition, generated as described in Example 2) at a ratio of T cells to beads of 1:1 in the presence or absence of Compound A (iberdomide, (S)-3-[4-(4-morpholin-4-ylmethyl-benzyloxy)-1-oxo-1,3-dihydro-isoindol-2-yl]-piperidine-2,6-dione) (1 nM or 10 nM), Compound B ((S)-4-(4-(4-(((2-(2,6-dioxopiperidin-3-yl)-1-oxoisoindolin-4-yl)oxy)methyl)benzyl)piperazin-1-yl)-3-fluorobenzonitrile) (0.1 nM or 1 nM), or DMSO vehicle control. The cells were then incubated at 37° C., 5% CO₂ for 7 days.

At day 7, anti-BCMA CAR cells of all the samples were counted using a cellometer machine and stained for flow cytometric analysis. Cells were stained with a viability dye and analyzed by flow cytometry. Viability and count of anti-BCMA CAR T cells was increased in the presence of Compound A or Compound B.

CAR T cells that had been stimulated for 7 days with cultured with 50 μg BCMA-coated magnetic beads concurrently in the presence of the cell immunomodulatory compound were washed free of compound and cells were debeaded. The anti-BCMA CAR T cells that had been stimulated for 7 days with BCMA-conjugated beads in the presence of the compounds were co-cultured with RPMI-8226 target cells (labeled with NucLightRed as described in Example 2) at a 1:1 (effector:target) ratio. Killing activity was monitored over 200 hours by measuring NucLightRed (NLR)-positive target cells. The total cell number of NLR-expressing RPMI targets cells was determined by normalizing cell number over time to the number of cells at the initiation of the co-culture (t=0) for each respective condition.

For cytokine measurements, cell-free supernatants were collected from the cytolytic assay described above 24 hours after plating. Cytokine levels of IFNg, IL-2, and TNFalpha (TNFα) was measured in the supernatant.

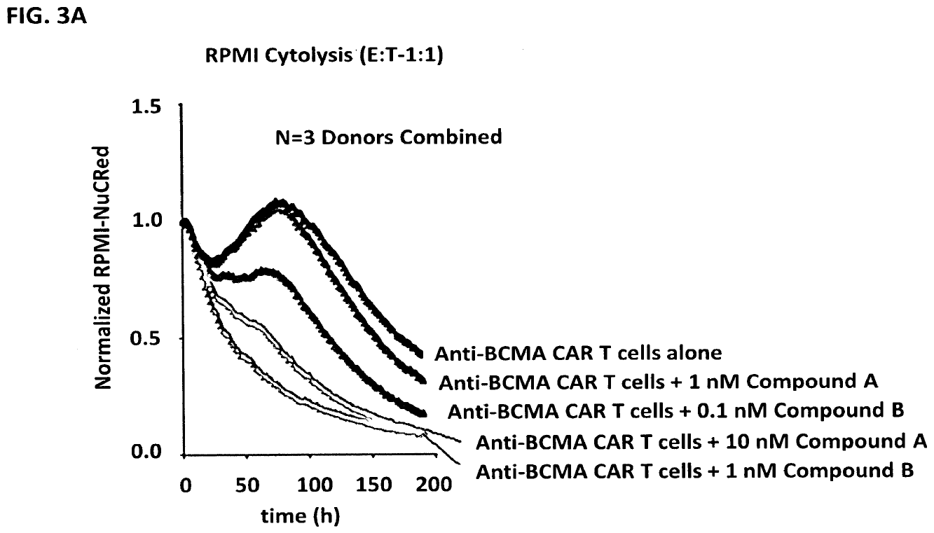

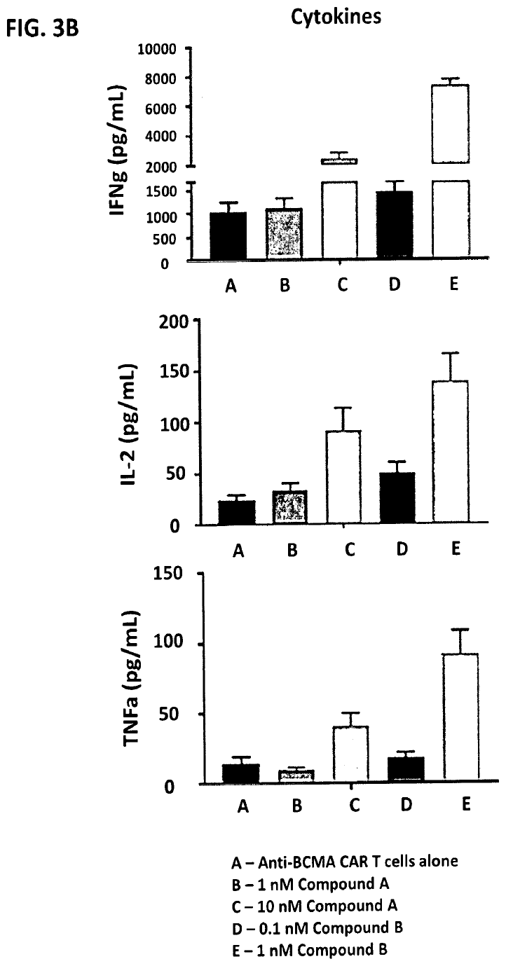

As shown in FIG. 3A and FIG. 3B, concurrent treatment with the cell immunomodulatory compound during chronic activation improved cytolytic activity and cytokine production, respectively, of the anti-BCMA CAR+ T cells following re-challenge with target cells.

### Example 4 Functionality of Chronically Activated Anti-BCMA CAR T Cells after Rechallenge in the Presence of Cell Immunomodulatory Compound

Anti-BCMA CAR-expressing T cells, produced as described in Example 1, were stimulated with 50 μg BCMA conjugated beads (diameter about 4.5 μm from a 50 μg/ml BCMA-conjugated bead composition) substantially as described in Example 3 to induce chronic stimulation (to produce hypofunctional, exhausted T cells). After the chronic stimulation, the CAR-T cells were rechallenged with RPMI-8226 target cells (labeled with NucLightRed as described in Example 2) at a 1:1 (effector:target) ratio in the presence of (rescued) with Compound A (iberdomide, (S)-3-[4-(4-morpholin-4-ylmethyl-benzyloxy)-1-oxo-1,3-dihydro-isoindol-2-yl]-piperidine-2,6-dione) (1 nM or 10 nM), Compound B ((S)-4-(4-(4-(((2-(2,6-dioxopiperidin-3-yl)-1-oxoisoindolin-4-yl)oxy)methyl)benzyl)piperazin-1-yl)-3-fluorobenzonitrile) (0.1 nM or 1 nM), or DMSO vehicle control.

Killing activity and cytokine production were assessed as described in Example 3.

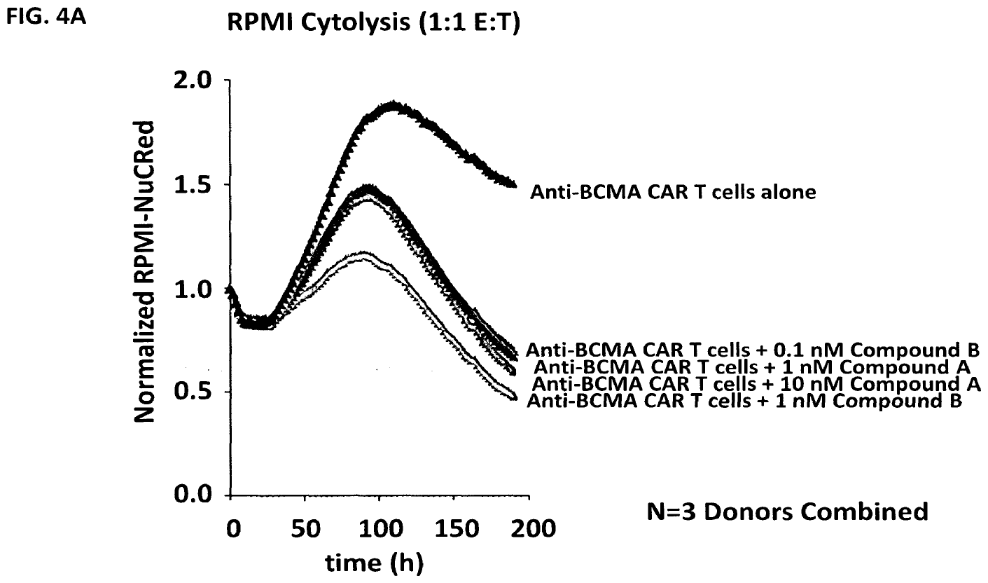

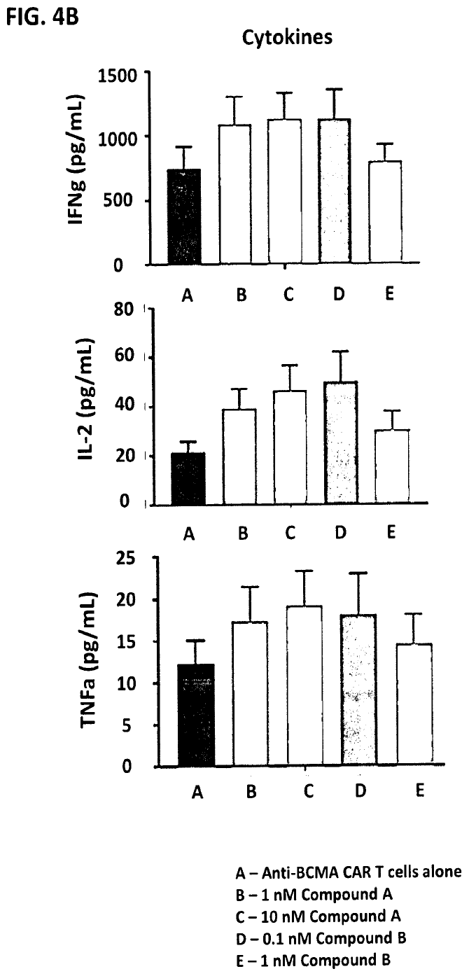

As shown in FIG. 4A and FIG. 4B, treatment with the cell immunomodulatory compound during re-challenge of anti-BCMA CAR T cells with antigen-expressing target cells improved cytolytic activity and cytokine production, respectively, of the anti-BCMA CAR T cells. This result demonstrates that the cell immunomodulatory compounds Compound A and Compound B were able to rescue chronically activated CAR T cells to improve functionality.

### Example 5 Effect of Cell Immunomodulatory Compounds on Anti-BCMA CAR T Cells During Serial Stimulation Assay

The ability of CAR T cells to expand and exhibit antigen-specific function ex vivo following repeated rounds of antigen stimulation can correlate with in vivo function and/or capacity of the cells to persist in vivo (e.g. following administration and initial activation in response to encounter with antigen) (Zhao et al. (2015) Cancer Cell, 28:415-28). A serial stimulation assay or serial re-challenge assay was used to assess activity of anti-BCMA CAR T cells following repeated rounds of antigen stimulation, in the presence and absence of Compound A (iberdomide, (S)-3-[4-(4-morpholin-4-ylmethyl-benzyloxy)-1-oxo-1,3-dihydro-isoindol-2-yl]-piperidine-2,6-dione).

Anti-BCMA CAR+ T cells (generated as described in Example 1) were plated in triplicate at 1×10⁵ cells/well on 96-well plates. Irradiated DF-15R target cells, which is a multiple myeloma (MM) cell line generated to be resistant to effect of cell immunomodulatory compounds (Lopez-Girona et al. Leukemia, 2012; 26:2326-2335), were added at an effector-to-target (E:T) ratio of 1:2 in the presence or absence of various concentrations (10 nM) of Iberdomide.

Every 3-4 days (start of each new round), CAR T cells were counted. Cells then were harvested and re-plated at the initial seeding density with fresh media, newly-added Iberdomide at the same concentration, where applicable, and newly-thawed, irradiated target cells. Five rounds of stimulation were carried out during a 19 day culture period. At day 5 and day 9 (24 hours after a re-plating (reset) cytokine production in the supernatant was assessed. Results were assessed in two different donors.

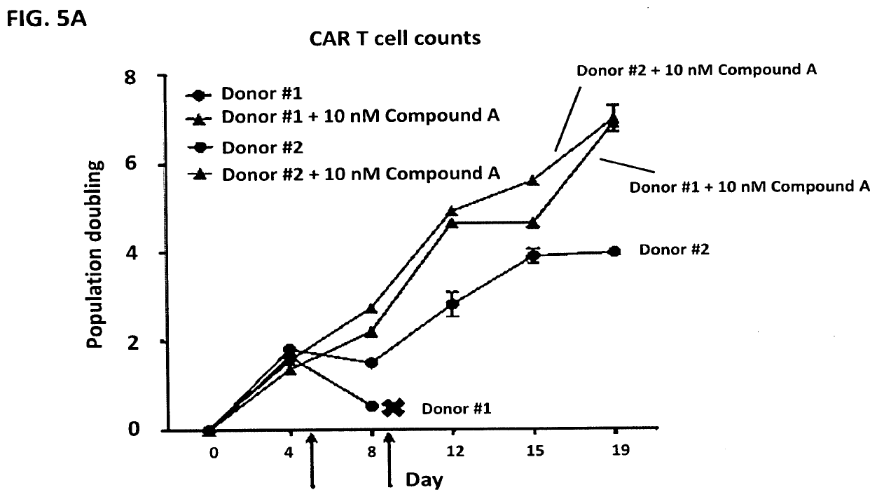

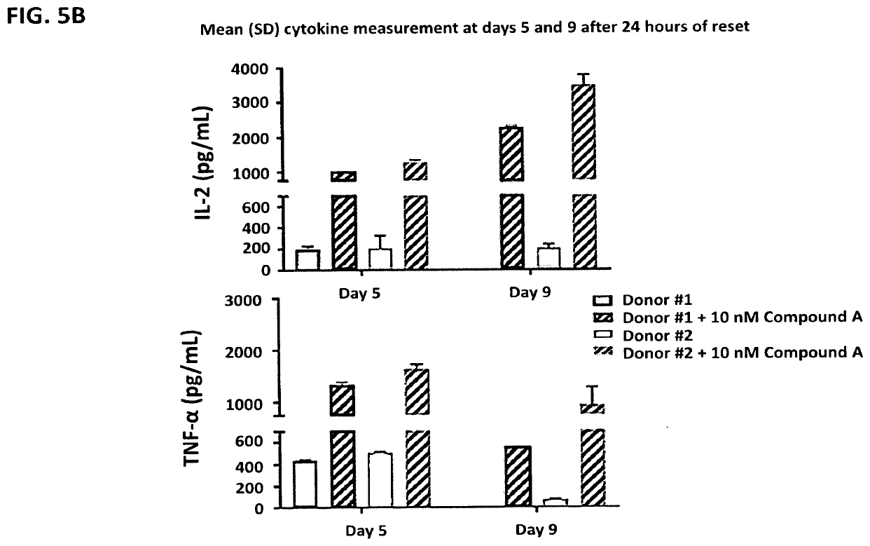

Results shown in FIG. 5A demonstrate that the addition of Compound A enhanced anti-BCMA CAR cell counts in the culture, as demonstrated by an increase in the number of population doublings during the serial stimulation when Compound A was added compared to when it is absent. As shown in FIG. 5B, the addition of Compound A also increased IL-2 and TNF-alpha cytokine production in the cultures 24 hours after a first reset (day 5) or second reset (day 9) following replating with fresh target cells in the serial stimulation assay. These results are further consistent with an observation that the pharmacological performance of anti-BCMA CAR T cells was enhanced with the addition of the cell immunomodulatory compound Compound A.

### Example 6 Effect of Iberdomide on CAR T-Cell Function In Vivo

Anti-tumor effects of anti-BCMA CAR T cells, alone and in combination with concurrent or delayed administration of Compound A (iberdomide, (S)-3-[4-(4-morpholin ylmethyl-benzyloxy)-1-oxo-1,3-dihydro-isoindol-2-yl]-piperidine-2,6-dione), were assessed in disseminated tumor models. The combinatorial effects were assessed in both tumor models sensitive to and resistant to the immunomodulatory compound. The anti-BCMA CAR T cells used in the study were produced as described in Example 1.

A. Tumor Model Sensitive to Cell Immunomodulatory Compound

The effects of anti-BCMA CAR-T cells in combination with Compound A was assessed in a murine orthotopic tumor model using OPM-2 cells, which is an tumor model sensitive to the immunomodulatory compound. Mice (NOD.Cg-Prkdc^{scid}IL-2rg^{tm1Wj1}/SzJ mice (NSG; Jackson Labs)) were injected intravenously (i.v.) with 2×10⁶ OPM2 (multiple myeloma) cells transfected with firefly luciferase (OPM2-ffluc). Tumor engraftment was allowed to occur for 13 days prior to staging (14 days before CAR-T cell administration) and verified using bioluminescence imaging.

In one study, mice were administered Compound A (1 mg/kg or 3 mg/kg) via oral administration once a day starting from the day before administration of 0.5×10⁶ anti-BCMA CAR T cells, and administration of Compound A was continued once a day until 32 days post-CAR T cell administration (concurrent dosing). In another study, mice were administered 0.5×10⁶ anti-BCMA CAR T cells, and then Compound A (1 mg/kg or 3 mg/kg) was administered via oral administration once a day starting 12 days after anti-CAR T cell administration, which was after the peak of CAR-expressing T cell expansion, and administration was continued for 21 days until 32 days post-CAR T cell administration (delayed dosing).

For bioluminescence imaging (BLI), mice received intraperitoneal (i.p.) injections of luciferin substrate (CaliperLife Sciences, Hopkinton, Mass.) resuspended in PBS (15 μg/g body weight). The average radiance (p/s/cm²/sr) was determined. Survival of mice treated as described above were assessed and compared over time post-infusion of CAR-expressing T cells. Survival curves were generated using the Kaplan-Meier method (GraphPad Prism 7.0, GraphPad Software, La Jolla).

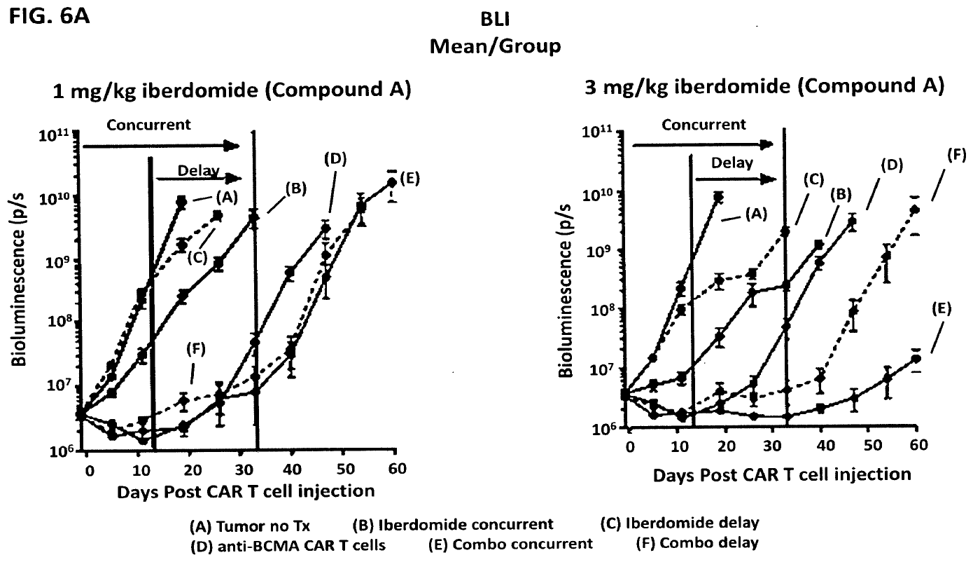

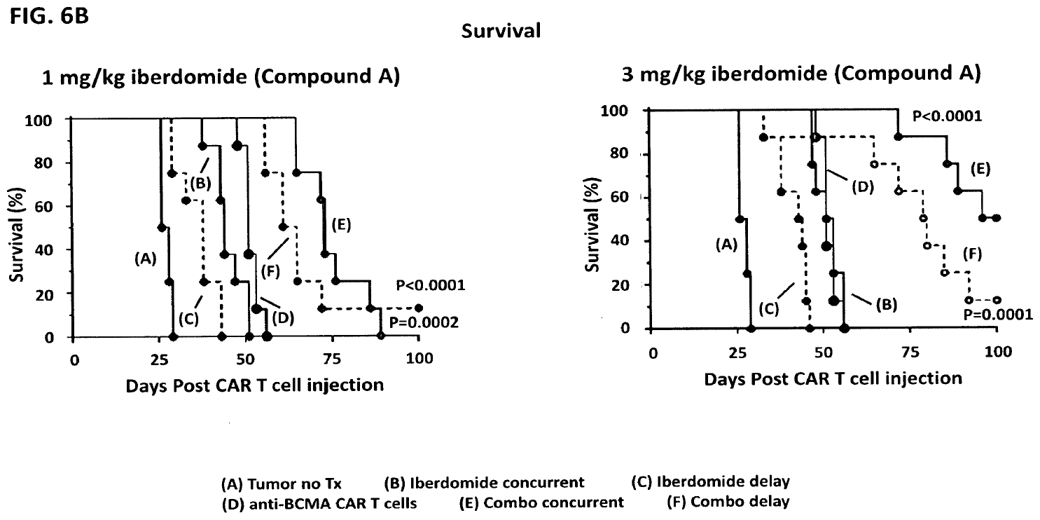

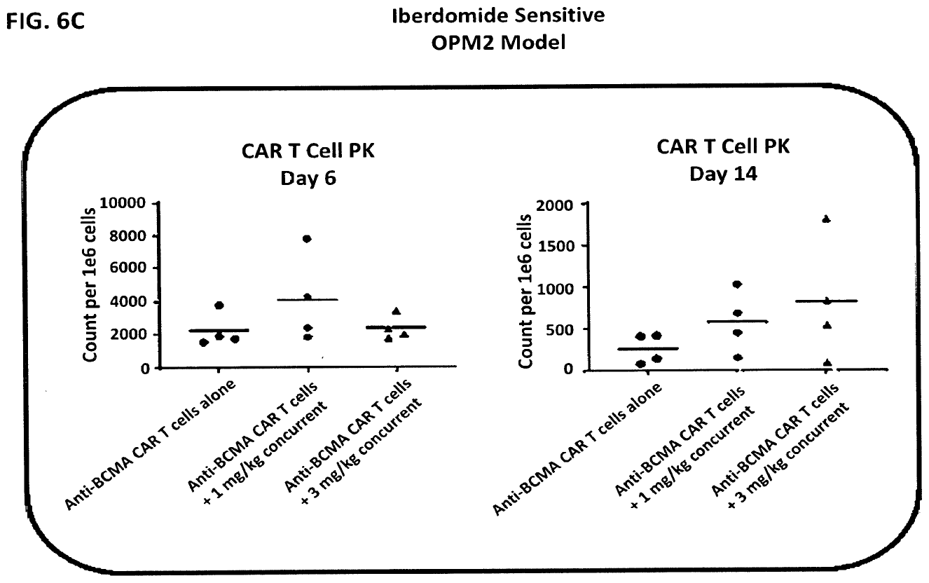

The results are shown in FIG. 6A (tumor volume) and FIG. 6B (survival). Compound A exhibited some single agent antitumor activity in the OPM-2 tumor model. The combination of anti-BCMA CAR T cell administration with Compound A was observed to reduce tumor burden and improve survival data in both the “Concurrent” group and the “Delayed” group, as compared to administration of the anti-BCMA CAR-expressing T cells alone. For example, Compound A, either administered delayed or concurrent with anti-BCMA CAR-T cells at the low and high dose, resulted in greater decrease in tumor as measured by BLI (FIG. 6A) and a greater percent survival of mice compared to mice receiving only administration of anti-BCMA CAR-T cells (FIG. 6B).

The numbers of CD3+ CAR T cells were determined in the blood at day 6 and day 14 by flow cytometry using antibodies directed against CD3 and the surrogate marker on the CAR-expressing cells. As shown in FIG. 6C, there was a trend towards increased numbers of CD3+ CAR+ T cells in the blood in mice having received the combination of anti-BCMA CAR+ T cells and Compound A in the concurrent regimen.

B. Tumor Model Resistant to Cell Immunomodulatory Compound

The effects of anti-BCMA CAR-T cells in combination with Compound A was assessed in a murine orthotopic tumor model using DF-15(R) cells, which is a tumor model that is resistant to the immunomodulatory compound. Mice (NOD.Cg-Prkdc^{scid}IL-2rg^{tm1Wj1}/SzJ mice (NSG; Jackson Labs)) were injected intravenously (i.v.) with 2×10⁶ DF-15(R) (multiple myeloma) cells transfected with firefly luciferase (OPM2-ffluc). Tumor engraftment was allowed to occur for 13 days prior to staging (14 days before CAR-T cell administration) and verified using bioluminescence imaging.

In one study, mice were administered Compound A (1 mg/kg or 3 mg/kg) via oral administration once a day starting from the day before administration of 0.5×10⁶ anti-BCMA CAR T cells, and administration of Compound A was continued once a day until 32 days post-CAR T cell administration (concurrent dosing). In another study, mice were administered 0.5×10⁶ anti-BCMA CAR T cells, and then Compound A (1 mg/kg or 3 mg/kg) was administered via oral administration once a day starting 12 days after anti-CAR T cell administration, which was after the peak of CAR-expressing T cell expansion, and administration was continued for 21 days until 32 days post-CAR T cell administration (delayed dosing).

For bioluminescence imaging (BLI), mice received intraperitoneal (i.p.) injections of luciferin substrate (CaliperLife Sciences, Hopkinton, Mass.) resuspended in PBS (15 μg/g body weight). The average radiance (p/s/cm²/sr) was determined. Survival of mice treated as described above were assessed and compared over time post-infusion of CAR-expressing T cells. Survival curves were generated using the Kaplan-Meier method (GraphPad Prism 7.0, GraphPad Software, La Jolla).

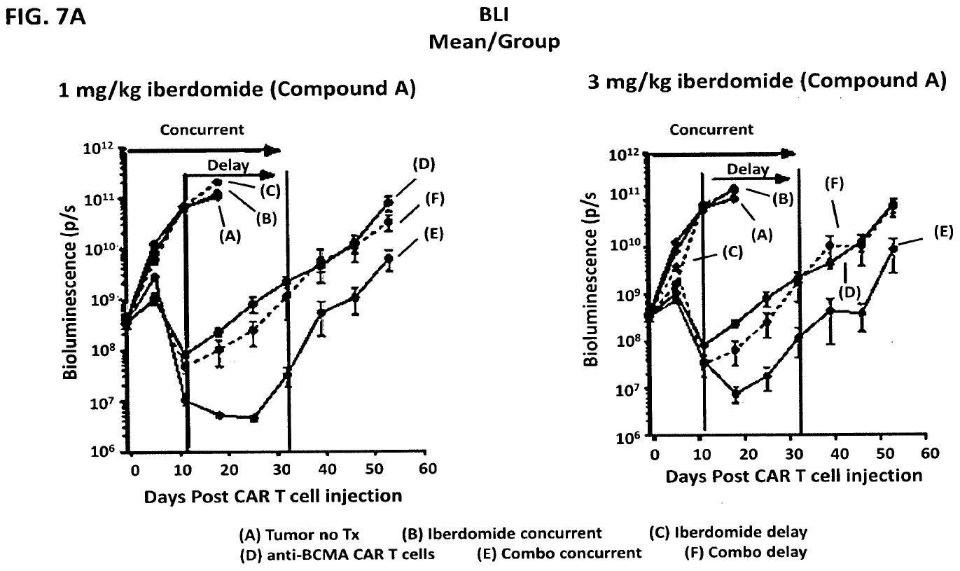

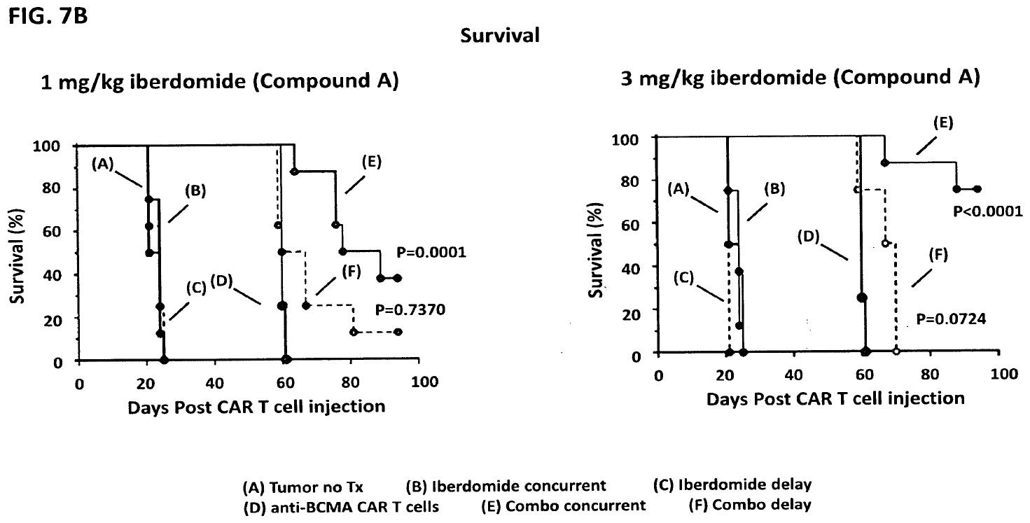

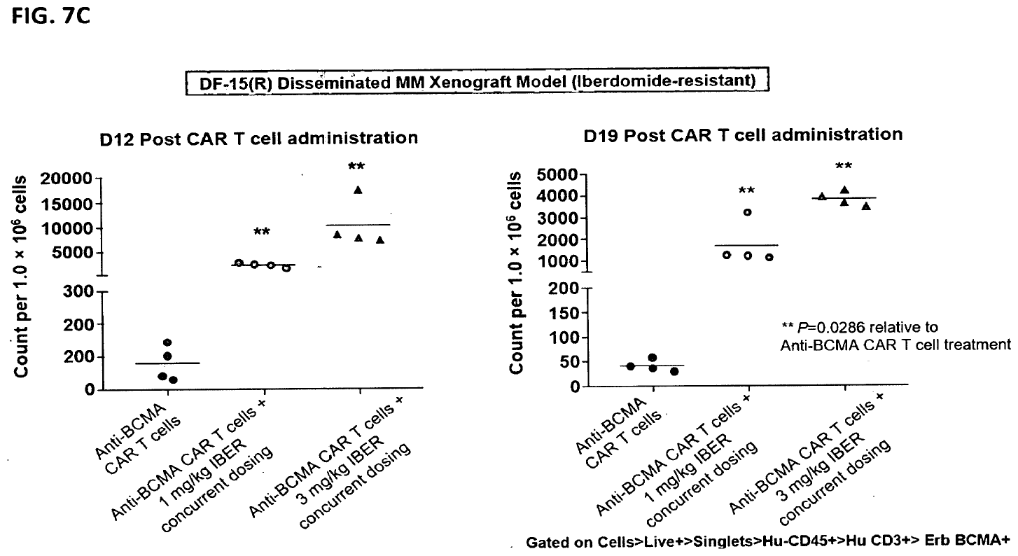

The results are shown in FIG. 7A (tumor volume) and FIG. 7B (survival). In this tumor resistant model, the combination of anti-BCMA CAR T cell administration with Compound A was observed to reduce tumor burden and improve survival data in the “Concurrent” group, as compared to administration of the anti-BCMA CAR-expressing cells alone.

The numbers of CD3+ CAR T cells were determined in the blood at day 6 and day 14 by flow cytometry using antibodies directed against CD3 and the surrogate marker on the CAR-expressing cells. As shown in FIG. 7C, there was a statistically significant increase in numbers of CD3+ CAR+ T cells in the blood in mice having received anti-BCMA CAR+ T cells at both the low and high dose in combination with Compound A in the concurrent regimen.

### Example 7 Cytolytic Function and Cytokine Production of Chronically Stimulated Anti-BCMA CAR T Cells Against BCMA-Expressing MM Target Cells in the Presence of Cell Immunomodulatory Compound

Cryofrozen anti-BCMA CAR T cells, produced substantially as described in Example 1 and formulated at a 1:1 ratio of CD4+ and CD8+ T cells, were thawed. Anti-BCMA CAR T cells were stimulated with BCMA conjugated beads (diameter about 4.5 μm from a 50 μg/ml BCMA-conjugated bead composition, generated as described in Example 9) at a ratio of T cells to beads of 1:1 in the presence or absence of lenalidomide (1000 nM), Compound A (iberdomide, (S)-3-[4-(4-morpholin-4-ylmethyl-benzyloxy)-1-oxo-1,3-dihydro-isoindol-2-yl]-piperidine-2,6-dione) (0.1 nM, 1 nM or 10 nM) or DMSO vehicle control. The cells were then incubated at 37° C., 5% CO2 for 7 days.

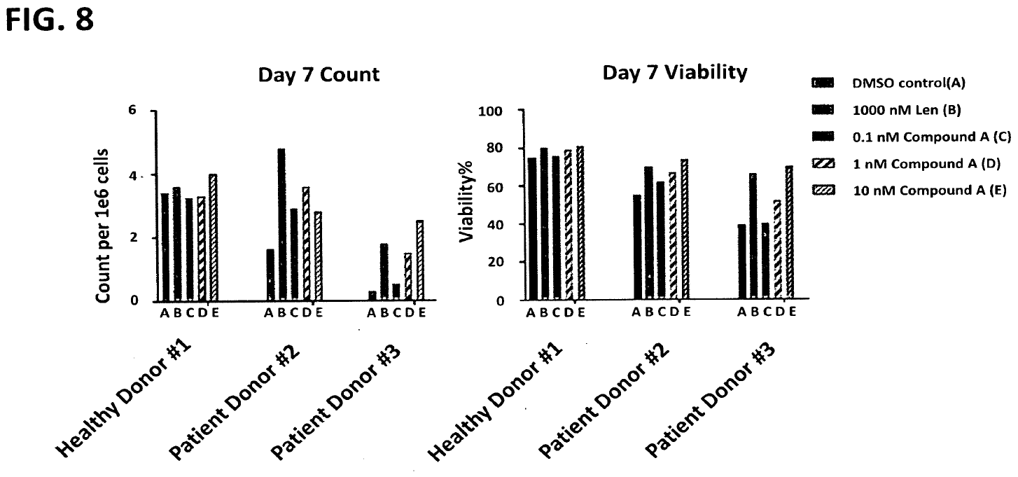

At day 7, anti-BCMA CAR cells of all the samples were counted using a cellometer machine and stained for flow cytometric analysis. Cells were stained with a viability dye and analyzed by flow cytometry. As shown in FIG. 8, viability and count of anti-BCMA CAR T cells was increased in the presence of lenalidomide or Compound A.

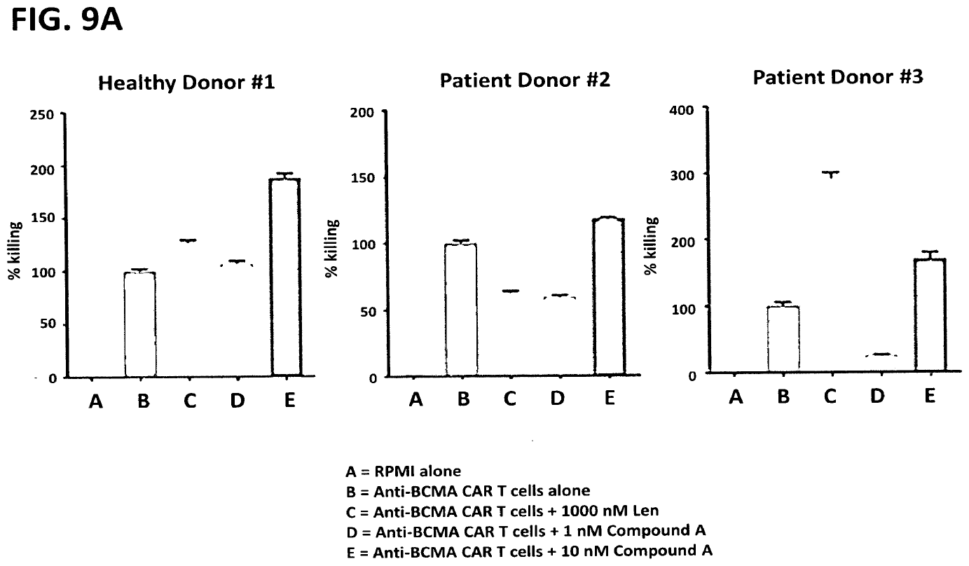

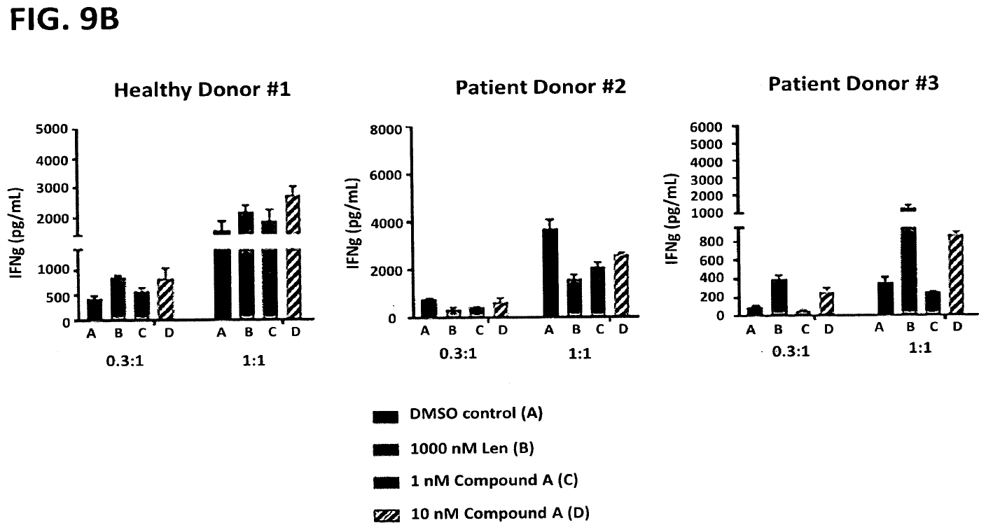

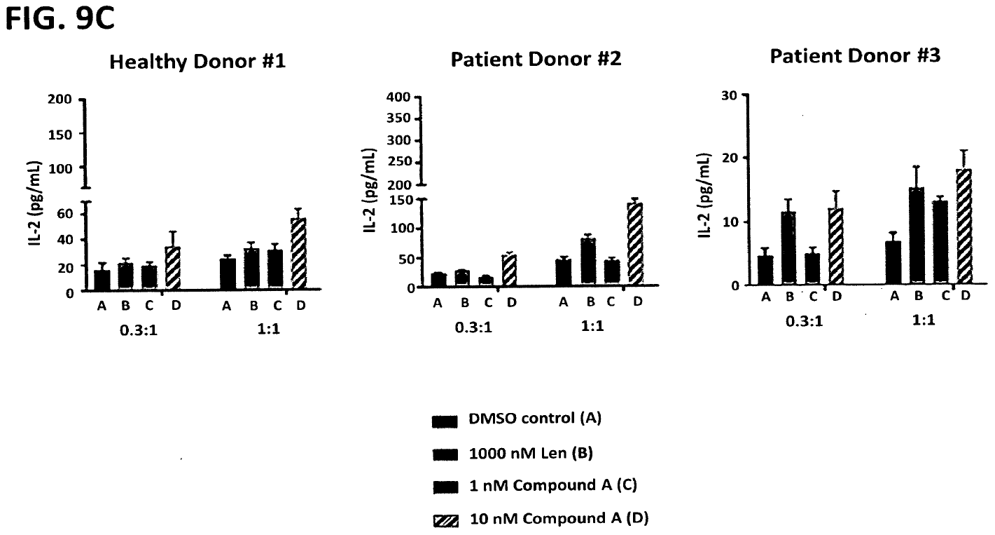

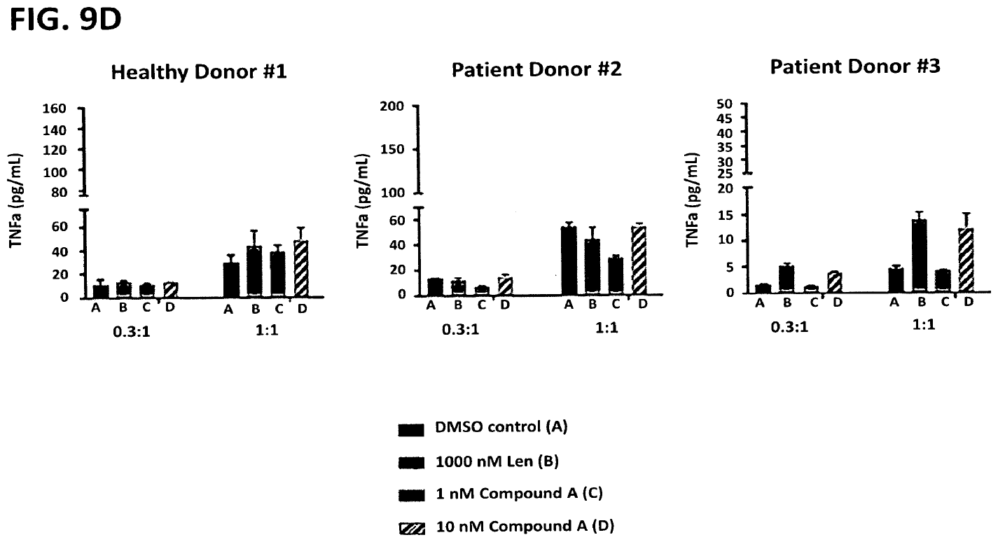

Cytolytic activity was assessed using OPM-2 and RPMI-8226 BCMA expressing target cells transduced with NucLight Red, a red fluorescent protein detectable by microscopy, to allow for measurement of target cell death. Anti-BCMA CAR T cells that had been stimulated for 7 days with BCMA-conjugated beads in the presence of the compounds were co-cultured with RPMI-8226 target cells at a 0.3:1 (effector:target) or 1:1 ratio. Cultures were incubated at 37° C., 5% CO2, and images were taken every 2 hours over 5-7 days with an Essen IncuCyte Zoom live-cell analysis system to track NucLightRed-positive target cells. When the long-term stimulation was carried out in the presence of lenalidomide or Compound A, anti-BCMA CAR T cells showed increased cytolytic activity (FIG. 9A, results shown for 0.3:1 E:T ratio).

For cytokine measurements, cell-free supernatants were collected from the cytolytic assay described above 24 hours after plating. Cytokine levels were measured using IFNg, IL-2, and TNFalpha Meso Scale Discovery cytokine kit (Mesoscale) according to manufacturer instructions. The data was analyzed using GraphPad Prism to calculate absolute changes in cytokine relative to the DMSO vehicle control. As shown in FIG. 9B-D, when the long-term stimulation was carried out in the presence of lenalidomide or Compound A, anti-BCMA CAR T cells showed increased production of IFN-gamma (FIG. 9B), IL-2 (FIG. 9C) or TNF-alpha (FIG. 9D).

These results demonstrate that lenalidomide or Compound A present during chronic stimulation increases anti-BCMA CAR T cell cytolytic activity and cytokine production following antigen rechallenge, and in the absence of the compound during rechallenge. These results further support the ability of cell immunomodulatory compounds, such as lenalidomide or Compound A, to reduce or prevent the development of an exhausted phenotype in response to chronic stimulation, thereby improving CAR-T cell function and limiting CAR T cell exhaustion.

### Example 8 Rescue of Cytolytic Function and Cytokine Production Following Chronic Stimulation of Anti-BCMA CAR T Cells by Cell Immunomodulatory Compound

Studies were undertaken to determine whether Compound A (iberdomide, (S)-3-[4-(4-morpholin-4-ylmethyl-benzyloxy)-1-oxo-1,3-dihydro-isoindol-2-yl]-piperidine-2,6-dione) rescued anti-BCMA CAR T cell function following chronic stimulation. To directly stimulate via CAR engagement to induce chronic stimulation of cells, anti-BCMA CAR T cells were stimulated with BCMA conjugated beads (diameter about 4.5 μm from a 50 μg/ml BCMA-conjugated bead composition, generated as described in Example 9) at a ratio of T cells to beads of 1:1. The cells were then incubated at 37° C., 5% CO₂ for 7 days.

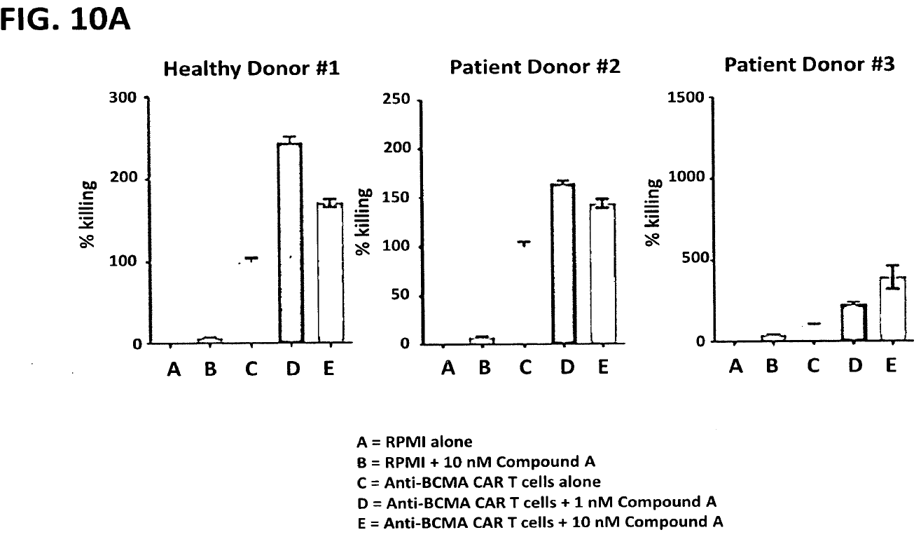

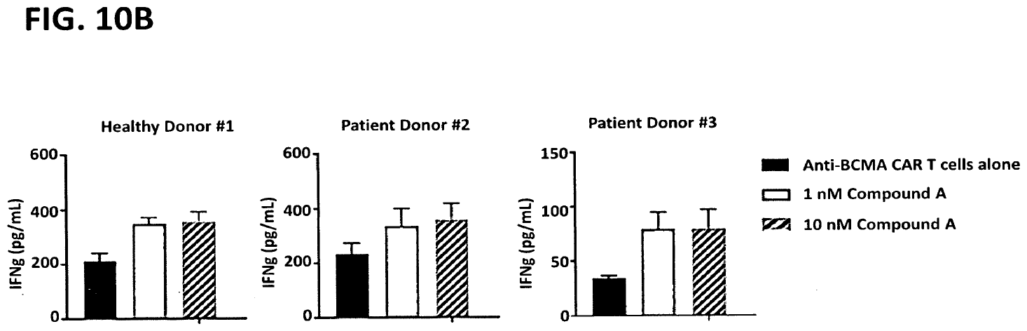

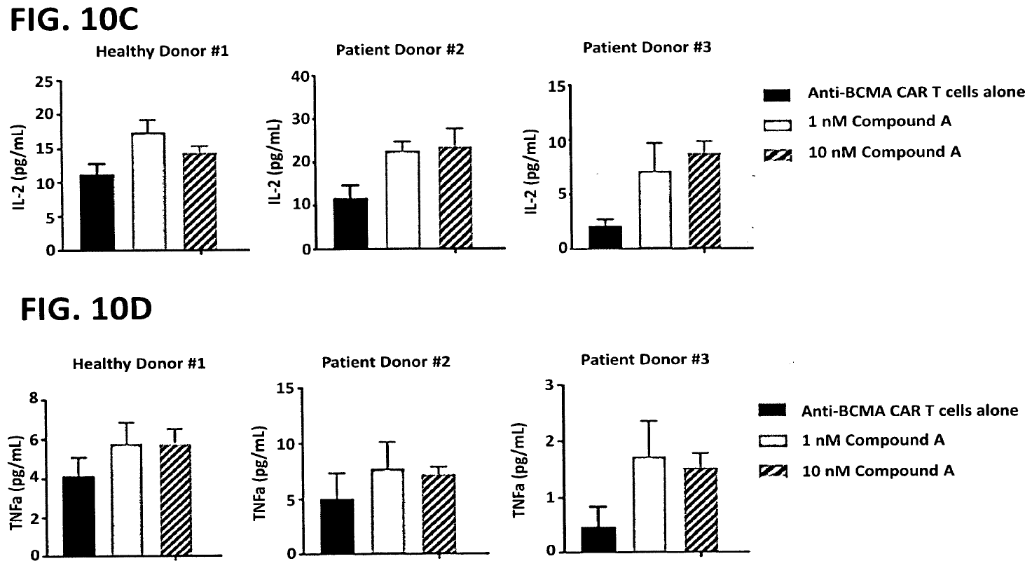

Anti-BCMA CAR T cells that had been stimulated for 7 days with BCMA-conjugated beads were re-challenged with BCMA-expressing RPMI-8226 MM cells at a 0.3:1 E:T ratio in the presence of Compound A (1 nm or 10 nM). Cultures were incubated at 37° C., 5% CO2, and images were taken every 2 hours over 5-7 days with an Essen IncuCyte Zoom live-cell analysis system to track NucLightRed-positive target cells. As shown in FIG. 10A, there was an improvement in cytolytic activity when chronically stimulated cells were re-challenged with BCMA-expressing cells in the presence of Compound A compared to absence of the compound (control). Cell-free supernatants were collected from the cytolytic assay described above 24 hours after plating, and used to measure IFNg, IL-2, and TNF by MSD, as described in Example 30. As shown in FIG. 10B-D, anti-BCMA CAR T cells showed increased production of IFN-gamma (FIG. 10B), IL-2 (FIG. 10C) or TNF-alpha (FIG. 10D) when chronically stimulated cells were re-challenged with BCMA-expressing cells in the presence of Compound A compared to absence of the compound (control).

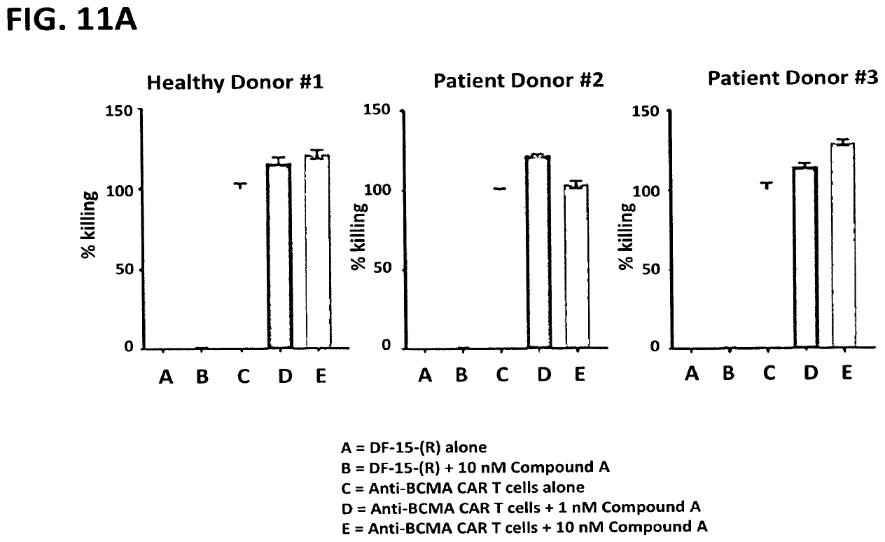

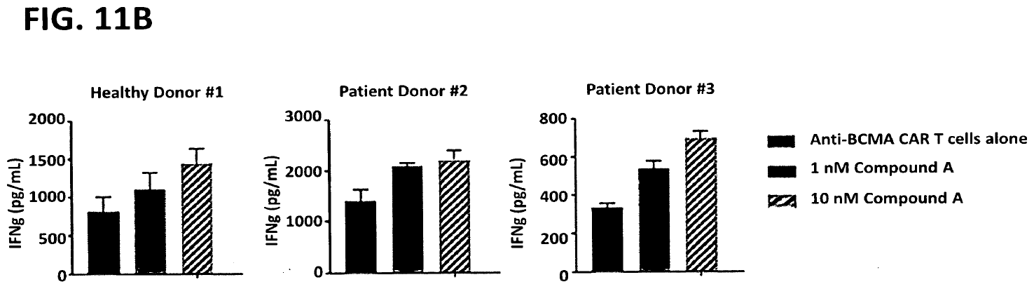

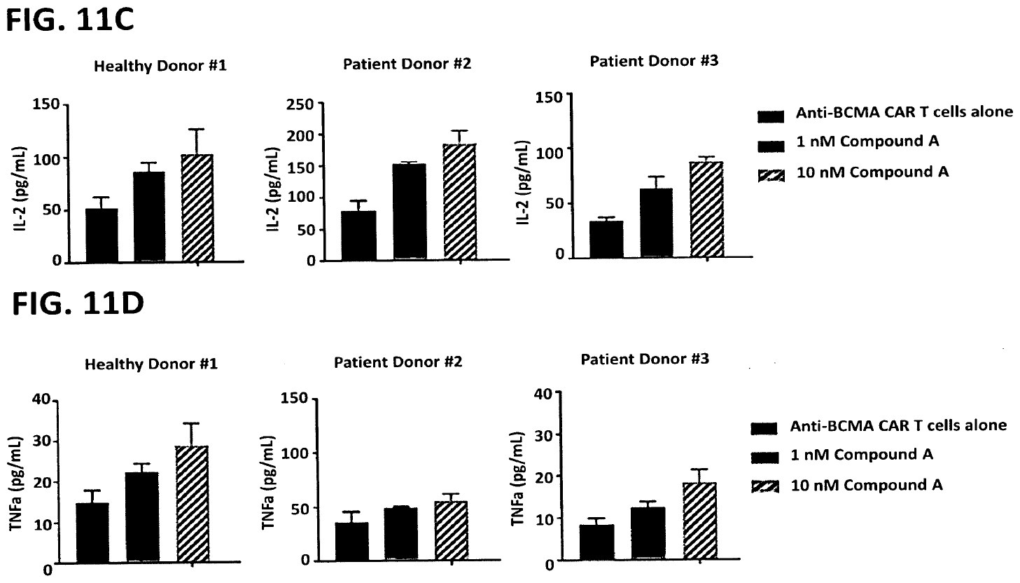

To further elucidate the role of Compound A on the target cells compared to the CAR T cell intrinsic effects, the IMiD/CELMoD resistant cell line DF-15R was also used to rechallenge anti-BCMA CAR T cells that had been chronically stimulated for 7 days. Cytolytic activity and cytokine production following the rechallenge were assessed as described above. Anti-BCMA CAR T cells showed both increased cytolytic activity (FIG. 11A) and cytokine production (FIG. 11B-D) in the presence of DF-15R, indicating a CAR T-intrinsic increase in functionality.

These results further demonstrate that, following chronic stimulation, the addition of cell immunomodulatory compounds, such as Compound A, during antigen rechallenge rescues exhausted anti-BCMA CAR T cells, as shown by increased cytolytic activity and cytokine production.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID | | |
| NO. | SEQUENCE | DESCRIPTION |
| 1 | ESKYGPPCPPCP | spacer |
| | | (IgG4hinge) (aa) |
| | | Homo sapiens |
| 2 | GAATCTAAGTACGGACCGCCCTGCCCCCCTTGCCCT | spacer |
| | | (IgG4hinge) (nt) |
| | | Homo sapiens |
| 3 | ESKYGPPCPPCPGQPREPQVYTLPPSQEEMTKNQVSLTCLVK | Hinge-CH3 |
| | GFYPSDIAVEWESNGQPENNYKTTPPVLDSDGSFFLYSRLTV | spacer |
| | DKSRWQEGNVFSCSVMHEALHNHYTQKSLSLSLGK | Homo sapiens |
| 4 | ESKYGPPCPPCPAPEFLGGPSVFLFPPKPKDTLMISRTPEVTCV | Hinge-CH2-CH3 |
| | VVDVSQEDPEVQFNWYVDGVEVHNAKTKPREEQFNSTYRV | spacer |
| | VSVLTVLHQDWLNGKEYKCKVSNKGLPSSIEKTISKAKGQP | Homo sapiens |
| | REPQVYTLPPSQEEMTKNQVSLTCLVKGFYPSDIAVEWESNG | |
| | QPENNYKTTPPVLDSDGSFFLYSRLTVDKSRWQEGNVFSCSV | |
| | MHEALHNHYTQKSLSLSLGK | |
| 5 | RWPESPKAQASSVPTAQPQAEGSLAKATTAPATTRNTGRGG | IgD-hinge-Fc |
| | EEKKKEKEKEEQEERETKTPECPSHTQPLGVYLLTPAVQDL | Homo sapiens |
| | WLRDKATFTCFVVGSDLKDAHLTWEVAGKVPTGGVEEGLL | |
| | ERHSNGSQSQHSRLTLPRSLWNAGTSVTCTLNHPSLPPQRLM | |
| | ALREPAAQAPVKLSLNLLASSDPPEAASWLLCEVSGFSPPNIL | |
| | LMWLEDQREVNTSGFAPARPPPQPGSTTFWAWSVLRVPAPP | |
| | SPQPATYTCVVSHEDSRTLLNASRSLEVSYVTDH | |
| 6 | LEGGGEGRGSLLTCGDVEENPGPR | T2A |
| | | artificial |
| 7 | RKVCNGIGIGEFKDSLSINATNIKHFKNCTSISGDLHILPVAFR | tEGFR |
| | GDSFTHTPPLDPQELDILKTVKEITGFLLIQAWPENRTDLHAF | artificial |
| | ENLEIIRGRTKQHGQFSLAVVSLNITSLGLRSLKEISDGDVIIS | |
| | GNKNLCYANTINWKKLFGTSGQKTKIISNRGENSCKATGQV | |
| | CHALCSPEGCWGPEPRDCVSCRNVSRGRECVDKCNLLEGEP | |
| | REFVENSECIQCHPECLPQAMNITCTGRGPDNCIQCAHYIDGP | |
| | HCVKTCPAGVMGENNTLVWKYADAGHVCHLCHPNCTYGC | |
| | TGPGLEGCPTNGPKIPSIATGMVGALLLLLVVALGIGLFM | |
| 8 | FWVLVVVGGVLACYSLLVTVAFIIFWV | CD28 (amino |
| | | acids 153-179 of |
| | | Accession No. |
| | | P10747) |
| | | Homo sapiens |
| 9 | IEVMYPPPYLDNEKSNGTIIHVKGKHLCPSPLFPGPSKPFWVL | CD28 (amino |
| | VVVGGVLACYSLLVTVAFIIFWV | acids 114-179 of |
| | | Accession No. |
| | | P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | P10747) |
| | | Homo sapiens |
| 11 | RSKRSRGGHSDYMNMTPRRPGPTRKHYQPYAPPRDFAAYRS | CD28 (LL to GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) |
| | | Homo sapiens |
| 13 | RVKFSRSADAPAYQQGQNQLYNELNLGRREEYDVLDKRRG | CD3 zeta |
| | RDPEMGGKPRRKNPQEGLYN ELQKDKMAEA | Homo sapiens |
| | YSEIGMKGER RRGKGHDGLY | |
| | QGLSTATKDTYDALHMQALP PR | |
| 14 | RVKFSRSAEPPAYQQGQNQLYNELNLGRREEYDVLDKRRGR | CD3 zeta |
| | DPEMGGKPRRKNPQEGLYN ELQKDKMAEA YSEIGMKGER | Homo sapiens |
| | RRGKGHDGLY QGLSTATKDTYDALHMQALP PR | |
| 15 | RVKFSRSADAPAYKQGQNQLYNELNLGRREEYDVLDKRRG | CD3 zeta |
| | RDPEMGGKPRRKNPQEGLYN ELQKDKMAEA | Homo sapiens |
| | YSEIGMKGER RRGKGHDGLY | |
| | QGLSTATKDTYDALHMQALP PR | |
| 16 | PGGG-(SGGGG)5-P- wherein P is proline, G is glycine and S is | linker |
| | serine | |
| 17 | GSADDAKKDAAKKDGKS | Linker |
| 18 | MLQMAGQCSQNEYFDSLLHACIPCQLRCSSNTPPLTCQRYC | Extracellular |
| | NASVTNSVKGTNA | domain of human |
| | | BCMA |
| | | (GenBank No. |
| | | NP_001183.2) |
| 19 | GGGGS | Linker sequence |
| 20 | PKSSDKTHTCPPCPAPEAEGAPSVFLFPPKPKDTLMISRTPEV | Modified Human |
| | TCVVVDVSHEDPEVKFNWYVDGVEVHNAKTKPREEQYNST | IgG1 Fc |
| | YRVVSVLTVLHQDWLNGKEYKCKVSNKALPSSIEKTISKAK | |
| | GQPREPQVYTLPPSRDELTKNQVSLTCLVKGFYPSDIAVEWE | |
| | SNGQPENNYKTTPPVLDSDGSFFLYSKLTVDKSRWQQGNVF | |
| | SCSVMHEALHNHYTQKSLSLSPGK | |
| 21 | MPLLLLLPLLWAGALA | CD33 Signal |
| | | peptide |
| 22 | MPLLLLLPLLWAGALAMLQMAGQCSQNEYFDSLLHACIPCQ | BCMA-Fc |
| | LRCSSNTPPLTCQRYCNASVTNSVKGTNAGGGGSPKSSDKT | construct |
| | HTCPPCPAPEAEGAPSVFLFPPKPKDTLMISRTPEVTCVVVDV | |
| | SHEDPEVKFNWYVDGVEVHNAKTKPREEQYNSTYRVVSVL | |
| | TVLHQDWLNGKEYKCKVSNKALPSSIEKTISKAKGQPREPQ | |
| | VYTLPPSRDELTKNQVSLTCLVKGFYPSDIAVEWESNGQPEN | |
| | NYKTTPPVLDSDGSFFLYSKLTVDKSRWQQGNVFSCSVMHE | |
| | ALHNHYTQKSLSLSPGK | |
| 23 | EGRGSLLTCGDVEENPGP | T2A |
| 24 | GSGATNFSLLKQAGDVEENPGP | P2A |
| 25 | ATNFSLLKQAGDVEENPGP | P2A |
| 26 | QCTNYALLKLAGDVESNPGP | E2A |
| 27 | VKQTLNFDLLKLAGDVESNPGP | F2A |
| 28 | RKVCNGIGIGEFKDSLSINATNIKHFKNCTSISGDLHILPVAFR | tEGFR |
| | GDSFTHTPPLDPQELDILKTVKEITGFLLIQAWPENRTDLHAF | artificial |
| | ENLEIIRGRTKQHGQFSLAVVSLNITSLGLRSLKEISDGDVIIS | |
| | GNKNLCYANTINWKKLFGTSGQKTKIISNRGENSCKATGQV | |
| | CHALCSPEGCWGPEPRDCVSCRNVSRGRECVDKCNLLEGEP | |
| | REFVENSECIQCHPECLPQAMNITCTGRGPDNCIQCAHYIDGP | |
| | HCVKTCPAGVMGENNTLVWKYADAGHVCHLCHPNCTYGC | |
| | TGPGLEGCPTNGPKIPSIATGMVGALLLLLVVALGIGLFM | |
| 29 | ESKYGPPCPPCPAPPVAGPSVFLFPPKPKDTLMISRTPEVTCV | Hinge-CH2-CH3 |
| | VVDVSQEDPEVQFNWYVDGVEVHNAKTKPREEQFQSTYRV | spacer |
| | VSVLTVLHQDWLNGKEYKCKVSNKGLPSSIEKTISKAKGQP | Homo sapiens |
| | REPQVYTLPPSQEEMTKNQVSLTCLVKGFYPSDIAVEWESNG | |
| | QPENNYKTTPPVLDSDGSFFLYSRLTVDKSRWQEGNVFSCSV | |
| | MHEALHNHYTQKSLSLSLGK | |
| 30 | QIQLVQSGPELKKPGETVKISCKASGYTFTDYSINWVKRAPG | Variable heavy |
| | KGLKWMGWINTETREPAYAYDFRGRFAFSLETSASTAYLQI | (VH) Anti- |
| | NNLKYEDTATYFCALDYSYAMDYWGQGTSVTVSS | BCMA |
| 31 | DIVLTQSPPSLAMSLGKRATISCRASESVTILGSHLIHWYQQK | Variable light |
| | PGQPPTLLIQLASNVQTGVPARFSGSGSRTDFTLTIDPVEEDD | (VL) Anti- |
| | VAVYYCLQSRTIPRTFGGGTKLEIK | BCMA |
| 32 | QIQLVQSGPDLKKPGETVKLSCKASGYTFTNFGMNWVKQAP | Variable heavy |
| | GKGFKWMAWINTYTGESYFADDFKGRFAFSVETSATTAYLQ | (VH) Anti- |
| | INNLKTEDTATYFCARGEIYYGYDGGFAYWGQGTLVTVSA | BCMA |
| 33 | DVVMTQSHRFMSTSVGDRVSITCRASQDVNTAVSWYQQKP | Variable light |
| | GQSPKLLIFSASYRYTGVPDRFTGSGSGADFTLTISSVQAEDL | (VL) Anti- |
| | AVYYCQQHYSTPWTFGGGTKLDIK | BCMA |
| 34 | EVQLVQSGAEVKKPGESLKISCKGSGYSFTSYWIGWVRQMP | Variable heavy |
| | GKGLEWMGIIYPGDSDTRYSPSFQGHVTISADKSISTAYLQW | (VH) Anti- |
| | SSLKASDTAMYYCARYSGSFDNWGQGTLVTVSS | BCMA |
| 35 | SYELTQPPSASGTPGQRVTMSCSGTSSNIGSHSVNWYQQLPG | Variable light |
| | TAPKLLIYTNNQRPSGVPDRFSGSKSGTSASLAISGLQSEDEA | (VL) Anti- |
| | DYYCAAWDGSLNGLVFGGGTKLTVLG | BCMA |
| 36 | EVQLVQSGAEMKKPGASLKLSCKASGYTFIDYYVYWMRQA | Variable heavy |
| | PGQGLESMGWINPNSGGTNYAQKFQGRVTMTRDTSISTAYM | (VH) Anti- |
| | ELSRLRSDDTAMYYCARSQRDGYMDYWGQGTLVTVSS | BCMA |
| 37 | QSALTQPASVSASPGQSIAISCTGTSSDVGWYQQHPGKAPKL | Variable light |
| | MIYEDSKRPSGVSNRFSGSKSGNTASLTISGLQAEDEADYYC | (VL) Anti- |
| | SSNTRSSTLVFGGGTKLTVLG | BCMA |
| 38 | EVQLVQSGAEVKKPGSSVKVSCKASGGTFSSYAISWVRQAP | Variable heavy |
| | GQGLEWMGRIIPILGIANYAQKFQGRVTMTEDTSTDTAYME | (VH) Anti- |
| | LSSLRSEDTAVYYCARSGYSKSIVSYMDYWGQGTLVTVSS | BCMA |
| 39 | LPVLTQPPSTSGTPGQRVTVSCSGSSSNIGSNVVFWYQQLPGT | Variable light |
| | APKLVIYRNNQRPSGVPDRFSVSKSGTSASLAISGLRSEDEAD | (VL) Anti- |
| | YYCAAWDDSLSGYVFGTGTKVTVLG | BCMA |
| 40 | QVQLVQSGAEVKKPGSSVKVSCKASGGTFSSYAISWVRQAP | Variable heavy |
| | GQGLEWMGRIIPILGTANYAQKFQGRVTITADESTSTAYMEL | (VH) Anti- |
| | SSLRSEDTAVYYCARSGYGSYRWEDSWGQGTLVTVSS | BCMA |
| 41 | QAVLTQPPSASGTPGQRVTISCSGSSSNIGSNYVFWYQQLPGT | Variable light |
| | APKLLIYSNNQRPSGVPDRFSGSKSGTSASLAISGLRSEDEAD | (VL) Anti- |
| | YYCAAWDDSLSASYVFGTGTKVTVLG | BCMA |
| 42 | QVQLVQSGAEVKKPGASVKVSCKASGYTFTDYYMHWVRQ | Variable heavy |
| | APGQRLEWMGWINPNSGGTNYAQKFQDRITVTRDTSSNTGY | (VH) Anti- |
| | MELTRLRSDDTAVYYCARSPYSGVLDKWGQGTLVTVSS | BCMA |
| 43 | QSVLTQPPSVSGAPGQRVTISCTGSSSNIGAGFDVHWYQQLP | Variable light |
| | GTAPKLLIYGNSNRPSGVPDRFSGSKSGTSASLAITGLQAEDE | (VL) Anti- |
| | ADYYCQSYDSSLSGYVFGTGTKVTVLG | BCMA |
| 44 | IYIWAPLAGTCGVLLLSLVITLYCNHRN | CD8a TM |
| 45 | IYIWAPLAGTCGVLLLSLVIT | CD8a TM |
| 46 | RAAA | linking peptide |
| 47 | EVQLVESGGGLVKPGGSLRLSCAASGFTFSDYYMSWIRQAP | Variable heavy |
| | GKGLEWVSYISSSGSTIYYADSVKGRFTISRDNAKNSLYLQM | (VH) Anti- |
| | NSLRAEDTAVYYCAKVDGDYTEDYWGQGTLVTVSS | BCMA |
| 48 | QSALTQPASVSGSPGQSITISCTGSSSDVGKYNLVSWYQQPPG | Variable light |
| | KAPKLIIYDVNKRPSGVSNRFSGSKSGNTATLTISGLQGDDEA | (VL) Anti- |
| | DYYCSSYGGSRSYVFGTGTKVTVL | BCMA |
| 49 | EVQLVQSGGGLVQPGRSLRLSCTASGFTFGDYAMSWFRQAP | Variable heavy |
| | GKGLEWVGFIRSKAYGGTTEYAASVKGRFTISRDDSKSIAYL | (VH) Anti- |
| | QMNSLKTEDTAVYYCAAWSAPTDYWGQGTLVTVSS | BCMA |
| 50 | DIQMTQSPAFLSASVGDRVTVTCRASQGISNYLAWYQQKPG | Variable light |
| | NAPRLLIYSASTLQSGVPSRFRGTGYGTEFSLTIDSLQPEDFAT | (VL) Anti- |
| | YYCQQSYTSRQTFGPGTRLDIK | BCMA |
| 51 | EVQLVESGGGLVKPGGSLRLSCAASGFTFSDYYMSWIRQAP | Variable heavy |
| | GKGLEWVSYISSSGSTIYYADSVKGRFTISRDNAKNSLYLQM | (VH) Anti- |
| | NSLRAEDTAVYYCAKVDGPPSFDIWGQGTMVTVSS | BCMA |
| 52 | SYVLTQPPSVSVAPGQTARITCGANNIGSKSVHWYQQKPGQ | Variable light |
| | APMLVVYDDDDRPSGIPERFSGSNSGNTATLTISGVEAGDEA | (VL) Anti- |
| | DYFCHLWDRSRDHYVFGTGTKLTVL | BCMA |
| 53 | EVQLVQSGAEVKKPGSSVKVSCKASGGTFSSYAISWVRQAP | Variable heavy |
| | GQGLEWMGRIIPILGIANYAQKFQGRVTMTEDTSTDTAYME | (VH) Anti- |
| | LSSLRSEDTAVYYCARSGYSKSIVSYMDYWGQGTLVTVSS | BCMA |
| 54 | LPVLTQPPSTSGTPGQRVTVSCSGSSSNIGSNVVFWYQQLPGT | Variable light |
| | APKLVIYRNNQRPSGVPDRFSVSKSGTSASLAISGLRSEDEAD | (VL) Anti- |
| | YYCAAWDDSLSGYVFGTGTKVTVLG | BCMA |
| 55 | ASGGGGSGGRASGGGGS | Linker |
| 56 | DYYVY | BCMA-55 CDR- |
| | | H1 (aa)-Kabat |
| | | numbering |
| 57 | WINPNSGGTNYAQKFQG | BCMA-55 CDR- |
| | | H2 (aa)-Kabat |
| | | numbering |
| 58 | SQRDGYMDY | BCMA-55 CDR- |
| | | H3 (aa)-Kabat, |
| | | Chothia, and |
| | | AbM numbering |
| 59 | TGTSSDVG | BCMA-55 CDR- |
| | | L1 (aa)-Kabat, |
| | | Chothia, and |
| | | AbM numbering |
| 60 | EDSKRPS | BCMA-55 CDR- |
| | | L2 (aa)-Kabat, |
| | | Chothia, and |
| | | AbM numbering |
| 61 | SSNTRSSTLV | BCMA-55 CDR- |
| | | L3 (aa)-Kabat, |
| | | Chothia, and |
| | | AbM numbering |
| 62 | DYSIN | CDR-H1 |
| 63 | WINTETREPAYAYDFRG | CDR-H2 |
| 64 | DYSYAMDY | CDR-H3 |
| 65 | RASESVTILGSHLIH | CDR-L1 |
| 66 | LASNVQT | CDR-L2 |
| 67 | LQSRTIPRT | CDR-L3 |
| 68 | DIVLTQSPPSLAMSLGKRATISCRASESVTILGSHLIHWYQQK | scFv |
| | PGQPPTLLIQLASNVQTGVPARFSGSGSRTDFTLTIDPVEEDD | |
| | VAVYYCLQSRTIPRTFGGGTKLEIKGSTSGSGKPGSGEGSTK | |
| | GQIQLVQSGPELKKPGETVKISCKASGYTFTDYSINWVKRAP | |
| | GKGLKWMGWINTETREPAYAYDFRGRFAFSLETSASTAYLQ | |
| | INNLKYEDTATYFCALDYSYAMDYWGQGTSVTVSS | |
| 69 | cagtctgccctgacacagcctgccagcgttagtgctagtcccggacagtctatcgccatcagctg | anti-BCMA CAR |
| | taccggcaccagctctgacgttggctggtatcagcagcaccctggcaaggcccctaagctgatg | |
| | atctacgaggacagcaagaggcccagcggcgtgtccaatagattcagcggcagcaagagcgg | |
| | caacaccgccagcctgacaattagcggactgcaggccgaggacgaggccgattactactgca | |
| | gcagcaacacccggtccagcacactggtttttggcggaggcaccaagctgacagtgctgggat | |
| | ctagaggtggcggaggatctggcggcggaggaagcggaggcggcggatctcttgaaatggct | |
| | gaagtgcagctggtgcagtctggcgccgagatgaagaaacctggcgcctctctgaagctgagc | |
| | tgcaaggccagcggctacaccttcatcgactactacgtgtactggatgcggcaggcccctggac | |
| | agggactcgaatctatgggctggatcaaccccaatagcggcggcaccaattacgcccagaaatt | |
| | ccagggcagagtgaccatgaccagagacaccagcatcagcaccgcctacatggaactgagcc | |
| | ggctgagatccgacgacaccgccatgtactactgcgccagatctcagcgcgacggctacatgg | |
| | attattggggccagggaaccctggtcaccgtgtccagcgagtctaaatacggaccgccttgtcct | |
| | ccttgtcccgctcctcctgttgccggaccttccgtgttcctgtttcctccaaagcctaaggacac | |
| | cctgatgatcagcaggacccctgaagtgacctgcgtggtggtggatgtgtcccaagaggatcccg | |
| | aggtgcagttcaactggtatgtggacggcgtggaagtgcacaacgccaagaccaagcctagaga | |
| | ggaacagttccagagcacctacagagtggtgtccgtgctgacagtgctgcaccaggattggctg | |
| | aacggcaaagagtacaagtgcaaggtgtccaacaagggcctgcctagcagcatcgagaaaac | |
| | catctccaaggccaagggccagccaagagagccccaggtttacacactgcctccaagccaaga | |
| | ggaaatgaccaagaatcaggtgtccctgacatgcctggtcaagggcttctacccctccgatatcg | |
| | ccgtggaatgggagagcaatggccagcctgagaacaactacaagaccacacctcctgtgctgg | |
| | acagcgacggcagtttcttcctgtatagtagactcaccgtggataaatcaagatggcaagagggc | |
| | aacgtgttcagctgcagcgtgatgcacgaggccctgcacaaccactacacccagaaaagcctg | |
| | agcctgtctctgggcaagatgttctgggtgctcgtggtcgttggcggagtgctggcctgttacagc | |
| | ctgctggttaccgtggccttcatcatcttttgggtcaagcggggcagaaagaagctgctctacatct | |
| | tcaagcagcccttcatgcggcccgtgcagaccacacaagaggaagatggctgctcctgcagatt | |
| | ccccgaggaagaagaaggcggctgcgagctgagagtgaagttcagcagatccgccgacgctc | |
| | cagcctatcagcagggccaaaaccagctgtacaacgagctgaacctggggagaagagaagag | |
| | tacgacgtgctggataagcggagaggcagagatcctgaaatgggcggcaagcccagacgga | |
| | agaatcctcaagagggcctgtataatgagctgcagaaagacaagatggccgaggcctacagcg | |
| | agatcggaatgaagggcgagcgcagaagaggcaagggacacgatggactgtaccagggcct | |
| | gagcaccgccaccaaggatacctatgacgcactgcacatgcaggccctgccacctaga | |
| 70 | GSTSGSGKPGSGEGSTKG | Linker |
| 71 | GGGS | Linker |
| 72 | GGGGSGGGGSGGGGS | Linker |
| 73 | GSTSGSGKPGSGEGSTKG | Linker |
| 74 | SRGGGGSGGGGSGGGGSLEMA | Linker |
| 75 | MALPVTALLLPLALLLHAARP | CD8a signal |
| | | peptide |
| 76 | METDTLLLWVLLLWVPGSTG | signal peptide |
| 77 | EVQLLESGGGLVQPGGSLRLSCAASGFTFSSYAMSWVRQAP | Variable heavy |
| | GKGLEWVSAISGSGGSTYYADSVKGRFTISRDNSKNTLYLQ | (VH) Anti- |
| | MNSLRAEDTAVYYCARAEMGAVFDIWGQGTMVTVSS | BCMA |
| 78 | EIVLTQSPATLSLSPGERATLSCRASQSVSRYLAWYQQKPGQ | Variable light |
| | APRLLIYDASNRATGIPARFSGSGSGTDFTLTISSLEPEDFAVY | (VL) Anti- |
| | YCQQRISWPFTFGGGTKVEIK | BCMA |
| 79 | QVQLVESGGGVVQPGRSLRLSCAASGFTFSSYGMHWVRQAP | Variable heavy |
| | GKGLEWVAVISYDGSNKYYADSVKGRFTISRDNSKNTLYLQ | (VH) Anti- |
| | MNSLRAEDTAVYYCARDGTYLGGLWYFDLWGRGTLVTVSS | BCMA |
| 80 | DIVMTQSPLSLPVTPGEPASISCRSSQSLLHSNGYNYLDWYLQ | Variable light |
| | KPGQSPQLLIYLGSNRASGVPDRFSGSGSGTDFTLKISRVEAE | (VL) Anti- |
| | DVGVYYCMQGLGLPLTFGGGTKVEIK | BCMA |
| 81 | QVQLVQSGAEVKKPGASVKVSCKASGYTFTSYYMHWVRQA | Variable heavy |
| | PGQGLEWMGIINPGGGSTSYAQKFQGRVTMTRDTSTSTVYM | (VH) Anti- |
| | ELSSLRSEDTAVYYCARESWPMDVWGQGTTVTVSS | BCMA |
| 82 | EIVMTQSPATLSVSPGERATLSCRASQSVSSNLAWYQQKPGQ | Variable light |
| | APRLLIYGASTRATGIPARFSGSGSGTEFTLTISSLQSEDFAVY | (VL) Anti- |
| | YCQQYAAYPTFGGGTKVEIK | BCMA |
| 83 | QLQLQESGPGLVKPSETLSLTCTVSGGSISSSSYYWGWIRQPP | Variable heavy |
| | GKGLEWIGSISYSGSTYYNPSLKSRVTISVDTSKNQFSLKLSS | (VH) Anti- |
| | VTAADTAVYYCARGRGYATSLAFDIWGQGTMVTVSS | BCMA |
| 84 | EIVLTQSPATLSLSPGERATLSCRASQSVSSYLAWYQQKPGQ | Variable light |
| | APRLLIYDASNRATGIPARFSGSGSGTDFTLTISSLEPEDFAVY | (VL) Anti- |
| | YCQQRHVWPPTFGGGTKVEIK | BCMA |
| 85 | EVQLVESGGGLVQPGGSLRLSCAASGFTFSSYSMNWVRQAP | Variable heavy |
| | GKGLEWVSTISSSSSTIYYADSVKGRFTISRDNAKNSLYLQM | (VH) Anti- |
| | NSLRAEDTAVYYCARGSQEHLIFDYWGQGTLVTVSS | BCMA |
| 86 | EIVLTQSPATLSLSPGERATLSCRASQSVSRYLAWYQQKPGQ | Variable light |
| | APRLLIYDASNRATGIPARFSGSGSGTDFTLTISSLEPEDFAVY | (VL) Anti- |
| | YCQQRFYYPWTFGGGTKVEIK | BCMA |
| 87 | QVQLVESGGGVVQPGRSLRLSCAASGFTFSSYGMHWVRQAP | Variable heavy |
| | GKGLEWVAVISYDGSNKYYADSVKGRFTISRDNSKNTLYLQ | (VH) Anti- |
| | MNSLRAEDTAVYYCARTDFWSGSPPGLDYWGQGTLVTVSS | BCMA |
| 88 | DIQLTQSPSSVSASVGDRVTITCRASQGISSWLAWYQQKPGK | Variable light |
| | APKLLIYGASSLQSGVPSRFSGSGSGTDFTLTISSLQPEDFATY | (VL) Anti- |
| | YCQQIYTFPFTFGGGTKVEIK | BCMA |
| 89 | QVQLVQSGAEVKKPGSSVKVSCKASGGTFSSYAISWVRQAP | Variable heavy |
| | GQGLEWMGGIIPIFGTANYAQKFQGRVTITADESTSTAYMEL | (VH) Anti- |
| | SSLRSEDTAVYYCARTPEYSSSIWHYYYGMDVWGQGTTVT | BCMA |
| | VSS | |
| 90 | DIVMTQSPDSLAVSLGERATINCKSSQSVLYSSNNKNYLAWY | Variable light |
| | QQKPGQPPKLLIYWASTRESGVPDRFSGSGSGTDFTLTISSLQ | (VL) Anti- |
| | AEDVAVYYCQQFAHTPFTFGGGTKVEIK | BCMA |
| 91 | QVQLVESGGGVVQPGRSLRLSCAASGFTFSSYGMHWVRQAP | Variable heavy |
| | GKGLEWVAVISYDGSNKYYADSVKGRFTISRDNSKNTLYLQ | (VH) Anti- |
| | MNSLRAEDTAVYYCVKGPLQEPPYDYGMDVWGQGTTVTV | BCMA |
| | SS | |
| 92 | EIVMTQSPATLSVSPGERATLSCRASQSVSSNLAWYQQKPGQ | Variable light |
| | APRLLIYSASTRATGIPARFSGSGSGTEFTLTISSLQSEDFAVY | (VL) Anti- |
| | YCQQHHVWPLTFGGGTKVEIK | BCMA |
| 93 | QVQLVQSGAEVKKPGSSVKVSCKASGGTFSSYAISWVRQAP | Variable heavy |
| | GQGLEWMGRIIPILGIANYAQKFQGRVTITADKSTSTAYMEL | (VH) Anti- |
| | SSLRSEDTAVYYCARGGYYSHDMWSEDWGQGTLVTVSS | BCMA |
| 94 | LPVLTQPPSASGTPGQRVTISCSGRSSNIGSNSVNWYRQLPGA | Variable light |
| | APKLLIYSNNQRPPGVPVRFSGSKSGTSASLAISGLQSEDEAT | (VL) Anti- |
| | YYCATWDDNLNVHYVFGTGTKVTVLG | BCMA |
| 95 | QVQLVQSGSELKKPGASVKVSCKASGYTFTDYSINWVRQAP | Variable heavy |
| | GQGLEWMGWINTETREPAYAYDFRGRFVFSLDTSVSTAYLQ | (VH) Anti- |
| | ISSLKAEDTAVYYCARDYSYAMDYWGQGTLVTVSS | BCMA |
| 96 | DIVLTQSPASLAVSLGERATINCRASESVSVIGAHLIHWYQQK | Variable light |
| | PGQPPKLLIYLASNLETGVPARFSGSGSGTDFTLTISSLQAED | (VL) Anti- |
| | AAIYYCLQSRIFPRTFGQGTKLEIK | BCMA |
| 97 | EVQLVESGGGLVQPGGSLRLSCAVSGFALSNHGMSWVRRAP | Variable heavy |
| | GKGLEWVSGIVYSGSTYYAASVKGRFTISRDNSRNTLYLQM | (VH) Anti- |
| | NSLRPEDTAIYYCSAHGGESDVWGQGTTVTVSS | BCMA |
| 98 | DIQLTQSPSSLSASVGDRVTITCRASQSISSYLNWYQQKPGKA | Variable light |
| | PKLLIYAASSLQSGVPSRFSGSGSGTDFTLTISSLQPEDFATYY | (VL) Anti- |
| | CQQSYSTPYTFGQGTKVEIK | BCMA |
| 99 | QVQLVESGGGLVQPGRSLRLSCAASGFTFSNYAMSWVRQAP | Variable heavy |
| | GKGLGWVSGISRSGENTYYADSVKGRFTISRDNSKNTLYLQ | (VH) Anti- |
| | MNSLRDEDTAVYYCARSPAHYYGGMDVWGQGTTVTVSS | BCMA |
| 100 | DIVLTQSPGTLSLSPGERATLSCRASQSISSSFLAWYQQKPGQ | Variable light |
| | APRLLIYGASRRATGIPDRFSGSGSGTDFTLTISRLEPEDSAVY | (VL) Anti- |
| | YCQQYHSSPSWTFGQGTKLEIK | BCMA |
| 101 | QVQLVESGGGLVQPGGSLRLSCAVSGFALSNHGMSWVRRA | Variable heavy |
| | PGKGLEWVSGIVYSGSTYYAASVKGRFTISRDNSRNTLYLQ | (VH) Anti- |
| | MNSLRPEDTAIYYCSAHGGESDVWGQGTTVTVSS | BCMA |
| 102 | DIRLTQSPSPLSASVGDRVTITCQASEDINKFLNWYHQTPGKA | Variable light |
| | PKLLIYDASTLQTGVPSRFSGSGSGTDFTLTINSLQPEDIGTYY | (VL) Anti- |
| | CQQYESLPLTFGGGTKVEIK | BCMA |
| 103 | EVQLVESGGGLVQPGGSLRLSCAVSGFALSNHGMSWVRRAP | Variable heavy |
| | GKGLEWVSGIVYSGSTYYAASVKGRFTISRDNSRNTLYLQM | (VH) Anti- |
| | NSLRPEDTAIYYCSAHGGESDVWGQGTTVTVSS | BCMA |
| 104 | EIVLTQSPGTLSLSPGERATLSCRASQSIGSSSLAWYQQKPGQ | Variable light |
| | APRLLMYGASSRASGIPDRFSGSGSGTDFTLTISRLEPEDFAV | (VL) Anti- |
| | YYCQQYAGSPPFTFGQGTKVEIK | BCMA |
| 105 | QIQLVQSGPELKKPGETVKISCKASGYTFRHYSMNWVKQAP | Variable heavy |
| | GKGLKWMGRINTESGVPIYADDFKGRFAFSVETSASTAYLVI | (VH) Anti- |
| | NNLKDEDTASYFCSNDYLYSLDFWGQGTALTVSS | BCMA |
| 106 | DIVLTQSPPSLAMSLGKRATISCRASESVTILGSHLIYWYQQK | Variable light |
| | PGQPPTLLIQLASNVQTGVPARFSGSGSRTDFTLTIDPVEEDD | (VL) Anti- |
| | VAVYYCLQSRTIPRTFGGGTKLEIK | BCMA |
| 107 | QIQLVQSGPELKKPGETVKISCKASGYTFTHYSMNWVKQAP | Variable heavy |
| | GKGLKWMGRINTETGEPLYADDFKGRFAFSLETSASTAYLVI | (VH) Anti- |
| | NNLKNEDTATFFCSNDYLYSCDYWGQGTTLTVSS | BCMA |
| 108 | DIVLTQSPASLAMSLGKRATISCRASESVSVIGAHLIHWYQQ | Variable light |
| | KPGQPPKLLIYLASNLETGVPARFSGSGSGTDFTLTIDPVEED | (VL) Anti- |
| | DVAIYSCLQSRIFPRTFGGGTKLEIK | BCMA |
| 109 | QVQLVQSGAEVKKPGASVKVSCKASGYSFPDYYINWVRQA | Variable heavy |
| | PGQGLEWMGWIYFASGNSEYNQKFTGRVTMTRDTSINTAY | (VH) Anti- |
| | MELSSLTSEDTAVYFCASLYDYDWYFDVWGQGTMVTVSS | BCMA |
| 110 | DIVMTQTPLSLSVTPGQPASISCKSSQSLVHSNGNTYLHWYL | Variable light |
| | QKPGQSPQLLIYKVSNRFSGVPDRFSGSGSGTDFTLKISRVEA | (VL) Anti- |
| | EDVGIYYCSQSSIYPWTFGQGTKLEIK | BCMA |
| 111 | QVQLVQSGAEVKKPGASVKVSCKASGYSFPDYYINWVRQA | Variable heavy |
| | PGQGLEWMGWIYFASGNSEYNQKFTGRVTMTRDTSSSTAY | (VH) Anti- |
| | MELSSLRSEDTAVYFCASLYDYDWYFDVWGQGTMVTVSS | BCMA |
| 112 | DIVMTQTPLSLSVTPGEPASISCKSSQSLVHSNGNTYLHWYL | Variable light |
| | QKPGQSPQLLIYKVSNRFSGVPDRFSGSGSGADFTLKISRVEA | (VL) Anti- |
| | EDVGVYYCAETSHVPWTFGQGTKLEIK | BCMA |
| 113 | QVQLVESGGGLVQPGGSLRLSCEASGFTLDYYAIGWFRQAP | Anti-BCMA |
| | GKEREGVICISRSDGSTYYADSVKGRFTISRDNAKKTVYLQM | sdAb |
| | ISLKPEDTAAYYCAAGADCSGYLRDYEFRGQGTQVTVSS | |
| 114 | IEVMYPPPYLDNEKSNGTIIHVKGKHLCPSPLFPGPSKP | CD28 spacer |
| 115 | IYIWAPLAGTCGVLLLSLVITLYCN | CD8a TM |
| 116 | LDNEKSNGTIIHVKGKHLCPSPLFPGPSKP | CD28 spacer |
| | | (truncated) |
| 117 | PTTTPAPRPPTPAPTIASQPLSLRPEACRPAAGGAVHTRGLDF | CD8a hinge |
| | ACD | |
| 118 | TTTPAPRPPTPAPTIASQPLSLRPEACRPAAGGAVHTRGLDFA | CD8a hinge |
| | CD | |
| 119 | FVPVFLPAKPTTTPAPRPPTPAPTIASQPLSLRPEACRPAAGGA | CD8a hinge |
| | VHTRGLDFACD | |
| 120 | DTGLYICKVELMYPPPYYLGIGNGTQIYVIDPEPCPDSD | CTLA4 hinge |
| 121 | FLLWILAAVSSGLFFYSFLLTAVS | CTLA4 TM |
| 122 | QIKESLRAELRVTERRAEVPTAHPSPSPRPAGQFQTLV | PD-1 hinge |
| 123 | VGVVGGLLGSLVLLVWVLAVI | PD-1 TM |
| 124 | GLAVSTISSFFPPGYQ | FcγRIIIa hinge |
| 125 | EPKSPDKTHTCPPCPAPPVAGPSVFLFPPKPKDTLMIARTPEV | IgGI hinge |
| | TCVWDVSHEDPEVKFNWYVDGVEVHNAKTKPREEQYNST | |
| | YRVVSVLTVLHQDWLNGKEYKCKVSNKALPAPIEKTISKAK | |
| | GQPREPQVYTLPPSRDELTKNQVSLTCLVKGFYPSDIAVEWE | |
| | SNGQPENNYKTTPPVLDSDGSFFLYSKLTVDKSRWQQGNVF | |
| | SCSVMHEALHNHYTQKSLSLSPGK | |
| 126 | EVQLLESGGGLVQPGGSLRLSCAASGFTFSSYAMSWVRQAP | anti-BCMA CAR |
| | GKGLEWVSAISGSGGSTYYADSVKGRFTISRDNSKNTLYLQ | |
| | MNSLRAEDTAVYYCARAEMGAVFDIWGQGTMVTVSSGSTS | |
| | GSGKPGSGEGSTKGEIVLTQSPATLSLSPGERATLSCRASQSV | |
| | SRYLAWYQQKPGQAPRLLIYDASNRATGIPARFSGSGSGTDF | |
| | TLTISSLEPEDFAVYYCQQRISWPFTFGGGTKVEIKRAAALDN | |
| | EKSNGTIIHVKGKHLCPSPLFPGPSKPFWVLVVVGGVLACYS | |
| | LLVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 127 | EIVLTQSPATLSLSPGERATLSCRASQSVSRYLAWYQQKPGQ | anti-BCMA CAR |
| | APRLLIYDASNRATGIPARFSGSGSGTDFTLTISSLEPEDFAVY | |
| | YCQQRISWPFTFGGGTKVEIKRGSTSGSGKPGSGEGSTKGEV | |
| | QLLESGGGLVQPGGSLRLSCAASGFTFSSYAMSWVRQAPGK | |
| | GLEWVSAISGSGGSTYYADSVKGRFTISRDNSKNTLYLQMNS | |
| | LRAEDTAVYYCARAEMGAVFDIWGQGTMVTVSSAAALDNE | |
| | KSNGTIIHVKGKHLCPSPLFPGPSKPFWVLVVVGGVLACYSL | |
| | LVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 128 | QVQLVESGGGVVQPGRSLRLSCAASGFTFSSYGMHWVRQAP | anti-BCMA CAR |
| | GKGLEWVAVISYDGSNKYYADSVKGRFTISRDNSKNTLYLQ | |
| | MNSLRAEDTAVYYCARDGTYLGGLWYFDLWGRGTLVTVSS | |
| | GSTSGSGKPGSGEGSTKGDIVMTQSPLSLPVTPGEPASISCRSS | |
| | QSLLHSNGYNYLDWYLQKPGQSPQLLIYLGSNRASGVPDRF | |
| | SGSGSGTDFTLKISRVEAEDVGVYYCMQGLGLPLTFGGGTK | |
| | VEIKRAAALDNEKSNGTIIHVKGKHLCPSPLFPGPSKPFWVLV | |
| | VVGGVLACYSLLVTVAFIIFWVRSKRSRLLHSDYMNMTPRR | |
| | PGPTRKHYQPYAPPRDFAAYRSRVKFSRSADAPAYQQGQNQ | |
| | LYNELNLGRREEYDVLDKRRGRDPEMGGKPRRKNPQEGLY | |
| | NELQKDKMAEAYSEIGMKGERRRGKGHDGLYQGLSTATKD | |
| | TYDALHMQALPPR | |
| 129 | DIVMTQSPLSLPVTPGEPASISCRSSQSLLHSNGYNYLDWYLQ | anti-BCMA CAR |
| | KPGQSPQLLIYLGSNRASGVPDRFSGSGSGTDFTLKISRVEAE | |
| | DVGVYYCMQGLGLPLTFGGGTKVEIKRGSTSGSGKPGSGEG | |
| | STKGQVQLVESGGGVVQPGRSLRLSCAASGFTFSSYGMHWV | |
| | RQAPGKGLEWVAVISYDGSNKYYADSVKGRFTISRDNSKNT | |
| | LYLQMNSLRAEDTAVYYCARDGTYLGGLWYFDLWGRGTL | |
| | VTVSSAAALDNEKSNGTIIHVKGKHLCPSPLFPGPSKPFWVL | |
| | VVVGGVLACYSLLVTVAFIIFWVRSKRSRLLHSDYMNMTPR | |
| | RPGPTRKHYQPYAPPRDFAAYRSRVKFSRSADAPAYQQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 130 | QVQLVQSGAEVKKPGASVKVSCKASGYTFTSYYMHWVRQA | anti-BCMA CAR |
| | PGQGLEWMGIINPGGGSTSYAQKFQGRVTMTRDTSTSTVYM | |
| | ELSSLRSEDTAVYYCARESWPMDVWGQGTTVTVSSGSTSGS | |
| | GKPGSGEGSTKGEIVMTQSPATLSVSPGERATLSCRASQSVSS | |
| | NLAWYQQKPGQAPRLLIYGASTRATGIPARFSGSGSGTEFTL | |
| | TISSLQSEDFAVYYCQQYAAYPTFGGGTKVEIKRAAALDNEK | |
| | SNGTIIHVKGKHLCPSPLFPGPSKPFWVLVVVGGVLACYSLL | |
| | VTVAFIIFWVRSKRSRLLHSDYMNMTPRRPGPTRKHYQPYAP | |
| | PRDFAAYRSRVKFSRSADAPAYQQGQNQLYNELNLGRREEY | |
| | DVLDKRRGRDPEMGGKPRRKNPQEGLYNELQKDKMAEAYS | |
| | EIGMKGERRRGKGHDGLYQGLSTATKDTYDALHMQALPPR | |
| 131 | EIVMTQSPATLSVSPGERATLSCRASQSVSSNLAWYQQKPGQ | anti-BCMA CAR |
| | APRLLIYGASTRATGIPARFSGSGSGTEFTLTISSLQSEDFAVY | |
| | YCQQYAAYPTFGGGTKVEIKRGSTSGSGKPGSGEGSTKGQV | |
| | QLVQSGAEVKKPGASVKVSCKASGYTFTSYYMHWVRQAPG | |
| | QGLEWMGIINPGGGSTSYAQKFQGRVTMTRDTSTSTVYMEL | |
| | SSLRSEDTAVYYCARESWPMDVWGQGTTVTVSSAAALDNE | |
| | KSNGTIIHVKGKHLCPSPLFPGPSKPFWVLVVVGGVLACYSL | |
| | LVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 132 | QLQLQESGPGLVKPSETLSLTCTVSGGSISSSSYYWGWIRQPP | anti-BCMA CAR |
| | GKGLEWIGSISYSGSTYYNPSLKSRVTISVDTSKNQFSLKLSS | |
| | VTAADTAVYYCARGRGYATSLAFDIWGQGTMVTVSSGSTS | |
| | GSGKPGSGEGSTKGEIVLTQSPATLSLSPGERATLSCRASQSV | |
| | SSYLAWYQQKPGQAPRLLIYDASNRATGIPARFSGSGSGTDF | |
| | TLTISSLEPEDFAVYYCQQRHVWPPTFGGGTKVEIKRAAALD | |
| | NEKSNGTIIHVKGKHLCPSPLFPGPSKPFWVLVVVGGVLACY | |
| | SLLVTVAFIIFWVRSKRSRLLHSDYMNMTPRRPGPTRKHYQP | |
| | YAPPRDFAAYRSRVKFSRSADAPAYQQGQNQLYNELNLGRR | |
| | EEYDVLDKRRGRDPEMGGKPRRKNPQEGLYNELQKDKMAE | |
| | AYSEIGMKGERRRGKGHDGLYQGLSTATKDTYDALHMQAL | |
| | PPR | |
| 133 | EIVLTQSPATLSLSPGERATLSCRASQSVSSYLAWYQQKPGQ | anti-BCMA CAR |
| | APRLLIYDASNRATGIPARFSGSGSGTDFTLTISSLEPEDFAVY | |
| | YCQQRHVWPPTFGGGTKVEIKRGSTSGSGKPGSGEGSTKGQ | |
| | LQLQESGPGLVKPSETLSLTCTVSGGSISSSSYYWGWIRQPPG | |
| | KGLEWIGSISYSGSTYYNPSLKSRVTISVDTSKNQFSLKLSSV | |
| | TAADTAVYYCARGRGYATSLAFDIWGQGTMVTVSSAAALD | |
| | NEKSNGTIIHVKGKHLCPSPLFPGPSKPFWVLVVVGGVLACY | |
| | SLLVTVAFIIFWVRSKRSRLLHSDYMNMTPRRPGPTRKHYQP | |
| | YAPPRDFAAYRSRVKFSRSADAPAYQQGQNQLYNELNLGRR | |
| | EEYDVLDKRRGRDPEMGGKPRRKNPQEGLYNELQKDKMAE | |
| | AYSEIGMKGERRRGKGHDGLYQGLSTATKDTYDALHMQAL | |
| | PPR | |
| 134 | EVQLVESGGGLVQPGGSLRLSCAASGFTFSSYSMNWVRQAP | anti-BCMA CAR |
| | GKGLEWVSTISSSSSTIYYADSVKGRFTISRDNAKNSLYLQM | |
| | NSLRAEDTAVYYCARGSQEHLIFDYWGQGTLVTVSSGSTSG | |
| | SGKPGSGEGSTKGEIVLTQSPATLSLSPGERATLSCRASQSVS | |
| | RYLAWYQQKPGQAPRLLIYDASNRATGIPARFSGSGSGTDFT | |
| | LTISSLEPEDFAVYYCQQRFYYPWTFGGGTKVEIKRAAALDN | |
| | EKSNGTIIHVKGKHLCPSPLFPGPSKPFWVLVVVGGVLACYS | |
| | LLVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 135 | EIVLTQSPATLSLSPGERATLSCRASQSVSRYLAWYQQKPGQ | anti-BCMA CAR |
| | APRLLIYDASNRATGIPARFSGSGSGTDFTLTISSLEPEDFAVY | |
| | YCQQRFYYPWTFGGGTKVEIKRGSTSGSGKPGSGEGSTKGE | |
| | VQLVESGGGLVQPGGSLRLSCAASGFTFSSYSMNWVRQAPG | |
| | KGLEWVSTISSSSSTIYYADSVKGRFTISRDNAKNSLYLQMNS | |
| | LRAEDTAVYYCARGSQEHLIFDYWGQGTLVTVSSAAALDNE | |
| | KSNGTIIHVKGKHLCPSPLFPGPSKPFWVLVVVGGVLACYSL | |
| | LVTVAFIIFWVRSKRSRLLHSDYMNMTPRRPGPTRKHYQPY | |
| | APPRDFAAYRSRVKFSRSADAPAYQQGQNQLYNELNLGRRE | |
| | EYDVLDKRRGRDPEMGGKPRRKNPQEGLYNELQKDKMAEA | |
| | YSEIGMKGERRRGKGHDGLYQGLSTATKDTYDALHMQALP | |
| | PR | |
| 136 | QVQLVESGGGVVQPGRSLRLSCAASGFTFSSYGMHWVRQAP | anti-BCMA CAR |
| | GKGLEWVAVISYDGSNKYYADSVKGRFTISRDNSKNTLYLQ | |
| | MNSLRAEDTAVYYCARTDFWSGSPPGLDYWGQGTLVTVSS | |
| | GSTSGSGKPGSGEGSTKGDIQLTQSPSSVSASVGDRVTITCRA | |
| | SQGISSWLAWYQQKPGKAPKLLIYGASSLQSGVPSRFSGSGS | |
| | GTDFTLTISSLQPEDFATYYCQQIYTFPFTFGGGTKVEIKRAA | |
| | ALDNEKSNGTIIHVKGKHLCPSPLFPGPSKPFWVLVVVGGVL | |
| | ACYSLLVTVAFIIFWVRSKRSRLLHSDYMNMTPRRPGPTRKH | |
| | YQPYAPPRDFAAYRSRVKFSRSADAPAYQQGQNQLYNELNL | |
| | GRREEYDVLDKRRGRDPEMGGKPRRKNPQEGLYNELQKDK | |
| | MAEAYSEIGMKGERRRGKGHDGLYQGLSTATKDTYDALHM | |
| | QALPPR | |
| 137 | DIQLTQSPSSVSASVGDRVTITCRASQGISSWLAWYQQKPGK | anti-BCMA CAR |
| | APKLLIYGASSLQSGVPSRFSGSGSGTDFTLTISSLQPEDFATY | |
| | YCQQIYTFPFTFGGGTKVEIKRGSTSGSGKPGSGEGSTKGQV | |
| | QLVESGGGVVQPGRSLRLSCAASGFTFSSYGMHWVRQAPGK | |
| | GLEWVAVISYDGSNKYYADSVKGRFTISRDNSKNTLYLQMN | |
| | SLRAEDTAVYYCARTDFWSGSPPGLDYWGQGTLVTVSSAA | |
| | ALDNEKSNGTIIHVKGKHLCPSPLFPGPSKPFWVLVVVGGVL | |
| | ACYSLLVTVAFIIFWVRSKRSRLLHSDYMNMTPRRPGPTRKH | |
| | YQPYAPPRDFAAYRSRVKFSRSADAPAYQQGQNQLYNELNL | |
| | GRREEYDVLDKRRGRDPEMGGKPRRKNPQEGLYNELQKDK | |
| | MAEAYSEIGMKGERRRGKGHDGLYQGLSTATKDTYDALHM | |
| | QALPPR | |
| 138 | QVQLVQSGAEVKKPGSSVKVSCKASGGTFSSYAISWVRQAP | anti-BCMA CAR |
| | GQGLEWMGGIIPIFGTANYAQKFQGRVTITADESTSTAYMEL | |
| | SSLRSEDTAVYYCARTPEYSSSIWHYYYGMDVWGQGTTVT | |
| | VSSGSTSGSGKPGSGEGSTKGDIVMTQSPDSLAVSLGERATIN | |
| | CKSSQSVLYSSNNKNYLAWYQQKPGQPPKLLIYWASTRESG | |
| | VPDRFSGSGSGTDFTLTISSLQAEDVAVYYCQQFAHTPFTFG | |
| | GGTKVEIKRAAALDNEKSNGTIIHVKGKHLCPSPLFPGPSKPF | |
| | WVLVVVGGVLACYSLLVTVAFIIFWVRSKRSRLLHSDYMN | |
| | MTPRRPGPTRKHYQPYAPPRDFAAYRSRVKFSRSADAPAYQ | |
| | QGQNQLYNELNLGRREEYDVLDKRRGRDPEMGGKPRRKNP | |
| | QEGLYNELQKDKMAEAYSEIGMKGERRRGKGHDGLYQGLS | |
| | TATKDTYDALHMQALPPR | |
| 139 | DIVMTQSPDSLAVSLGERATINCKSSQSVLYSSNNKNYLAWY | anti-BCMA CAR |
| | QQKPGQPPKLLIYWASTRESGVPDRFSGSGSGTDFTLTISSLQ | |
| | AEDVAVYYCQQFAHTPFTFGGGTKVEIKRGSTSGSGKPGSGE | |
| | GSTKGQVQLVQSGAEVKKPGSSVKVSCKASGGTFSSYAISW | |
| | VRQAPGQGLEWMGGIIPIFGTANYAQKFQGRVTITADESTST | |
| | AYMELSSLRSEDTAVYYCARTPEYSSSIWHYYYGMDVWGQ | |
| | GTTVTVSSAAALDNEKSNGTIIHVKGKHLCPSPLFPGPSKPFW | |
| | VLVVVGGVLACYSLLVTVAFIIFWVRSKRSRLLHSDYMNMT | |
| | PRRPGPTRKHYQPYAPPRDFAAYRSRVKFSRSADAPAYQQG | |
| | QNQLYNELNLGRREEYDVLDKRRGRDPEMGGKPRRKNPQE | |
| | GLYNELQKDKMAEAYSEIGMKGERRRGKGHDGLYQGLSTA | |
| | TKDTYDALHMQALPPR | |
| 140 | QVQLVESGGGVVQPGRSLRLSCAASGFTFSSYGMHWVRQAP | anti-BCMA CAR |
| | GKGLEWVAVISYDGSNKYYADSVKGRFTISRDNSKNTLYLQ | |
| | MNSLRAEDTAVYYCVKGPLQEPPYDYGMDVWGQGTTVTV | |
| | SSGSTSGSGKPGSGEGSTKGEIVMTQSPATLSVSPGERATLSC | |
| | RASQSVSSNLAWYQQKPGQAPRLLIYSASTRATGIPARFSGS | |
| | GSGTEFTLTISSLQSEDFAVYYCQQHHVWPLTFGGGTKVEIK | |
| | RAAALDNEKSNGTIIHVKGKHLCPSPLFPGPSKPFWVLVVVG | |
| | GVLACYSLLVTVAFIIFWVRSKRSRLLHSDYMNMTPRRPGPT | |
| | RKHYQPYAPPRDFAAYRSRVKFSRSADAPAYQQGQNQLYN | |
| | ELNLGRREEYDVLDKRRGRDPEMGGKPRRKNPQEGLYNEL | |
| | QKDKMAEAYSEIGMKGERRRGKGHDGLYQGLSTATKDTYD | |
| | ALHMQALPPR | |
| 141 | EIVMTQSPATLSVSPGERATLSCRASQSVSSNLAWYQQKPGQ | anti-BCMA CAR |
| | APRLLIYSASTRATGIPARFSGSGSGTEFTLTISSLQSEDFAVY | |
| | YCQQHHVWPLTFGGGTKVEIKRGSTSGSGKPGSGEGSTKGQ | |
| | VQLVESGGGVVQPGRSLRLSCAASGFTFSSYGMHWVRQAPG | |
| | KGLEWVAVISYDGSNKYYADSVKGRFTISRDNSKNTLYLQM | |
| | NSLRAEDTAVYYCVKGPLQEPPYDYGMDVWGQGTTVTVSS | |
| | AAALDNEKSNGTIIHVKGKHLCPSPLFPGPSKPFWVLVVVGG | |
| | VLACYSLLVTVAFIIFWVRSKRSRLLHSDYMNMTPRRPGPTR | |
| | KHYQPYAPPRDFAAYRSRVKFSRSADAPAYQQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 142 | QSALTQPASVSASPGQSIAISCTGTSSDVGWYQQHPGKAPKL | anti-BCMA CAR |
| | MIYEDSKRPSGVSNRFSGSKSGNTASLTISGLQAEDEADYYC | |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSSA | |
| | AAIEVMYPPPYLDNEKSNGTIIHVKGKHLCPSPLFPGPSKPFW | |
| | VLVVVGGVLACYSLLVTVAFIIFWVRSKRSRLLHSDYMNMT | |
| | PRRPGPTRKHYQPYAPPRDFAAYRSRVKFSRSADAPAYQQG | |
| | QNQLYNELNLGRREEYDVLDKRRGRDPEMGGKPRRKNPQE | |
| | GLYNELQKDKMAEAYSEIGMKGERRRGKGHDGLYQGLSTA | |
| | TKDTYDALHMQALPPR | |
| 143 | QSVLTQPPSVSGAPGQRVTISCTGSSSNIGAGFDVHWYQQLP | anti-BCMA CAR |
| | GTAPKLLIYGNSNRPSGVPDRFSGSKSGTSASLAITGLQAEDE | |
| | ADYYCQSYDSSLSGYVFGTGTKVTVLGSRGGGGSGGGGSG | |
| | GGGSLEMAQVQLVQSGAEVKKPGASVKVSCKASGYTFTDY | |
| | YMHWVRQAPGQRLEWMGWINPNSGGTNYAQKFQDRITVT | |
| | RDTSSNTGYMELTRLRSDDTAVYYCARSPYSGVLDKWGQG | |
| | TLVTVSSAAAIEVMYPPPYLDNEKSNGTIIHVKGKHLCPSPLF | |
| | PGPSKPFWVLVVVGGVLACYSLLVTVAFIIFWVRSKRSRLLH | |
| | SDYMNMTPRRPGPTRKHYQPYAPPRDFAAYRSRVKFSRSAD | |
| | APAYQQGQNQLYNELNLGRREEYDVLDKRRGRDPEMGGKP | |
| | RRKNPQEGLYNELQKDKMAEAYSEIGMKGERRRGKGHDGL | |
| | YQGLSTATKDTYDALHMQALPPR | |
| 144 | SYELTQPPSASGTPGQRVTMSCSGTSSNIGSHSVNWYQQLPG | anti-BCMA CAR |
| | TAPKLLIYTNNQRPSGVPDRFSGSKSGTSASLAISGLQSEDEA | |
| | DYYCAAWDGSLNGLVFGGGTKLTVLGSRGGGGSGGGGSGG | |
| | GGSLEMAEVQLVQSGAEVKKPGESLKISCKGSGYSFTSYWIG | |
| | WVRQMPGKGLEWMGIIYPGDSDTRYSPSFQGHVTISADKSIS | |
| | TAYLQWSSLKASDTAMYYCARYSGSFDNWGQGTLVTVSSA | |
| | AAIEVMYPPPYLDNEKSNGTIIHVKGKHLCPSPLFPGPSKPFW | |
| | VLVVVGGVLACYSLLVTVAFIIFWVRSKRSRLLHSDYMNMT | |
| | PRRPGPTRKHYQPYAPPRDFAAYRSRVKFSRSADAPAYQQG | |
| | QNQLYNELNLGRREEYDVLDKRRGRDPEMGGKPRRKNPQE | |
| | GLYNELQKDKMAEAYSEIGMKGERRRGKGHDGLYQGLSTA | |
| | TKDTYDALHMQALPPR | |
| 145 | LPVLTQPPSASGTPGQRVTISCSGRSSNIGSNSVNWYRQLPGA | anti-BCMA CAR |
| | APKLLIYSNNQRPPGVPVRFSGSKSGTSASLAISGLQSEDEAT | |
| | YYCATWDDNLNVHYVFGTGTKVTVLGSRGGGGSGGGGSG | |
| | GGGSLEMAQVQLVQSGAEVKKPGSSVKVSCKASGGTFSSYA | |
| | ISWVRQAPGQGLEWMGRIIPILGIANYAQKFQGRVTITADKS | |
| | TSTAYMELSSLRSEDTAVYYCARGGYYSHDMWSEDWGQGT | |
| | LVTVSSAAAIEVMYPPPYLDNEKSNGTIIHVKGKHLCPSPLFP | |
| | GPSKPFWVLVVVGGVLACYSLLVTVAFIIFWVRSKRSRLLHS | |
| | DYMNMTPRRPGPTRKHYQPYAPPRDFAAYRSRVKFSRSADA | |
| | PAYQQGQNQLYNELNLGRREEYDVLDKRRGRDPEMGGKPR | |
| | RKNPQEGLYNELQKDKMAEAYSEIGMKGERRRGKGHDGLY | |
| | QGLSTATKDTYDALHMQALPPR | |
| 146 | QAVLTQPPSASGTPGQRVTISCSGSSSNIGSNYVFWYQQLPGT | anti-BCMA CAR |
| | APKLLIYSNNQRPSGVPDRFSGSKSGTSASLAISGLRSEDEAD | |
| | YYCAAWDDSLSASYVFGTGTKVTVLGSRGGGGSGGGGSGG | |
| | GGSLEMAQVQLVQSGAEVKKPGSSVKVSCKASGGTFSSYAI | |
| | SWVRQAPGQGLEWMGRIIPILGTANYAQKFQGRVTITADEST | |
| | STAYMELSSLRSEDTAVYYCARSGYGSYRWEDSWGQGTLV | |
| | TVSSAAAIEVMYPPPYLDNEKSNGTIIHVKGKHLCPSPLFPGP | |
| | SKPFWVLVVVGGVLACYSLLVTVAFIIFWVRSKRSRLLHSDY | |
| | MNMTPRRPGPTRKHYQPYAPPRDFAAYRSRVKFSRSADAPA | |
| | YQQGQNQLYNELNLGRREEYDVLDKRRGRDPEMGGKPRRK | |
| | NPQEGLYNELQKDKMAEAYSEIGMKGERRRGKGHDGLYQG | |
| | LSTATKDTYDALHMQALPPR | |
| 147 | LPVLTQPPSASGTPGQRVTISCSGRSSNIGSNSVNWYRQLPGA | anti-BCMA CAR |
| | APKLLIYSNNQRPPGVPVRFSGSKSGTSASLAISGLQSEDEAT | |
| | YYCATWDDNLNVHYVFGTGTKVTVLGSRGGGGSGGGGSG | |
| | GGGSLEMAQVQLVQSGAEVKKPGSSVKVSCKASGGTFSSYA | |
| | ISWVRQAPGQGLEWMGRIIPILGIANYAQKFQGRVTITADKS | |
| | TSTAYMELSSLRSEDTAVYYCARGGYYSHDMWSEDWGQGT | |
| | LVTVSSAAAPTTTPAPRPPTPAPTIASQPLSLRPEACRPAAGG | |
| | AVHTRGLDFACDIYIWAPLAGTCGVLLLSLVITLYCNKRGRK | |
| | KLLYIFKQPFMRPVQTTQEEDGCSCRFPEEEEGGCELRVKFS | |
| | RSAEPPAYQQGQNQLYNELNLGRREEYDVLDKRRGRDPEM | |
| | GGKPRRKNPQEGLYNELQKDKMAEAYSEIGMKGERRRGKG | |
| | HDGLYQGLSTATKDTYDALHMQALPPR | |
| 148 | SYELTQPPSASGTPGQRVTMSCSGTSSNIGSHSVNWYQQLPG | anti-BCMA CAR |
| | TAPKLLIYTNNQRPSGVPDRFSGSKSGTSASLAISGLQSEDEA | |
| | DYYCAAWDGSLNGLVFGGGTKLTVLGSRGGGGSGGGGSGG | |
| | GGSLEMAEVQLVQSGAEVKKPGESLKISCKGSGYSFTSYWIG | |
| | WVRQMPGKGLEWMGIIYPGDSDTRYSPSFQGHVTISADKSIS | |
| | TAYLQWSSLKASDTAMYYCARYSGSFDNWGQGTLVTVSSA | |
| | AAPTTTPAPRPPTPAPTIASQPLSLRPEACRPAAGGAVHTRGL | |
| | DFACDIYIWAPLAGTCGVLLLSLVITLYCNKRGRKKLLYIFK | |
| | QPFMRPVQTTQEEDGCSCRFPEEEEGGCELRVKFSRSAEPPA | |
| | YQQGQNQLYNELNLGRREEYDVLDKRRGRDPEMGGKPRRK | |
| | NPQEGLYNELQKDKMAEAYSEIGMKGERRRGKGHDGLYQG | |
| | LSTATKDTYDALHMQALPPR | |
| 149 | QAVLTQPPSASGTPGQRVTISCSGSSSNIGSNYVFWYQQLPGT | anti-BCMA CAR |
| | APKLLIYSNNQRPSGVPDRFSGSKSGTSASLAISGLRSEDEAD | |
| | YYCAAWDDSLSASYVFGTGTKVTVLGSRGGGGSGGGGSGG | |
| | GGSLEMAQVQLVQSGAEVKKPGSSVKVSCKASGGTFSSYAI | |
| | SWVRQAPGQGLEWMGRIIPILGTANYAQKFQGRVTITADEST | |
| | STAYMELSSLRSEDTAVYYCARSGYGSYRWEDSWGQGTLV | |
| | TVSSAAAPTTTPAPRPPTPAPTIASQPLSLRPEACRPAAGGAV | |
| | HTRGLDFACDIYIWAPLAGTCGVLLLSLVITLYCNKRGRKKL | |
| | LYIFKQPFMRPVQTTQEEDGCSCRFPEEEEGGCELRVKFSRSA | |
| | EPPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 150 | QSVLTQPPSVSGAPGQRVTISCTGSSSNIGAGFDVHWYQQLP | anti-BCMA CAR |
| | GTAPKLLIYGNSNRPSGVPDRFSGSKSGTSASLAITGLQAEDE | |
| | ADYYCQSYDSSLSGYVFGTGTKVTVLGSRGGGGSGGGGSG | |
| | GGGSLEMAQVQLVQSGAEVKKPGASVKVSCKASGYTFTDY | |
| | YMHWVRQAPGQRLEWMGWINPNSGGTNYAQKFQDRITVT | |
| | RDTSSNTGYMELTRLRSDDTAVYYCARSPYSGVLDKWGQG | |
| | TLVTVSSAAAPTTTPAPRPPTPAPTIASQPLSLRPEACRPAAG | |
| | GAVHTRGLDFACDIYIWAPLAGTCGVLLLSLVITLYCNKRGR | |
| | KKLLYIFKQPFMRPVQTTQEEDGCSCRFPEEEEGGCELRVKF | |
| | SRSAEPPAYQQGQNQLYNELNLGRREEYDVLDKRRGRDPE | |
| | MGGKPRRKNPQEGLYNELQKDKMAEAYSEIGMKGERRRGK | |
| | GHDGLYQGLSTATKDTYDALHMQALPPR | |
| 151 | QSALTQPASVSASPGQSIAISCTGTSSDVGWYQQHPGKAPKL | anti-BCMA CAR |
| | MIYEDSKRPSGVSNRFSGSKSGNTASLTISGLQAEDEADYYC | |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSSA | |
| | AAPTTTPAPRPPTPAPTIASQPLSLRPEACRPAAGGAVHTRGL | |
| | DFACDIYIWAPLAGTCGVLLLSLVITLYCNKRGRKKLLYIFK | |
| | QPFMRPVQTTQEEDGCSCRFPEEEEGGCELRVKFSRSAEPPA | |
| | YQQGQNQLYNELNLGRREEYDVLDKRRGRDPEMGGKPRRK | |
| | NPQEGLYNELQKDKMAEAYSEIGMKGERRRGKGHDGLYQG | |
| | LSTATKDTYDALHMQALPPR | |
| 152 | DIVLTQSPPSLAMSLGKRATISCRASESVTILGSHLIHWYQQK | anti-BCMA CAR |
| | PGQPPTLLIQLASNVQTGVPARFSGSGSRTDFTLTIDPVEEDD | |
| | VAVYYCLQSRTIPRTFGGGTKLEIKGSTSGSGKPGSGEGSTK | |
| | GQIQLVQSGPELKKPGETVKISCKASGYTFTDYSINWVKRAP | |
| | GKGLKWMGWINTETREPAYAYDFRGRFAFSLETSASTAYLQ | |
| | INNLKYEDTATYFCALDYSYAMDYWGQGTSVTVSSAAATT | |
| | TPAPRPPTPAPTIASQPLSLRPEACRPAAGGAVHTRGLDFACD | |
| | IYIWAPLAGTCGVLLLSLVITLYCKRGRKKLLYIFKQPFMRPV | |
| | QTTQEEDGCSCRFPEEEEGGCELRVKFSRSADAPAYQQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 153 | DIVLTQSPASLAVSLGERATINCRASESVSVIGAHLIHWYQQK | anti-BCMA CAR |
| | PGQPPKLLIYLASNLETGVPARFSGSGSGTDFTLTISSLQAED | |
| | AAIYYCLQSRIFPRTFGQGTKLEIKGSTSGSGKPGSGEGSTKG | |
| | QVQLVQSGSELKKPGASVKVSCKASGYTFTDYSINWVRQAP | |
| | GQGLEWMGWINTETREPAYAYDFRGRFVFSLDTSVSTAYLQ | |
| | ISSLKAEDTAVYYCARDYSYAMDYWGQGTLVTVSSAAATT | |
| | TPAPRPPTPAPTIASQPLSLRPEACRPAAGGAVHTRGLDFACD | |
| | IYIWAPLAGTCGVLLLSLVITLYCKRGRKKLLYIFKQPFMRPV | |
| | QTTQEEDGCSCRFPEEEEGGCELRVKFSRSADAPAYQQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 154 | DIVLTQSPASLAVSLGERATINCRASESVSVIGAHLIHWYQQK | anti-BCMA CAR |
| | PGQPPKLLIYLASNLETGVPARFSGSGSGTDFTLTISSLQAED | |
| | AAIYYCLQSRIFPRTFGQGTKLEIKGSTSGSGKPGSGEGSTKG | |
| | QVQLVQSGSELKKPGASVKVSCKASGYTFTDYSINWVRQAP | |
| | GQGLEWMGWINTETREPAYAYDFRGRFVFSLDTSVSTAYLQ | |
| | ISSLKAEDTAVYYCARDYSYAMDYWGQGTLVTVSSAAADT | |
| | GLYICKVELMYPPPYYLGIGNGTQIYVIDPEPCPDSDFLLWIL | |
| | AAVSSGLFFYSFLLTAVSKRGRKKLLYIFKQPFMRPVQTTQE | |
| | EDGCSCRFPEEEEGGCELRVKFSRSADAPAYQQGQNQLYNE | |
| | LNLGRREEYDVLDKRRGRDPEMGGKPRRKNPQEGLYNELQ | |
| | KDKMAEAYSEIGMKGERRRGKGHDGLYQGLSTATKDTYDA | |
| | LHMQALPPR | |
| 155 | DIVLTQSPASLAVSLGERATINCRASESVSVIGAHLIHWYQQK | anti-BCMA CAR |
| | PGQPPKLLIYLASNLETGVPARFSGSGSGTDFTLTISSLQAED | |
| | AAIYYCLQSRIFPRTFGQGTKLEIKGSTSGSGKPGSGEGSTKG | |
| | QVQLVQSGSELKKPGASVKVSCKASGYTFTDYSINWVRQAP | |
| | GQGLEWMGWINTETREPAYAYDFRGRFVFSLDTSVSTAYLQ | |
| | ISSLKAEDTAVYYCARDYSYAMDYWGQGTLVTVSSAAAQI | |
| | KESLRAELRVTERRAEVPTAHPSPSPRPAGQFQTLVVGVVGG | |
| | LLGSLVLLVWVLAVICSKRGRKKLLYIFKQPFMRPVQTTQEE | |
| | DGCSCRFPEEEEGGCELRVKFSRSADAPAYQQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 156 | EVQLVESGGGLVKPGGSLRLSCAASGFTFSDYYMSWIRQAP | anti-BCMA CAR |
| | GKGLEWVSYISSSGSTIYYADSVKGRFTISRDNAKNSLYLQM | |
| | NSLRAEDTAVYYCAKVDGDYTEDYWGQGTLVTVSSGGGGS | |
| | GGGGSGGGGSQSALTQPASVSGSPGQSITISCTGSSSDVGKY | |
| | NLVSWYQQPPGKAPKLIIYDVNKRPSGVSNRFSGSKSGNTAT | |
| | LTISGLQGDDEADYYCSSYGGSRSYVFGTGTKVTVLESKYGP | |
| | PCPPCPAPPVAGPSVFLFPPKPKDTLMISRTPEVTCVVVDVSQ | |
| | EDPEVQFNWYVDGVEVHNAKTKPREEQFQSTYRVVSVLTV | |
| | LHQDWLNGKEYKCKVSNKGLPSSIEKTISKAKGQPREPQVY | |
| | TLPPSQEEMTKNQVSLTCLVKGFYPSDIAVEWESNGQPENNY | |
| | KTTPPVLDSDGSFFLYSRLTVDKSRWQEGNVFSCSVMHEAL | |
| | HNHYTQKSLSLSLGKMFWVLVVVGGVLACYSLLVTVAFIIF | |
| | WVKRGRKKLLYIFKQPFMRPVQTTQEEDGCSCRFPEEEEGG | |
| | CELRVKFSRSADAPAYQQGQNQLYNELNLGRREEYDVLDK | |
| | RRGRDPEMGGKPRRKNPQEGLYNELQKDKMAEAYSEIGMK | |
| | GERRRGKGHDGLYQGLSTATKDTYDALHMQALPPR | |
| 157 | EVQLVQSGGGLVQPGRSLRLSCTASGFTFGDYAMSWFKQAP | anti-BCMA CAR |
| | GKGLEWVGFIRSKAYGGTTEYAASVKGRFTISRDDSKSIAYL | |
| | QMNSLKTEDTAVYYCAAWSAPTDYWGQGTLVTVSSGGGGS | |
| | GGGGSGGGGSDIQMTQSPAFLSASVGDRVTVTCRASQGISN | |
| | YLAWYQQKPGNAPRLLIYSASTLQSGVPSRFRGTGYGTEFSL | |
| | TIDSLQPEDFATYYCQQSYTSRQTFGPGTRLDIKESKYGPPCP | |
| | PCPAPPVAGPSVFLFPPKPKDTLMISRTPEVTCVVVDVSQEDP | |
| | EVQFNWYVDGVEVHNAKTKPREEQFQSTYRVVSVLTVLHQ | |
| | DWLNGKEYKCKVSNKGLPSSIEKTISKAKGQPREPQVYTLPP | |
| | SQEEMTKNQVSLTCLVKGFYPSDIAVEWESNGQPENNYKTT | |
| | PPVLDSDGSFFLYSRLTVDKSRWQEGNVFSCSVMHEALHNH | |
| | YTQKSLSLSLGKMFWVLVVVGGVLACYSLLVTVAFIIFWVK | |
| | RGRKKLLYIFKQPFMRPVQTTQEEDGCSCRFPEEEEGGCELR | |
| | VKFSRSADAPAYQQGQNQLYNELNLGRREEYDVLDKRRGR | |
| | DPEMGGKPRRKNPQEGLYNELQKDKMAEAYSEIGMKGERR | |
| | RGKGHDGLYQGLSTATKDTYDALHMQALPPR | |
| 158 | EVQLVESGGGLVKPGGSLRLSCAASGFTFSDYYMSWIRQAP | anti-BCMA CAR |
| | GKGLEWVSYISSSGSTIYYADSVKGRFTISRDNAKNSLYLQM | |
| | NSLRAEDTAVYYCAKVDGPPSFDIWGQGTMVTVSSGGGGS | |
| | GGGGSGGGGSSYVLTQPPSVSVAPGQTARITCGANNIGSKSV | |
| | HWYQQKPGQAPMLVVYDDDDRPSGIPERFSGSNSGNTATLT | |
| | ISGVEAGDEADYFCHLWDRSRDHYVFGTGTKLTVLESKYGP | |
| | PCPPCPAPPVAGPSVFLFPPKPKDTLMISRTPEVTCVVVDVSQ | |
| | EDPEVQFNWYVDGVEVHNAKTKPREEQFQSTYRVVSVLTV | |
| | LHQDWLNGKEYKCKVSNKGLPSSIEKTISKAKGQPREPQVY | |
| | TLPPSQEEMTKNQVSLTCLVKGFYPSDIAVEWESNGQPENNY | |
| | KTTPPVLDSDGSFFLYSRLTVDKSRWQEGNVFSCSVMHEAL | |
| | HNHYTQKSLSLSLGKMFWVLVVVGGVLACYSLLVTVAFIIF | |
| | WVKRGRKKLLYIFKQPFMRPVQTTQEEDGCSCRFPEEEEGG | |
| | CELRVKFSRSADAPAYQQGQNQLYNELNLGRREEYDVLDK | |
| | RRGRDPEMGGKPRRKNPQEGLYNELQKDKMAEAYSEIGMK | |
| | GERRRGKGHDGLYQGLSTATKDTYDALHMQALPPR | |
| 159 | SYELTQPPSASGTPGQRVTMSCSGTSSNIGSHSVNWYQQLPG | anti-BCMA CAR |
| | TAPKLLIYTNNQRPSGVPDRFSGSKSGTSASLAISGLQSEDEA | |
| | DYYCAAWDGSLNGLVFGGGTKLTVLGSRGGGGSGGGGSGG | |
| | GGSLEMAEVQLVQSGAEVKKPGESLKISCKGSGYSFTSYWIG | |
| | WVRQMPGKGLEWMGIIYPGDSDTRYSPSFQGHVTISADKSIS | |
| | TAYLQWSSLKASDTAMYYCARYSGSFDNWGQGTLVTVSSE | |
| | SKYGPPCPPCPAPPVAGPSVFLFPPKPKDTLMISRTPEVTCVV | |
| | VDVSQEDPEVQFNWYVDGVEVHNAKTKPREEQFQSTYRVV | |
| | SVLTVLHQDWLNGKEYKCKVSNKGLPSSIEKTISKAKGQPRE | |
| | PQVYTLPPSQEEMTKNQVSLTCLVKGFYPSDIAVEWESNGQP | |
| | ENNYKTTPPVLDSDGSFFLYSRLTVDKSRWQEGNVFSCSVM | |
| | HEALHNHYTQKSLSLSLGKMFWVLVVVGGVLACYSLLVTV | |
| | AFIIFWVKRGRKKLLYIFKQPFMRPVQTTQEEDGCSCRFPEEE | |
| | EGGCELRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 160 | QSALTQPASVSASPGQSIAISCTGTSSDVGWYQQHPGKAPKL | anti-BCMA CAR |
| | MIYEDSKRPSGVSNRFSGSKSGNTASLTISGLQAEDEADYYC | |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSSES | |
| | KYGPPCPPCPAPPVAGPSVFLFPPKPKDTLMISRTPEVTCVVV | |
| | DVSQEDPEVQFNWYVDGVEVHNAKTKPREEQFQSTYRVVS | |
| | VLTVLHQDWLNGKEYKCKVSNKGLPSSIEKTISKAKGQPREP | |
| | QVYTLPPSQEEMTKNQVSLTCLVKGFYPSDIAVEWESNGQPE | |
| | NNYKTTPPVLDSDGSFFLYSRLTVDKSRWQEGNVFSCSVMH | |
| | EALHNHYTQKSLSLSLGKMFWVLVVVGGVLACYSLLVTVA | |
| | FIIFWVKRGRKKLLYIFKQPFMRPVQTTQEEDGCSCRFPEEEE | |
| | GGCELRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 161 | QSALTQPASVSASPGQSIAISCTGTSSDVGWYQQHPGKAPKL | anti-BCMA CAR |
| | MIYEDSKRPSGVSNRFSGSKSGNTASLTISGLQAEDEADYYC | |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSSES | |
| | KYGPPCPPCPAPPVAGPSVFLFPPKPKDTLMISRTPEVTCVVV | |
| | DVSQEDPEVQFNWYVDGVEVHNAKTKPREEQFQSTYRVVS | |
| | VLTVLHQDWLNGKEYKCKVSNKGLPSSIEKTISKAKGQPREP | |
| | QVYTLPPSQEEMTKNQVSLTCLVKGFYPSDIAVEWESNGQPE | |
| | NNYKTTPPVLDSDGSFFLYSRLTVDKSRWQEGNVFSCSVMH | |
| | EALHNHYTQKSLSLSLGKMFWVLVVVGGVLACYSLLVTVA | |
| | FIIFWVRSKRSRLLHSDYMNMTPRRPGPTRKHYQPYAPPRDF | |
| | AAYRSRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 162 | EVQLVESGGGLVQPGGSLRLSCAVSGFALSNHGMSWVRRAP | anti-BCMA CAR |
| | GKGLEWVSGIVYSGSTYYAASVKGRFTISRDNSRNTLYLQM | |
| | NSLRPEDTAIYYCSAHGGESDVWGQGTTVTVSSASGGGGSG | |
| | GRASGGGGSDIQLTQSPSSLSASVGDRVTITCRASQSISSYLN | |
| | WYQQKPGKAPKLLIYAASSLQSGVPSRFSGSGSGTDFTLTISS | |
| | LQPEDFATYYCQQSYSTPYTFGQGTKVEIKTTTPAPRPPTPAP | |
| | TIASQPLSLRPEACRPAAGGAVHTRGLDFACDIYIWAPLAGT | |
| | CGVLLLSLVITLYCKRGRKKLLYIFKQPFMRPVQTTQEEDGC | |
| | SCRFPEEEEGGCELRVKFSRSADAPAYKQGQNQLYNELNLG | |
| | RREEYDVLDKRRGRDPEMGGKPRRKNPQEGLYNELQKDKM | |
| | AEAYSEIGMKGERRRGKGHDGLYQGLSTATKDTYDALHMQ | |
| | ALPPR | |
| 163 | QVQLVESGGGLVQPGRSLRLSCAASGFTFSNYAMSWVRQAP | anti-BCMA CAR |
| | GKGLGWVSGISRSGENTYYADSVKGRFTISRDNSKNTLYLQ | |
| | MNSLRDEDTAVYYCARSPAHYYGGMDVWGQGTTVTVSSA | |
| | SGGGGSGGRASGGGGSDIVLTQSPGTLSLSPGERATLSCRAS | |
| | QSISSSFLAWYQQKPGQAPRLLIYGASRRATGIPDRFSGSGSG | |
| | TDFTLTISRLEPEDSAVYYCQQYHSSPSWTFGQGTKLEIKTTT | |
| | PAPRPPTPAPTIASQPLSLRPEACRPAAGGAVHTRGLDFACDI | |
| | YIWAPLAGTCGVLLLSLVITLYCKRGRKKLLYIFKQPFMRPV | |
| | QTTQEEDGCSCRFPEEEEGGCELRVKFSRSADAPAYKQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 164 | QVQLVESGGGLVQPGGSLRLSCAVSGFALSNHGMSWVRRA | anti-BCMA CAR |
| | PGKGLEWVSGIVYSGSTYYAASVKGRFTISRDNSRNTLYLQ | |
| | MNSLRPEDTAIYYCSAHGGESDVWGQGTTVTVSSASGGGGS | |
| | GGRASGGGGSDIRLTQSPSPLSASVGDRVTITCQASED INKFL | |
| | NWYHQTPGKAPKLLIYDASTLQTGVPSRFSGSGSGTDFTLTI | |
| | NSLQPEDIGTYYCQQYESLPLTFGGGTKVEIKTTTPAPRPPTP | |
| | APTIASQPLSLRPEACRPAAGGAVHTRGLDFACDIYIWAPLA | |
| | GTCGVLLLSLVITLYCKRGRKKLLYIFKQPFMRPVQTTQEED | |
| | GCSCRFPEEEEGGCELRVKFSRSADAPAYKQGQNQLYNELN | |
| | LGRREEYDVLDKRRGRDPEMGGKPRRKNPQEGLYNELQKD | |
| | KMAEAYSEIGMKGERRRGKGHDGLYQGLSTATKDTYDALH | |
| | MQALPPR | |
| 165 | EVQLVESGGGLVQPGGSLRLSCAVSGFALSNHGMSWVRRAP | anti-BCMA CAR |
| | GKGLEWVSGIVYSGSTYYAASVKGRFTISRDNSRNTLYLQM | |
| | NSLRPEDTAIYYCSAHGGESDVWGQGTTVTVSSASGGGGSG | |
| | GRASGGGGSEIVLTQSPGTLSLSPGERATLSCRASQSIGSSSLA | |
| | WYQQKPGQAPRLLMYGASSRASGIPDRFSGSGSGTDFTLTIS | |
| | RLEPEDFAVYYCQQYAGSPPFTFGQGTKVEIKTTTPAPRPPTP | |
| | APTIASQPLSLRPEACRPAAGGAVHTRGLDFACDIYIWAPLA | |
| | GTCGVLLLSLVITLYCKRGRKKLLYIFKQPFMRPVQTTQEED | |
| | GCSCRFPEEEEGGCELRVKFSRSADAPAYKQGQNQLYNELN | |
| | LGRREEYDVLDKRRGRDPEMGGKPRRKNPQEGLYNELQKD | |
| | KMAEAYSEIGMKGERRRGKGHDGLYQGLSTATKDTYDALH | |
| | MQALPPR | |
| 166 | QIQLVQSGPDLKKPGETVKLSCKASGYTFTNFGMNWVKQAP | anti-BCMA CAR |
| | GKGFKWMAWINTYTGESYFADDFKGRFAFSVETSATTAYLQ | |
| | INNLKTEDTATYFCARGEIYYGYDGGFAYWGQGTLVTVSAG | |
| | GGGSGGGGSGGGGSDVVMTQSHRFMSTSVGDRVSITCRASQ | |
| | DVNTAVSWYQQKPGQSPKLLIFSASYRYTGVPDRFTGSGSG | |
| | ADFTLTISSVQAEDLAVYYCQQHYSTPWTFGGGTKLDIKTTT | |
| | PAPRPPTPAPTIASQPLSLRPEACRPAAGGAVHTRGLDFACDI | |
| | YIWAPLAGTCGVLLLSLVITLYCKRGRKKLLYIFKQPFMRPV | |
| | QTTQEEDGCSCRFPEEEEGGCELRVKFSRSADAPAYKQGQN | |
| | QLYNELNLGRREEYDVLDKRRGRDPEMGGKPRRKNPQEGL | |
| | YNELQKDKMAEAYSEIGMKGERRRGKGHDGLYQGLSTATK | |
| | DTYDALHMQALPPR | |
| 167 | QIQLVQSGPELKKPGETVKISCKASGYTFTDYSINWVKRAPG | anti-BCMA CAR |
| | KGLKWMGWINTETREPAYAYDFRGRFAFSLETSASTAYLQI | |
| | NNLKYEDTATYFCALDYSYAMDYWGQGTSVTVSSGGGGSG | |
| | GGGSGGGGSQIQLVQSGPELKKPGETVKISCKASGYTFTDYSI | |
| | NWVKRAPGKGLKWMGWINTETREPAYAYDFRGRFAFSLET | |
| | SASTAYLQINNLKYEDTATYFCALDYSYAMDYWGQGTSVT | |
| | VSSTTTPAPRPPTPAPTIASQPLSLRPEACRPAAGGAVHTRGL | |
| | DFACDIYIWAPLAGTCGVLLLSLVITLYCKRGRKKLLYIFKQP | |
| | FMRPVQTTQEEDGCSCRFPEEEEGGCELRVKFSRSADAPAYK | |
| | QGQNQLYNELNLGRREEYDVLDKRRGRDPEMGGKPRRKNP | |
| | QEGLYNELQKDKMAEAYSEIGMKGERRRGKGHDGLYQGLS | |
| | TATKDTYDALHMQALPPR | |
| 168 | QIQLVQSGPELKKPGETVKISCKASGYTFTDYSINWVKRAPG | anti-BCMA CAR |
| | KGLKWMGWINTETREPAYAYDFRGRFAFSLETSASTAYLQI | |
| | NNLKYEDTATYFCALDYSYAMDYWGQGTSVTVSSGGGGSG | |
| | GGGSGGGGSDIVLTQSPASLAMSLGKRATISCRASESVSVIGA | |
| | HLIHWYQQKPGQPPKLLIYLASNLETGVPARFSGSGSGTDFT | |
| | LTIDPVEEDDVAIYSCLQSRIFPRTFGGGTKLEIKTTTPAPRPP | |
| | TPAPTIASQPLSLRPEACRPAAGGAVHTRGLDFACDIYIWAPL | |
| | AGTCGVLLLSLVITLYCKRGRKKLLYIFKQPFMRPVQTTQEE | |
| | DGCSCRFPEEEEGGCELRVKFSRSADAPAYKQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 169 | QIQLVQSGPELKKPGETVKISCKASGYTFRHYSMNWVKQAP | anti-BCMA CAR |
| | GKGLKWMGRINTESGVPIYADDFKGRFAFSVETSASTAYLVI | |
| | NNLKDEDTASYFCSNDYLYSLDFWGQGTALTVSSGGGGSGG | |
| | GGSGGGGSDIVLTQSPPSLAMSLGKRATISCRASESVTILGSH | |
| | LIYWYQQKPGQPPTLLIQLASNVQTGVPARFSGSGSRTDFTL | |
| | TIDPVEEDDVAVYYCLQSRTIPRTFGGGTKLEIKTTTPAPRPP | |
| | TPAPTIASQPLSLRPEACRPAAGGAVHTRGLDFACDIYIWAPL | |
| | AGTCGVLLLSLVITLYCKRGRKKLLYIFKQPFMRPVQTTQEE | |
| | DGCSCRFPEEEEGGCELRVKFSRSADAPAYKQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 170 | QIQLVQSGPELKKPGETVKISCKASGYTFTHYSMNWVKQAP | anti-BCMA CAR |
| | GKGLKWMGRINTETGEPLYADDFKGRFAFSLETSASTAYLVI | |
| | NNLKNEDTATFFCSNDYLYSCDYWGQGTTLTVSSGGGGSGG | |
| | GGSGGGGSDIVLTQSPPSLAMSLGKRATISCRASESVTILGSH | |
| | LIYWYQQKPGQPPTLLIQLASNVQTGVPARFSGSGSRTDFTL | |
| | TIDPVEEDDVAVYYCLQSRTIPRTFGGGTKLEIKTTTPAPRPP | |
| | TPAPTIASQPLSLRPEACRPAAGGAVHTRGLDFACDIYIWAPL | |
| | AGTCGVLLLSLVITLYCKRGRKKLLYIFKQPFMRPVQTTQEE | |
| | DGCSCRFPEEEEGGCELRVKFSRSADAPAYKQGQNQLYNEL | |
| | NLGRREEYDVLDKRRGRDPEMGGKPRRKNPQEGLYNELQK | |
| | DKMAEAYSEIGMKGERRRGKGHDGLYQGLSTATKDTYDAL | |
| | HMQALPPR | |
| 171 | DIVLTQSPPSLAMSLGKRATISCRASESVTILGSHLIHWYQQK | anti-BCMA CAR |
| | PGQPPTLLIQLASNVQTGVPARFSGSGSRTDFTLTIDPVEEDD | |
| | VAVYYCLQSRTIPRTFGGGTKLEIKGSTSGSGKPGSGEGSTK | |
| | GQIQLVQSGPELKKPGETVKISCKASGYTFTDYSINWVKRAP | |
| | GKGLKWMGWINTETREPAYAYDFRGRFAFSLETSASTAYLQ | |
| | INNLKYEDTATYFCALDYSYAMDYWGQGTSVTVSSFVPVFL | |
| | PAKPTTTPAPRPPTPAPTIASQPLSLRPEACRPAAGGAVHTRG | |
| | LDFACDIYIWAPLAGTCGVLLLSLVITLYCNHRNRSKRSRLL | |
| | HSDYMNMTPRRPGPTRKHYQPYAPPRDFAAYRSRVKFSRSA | |
| | DAPAYQQGQNQLYNELNLGRREEYDVLDKRRGRDPEMGGK | |
| | PRRKNPQEGLYNELQKDKMAEAYSEIGMKGERRRGKGHDG | |
| | LYQGLSTATKDTYDALHMQALPPR | |
| 172 | QVQLVQSGAEVKKPGASVKVSCKASGYSFPDYYINWVRQA | anti-BCMA CAR |
| | PGQGLEWMGWIYFASGNSEYNQKFTGRVTMTRDTSINTAY | |
| | MELSSLTSEDTAVYFCASLYDYDWYFDVWGQGTMVTVSSG | |
| | GGGSGGGGSGGGGSDIVMTQTPLSLSVTPGQPASISCKSSQSL | |
| | VHSNGNTYLHWYLQKPGQSPQLLIYKVSNRFSGVPDRFSGS | |
| | GSGTDFTLKISRVEAEDVGIYYCSQSSIYPWTFGQGTKLEIKG | |
| | LAVSTISSFFPPGYQIYIWAPLAGTCGVLLLSLVITLYCKRGR | |
| | KKLLYIFKQPFMRPVQTTQEEDGCSCRFPEEEEGGCELRVKF | |
| | SRSADAPAYQQGQNQLYNELNLGRREEYDVLDKRRGRDPE | |
| | MGGKPRRKNPQEGLYNELQKDKMAEAYSEIGMKGERRRGK | |
| | GHDGLYQGLSTATKDTYDALHMQALPPR | |
| 173 | QVQLVQSGAEVKKPGASVKVSCKASGYSFPDYYINWVRQA | anti-BCMA CAR |
| | PGQGLEWMGWIYFASGNSEYNQKFTGRVTMTRDTSINTAY | |
| | MELSSLTSEDTAVYFCASLYDYDWYFDVWGQGTMVTVSSG | |
| | GGGSGGGGSGGGGSDIVMTQTPLSLSVTPGQPASISCKSSQSL | |
| | VHSNGNTYLHWYLQKPGQSPQLLIYKVSNRFSGVPDRFSGS | |
| | GSGTDFTLKISRVEAEDVGIYYCSQSSIYPWTFGQGTKLEIKT | |
| | TTPAPRPPTPAPTIASQPLSLRPEACRPAAGGAVHTRGLDFAC | |
| | DIYIWAPLAGTCGVLLLSLVITLYCKRGRKKLLYIFKQPFMRP | |
| | VQTTQEEDGCSCRFPEEEEGGCELRVKFSRSADAPAYQQGQ | |
| | NQLYNELNLGRREEYDVLDKRRGRDPEMGGKPRRKNPQEG | |
| | LYNELQKDKMAEAYSEIGMKGERRRGKGHDGLYQGLSTAT | |
| | KDTYDALHMQALPPR | |
| 174 | QVQLVQSGAEVKKPGASVKVSCKASGYSFPDYYINWVRQA | anti-BCMA CAR |
| | PGQGLEWMGWIYFASGNSEYNQKFTGRVTMTRDTSINTAY | |
| | MELSSLTSEDTAVYFCASLYDYDWYFDVWGQGTMVTVSSG | |
| | GGGSGGGGSGGGGSDIVMTQTPLSLSVTPGQPASISCKSSQSL | |
| | VHSNGNTYLHWYLQKPGQSPQLLIYKVSNRFSGVPDRFSGS | |
| | GSGTDFTLKISRVEAEDVGIYYCSQSSIYPWTFGQGTKLEIKE | |
| | PKSPDKTHTCPPCPAPPVAGPSVFLFPPKPKDTLMIARTPEVT | |
| | CVVVDVSHEDPEVKFNWYVDGVEVHNAKTKPREEQYNSTY | |
| | RVVSVLTVLHQDWLNGKEYKCKVSNKALPAPIEKTISKAKG | |
| | QPREPQVYTLPPSRDELTKNQVSLTCLVKGFYPSDIAVEWES | |
| | NGQPENNYKTTPPVLDSDGSFFLYSKLTVDKSRWQQGNVFS | |
| | CSVMHEALHNHYTQKSLSLSPGKIYIWAPLAGTCGVLLLSLV | |
| | ITLYCKRGRKKLLYIFKQPFMRPVQTTQEEDGCSCRFPEEEEG | |
| | GCELRVKFSRSADAPAYQQGQNQLYNELNLGRREEYDVLD | |
| | KRRGRDPEMGGKPRRKNPQEGLYNELQKDKMAEAYSEIGM | |
| | KGERRRGKGHDGLYQGLSTATKDTYDALHMQALPPR | |
| 175 | QVQLVQSGAEVKKPGASVKVSCKASGYSFPDYYINWVRQA | anti-BCMA CAR |
| | PGQGLEWMGWIYFASGNSEYNQKFTGRVTMTRDTSSSTAY | |
| | MELSSLRSEDTAVYFCASLYDYDWYFDVWGQGTMVTVSSG | |
| | GGGSGGGGSGGGGSDIVMTQTPLSLSVTPGEPASISCKSSQSL | |
| | VHSNGNTYLHWYLQKPGQSPQLLIYKVSNRFSGVPDRFSGS | |
| | GSGADFTLKISRVEAEDVGVYYCAETSHVPWTFGQGTKLEIK | |
| | GLAVSTISSFFPPGYQIYIWAPLAGTCGVLLLSLVITLYCKRG | |
| | RKKLLYIFKQPFMRPVQTTQEEDGCSCRFPEEEEGGCELRVK | |
| | FSRSADAPAYQQGQNQLYNELNLGRREEYDVLDKRRGRDPE | |
| | MGGKPRRKNPQEGLYNELQKDKMAEAYSEIGMKGERRRGK | |
| | GHDGLYQGLSTATKDTYDALHMQALPPR | |
| 176 | QVQLVQSGAEVKKPGASVKVSCKASGYSFPDYYINWVRQA | anti-BCMA CAR |
| | PGQGLEWMGWIYFASGNSEYNQKFTGRVTMTRDTSSSTAY | |
| | MELSSLRSEDTAVYFCASLYDYDWYFDVWGQGTMVTVSSG | |
| | GGGSGGGGSGGGGSDIVMTQTPLSLSVTPGEPASISCKSSQSL | |
| | VHSNGNTYLHWYLQKPGQSPQLLIYKVSNRFSGVPDRFSGS | |
| | GSGADFTLKISRVEAEDVGVYYCAETSHVPWTFGQGTKLEIK | |
| | TTTPAPRPPTPAPTIASQPLSLRPEACRPAAGGAVHTRGLDFA | |
| | CDIYIWAPLAGTCGVLLLSLVITLYCKRGRKKLLYIFKQPFM | |
| | RPVQTTQEEDGCSCRFPEEEEGGCELRVKFSRSADAPAYQQG | |
| | QNQLYNELNLGRREEYDVLDKRRGRDPEMGGKPRRKNPQE | |
| | GLYNELQKDKMAEAYSEIGMKGERRRGKGHDGLYQGLSTA | |
| | TKDTYDALHMQALPPR | |
| 177 | QVQLVQSGAEVKKPGASVKVSCKASGYSFPDYYINWVRQA | anti-BCMA CAR |
| | PGQGLEWMGWIYFASGNSEYNQKFTGRVTMTRDTSSSTAY | |
| | MELSSLRSEDTAVYFCASLYDYDWYFDVWGQGTMVTVSSG | |
| | GGGSGGGGSGGGGSDIVMTQTPLSLSVTPGEPASISCKSSQSL | |
| | VHSNGNTYLHWYLQKPGQSPQLLIYKVSNRFSGVPDRFSGS | |
| | GSGADFTLKISRVEAEDVGVYYCAETSHVPWTFGQGTKLEIK | |
| | EPKSPDKTHTCPPCPAPPVAGPSVFLFPPKPKDTLMIARTPEV | |
| | TCVVVDVSHEDPEVKFNWYVDGVEVHNAKTKPREEQYNST | |
| | YRVVSVLTVLHQDWLNGKEYKCKVSNKALPAPIEKTISKAK | |
| | GQPREPQVYTLPPSRDELTKNQVSLTCLVKGFYPSDIAVEWE | |
| | SNGQPENNYKTTPPVLDSDGSFFLYSKLTVDKSRWQQGNVF | |
| | SCSVMHEALHNHYTQKSLSLSPGKIYIWAPLAGTCGVLLLSL | |
| | VITLYCKRGRKKLLYIFKQPFMRPVQTTQEEDGCSCRFPEEEE | |
| | GGCELRVKFSRSADAPAYQQGQNQLYNELNLGRREEYDVL | |
| | DKRRGRDPEMGGKPRRKNPQEGLYNELQKDKMAEAYSEIG | |
| | MKGERRRGKGHDGLYQGLSTATKDTYDALHMQALPPR | |
| 178 | IYIWAPLAGTCGVLLLSLVITLYC | CD8 TM |
| 179 | gaatctaagtacggaccgccctgccctccctgccctgctcctcctgtggctggaccaagcgtgtt | Modified IgG4 |
| | cctgtttccacctaagcctaaagataccctgatgatttcccgcacacctgaagtgacttgcgtgg | hinge-IgG2/IgG4 |
| | tcgtggacgtgagccaggaggatccagaagtgcagttcaactggtacgtggacggcgtggaagtc | CH2-IgG4 CH3 |
| | cacaatgctaagactaaaccccgagaggaacagtttcagtcaacttaccgggtcgtgagcgtgct | spacer (nt) |
| | gaccgtcctgcatcaggattggctgaacgggaaggagtataagtgcaaagtgtctaataaggga | |
| | ctgcctagctccatcgagaaaacaattagtaaggcaaaagggcagcctcgagaaccacaggtg | |
| | tataccctgccccctagccaggaggaaatgaccaagaaccaggtgtccctgacatgtctggtca | |
| | aaggcttctatccaagtgacatcgccgtggagtgggaatcaaatgggcagcccgagaacaatta | |
| | caagaccacaccacccgtgctggactctgatggaagtttctttctgtattccaggctgaccgtgga | |
| | taaatctcgctggcaggagggcaacgtgttctcttgcagtgtcatgcacgaagccctgcacaatc | |
| | attatacacagaagtcactgagcctgtccctgggcaaa | |
| 180 | QSALTQPASVSASPGQSIAISCTGTSSDVGWYQQHPGKAPKL | BCMA-55 scFv |
| | MIYEDSKRPSGVSNRFSGSKSGNTASLTISGLQAEDEADYYC | (aa) |
| | SSNTRSSTLVFGGGTKLTVLGSRGGGGSGGGGSGGGGSLEM | |
| | AEVQLVQSGAEMKKPGASLKLSCKASGYTFIDYYVYWMRQ | |
| | APGQGLESMGWINPNSGGTNYAQKFQGRVTMTRDTSISTAY | |
| | MELSRLRSDDTAMYYCARSQRDGYMDYWGQGTLVTVSS | |
| 181 | ASTKGPSVFPLAPCSRSTSESTAALGCLVKDYFPEPVTVSWN | Human IgG2 Fc |
| | SGALTSGVHTFPAVLQSSGLYSLSSVVTVPSSNFGTQTYTCN | (Uniprot P01859) |
| | VDHKPSNTKVDKTVERKCCVECPPCPAPPVAGPSVFLFPPKP | |
| | KDTLMISRTPEVTCVVVDVSHEDPEVQFNWYVDGVEVHNA | |
| | KTKPREEQFNSTFRVVSVLTVVHQDWLNGKEYKCKVSNKG | |
| | LPAPIEKTISKTKGQPREPQVYTLPPSREEMTKNQVSLTCLVK | |
| | GFYPSDISVEWESNGQPENNYKTTPPMLDSDGSFFLYSKLTV | |
| | DKSRWQQGNVFSCSVMHEALHNHYTQKSLSLSPGK | |
| 182 | ASTKGPSVFPLAPCSRSTSESTAALGCLVKDYFPEPVTVSWN | Human IgG4 Fc |
| | SGALTSGVHTFPAVLQSSGLYSLSSVVTVPSSSLGTKTYTCN | (Uniprot P01861) |
| | VDHKPSNTKVDKRVESKYGPPCPSCPAPEFLGGPSVFLFPPKP | |
| | KDTLMISRTPEVTCVVVDVSQEDPEVQFNWYVDGVEVHNA | |
| | KTKPREEQFNSTYRVVSVLTVLHQDWLNGKEYKCKVSNKG | |
| | LPSSIEKTISKAKGQPREPQVYTLPPSQEEMTKNQVSLTCLVK | |
| | GFYPSDIAVEWESNGQPENNYKTTPPVLDSDGSFFLYSRLTV | |
| | DKSRWQEGNVFSCSVMHEALHNHYTQKSLSLSLGK | |
| 183 | gagtctaaatacggaccgccttgtcctccttgtcccgctcctcctgttgccggaccttccgtgtt | optimized SSE |
| | cctgtttcctccaaagcctaaggacaccctgatgatcagcaggacccctgaagtgacctgcgtgg | modified IgG4 |
| | tggtggatgtgtcccaagaggatcccgaggtgcagttcaactggtatgtggacggcgtggaagtg | hinge-IgG2/IgG4 |
| | cacaacgccaagaccaagcctagagaggaacagttccagagcacctacagagtggtgtccgtgc | Ch2-IgG4 Ch3 |
| | tgacagtgctgcaccaggattggctgaacggcaaagagtacaagtgcaaggtgtccaacaagg | spacer (nt) |
| | gcctgcctagcagcatcgagaaaaccatctccaaggccaagggccagccaagagagccccag | |
| | gtttacacactgcctccaagccaagaggaaatgaccaagaatcaggtgtccctgacatgcctggt | |
| | caagggcttctacccctccgatatcgccgtggaatgggagagcaatggccagcctgagaacaa | |
| | ctacaagaccacacctcctgtgctggacagcgacggcagtttcttcctgtatagtagactcaccgt | |
| | ggataaatcaagatggcaagagggcaacgtgttcagctgcagcgtgatgcacgaggccctgca | |
| | caaccactacacccagaaaagcctgagcctgtctctgggcaag | |

What is claimed:

1. A method of treating multiple myeloma, the method comprising:
(a) administering a T cell therapy to a subject having a relapsed or refractory multiple myeloma (R/R MM), said T cell therapy comprising a dose of genetically engineered T cells expressing a chimeric antigen receptor (CAR) that specifically binds to BCMA; and
(b) administering to the subject an immunomodulatory compound that is (S)-3-[4-(4-morpholin-4-ylmethyl-benzyloxy)-1-oxo-1,3-dihydro-isoindol-2-yl]-piperidine-2,6-dione having the following structure: 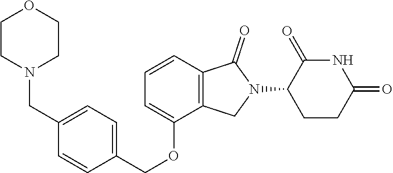 [SMILES: O=C1CC[C@H](N2Cc3c(OCc4ccc(CN5CCOCC5)cc4)cccc3C2=O)C(=O)N1]
or a pharmaceutically acceptable salt, solvate, hydrate, co-crystal, clathrate, or polymorph thereof,
wherein administration of the immunomodulatory compound is initiated after administration of the T cell therapy.

2. The method of claim 1, wherein prior to initiation of the administration of the T cell therapy and the immunomodulatory compound, the subject has received one or more prior therapies for treating the R/R MM, said one or more prior therapies comprising an immunomodulatory agent.

3. A method of treating multiple myeloma, the method comprising:
(a) administering a T cell therapy to a subject having a relapsed or refractory multiple myeloma (R/R MM), said T cell therapy comprising a dose of genetically engineered T cells expressing a chimeric antigen receptor (CAR) that specifically binds to BCMA; and
(b) administering to the subject an immunomodulatory compound that is (S)-3-[4-(4-morpholin-4-ylmethyl-benzyloxy)-1-oxo-1,3-dihydro-isoindol-2-yl]-piperidine-2,6-dione having the following structure: 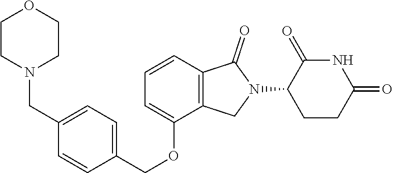 [SMILES: O=C1CC[C@H](N2Cc3c(OCc4ccc(CN5CCOCC5)cc4)cccc3C2=O)C(=O)N1]
or a pharmaceutically acceptable salt, solvate, hydrate, co-crystal, clathrate, or polymorph thereof;
wherein prior to initiation of the administration of the T cell therapy and the immunomodulatory compound, the subject has received one or more prior therapies for treating the R/R MM, said one or more prior therapies comprising an immunomodulatory agent.

4. The method of any of claims 1-3, wherein the immunomodulatory compound is or comprises a pharmaceutically acceptable salt of (S)-3-[4-(4-morpholin-4-ylmethyl-benzyloxy)-1-oxo-1,3-dihydro-isoindol-2-yl]-piperidine-2,6-dione.

5. The method of any of claims 1-3, wherein the immunomodulatory compound is or comprises a hydrate of (S)-3-[4-(4-morpholin-4-ylmethyl-benzyloxy)-1-oxo-1,3-dihydro-isoindol-2-yl]-piperidine-2,6-dione.

6. The method of any of claims 1-3, wherein the immunomodulatory compound is or comprises a solvate of (S)-3-[4-(4-morpholin-4-ylmethyl-benzyloxy)-1-oxo-1,3-dihydro-isoindol-2-yl]-piperidine-2,6-dione.

7. The method of any of claims 1-3, wherein the immunomodulatory compound is or comprises (S)-3-[4-(4-morpholin-4-ylmethyl-benzyloxy)-1-oxo-1,3-dihydro-isoindol-2-yl]-piperidine-2,6-dione.

8. A method of treating multiple myeloma, the method comprising:
(a) administering a T cell therapy to a subject having a relapsed or refractory multiple myeloma (R/R MM), said T cell therapy comprising a dose of genetically engineered T cells expressing a chimeric antigen receptor (CAR) that specifically binds to BCMA; and
(b) administering to the subject an immunomodulatory compound that is (S)-4-(4-(4-(((2-(2,6-dioxopiperidin-3-yl)-1-oxoisoindolin-4-yl)oxy)methyl)benzyl)piperazin-1-yl)-3-fluorobenzonitrile having the following structure: 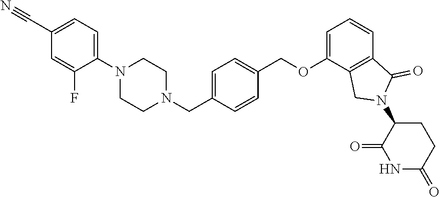 [SMILES: N#Cc1ccc(N2CCN(Cc3ccc(COc4cccc5c4CN([C@H]4CCC(=O)NC4=O)C5=O)cc3)CC2)c(F)c1]
or a pharmaceutically acceptable salt, solvate, hydrate, co-crystal, clathrate, or polymorph thereof,
wherein administration of the immunomodulatory compound is initiated after administration of the T cell therapy.

9. The method of claim 8, wherein prior to initiation of the administration of the T cell therapy and the immunomodulatory compound, the subject has received one or more prior therapies for treating the R/R MM, said one or more prior therapies comprising an immunomodulatory agent.

10. A method of treating multiple myeloma, the method comprising:
(a) administering a T cell therapy to a subject having a relapsed or refractory multiple myeloma (R/R MM), said T cell therapy comprising a dose of genetically engineered T cells expressing a chimeric antigen receptor (CAR) that specifically binds to BCMA; and
(b) a administering to the subject an immunomodulatory compound that is (S)-4-(4-(4-(((2-(2,6-dioxopiperidin-3-yl)-1-oxoisoindolin-4-yl)oxy)methyl)benzyl)piperazin-1-yl)-3-fluorobenzonitrile having the following structure: 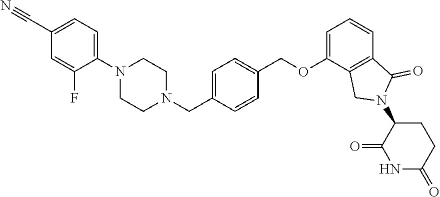 [SMILES: N#Cc1ccc(N2CCN(Cc3ccc(COc4cccc5c4CN([C@H]4CCC(=O)NC4=O)C5=O)cc3)CC2)c(F)c1]
or a pharmaceutically acceptable salt, solvate, hydrate, co-crystal, clathrate, or polymorph thereof, wherein prior to initiation of the administration of the T cell therapy and the immunomodulatory compound, the subject has received one or more prior therapies for treating the R/R MM, said one or more prior therapies comprising an immunomodulatory agent.

11. The method of any of claims 8-10, wherein the immunomodulatory compound is or comprises a pharmaceutically acceptable salt of (S)-4-(4-(4-(((2-(2,6-dioxopiperidin-3-yl)-1-oxoisoindolin-4-yl)oxy)methyl)benzyl)piperazin-1-yl)-3-fluorobenzonitrile.

12. The method of any of claims 8-10, wherein the immunomodulatory compound is or comprises a hydrate of (S)-4-(4-(4-(((2-(2,6-dioxopiperidin-3-yl)-1-oxoisoindolin-4-yl)oxy)methyl)benzyl)piperazin-1-yl)-3-fluorobenzonitrile.

13. The method of any of claims 8-10, wherein the immunomodulatory compound is or comprises a solvate of (S)-4-(4-(4-(((2-(2,6-dioxopiperidin-3-yl)-1-oxoisoindolin-4-yl)oxy)methyl)benzyl)piperazin-1-yl)-3-fluorobenzonitrile.

14. The method of any of claims 8-10, wherein the immunomodulatory compound is or comprises (S)-4-(4-(4-(((2-(2,6-dioxopiperidin-3-yl)-1-oxoisoindolin-4-yl)oxy)methyl)benzyl)piperazin-1-yl)-3-fluorobenzonitrile.

15. The method of any of claims 1-14, wherein the subject has relapsed or been refractory following at least 3 or at least 4 prior therapies for multiple myeloma.

16. The method of any of claims 1-15, wherein the subject has received, and has relapsed or been refractory to, three or more therapies selected from among:
autologous stem cell transplant (ASCT);
an immunomodulatory agent;
a proteasome inhibitor; and
an anti-CD38 antibody; unless the subject was not a candidate for or was contraindicated for one or more of the therapies.

17. The method of any of claims 2-7 and 9-16, wherein the immunomodulatory agent is selected from among thalidomide, lenalidomide and pomalidomide.

18. The method of claim 16, wherein the proteasome inhibitor is selected from among bortezomib, carfilzomib and ixazomib.

19. The method of claim 16, wherein the anti-CD38 antibody is or comprises daratumumab.

20. The method of any of claims 1-19, wherein, at the time of administration, the subject has been refractory to or not responded to bortezomib, carfilzomib, lenalidomide, pomalidomide and/or an anti-CD38 monoclonal antibody.

21. The method of any of claims 1-20, wherein, at the time of administration, the subject has IMWG high risk cytogenetics.

22. The method of any of claims 1-2, 4-9, and 11-21, wherein administration of the immunomodulatory compound is initiated at or prior to peak expansion of the T cell therapy in the subject.

23. The method of claim 22, wherein peak expansion of the T cell therapy is between at or about 11 days and at or about 15 days after administering the T cell therapy.

24. The method of any of claims 1-2, 4-9, and 11-23, wherein administration of the immunomodulatory compound is initiated between at or about 1 day and at or about 15 days, inclusive, after administering the T cell therapy.

25. The method of any of claims 1-2, 4-9, and 11-24, wherein administration of the immunomodulatory compound is initiated between at or about 1 day and at or about 11 days, inclusive, after administering the T cell therapy.

26. The method of any of claims 1-2, 4-9, and 11-25, wherein the administration of the immunomodulatory compound is initiated between at or about 8 days and at or about 15 days, inclusive, after administering the T cell therapy.

27. The method of any of claims 1-2, 4-9, and 11-26, wherein administration of the immunomodulatory compound is initiated at or about 1 day after administering the T cell therapy.

28. The method of any of claims 1-2, 4-9, and 11-26, wherein administration of the immunomodulatory compound is initiated at or about 8 days after administering the T cell therapy.

29. The method of any of claims 1-2, 4-9, and 11-26, wherein the administration of the immunomodulatory compound is initiated at or about 15 days after administering the T cell therapy.

30. The method of any of claims 1-2, 4-9, and 11-21, wherein the administration of the immunomodulatory compound is initiated about 14 to about 35 days after initiation of administration of the T cell therapy.

31. The method of any of claims 1-2, 4-9, 11-21, and 30, wherein the administration of the immunomodulatory compound is initiated about 21 to about 35 days after initiation of administration of the T cell therapy.

32. The method of any of claims 1-2, 4-9, 11-21, 30, and 31, wherein the administration of the immunomodulatory compound is initiated about 21 to about 28 days after initiation of administration of the T cell therapy.

33. The method of any of claims 1-2, 4-9, 11-21, and 30-32, wherein the administration of the immunomodulatory compound is initiated at or about 21 days, at or about 22 days, at or about 23 days, at or about 24 days, at or about 25 days, at or about 26 days, at or about 27 days, or at or about 28 days after initiation of administration of the T cell therapy.

34. The method of any of claims 1-2, 4-9, 11-21, and 30-33, wherein the administration of the immunomodulatory compound is initiated at or about 28 days after the initiation of the administration of the T cell therapy.

35. The method of any of claims 3-7 and 10-21, wherein the immunomodulatory compound is administered from or from about 0 to 30 days, 0 to 15 days, 0 to 6 days, 0 to 96 hours, 2 hours to 15 days, 2 hours to 6 days, 2 hours to 96 hours, 6 hours to 30 days, 6 hours to 15 days, 6 hours to 6 days, 6 hours to 96 hours, 12 hours to 30 days, 12 hours to 15 days, 12 hours to 6 days, or 12 hours to 96 hours prior to initiation of the T cell therapy.

36. The method of any of claims 3-7, 10-21, and 35, wherein the immunomodulatory compound is administered no more than about 96 hours, 72 hours, 48 hours, or 24 hours prior to initiation of the T cell therapy.

37. The method of any of claims 1-36, wherein the immunomodulatory compound is administered at least once daily in a cycle regimen.

38. The method of claim 37, wherein the immunomodulatory compound is administered in a cycle regimen comprising the administration of the immunomodulatory compound for a plurality of consecutive days followed by a rest period during which the immunomodulatory compound is not administered.

39. The method of claim 38, wherein the plurality of consecutive days is up to 21 days.

40. The method of any of claims 37-39, wherein the cycle regimen is a four-week (28-day) cycle wherein the immunomodulatory compound is administered daily for three consecutive weeks in the four-week cycle and is not administered for the last week.

41. The method of any of claims 37-40, wherein the cycle regimen is a four-week (28-day) cycle wherein the immunomodulatory compound is administered daily for days 1 through 21 of each four-week cycle.

42. The method of any of claims 37-41, wherein the cycle regimen is repeated a plurality of times.

43. The method of any of claims 1-42, wherein the immunomodulatory compound is administered up to at or about six months after initiation of administration of the T cell therapy.

44. The method of any of claims 1-43, wherein the immunomodulatory compound is administered in an amount that is at or about 0.1 mg to about 1.0 mg per day.

45. The method of any of claims 1-44, wherein the immunomodulatory compound is administered in an amount that is at or about 0.3 mg to about 0.6 mg.

46. The method of any of claims 1-45, wherein the immunomodulatory compound is administered in an amount that is at or about 0.3 mg.

47. The method of any of claims 1-45, wherein the immunomodulatory compound is administered in an amount that is at or about 0.45 mg.

48. The method of any of claims 1-45, wherein the immunomodulatory compound is administered in an amount that is at or about 0.6 mg.

49. The method of any of claims 1-48, wherein the immunomodulatory compound is administered orally.

50. The method of any of claims 1-49, wherein at the time of the initiation of the administration of the immunomodulatory compound, the subject does not exhibit a severe toxicity following the administration of the T cell therapy.

51. The method of claim 50, wherein:
the severe toxicity is severe cytokine release syndrome (CRS), optionally grade 3 or higher, prolonged grade 3 or higher or grade 4 or 5 CRS; and/or
the severe toxicity is severe neurotoxicity, optionally grade 3 or higher, prolonged grade 3 or higher or grade 4 or 5 neurotoxicity.

52. The method of any one of claims 1-51, wherein the administration of the immunomodulatory compound is suspended and/or the cycling regimen is modified if the subject exhibits a toxicity following the administration of the immunomodulatory compound, optionally a hematologic toxicity.

53. The method of claim 52, wherein the toxicity is selected from severe neutropenia, optionally febrile neutropenia, prolonged grade 3 or higher neutropenia.

54. The method of any of claims 1-53, wherein the administration of the immunomodulatory compound:
reverses an exhaustion phenotype in CAR-expressing T cells in the subject;
prevents, inhibits or delays the onset of an exhaustion phenotype in CAR-expressing T cells in the subject;
or reduces the level or degree of an exhaustion phenotype in CAR-expressing T cells in the subject; or
reduces the percentage, of the total number of CAR-expressing T cells in the subject, that have an exhaustion phenotype.

55. The method of any of claims 1-54, wherein following administration of the immunomodulatory compound or initiation thereof, the subject exhibits a restoration or rescue of an antigen- or tumor-specific activity or function of CAR-expressing T cells in said subject, optionally wherein said restoration, rescue, and/or initiation of administration of said compound, is at a point in time after CAR-expressing T cells in the subject or the in the blood of the subject have exhibited an exhausted phenotype.

56. The method of any of claims 1-55, wherein the administration of the immunomodulatory compound comprises administration at an amount, frequency and/or duration effective to:
(a) effect an increase in antigen-specific or antigen receptor-driven activity of naïve or non-exhausted T cells in the subject, which optionally comprise T cells expressing said CAR, following exposure of the T cells to BCMA or to an agonist of the CAR, optionally wherein the agonist is an anti-idiotypic antibody, as compared to the absence of said administration of said compound; or
(b) prevent, inhibit or delay the onset of an exhaustion phenotype, in naïve or non-exhausted T cells T cells in the subject, which optionally comprise T cells expressing said CAR, following exposure of the T cells to BCMA or to an agonist of the CAR, optionally wherein the agonist is an anti-idiotypic antibody, as compared to the absence of said administration of said compound; or
(c) reverse an exhaustion phenotype in exhausted T cells, optionally comprising T cells expressing said CAR, in the subject, as compared to the absence of said administration of said subject.

57. The method of any of claims 1-56, wherein at the time of the administration of the immunomodulatory compound an exhausted phenotype of one or more of the CAR-expressing T cells, or a marker or parameter indicative thereof, has been detected or measured in the subject or in a biological sample from the subject.

58. The method of claim 57, wherein at least at or about 10%, at least at or about 20%, at least at or about 30%, at least at or about 40%, or at least at or about 50% of the total CAR-expressing T cells in a biological sample from the subject has an exhausted phenotype.

59. The method of claim 57 or claim 58, wherein greater than at or about 10%, greater than at or about 20%, greater than at or about 30%, greater than at or about 40%, or greater than at or about 50% of the CAR-expressing T cells in a biological sample from the subject has an exhausted phenotype compared to the percentage of the CAR-expressing T cells having the exhausted phenotype in a comparable biological sample at a prior time point.

60. The method of any of claims 57-59, wherein the exhaustion phenotype, with reference to a T cell or population of T cells, comprises:
an increase in the level or degree of surface expression on the T cell or T cells, or in the percentage of T said population of T cells exhibiting surface expression, of one or more exhaustion marker, optionally 2, 3, 4, 5 or 6 exhaustion markers, compared to a reference T cell population under the same conditions; or
a decrease in the level or degree of an activity exhibited by said T cells or population of T cells upon exposure to BCMA or an agonist of the CAR, optionally wherein the agonist is an anti-idiotypic antibody, compared to a reference T cell population, under the same conditions.

61. The method of claim 60, wherein the increase in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

62. The method of claim 60, wherein the decrease in the level, degree or percentage is by greater than at or about 1.2-fold, at or about 1.5-fold, at or about 2.0-fold, at or about 3-fold, at or about 4-fold, at or about 5-fold, at or about 6-fold, at or about 7-fold, at or about 8-fold, at or about 9-fold, at or about 10-fold or more.

63. The method of any of claims 60-62, wherein the reference T cell population is a population of T cells known to have a non-exhausted phenotype, is a population of naïve T cells, is a population of central memory T cells, or is a population of stem central memory T cells, optionally from the same subject, or of the same species as the subject, from which the T cell or T cells having the exhausted phenotype are derived.

64. The method of any of claims 60-63, wherein the reference T cell population (a) is a subject-matched population comprising bulk T cells isolated from the blood of the subject from which the T cell or T cells having the exhausted phenotype is derived, optionally wherein the bulk T cells do not express the CAR and/or (b) is obtained from the subject from which the T cell or T cells having the exhausted phenotype is derived, prior to receiving administration of a dose of T cells expressing the CAR.

65. The method of any of claims 60-64, wherein the reference T cell population is a composition comprising a sample of the T cell therapy, or pharmaceutical composition comprising T cells expressing the CAR, prior to its administration to the subject, optionally wherein the composition is a cryopreserved sample.

66. The method of any of claims 60-65, wherein one or more of the one or more exhaustion marker is an inhibitory receptor.

67. The method of any of claims 60-66, wherein one or more of the one or more exhaustion marker is selected from among PD-1, CTLA-4, TIM-3, LAG-3, BTLA, 2B4, CD160, CD39, VISTA, and TIGIT.

68. The method of any of claims 60-67, wherein the activity or is one or more of proliferation, cytotoxicity or production of one or a combination of inflammatory cytokines, optionally wherein the one or a combination of cytokines is selected from the group consisting of IL-2, IFN-gamma and TNF-alpha.

69. The method of any of claims 60-68, wherein the exposure to BCMA or an agonist of the CAR, optionally wherein the agonist is an anti-idiotypic antibody, comprises incubation with BCMA or the agonist of the CAR.

70. The method of claim 69, wherein the antigen is comprised on the surface of antigen-expressing target cells, optionally multiple myeloma cells or cell line.

71. The method of any of claims 1-70, wherein the dose of T cells is between at or about 5×10⁷ CAR+ T cells and at or about 1×10⁹ CAR+ T cells.

72. The method of any of claims 1-70, wherein the dose of T cells is between at or about 1×10⁸ CAR+ T cells and at or about 1×10⁹ CAR+ T cells.

73. The method of any of claims 1-70, wherein the dose of T cells is at or about 1.5×10⁸ cells or CAR+ T cells.

74. The method of any of claims 1-70, wherein the dose of T cells is at or about 3×10⁸ cells or CAR+ T cells.

75. The method of any of claims 1-70, wherein the dose of T cells is at or about 4.5×10⁸ cells or CAR+ T cells.

76. The method of any of claims 1-70, wherein the dose of T cells is at or about 6×10⁸ cells or CAR+ T cells.

77. The method of any of claims 1-76, wherein the dose comprises CD3⁺ CAR-expressing T cells.

78. The method of any of claims 1-77, wherein the dose comprises a combination of CD4⁺ T cells and CD8⁺ T cells and/or a combination of CD4⁺ CAR-expressing T cells and CD8⁺ CAR-expressing T cells.

79. The method of claim 78, wherein the ratio of CD4⁺ CAR-expressing T cells to CD8⁺ CAR-expressing T cells and/or of CD4⁺ T cells to CD8⁺ T cells, is or is approximately 1:1 or is between at or approximately 1:3 and at or approximately 3:1.

80. The method of any of claims 1-79, wherein prior to the administration of the dose of T cells, the subject has received a lymphodepleting therapy comprising the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.

81. The method of any of claims 1-80, wherein the subject has received a lymphodepleting therapy comprising the administration of fludarabine at or about 30 mg/m² body surface area of the subject, daily, and cyclophosphamide at or about 300 mg/m² body surface area of the subject, daily, for 3 days.

82. The method of any of claims 1-81, wherein the CAR comprises an antigen binding domain that binds to BCMA, a transmembrane domain, and an intracellular signaling region comprising a CD3-zeta (CD3ζ) chain.

83. The method of claim 82, wherein the antigen binding domain is a single chain variable fragment (scFv).

84. The method of claim 82 or claim 83, wherein the antigen binding domain comprises a V_{H} and a V_{L} region, wherein the V_{H} region comprises a CDR-H1 set forth in SEQ ID NO: 56, a CDR-H2 set forth in SEQ ID NO:57 and a CDR-H3 set forth in SEQ ID NO:58, and the V_{L} region comprises a CDR-L1 set forth in SEQ ID NO: 59, a CDR-L2 set forth in SEQ ID NO:60 and a CDR-H3 set forth in SEQ ID NO:61.

85. The method of any of claims 82-84, wherein the antigen binding domain comprises a V_{H} region that has the sequence of amino acids set forth in SEQ ID NO:36 or a sequence of amino acids that exhibits at least 90%, at least 91%, at least 92%, at least 93%, at least 94%, at least 95%, at least 96%, at least 97%, at least 98%, at least 99% to SEQ ID NO:36, and a V_{L} region has the sequence of amino acids set forth in SEQ ID NO:37 or a sequence of amino acids that exhibits at least 90%, at least 91%, at least 92%, at least 93%, at least 94%, at least 95%, at least 96%, at least 97%, at least 98%, at least 99% to SEQ ID NO:37.

86. The method of any of claims 82-84, wherein the antigen binding domain comprises the V_{H} region sequence of amino acids set forth in SEQ ID NO:36 and the V_{L} region sequence of amino acids set forth in SEQ ID NO:37.

87. The method of any of claims 82-86, wherein the antigen-binding domain is an scFv that has the sequence of amino acids set forth in SEQ ID NO:180 or a sequence of amino acids exhibits at least 90%, at least 91%, at least 92%, at least 93%, at least 94%, at least 95%, at least 96%, at least 97%, at least 98%, at least 99% to SEQ ID NO:180.

88. The method of any of claims 82-87, wherein the antigen-binding domain is an scFv that has the sequence of amino acids set forth in SEQ ID NO:180.

89. The method of claim 82 or claim 83, wherein the anti-BCMA CAR comprises a V_{H} and a V_{L} region, wherein the V_{H} region comprises a CDR-H1 set forth in SEQ ID NO: 62, a CDR-H2 set forth in SEQ ID NO:63 and a CDR-H3 set forth in SEQ ID NO:64, and the V_{L} region comprises a CDR-L1 set forth in SEQ ID NO: 65, a CDR-L2 set forth in SEQ ID NO:66 and a CDR-H3 set forth in SEQ ID NO:67.

90. The method of any of claims 82, 83, and 89, wherein the antigen binding domain comprises a V_{H} region that has the sequence of amino acids set forth in SEQ ID NO:30 or a sequence of amino acids that exhibits at least 90%, at least 91%, at least 92%, at least 93%, at least 94%, at least 95%, at least 96%, at least 97%, at least 98%, at least 99% to SEQ ID NO:30, and the V_{L} region has the sequence of amino acids set forth in SEQ ID NO:31 or a sequence of amino acids that exhibits at least 90%, at least 91%, at least 92%, at least 93%, at least 94%, at least 95%, at least 96%, at least 97%, at least 98%, at least 99% to SEQ ID NO:31.

91. The method of any of claims 82, 83, 89, and 90, wherein the antigen binding domain comprises the V_{H} region that has the sequence of amino acids set forth in SEQ ID NO:30 and the V_{L} region has the sequence of amino acids set forth in SEQ ID NO:31.

92. The method of any of claims 82, 83, and 89-91, wherein the antigen binding domain is an scFv that has the sequence of amino acids set forth in SEQ ID NO:68 or a sequence of amino acids exhibits at least 90%, at least 91%, at least 92%, at least 93%, at least 94%, at least 95%, at least 96%, at least 97%, at least 98%, at least 99% to SEQ ID NO:68.

93. The method of any of claims 82, 83, and 89-91, wherein the antigen binding domain is an scFv set forth in SEQ ID NO:68.

94. The method of any of claims 82-93, wherein the intracellular signaling region further comprises a costimulatory signaling domain.

95. The method of claim 94, wherein the costimulatory signaling region comprises an intracellular signaling domain of CD28, 4-1BB, or ICOS, or a signaling portion thereof.

96. The method of claim 94 or claim 95, wherein the costimulatory signaling region comprises an intracellular signaling domain of 4-1BB, optionally human 4-1BB.

97. The method of any of claims 94-96, wherein the costimulatory signaling region is between the transmembrane domain and the cytoplasmic signaling domain of a CD3-zeta (CD3ζ) chain.

98. The method of any of claims 82-97, wherein the transmembrane domain is or comprises a transmembrane domain from human CD28.

99. The method of any of claims 82-97, wherein the transmembrane domain is or comprises a transmembrane domain from human CD8.

100. The method of any of claims 82-99, wherein the CAR further comprises an extracellular spacer between the antigen binding domain and the transmembrane domain.

101. The method of any of claim 100, wherein the spacer is between at or about 50 amino acids and at or about 250 amino acids.

102. The method of claim 100 or claim 101, wherein the spacer is between at or about 125 amino acids and at or about 250 amino acids, optionally wherein the spacer is at or about 228 amino acids.

103. The method of any of claims 100-102, wherein the spacer is an immunoglobulin spacer comprising all or a portion of an immunoglobulin constant domain or a modified form thereof.

104. The method of any of claims 100-103, wherein the spacer comprises an IgG4/2 chimeric hinge or a modified IgG4 hinge; an IgG2/4 chimeric C_{H}2 region; and an IgG4 C_{H}3 region.

105. The method of any of claims 100-104, wherein the spacer is set forth in SEQ ID NO: 29 or is encoded by a sequence of nucleotides set forth in SEQ ID NO:179.

106. The method of claim 100 or claim 101, wherein the spacer is a CD8 hinge.

107. The method of any of claims 1-106, wherein the anti-BCMA CAR has a sequence set forth in any one of SEQ ID NOS: 126-177 or a sequence of amino acids that exhibits at least 90%, at least 91%, at least 92%, at least 93%, at least 94%, at least 95%, at least 96%, at least 97%, at least 98%, or at least 99% sequence identity to any one of SEQ ID NOS: 126-177.

108. The method of any of claims 1-107, wherein the anti-BCMA CAR has the sequence of amino acids set forth in SEQ ID NO:160 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:160.

109. The method of any of claims 1-108, wherein the CAR is encoded by the sequence of nucleotides set forth in SEQ ID NO:69.

110. The method of any of claims 1-107, wherein the anti-BCMA CAR has the sequence of amino acids set forth in SEQ ID NO:161 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:161.

111. The method of any of claims 1-107, wherein the anti-BCMA CAR has the sequence of amino acids set forth in SEQ ID NO:152 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:152.

112. The method of any of claims 1-107, wherein the anti-BCMA CAR has the sequence of amino acids set forth in SEQ ID NO:168 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:168.

113. The method of any of claims 1-107, wherein the anti-BCMA CAR has the sequence of amino acids set forth in SEQ ID NO:171 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO:171.as

114. The method of any of claims 1-113, wherein the anti-BCMA CAR binds BCMA, optionally wherein the BCMA is human BCMA.

115. The method of claim 114, wherein the BCMA is membrane-bound BCMA expressed on the surface of a cell.

116. The method of claim 114 or claim 115, wherein the anti-BCMA CAR has a greater binding affinity for membrane-bound BCMA than soluble BCMA, optionally wherein the ratio of dissociation constant (K_{D}) for soluble BCMA and the K_{D} for membrane-bound BCMA is more than 10, 15, 20, 25, 30, 40, 50, 60, 70, 80, 90, 100, 200, 500, 1000, 2000 or more.


SEQUENCE LISTING (183 sequences)

SEQ ID NO: 1 - protein, 12 aa, Homo sapiens
  spacer (IgG4hinge)
ESKYGPPCPPCP

SEQ ID NO: 2 - DNA, 36 bp, Homo sapiens
  spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A
LEGGGEGRGSLLTCGDVEENPGPR

SEQ ID NO: 7 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28 (amino acids 153-179 of Accession No. P10747)
FWVLVVVGGVLACYSLLVTVAFIIFWV

SEQ ID NO: 9 - protein, 66 aa, Homo sapiens
  CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28 (amino acids 180-220 of P10747)
RSKRSRLLHSDYMNMTPRRPGPTRKHYQPYAPPRDFAAYRS

SEQ ID NO: 11 - protein, 41 aa, Homo sapiens
  CD28 (LL to GG)
RSKRSRGGHSDYMNMTPRRPGPTRKHYQPYAPPRDFAAYRS

SEQ ID NO: 12 - protein, 42 aa, Homo sapiens
  4-1BB (amino acids 214-255 of Q07011.1)
KRGRKKLLYIFKQPFMRPVQTTQEEDGCSCRFPEEEEGGCEL

SEQ ID NO: 13 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 10 aa, Artificial Sequence
  Linker
  REPEAT (5)...(9): SGGGG is repeated 5 times
PGGGSGGGGP

SEQ ID NO: 17 - protein, 17 aa, Artificial Sequence
  Linker
GSADDAKKDAAKKDGKS

SEQ ID NO: 18 - protein, 54 aa, Homo sapiens
  Extracellular domain of human BCMA (GenBank No. NP_001183.2)
MLQMAGQCSQNEYFDSLLHACIPCQLRCSSNTPPLTCQRYCNASVTNSVKGTNA

SEQ ID NO: 19 - protein, 5 aa, Artificial Sequence
  Linker
GGGGS

SEQ ID NO: 20 - protein, 231 aa, Artificial Sequence
  Modified Human IgG1 Fc
PKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 16 aa, Artificial Sequence
  CD33 Signal peptide
MPLLLLLPLLWAGALA

SEQ ID NO: 22 - protein, 306 aa, Artificial Sequence
  BCMA-Fc construct
MPLLLLLPLLWAGALAMLQMAGQCSQNEYFDSLLHACIPCQLRCSSNTPPLTCQRYCNASVTNSVKGTNAGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 18 aa, Artificial Sequence
  T2A
EGRGSLLTCGDVEENPGP

SEQ ID NO: 24 - protein, 22 aa, Artificial Sequence
  P2A
GSGATNFSLLKQAGDVEENPGP

SEQ ID NO: 25 - protein, 19 aa, Artificial Sequence
  P2A
ATNFSLLKQAGDVEENPGP

SEQ ID NO: 26 - protein, 20 aa, Artificial Sequence
  E2A
QCTNYALLKLAGDVESNPGP

SEQ ID NO: 27 - protein, 22 aa, Artificial Sequence
  F2A
VKQTLNFDLLKLAGDVESNPGP

SEQ ID NO: 28 - protein, 335 aa, Artificial Sequence; identical to SEQ ID NO: 7
  tEGFR

SEQ ID NO: 29 - protein, 228 aa, Homo sapiens
  Hinge-CH2-CH3 spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 117 aa, Artificial Sequence
  Variable heavy (VH) Anti-BCMA
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 31 - protein, 111 aa, Artificial Sequence
  Variable light (VL) Anti-BCMA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 32 - protein, 122 aa, Artificial Sequence
  Variable heavy (VH) Anti-BCMA
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 33 - protein, 107 aa, Artificial Sequence
  Variable light (VL) Anti-BCMA
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 34 - protein, 116 aa, Artificial Sequence
  Variable heavy (VH) Anti-BCMA
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 35 - protein, 111 aa, Artificial Sequence
  Variable light (VL) Anti-BCMA
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 36 - protein, 118 aa, Artificial Sequence
  Variable heavy (VH) Anti-BCMA
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 37 - protein, 105 aa, Artificial Sequence
  Variable light (VL) Anti-BCMA
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 38 - protein, 122 aa, Artificial Sequence
  Variable heavy (VH) Anti-BCMA
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 39 - protein, 111 aa, Artificial Sequence
  Variable light (VL) Anti-BCMA
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 40 - protein, 120 aa, Artificial Sequence
  Variable heavy (VH) Anti-BCMA
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 41 - protein, 112 aa, Artificial Sequence
  Variable light (VL) Anti-BCMA
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 42 - protein, 118 aa, Artificial Sequence
  Variable heavy (VH) Anti-BCMA
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 43 - protein, 112 aa, Artificial Sequence
  Variable light (VL) Anti-BCMA
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 44 - protein, 28 aa, Artificial Sequence
  CD8a TM
IYIWAPLAGTCGVLLLSLVITLYCNHRN

SEQ ID NO: 45 - protein, 21 aa, Artificial Sequence
  CD8a TM
IYIWAPLAGTCGVLLLSLVIT

SEQ ID NO: 46 - protein, 4 aa, Artificial Sequence
  Linking Peptide
RAAA

SEQ ID NO: 47 - protein, 118 aa, Artificial Sequence
  Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSS

SEQ ID NO: 48 - protein, 110 aa, Artificial Sequence
  Variable light (VL) Anti-BCMA
QSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVL

SEQ ID NO: 49 - protein, 118 aa, Artificial Sequence
  Variable heavy (VH) Anti-BCMA
EVQLVQSGGGLVQPGRSLRLSCTASGFTFGDYAMSWFRQAPGKGLEWVGFIRSKAYGGTTEYAASVKGRFTISRDDSKSIAYLQMNSLKTEDTAVYYCAAWSAPTDYWGQGTLVTVSS

SEQ ID NO: 50 - protein, 107 aa, Artificial Sequence
  Variable light (VL) Anti-BCMA
DIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIK

SEQ ID NO: 51 - protein, 118 aa, Artificial Sequence
  Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSS

SEQ ID NO: 52 - protein, 108 aa, Artificial Sequence
  Variable light (VL) Anti-BCMA
SYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVL

SEQ ID NO: 53 - protein, 122 aa, Artificial Sequence; identical to SEQ ID NO: 38
  Variable heavy (VH) Anti-BCMA

SEQ ID NO: 54 - protein, 111 aa, Artificial Sequence; identical to SEQ ID NO: 39
  Variable light (VL) Anti-BCMA

SEQ ID NO: 55 - protein, 17 aa, Artificial Sequence
  Linker
ASGGGGSGGRASGGGGS

SEQ ID NO: 56 - protein, 5 aa, Artificial Sequence
  BCMA-55 CDR-H1 (aa) - Kabat numbering
DYYVY

SEQ ID NO: 57 - protein, 17 aa, Artificial Sequence
  BCMA-55 CDR-H2 (aa) - Kabat numbering
WINPNSGGTNYAQKFQG

SEQ ID NO: 58 - protein, 9 aa, Artificial Sequence
  BCMA-55 CDR-H3 (aa) - Kabat, Chothia, and AbM numbering
SQRDGYMDY

SEQ ID NO: 59 - protein, 8 aa, Artificial Sequence
  BCMA-55 CDR-L1 (aa) - Kabat, Chothia, and AbM numbering
TGTSSDVG

SEQ ID NO: 60 - protein, 7 aa, Artificial Sequence
  BCMA-55 CDR-L2 (aa) - Kabat, Chothia, and AbM numbering
EDSKRPS

SEQ ID NO: 61 - protein, 10 aa, Artificial Sequence
  BCMA-55 CDR-L3 (aa) - Kabat, Chothia, and AbM numbering
SSNTRSSTLV

SEQ ID NO: 62 - protein, 5 aa, Artificial Sequence
  CDR-H1
DYSIN

SEQ ID NO: 63 - protein, 17 aa, Artificial Sequence
  CDR-H2
WINTETREPAYAYDFRG

SEQ ID NO: 64 - protein, 8 aa, Artificial Sequence
  CDR-H3
DYSYAMDY

SEQ ID NO: 65 - protein, 15 aa, Artificial Sequence
  CDR-L1
RASESVTILGSHLIH

SEQ ID NO: 66 - protein, 7 aa, Artificial Sequence
  CDR-L2
LASNVQT

SEQ ID NO: 67 - protein, 9 aa, Artificial Sequence
  CDR-L3
LQSRTIPRT

SEQ ID NO: 68 - protein, 246 aa, Artificial Sequence
  scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 69 - DNA, 1962 bp, Artificial Sequence
  anti-BCMA CAR
cagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 70 - protein, 18 aa, Artificial Sequence
  Linker
GSTSGSGKPGSGEGSTKG

SEQ ID NO: 71 - protein, 4 aa, Artificial Sequence
  Linker
GGGS

SEQ ID NO: 72 - protein, 15 aa, Artificial Sequence
  Linker
GGGGSGGGGSGGGGS

SEQ ID NO: 73 - protein, 18 aa, Artificial Sequence
  Linker
GSTSGSGKPGSGEGSTKG

SEQ ID NO: 74 - protein, 21 aa, Artificial Sequence
  Linker
SRGGGGSGGGGSGGGGSLEMA

SEQ ID NO: 75 - protein, 21 aa, Artificial Sequence
  CD8a signal peptide
MALPVTALLLPLALLLHAARP

SEQ ID NO: 76 - protein, 20 aa, Artificial Sequence
  signal peptide
METDTLLLWVLLLWVPGSTG

SEQ ID NO: 77 - protein, 118 aa, Artificial Sequence
  Variable heavy (VH) Anti-BCMA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 78 - protein, 107 aa, Artificial Sequence
  Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 79 - protein, 122 aa, Artificial Sequence
  Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 80 - protein, 112 aa, Artificial Sequence
  Variable light (VL) Anti-BCMA
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 81 - protein, 116 aa, Artificial Sequence
  Variable heavy (VH) Anti-BCMA
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 82 - protein, 106 aa, Artificial Sequence
  Variable light (VL) Anti-BCMA
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 83 - protein, 122 aa, Artificial Sequence
  Variable heavy (VH) Anti-BCMA
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 84 - protein, 107 aa, Artificial Sequence
  Variable light (VL) Anti-BCMA
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 85 - protein, 119 aa, Artificial Sequence
  Variable heavy (VH) Anti-BCMA
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 86 - protein, 107 aa, Artificial Sequence
  Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 87 - protein, 122 aa, Artificial Sequence
  Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 88 - protein, 107 aa, Artificial Sequence
  Variable light (VL) Anti-BCMA
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 89 - protein, 126 aa, Artificial Sequence
  Variable heavy (VH) Anti-BCMA
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 90 - protein, 113 aa, Artificial Sequence
  Variable light (VL) Anti-BCMA
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 91 - protein, 123 aa, Artificial Sequence
  Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 92 - protein, 107 aa, Artificial Sequence
  Variable light (VL) Anti-BCMA
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 93 - protein, 121 aa, Artificial Sequence
  Variable heavy (VH) Anti-BCMA
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 94 - protein, 112 aa, Artificial Sequence
  Variable light (VL) Anti-BCMA
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLG

SEQ ID NO: 95 - protein, 117 aa, Artificial Sequence
  Variable heavy (VH) Anti-BCMA
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 96 - protein, 111 aa, Artificial Sequence
  Variable light (VL) Anti-BCMA
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 97 - protein, 115 aa, Artificial Sequence
  Variable heavy (VH) Anti-BCMA
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 98 - protein, 107 aa, Artificial Sequence
  Variable light (VL) Anti-BCMA
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 99 - protein, 120 aa, Artificial Sequence
  Variable heavy (VH) Anti-BCMA
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSS

SEQ ID NO: 100 - protein, 109 aa, Artificial Sequence
  Variable light (VL) Anti-BCMA
DIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 101 - protein, 115 aa, Artificial Sequence
  Variable heavy (VH) Anti-BCMA
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 102 - protein, 107 aa, Artificial Sequence
  Variable light (VL) Anti-BCMA
DIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 103 - protein, 115 aa, Artificial Sequence; identical to SEQ ID NO: 97
  Variable heavy (VH) Anti-BCMA

SEQ ID NO: 104 - protein, 109 aa, Artificial Sequence
  Variable light (VL) Anti-BCMA
EIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 105 - protein, 117 aa, Artificial Sequence
  Variable heavy (VH) Anti-BCMA
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 106 - protein, 111 aa, Artificial Sequence
  Variable light (VL) Anti-BCMA
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 107 - protein, 117 aa, Artificial Sequence
  Variable heavy (VH) Anti-BCMA
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 108 - protein, 111 aa, Artificial Sequence
  Variable light (VL) Anti-BCMA
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 109 - protein, 119 aa, Artificial Sequence
  Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 110 - protein, 112 aa, Artificial Sequence
  Variable light (VL) Anti-BCMA
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 111 - protein, 119 aa, Artificial Sequence
  Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 112 - protein, 112 aa, Artificial Sequence
  Variable light (VL) Anti-BCMA
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 113 - protein, 122 aa, Artificial Sequence
  Anti-BCMA sdAb
QVQLVESGGGLVQPGGSLRLSCEASGFTLDYYAIGWFRQAPGKEREGVICISRSDGSTYYADSVKGRFTISRDNAKKTVYLQMISLKPEDTAAYYCAAGADCSGYLRDYEFRGQGTQVTVSS

SEQ ID NO: 114 - protein, 39 aa, Artificial Sequence
  CD28 spacer
IEVMYPPPYLDNEKSNGTIIHVKGKHLCPSPLFPGPSKP

SEQ ID NO: 115 - protein, 25 aa, Artificial Sequence
  CD8a TM
IYIWAPLAGTCGVLLLSLVITLYCN

SEQ ID NO: 116 - protein, 30 aa, Artificial Sequence
  CD28 spacer (truncated)
LDNEKSNGTIIHVKGKHLCPSPLFPGPSKP

SEQ ID NO: 117 - protein, 46 aa, Artificial Sequence
  CD8a hinge
PTTTPAPRPPTPAPTIASQPLSLRPEACRPAAGGAVHTRGLDFACD

SEQ ID NO: 118 - protein, 45 aa, Artificial Sequence
  CD8a hinge
TTTPAPRPPTPAPTIASQPLSLRPEACRPAAGGAVHTRGLDFACD

SEQ ID NO: 119 - protein, 55 aa, Artificial Sequence
  CD8a hinge
FVPVFLPAKPTTTPAPRPPTPAPTIASQPLSLRPEACRPAAGGAVHTRGLDFACD

SEQ ID NO: 120 - protein, 39 aa, Artificial Sequence
  CTLA4 hinge
DTGLYICKVELMYPPPYYLGIGNGTQIYVIDPEPCPDSD

SEQ ID NO: 121 - protein, 24 aa, Artificial Sequence
  CTLA4 TM
FLLWILAAVSSGLFFYSFLLTAVS

SEQ ID NO: 122 - protein, 38 aa, Artificial Sequence
  PD-1 hinge
QIKESLRAELRVTERRAEVPTAHPSPSPRPAGQFQTLV

SEQ ID NO: 123 - protein, 21 aa, Artificial Sequence
  PD-1 TM
VGVVGGLLGSLVLLVWVLAVI

SEQ ID NO: 124 - protein, 16 aa, Artificial Sequence
  FcgRIIIa hinge
GLAVSTISSFFPPGYQ

SEQ ID NO: 125 - protein, 231 aa, Artificial Sequence
  IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 457 aa, Artificial Sequence
  anti-BCMA CAR
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 457 aa, Artificial Sequence
  anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 466 aa, Artificial Sequence
  anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 466 aa, Artificial Sequence
  anti-BCMA CAR
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 454 aa, Artificial Sequence
  anti-BCMA CAR
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 454 aa, Artificial Sequence
  anti-BCMA CAR
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 461 aa, Artificial Sequence
  anti-BCMA CAR
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 461 aa, Artificial Sequence
  anti-BCMA CAR
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 458 aa, Artificial Sequence
  anti-BCMA CAR
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 458 aa, Artificial Sequence
  anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 461 aa, Artificial Sequence
  anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 461 aa, Artificial Sequence
  anti-BCMA CAR
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 471 aa, Artificial Sequence
  anti-BCMA CAR
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 471 aa, Artificial Sequence
  anti-BCMA CAR
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 462 aa, Artificial Sequence
  anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 462 aa, Artificial Sequence
  anti-BCMA CAR
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 466 aa, Artificial Sequence
  anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 473 aa, Artificial Sequence
  anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 470 aa, Artificial Sequence
  anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 476 aa, Artificial Sequence
  anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 475 aa, Artificial Sequence
  anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 482 aa, Artificial Sequence
  anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 476 aa, Artificial Sequence
  anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 481 aa, Artificial Sequence
  anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 479 aa, Artificial Sequence
  anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 472 aa, Artificial Sequence
  anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 472 aa, Artificial Sequence
  anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - protein, 472 aa, Artificial Sequence
  anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 466 aa, Artificial Sequence
  anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAADTGLYICKVELMYPPPYYLGIGNGTQIYVIDPEPCPDSDFLLWILAAVSSGLFFYSFLLTAVSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 464 aa, Artificial Sequence
  anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAAQIKESLRAELRVTERRAEVPTAHPSPSPRPAGQFQTLVVGVVGGLLGSLVLLVWVLAVICSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 653 aa, Artificial Sequence
  anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSSGGGGSGGGGSGGGGSQSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 650 aa, Artificial Sequence
  anti-BCMA CAR
EVQLVQSGGGLVQPGRSLRLSCTASGFTFGDYAMSWFKQAPGKGLEWVGFIRSKAYGGTTEYAASVKGRFTISRDDSKSIAYLQMNSLKTEDTAVYYCAAWSAPTDYWGQGTLVTVSSGGGGSGGGGSGGGGSDIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 651 aa, Artificial Sequence
  anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSSGGGGSGGGGSGGGGSSYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 658 aa, Artificial Sequence
  anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 654 aa, Artificial Sequence
  anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 653 aa, Artificial Sequence
  anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 462 aa, Artificial Sequence
  anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 469 aa, Artificial Sequence
  anti-BCMA CAR
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 462 aa, Artificial Sequence
  anti-BCMA CAR
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 464 aa, Artificial Sequence
  anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 467 aa, Artificial Sequence
  anti-BCMA CAR
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 472 aa, Artificial Sequence
  anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 466 aa, Artificial Sequence
  anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 466 aa, Artificial Sequence
  anti-BCMA CAR
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 466 aa, Artificial Sequence
  anti-BCMA CAR
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - protein, 482 aa, Artificial Sequence
  anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - protein, 440 aa, Artificial Sequence
  anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 173 - protein, 469 aa, Artificial Sequence
  anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 174 - protein, 655 aa, Artificial Sequence
  anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - protein, 440 aa, Artificial Sequence
  anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 176 - protein, 469 aa, Artificial Sequence
  anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 177 - protein, 655 aa, Artificial Sequence
  anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - protein, 24 aa, Artificial Sequence
  CD8 TM
IYIWAPLAGTCGVLLLSLVITLYC

SEQ ID NO: 179 - DNA, 684 bp, Artificial Sequence
  Modified IgG4 hinge- IgG2/IgG4 CH2- IgG4 CH3 spacer
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtgacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaa

SEQ ID NO: 180 - protein, 244 aa, Artificial Sequence
  BCMA-55 scFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 181 - protein, 326 aa, Homo sapiens
  Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 182 - protein, 327 aa, Homo sapiens
  Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 183 - DNA, 684 bp, Artificial Sequence
  optimized SSE modified IgG4 hinge- IgG2/IgG4 CH2- IgG4 CH3 spacer
gagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag